SEQUENCE LISTING (8 sequences)

SEQ ID NO: 1 - protein, 526 aa, Homo sapiens [sequence omitted]

SEQ ID NO: 2 - protein, 500 aa, Homo sapiens
METEAIDGYITCDNELSPEREHSNMAIDLTSSTPNGQHASPSHMTSTNSVKLEMQSDEECDRKPLSREDEIRGHDEGSSLEEPLIESSEVADNRKVQELQGEGGIRLPNGERPFHCNQCGASFTQKGNLLRHIKLHSGEKPFKCPFCSYACRRRDALTGHLRTHSVGKPHKCNYCGRSYKQRSSLEEHKERCHNYLQNVSMEAAGQVMSHHVPPMEDCKEQEPIMDNNISLVPFERPAVIEKLTGNMGKRKSSTPQKFVGEKLMRFSYPDIHFDMNLTYEKEAELMQSHMMDQAINNAITYLGAEALHPLMQHPPSTIAEVAPVISSAYSQVYHPNRIERPISRETADSHENNMDGPISLIRPKSRPQEREASPSNSCLDSTDSESSHDDHQSYQGHPALNPKRKQSPAYMKEDVKALDTTKAPKGSLKDIYKVFNGEGEQIRAFKCEHCRVLFLDHVMYTIHMGCHGYRDPLECNICGYRSQDRYEFSSHIVRGEHTFH

SEQ ID NO: 3 - protein, 452 aa, Homo sapiens
METEAIDGYITCDNELSPEREHSNMAIDLTSSTPNGQHASPSHMTSTNSVKLEMQSDEECDRKPLSREDEIRGHDEGSSLEEPLIESSEVADNRKVQELQGEGGIRLPNGERPFHCNQCGASFTQKGNLLRHIKLHSGEKPFKCPFCSYACRRRDALTGHLRTHSVGKPHKCNYCGRSYKQRSSLEEHKERCHNYLQNVSMEAAGQVMSHHGEKLMRFSYPDIHFDMNLTYEKEAELMQSHMMDQAINNAITYLGAEALHPLMQHPPSTIAEVAPVISSAYSQVYHPNRIERPISRETADSHENNMDGPISLIRPKSRPQEREASPSNSCLDSTDSESSHDDHQSYQGHPALNPKRKQSPAYMKEDVKALDTTKAPKGSLKDIYKVFNGEGEQIRAFKCEHCRVLFLDHVMYTIHMGCHGYRDPLECNICGYRSQDRYEFSSHIVRGEHTFH

SEQ ID NO: 4 - protein, 239 aa, Homo sapiens
METEAIDGYITCDNELSPEREHSNMAIDLTSSTPNGQHASPSHMTSTNSVKLEMQSDEECDRKPLSREDEIRGHDEGSSLEEPLIESSEVADNRKVQELQGEGGIRLPNGKLKCDVCGMVCIGPNVLMVHKRSHTGERPFHCNQCGASFTQKGNLLRHIKLHSGEKPFKCPFCSYACRRRDALTGHLRTHSVGKPHKCNYCGRSYKQRSSLEEHKERCHNYLQNVSMEAAGQVMSHHDS

SEQ ID NO: 5 - protein, 454 aa, Homo sapiens
METEAIDGYITCDNELSPEREHSNMAIDLTSSTPNGQHASPSHMTSTNSVKLEMQSDEECDRKPLSREDEIRGHDEGSSLEEPLIESSEVADNRKVQELQGEGGIRLPNGERPFHCNQCGASFTQKGNLLRHIKLHSGEKPFKCPFCSYACRRRDALTGHLRTHSVPPMEDCKEQEPIMDNNISLVPFERPAVIEKLTGNMGKRKSSTPQKFVGEKLMRFSYPDIHFDMNLTYEKEAELMQSHMMDQAINNAITYLGAEALHPLMQHPPSTIAEVAPVISSAYSQVYHPNRIERPISRETADSHENNMDGPISLIRPKSRPQEREASPSNSCLDSTDSESSHDDHQSYQGHPALNPKRKQSPAYMKEDVKALDTTKAPKGSLKDIYKVFNGEGEQIRAFKCEHCRVLFLDHVMYTIHMGCHGYRDPLECNICGYRSQDRYEFSSHIVRGEHTFH

SEQ ID NO: 6 - protein, 23 aa, Homo sapiens
FHCNQCGASFTQKGNLLRHIKLH

SEQ ID NO: 7 - protein, 585 aa, Homo sapiens
MHTPPALPRRFQGGGRVRTPGSHRQGKDNLERDPSGGCVPDFLPQAQDSNHFIMESLFCESSGDSSLEKEFLGAPVGPSVSTPNSQHSSPSRSLSANSIKVEMYSDEESSRLLGPDERLLEKDDSVIVEDSLSEPLGYCDGSGPEPHSPGGIRLPNGKLKCDVCGMVCIGPNVLMVHKRSHTGERPFHCNQCGASFTQKGNLLRHIKLHSGEKPFKCPFCNYACRRRDALTGHLRTHSVSSPTVGKPYKCNYCGRSYKQQSTLEEHKERCHNYLQSLSTEAQALAGQPGDEIRDLEMVPDSMLHSSSERPTFIDRLANSLTKRKRSTPQKFVGEKQMRFSLSDLPYDVNSGGYEKDVELVAHHSLEPGFGSSLAFVGAEHLRPLRLPPTNCISELTPVISSVYTQMQPLPGRLELPGSREAGEGPEDLADGGPLLYRPRGPLTDPGASPSNGCQDSTDTESNHEDRVAGVVSLPQGPPPQPPPTIVVGRHSPAYAKEDPKPQEGLLRGTPGPSKEVLRVVGESGEPVKAFKCEHCRILFLDHVMFTIHMGCHGFRDPFECNICGYHSQDRYEFSSHIVRGEHKVG

SEQ ID NO: 8 - protein, 544 aa, Homo sapiens
MDSRYLQLQLYLPSCSLLQGSGDSSLEKEFLGAPVGPSVSTPNSQHSSPSRSLSANSIKVEMYSDEESSRLLGPDERLLEKDDSVIVEDSLSEPLGYCDGSGPEPHSPGGIRLPNGKLKCDVCGMVCIGPNVLMVHKRSHTGERPFHCNQCGASFTQKGNLLRHIKLHSGEKPFKCPFCNYACRRRDALTGHLRTHSVSSPTVGKPYKCNYCGRSYKQQSTLEEHKERCHNYLQSLSTEAQALAGQPGDEIRDLEMVPDSMLHSSSERPTFIDRLANSLTKRKRSTPQKFVGEKQMRFSLSDLPYDVNSGGYEKDVELVAHHSLEPGFGSSLAFVGAEHLRPLRLPPTNCISELTPVISSVYTQMQPLPGRLELPGSREAGEGPEDLADGGPLLYRPRGPLTDPGASPSNGCQDSTDTESNHEDRVAGVVSLPQGPPPQPPPTIVVGRHSPAYAKEDPKPQEGLLRGTPGPSKEVLRVVGESGEPVKAFKCEHCRILFLDHVMFTIHMGCHGFRDPFECNICGYHSQDRYEFSSHIVRGEHKVG

(19) United States
(12) Patent Application Publication
(10) Pub. No.: US 2025/0346574 A1
(43) Pub. Date: Nov. 13, 2025

(54) PYRIDINYL SUBSTITUTED OXOISOINDOLINE COMPOUNDS FOR THE TREATMENT OF CANCER

(71) Applicant: Bristol-Myers Squibb Company, Princeton, NJ (US)

(72) Inventors: Godwin Kwame KUMI, Morrisville, PA (US); Ashok Vinayak PURANDARE, Pennington, NJ (US); Guo LI, Newtown, PA (US); Weifang SHAN, Princeton, NJ (US); James Aaron BALOG, Lambertville, NJ (US); Emily Charlotte CHERNEY, Newtown, PA (US); Yan CHEN, Yardley, PA (US)

(73) Assignee: Bristol-Myers Squibb Company, Princeton, NJ (US)

(21) Appl. No.: 18/553,999

(22) PCT Filed: Apr. 4, 2022
(86) PCT No.: PCT/US2022/023236

§ 371 Date: Oct. 5, 2023

Related U.S. Application Data
(60) Provisional application No. 63/170,865, filed on Apr. 5, 2021.
(60) Provisional application No. 63/189,318, filed on May 17, 2021.

(51) Int. Cl. C07D 401/14; C07D 409/14; C07D 413/14; C07D 471/04; C07D 471/10; C07D 487/04; C07D 491/044; C07D 491/107; C07D 498/08; C07D 498/10; C07F 9/6558; C07F 9/6584

(52) U.S. Cl. CPC C07D 401/14; C07D 409/14; C07D 413/14; C07D 471/04; C07D 471/10; C07D 487/04; C07D 491/044; C07D 491/107; C07D 498/08; C07D 498/10; C07F 9/65583; C07F 9/6584

(57) ABSTRACT

Disclosed are compounds of Formula (I) or a salt thereof, wherein R₁, R₂, R₃, R₄, Z, m, n, p, and q are defined herein. Also disclosed are methods of using such compounds to inhibit Helios protein, and pharmaceutical compositions comprising such compounds. These compounds are useful in the treatment of viral infections and proliferative disorders, such as cancer.

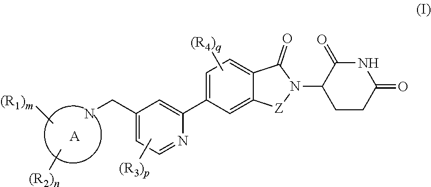

### CROSS REFERENCE

This application claims the benefit of U.S. Provisional Application Ser. No. 63/170,865 filed Apr. 5, 2021 and U.S. Provisional Application Ser. No. 63/189,318, filed May 17, 2021, each incorporated herein in its entirety.

### DESCRIPTION

The present invention generally relates to pyridinyl substituted oxoisoindoline compounds that inhibit Helios protein. Provided herein are pyridinyl substituted oxoisoindoline compounds, compositions comprising such compounds, and methods of their use. The invention further pertains to pharmaceutical compositions comprising at least one compound according to the invention that are useful for the treatment of proliferative disorders, such as cancer, and viral infections.

### BACKGROUND OF THE INVENTION

Regulatory T cells (Tregs) play an essential role in controlling self-tolerance and immune homeostasis via maintenance of inhibitory activity and anergy in the face of vigorous immune and inflammatory responses. Through the preservation of a stable, anergic and suppressive phenotype, Tregs attenuate excessive immune responses and prevent or ameliorate autoimmunity. A number of reports have documented the presence of Tregs within human tumor tissues. Studies demonstrated a clear negative correlation between the number of Tregs and T cell infiltration into the tumor and survival (Curiel et al., 2004, Nat. Med. 10: 942-949; Viguier et al., 2004, J Immuno. 1173:1444-1453; Beyer et al., 2006, Blood 108: 804-811; Zou et al., 2006, Nat. Rev. Immunol. 6: 295-307), implying a potential critical role of Tregs in preventing the development of effective anti-tumor immunity. Accumulated evidence indicates that Foxp3+CD25+CD4+Tregs dominantly infiltrate into tumors and apparently hinder immune responses to tumor cells in rodents and humans. Once activated by a specific antigen, Tregs suppress responder T cells in an antigen-nonspecific and bystander manner in vitro (Takahashi et al., 1998, Int Immunol. 10:1969-80; Thornton et al., 1998, J Exp. Med. 188:287-96). Foxp3+CD25+CD4+Tregs are apparently capable of suppressing a wide range of antitumor immune responses involving CD4+ helper T cells, CD8+ T cells, natural killer cells, and natural killer T cells (Tanaka et al., 2017, Cell Research 27:109-118). Intratumoral depletion of CD25+CD4+Tregs induced regression of established tumors with a change in the cytokine milieu at tumor sites (Yu et al., 2005, J Exp Med. 201: 779-91). In addition, transfer of Treg-depleted CD4+ T cells markedly augmented antitumor immune responses compared with Tregs containing T-cell transfer (Antony et al., 2005, J Immunol 174:2591-601). Tumor-infiltrating Tregs activated by either tumor-derived self-antigens or tumor-associated antigens can similarly suppress specific antitumor immune responses. Modulation of the activities of key factors to control Treg differentiation could represent a potential therapeutic strategy for the treatment of certain diseases, including cancer and viral infections.

FoxP3+CD4 Tregs are remarkably stable. Studies are still evolving to understand the genetic mechanisms that ensure their phenotypic stability after expansion during inflammation, infection or autoimmunity. Transcription factors (TF) responsible for maintaining the stable immunosuppressive phenotype of Tregs likely contribute to this process. The Helios (IKZF2) gene, a member of the Ikaros family of TFs, differs from other Ikaros family members based on its selective expression by thymocytes undergoing negative selection, as well as by regulatory lineages of CD4 and CD8 T cells. Helios is expressed by two regulatory T-cell lineages, FoxP3+CD4+ and Ly49+CD8+Tregs, which are essential to maintain self-tolerance (Kim et al., 2015, Science 350:334-339; Sebastian et al., 2016, J Immunol 196:144-155). Interestingly, recent studies suggest that although Helios is largely dispensable for Treg activity in the steady state, control of the genetic program of FoxP3+CD4 Tregs by Helios in the context of inflammation is essential to maintain a stable phenotype and potentiate suppressive function (Thornton et al., 2010, J Immunol. 184:3433-3441; Kim et al., 2015). Helios expression by Tregs was demonstrated to be crucial in their capability to maintain a suppressive and anergic phenotype in the face of intense inflammatory responses. Activation of the IL-2Rα-STAT5 pathway was demonstrated to be a key contributor by ensuring Treg survival and stability (Kim et al., 2015). Helios plays an indispensable role in maintaining the phenotype of FoxP3+CD4 Tregs by exerting dominant, lymphocyte-intrinsic inhibition to prevent autoimmune disease in the presence of highly activated self-reactive T cells from scurfy mice, which have no FoxP3 fork head domain. Bone marrow (BM) chimeras reconstituted with Helios−/−/Scurfy BM but not Helios+/+/Scurfy BM cells rapidly developed autoimmunity (Kim et al., 2015). These observations indicate the critical contribution of Helios to self-reactive T cell selection, differentiation, and function.

Immune suppression exerted by Tregs can impede antitumor immune responses. A selective deficiency of Helios in FoxP3+CD4 Tregs results in increased Treg instability and conversion of intratumoral CD4 Treg to effector T cells (Teff). Instability of intratumoral Tregs may increase the numbers of Teff cells within tumors as a combined result of Treg conversion and reduced Treg suppressive activities. In addition, defective IL-2 responses were observed in Helios-deficient intratumoral Tregs, which results in decreased numbers of activated Tregs and may also contribute to the increased intratumoral Teff activities. Interaction between tumor cells and infiltrating immune cells leads to secretion of inflammatory mediators, including TNF-α, IL-6, IL-17, IL-1, and TGF-β, and the formation of a local inflammatory environment (Kim et al., 2015).

Lineage instability of Helios-deficient Tregs is also accompanied by diminished FoxP3 expression and results in the acquisition of an effector phenotype by producing proinflammatory cytokines. Effector cell conversion of Helios-deficient Tregs within the tumor-tissue microenvironment is associated with increased expression of genes that control Teff phenotype (Yates et al., 2018, PNAS, 2018, 115: 2162-2167). Acquisition of an unstable phenotype by Helios deficiency only occurs within the tumor microenvironment (TME), but not in peripheral lymphoid organs (Nakagawa et al., 2016, PNAS 113: 6248-6253). Within the chronic inflammatory TME, Helios deficiency in Tregs could drastically alleviate the repressed genetic programs associated with T helper cell differentiation by up-regulating T helper cell associated TFs and effector cytokines. These genetic changes of Helios-deficient Tregs are most apparent in a Treg subpopulation with high affinity for self-antigens, as shown by enhanced GITR/PD-1 expression and increased responsiveness to self-antigens. Their combined effects may promote a phenotype conversion of Tregs into Teff within the TME with increased T-cell receptor (TCR) engagement and costimulatory receptor expression by Tregs, suggesting that the alterations in gene expression, as a central feature of Treg conversion, are immune milieu dependent (Yates et al., 2018).

Reduced Helios expression in FoxP3+CD4 Tregs may allow conversion of memory Tregs into Teff cells that express self-reactive T-cell receptors with specificity for tumor antigens. An altered Treg signature might be selectively induced within the chronic inflammatory conditions of growing tumor. Helios-deficient Tregs may display a TCR repertoire skewed toward high-affinity against self-peptides/MHC, which can promote robust activation in TME (Yates et al., 2018). In view of the increased self-reactivity of TCR in CD4 Tregs compared with conventional T cells, conversion of Tregs could generate highly potent effector CD4 T cells accompanied by attenuated Treg-mediated suppression within the TME. A more effective strategy may depend on approaches that selectively convert intratumoral Tregs into Teff cells without affecting the systemic Treg population. As a key player in the maintenance of Treg size and functional stability in response to diverse immunological perturbations, pharmacological intervention of Helios could be relevant to the strategies that strengthen current tumor immunotherapy. Since Treg to Teff conversion may be confined to inflammatory intratumoral microenvironments, antibody or small molecule-based approaches that target Helios may lead to improved Treg dependent cancer immunotherapy. Importantly, conversion of Helios-deficient Tregs only occurs within the local inflammatory environment of the tumor. This approach may not provoke the autoimmune side effects associated with systemic reduction of Tregs. Therefore, strategies that specifically harness Helios-dependent control of the intratumoral Treg phenotype represent a significant promise to improve cancer immunotherapy. Furthermore, removal of Foxp3+Tregs was also reported to enhance vaccine-induced antitumor T-cell responses (Nishikawa et al., 2010, Int. J. Cancer 127: 759-767), suggesting that decreasing Helios levels could be beneficial in boosting the efficacy of cancer vaccines.

Besides anti-tumor immunotherapy, during viral infections, Treg cells can limit the immunopathology resulting from excessive inflammation, yet potentially inhibit effective antiviral T cell responses and promote virus persistence (Schmitz et al., 2013, PLOS Pathogens 9: e1003362). Chronic, but not acute, infection of mice with lymphocytic choriomeningitis virus results in a marked expansion of Foxp3+ Tregs, implying a potential mechanism that certain infectious agents could evade host immune responses by activation and expansion of Tregs (Punkosdy et al., 2011, PNAS 108: 3677-3682). Treatment benefits could be achieved by decreasing Helios levels in activated Tregs in the context relevant to chronic viral infections.

There is a need for compounds useful as inhibitors of Helios protein.

### SUMMARY OF THE INVENTION

The present invention provides pyridinyl substituted oxoisoindoline compounds of Formula (I) or salts thereof, which are useful to decrease Helios protein levels, decrease Helios activity levels and/or inhibit Helios expression levels in the cells.

The present invention also provides pharmaceutical compositions comprising a compound of Formula (I) and/or a pharmaceutically acceptable salt thereof; and a pharmaceutically acceptable carrier.

The present invention also provides a method of treating a disease or disorder by decreasing the activity of Helios protein, the method comprising administering to a patient a compound of Formula (I) and/or a pharmaceutically acceptable salt thereof.

The present invention also provides processes and intermediates for making the compounds of Formula (I) and/or salts thereof.

The present invention also provides a compound of Formula (I) and/or a pharmaceutically acceptable salt thereof, for use in therapy.

The present invention also provides the use of the compounds of Formula (I) and/or pharmaceutically acceptable salts thereof, for the manufacture of a medicament to decrease Helios protein levels, decrease Helios activity levels and/or inhibit Helios expression levels in cells to control Treg differentiation, for the treatment of certain diseases, including cancer and viral infections.

The compounds of Formula (I) and compositions comprising the compounds of Formula (I) may be used in treating, preventing, or curing viral infections and various proliferative disorders, such as cancer. Pharmaceutical compositions comprising these compounds are useful in treating, preventing, or slowing the progression of diseases or disorders in a variety of therapeutic areas, such as viral infections and cancer.

These and other features of the invention will be set forth in expanded form as the disclosure continues.

### DETAILED DESCRIPTION

Applicants have found substituted oxoisoindoline compounds that inhibit Helios protein by facilitating the interaction of Helios protein and the corresponding E3 ubiquitin ligase complex (Cullin4-Cereblon, CUL4-CRBN). These compounds decrease Helios protein levels, decrease Helios activity levels and/or inhibit Helios expression levels in the cells to control Treg differentiation. These compounds are useful for the treatment of certain diseases, including cancer and viral infections. The compounds are provided to be useful as pharmaceuticals with desirable stability, bioavailability, therapeutic index, and toxicity values that are important to their drugability.

The first aspect of the present invention provides at least one compound of Formula (I):

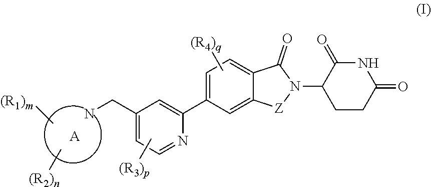

or a salt thereof, wherein:
Z is CR₆R₆ or C═O;
Ring A is azetidinyl, pyrrolidinyl, piperidinyl, azepanyl, tetrahydropyridazinyl, 1,4-azaphosphinane 4-oxide, pyrazolyl, isoindolinyl, dihydropyrrolo[3,4-c]pyridinyl, decahydroquinolinyl, tetrahydropyridinyl, tetrahydroisoquinolinyl, tetrahydronaphthyridinyl, hexahydrocyclopenta[c]pyrrolyl, hexahydrofuro[3,4-c]pyrrolyl, tetrahydropyrazolo[4,3-c]pyridinyl, tetrahydroisoxazolo[4,5-c]pyridinyl, tetrahydro[1,2,4]triazolo[4,3-a]pyrazinyl, octahydroisoindolyl, octahydropyrrolo[3,4-b]pyridinyl, octahydrocyclopenta[c]pyrrolyl, octahydropyrrolo[3,4-c]pyrrolyl, azaspiro[3.3]heptanyl, diazaspiro[3.3]heptanyl, oxaazaspiro[3.3]heptanyl, oxaazabicyclo[3.1.1]heptanyl, diazaspiro[3.4]octanyl, oxaazaspiro[3.4]octanyl, azaspiro[3.5]nonanyl, oxaazaspiro[3.5]nonanyl, diazaspiro[3.5]nonanyl, diazaspiro[4.4]nonanyl, azaspiro[4.5]decanyl, diazaspiro[4.5]decanyl, oxaazaspiro[4.5]decanyl, diazaspiro[4.5]decanonyl, oxadiazaspiro[4.5]decanyl, spiro[indoline-3,4′-piperidinyl], 2H-spiro[benzofuran-3,4′-piperidinyl], 3H-spiro[isobenzofuran-1,4′-piperidinyl], 2,3-dihydrospiro[indene-1,4′-piperidinyl], azabicyclo[3.1.0]hexanyl, azabicyclo[3.1.1]heptanyl, oxaazabicyclo[3.1.1]heptanyl, azabicyclo[3.2.1]octanyl, diazabicyclo[3.2.1]octanyl, or oxaazabicyclo[3.2.1]octanyl;
R₁ is F, —CN, —OH, C₁₋₄ alkyl, C₁₋₃ fluoroalkyl, C₁₋₃ hydroxyalkyl, —CH₂OCH₃, —C(O)(C₁₋₄ alkyl), —C(O)OH, —C(O)O(C₁₋₄ alkyl), —C(O)NR_{y}R_{y}, —CH₂NRₓC(O)OC(CH₃)₃, —NRₓC(O)OCH₃, —NRₓC(O)OC(CH₃)₃, —C(O)NRₓ(cyclohexyl), —S(O)₂CH₃, —P(O)(OH)O(phenyl), —CH₂(phenyl), —C(O)(cyclopropyl), —C(O)(phenyl), —NRₓ(phenyl), —CH₂NRₓC(O)(phenyl), —NRₓC(O)(phenyl), —CH₂O(fluorophenyl), —CH₂O(chloropyridinyl), —NRₓS(O)₂CH₃, —NRₓS(O)₂(phenyl), cyclopropyl, methyl oxadiazolyl, methylisoxazolyl, thiophenyl, —O(phenyl), —O(tert-butoxycarbonyl)phenyl), —O((tert-butoxycarbonyl)amino)phenyl), or phenyl substituted with zero, 1, or 2 R^{1a};
each R^{1a} is independently F, Cl, —OH, —CH₃, —CHF₂, —CF₃, or —S(O)₂CH₃;
each R₂ is independently F, —CN, —OH, —CH₃, —CF₃, —C(CH₃)₂OH, or —C(O)C(CH₃)₃;
each R₃ is independently F, Cl, —CH₃, —CF₃, —OCH₃, —OCF₃, or —NH₂;
each R₄ is independently F, Cl, or —CH₃;
each R₆ is independently hydrogen or C₁₋₃ alkyl;
Rₓ is hydrogen or —CH₃;
each R_{y} is independently hydrogen or C₁₋₄ alkyl;
m is zero or 1;
n is zero, 1, or 2;
p is zero, 1, or 2; and
q is zero, 1, 2, or 3;
with the provisos that:
(1) if Ring A is azetidinyl, pyrrolidinyl, or piperidinyl, then m is 1;
(2) if Ring A is azetidinyl, pyrrolidinyl, or piperidinyl; m is 1; and R₁ is C₁₋₄ alkyl, C₁₋₂ fluoroalkyl, —C(O)(C₁₋₃ alkyl), —C(O)NR_{y}R_{y}, —CH₂(phenyl), phenyl, or methylphenyl; then R₂ is F, —CN, —OH, —CF₃, —C(CH₃)₂OH, or —C(O)C(CH₃)₃.

The second aspect of the present invention provides at least one compound of Formula (II):

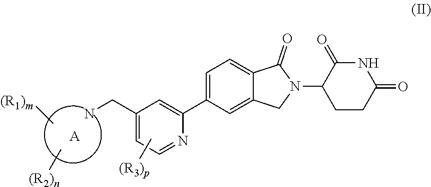

or a salt thereof, wherein:
Ring A is azetidinyl, pyrrolyl, piperidinyl, tetrahydropyridinyl, 1,4-azaphosphinane 4-oxide, 2,3-dihydrospiro[indene-1,4′-piperidinyl], 2H-spiro[benzofuran-3,4′-piperidinyl], 2-oxa-6-azaspiro[3.3]heptanyl, 3-azabicyclo[3.1.0]hexanyl, 3-oxa-6-azabicyclo[3.1.1]heptanyl, 6-oxa-2,9-diazaspiro[4.5]decane, 6-oxa-2,9-diazaspiro[4.5]decanyl, 6-oxa-2-azaspiro[3.4]octanyl, 6-oxa-3-azabicyclo[3.1.1]heptanyl, 8-oxa-3-azabicyclo[3.2.1]octanyl, azaspiro[3.3]heptyl, azaspiro[3.5]nonanyl, or spiro[indoline-3,4′-piperidinyl];
R₁ is —C(CH₃)₃, —C(CH₃)₂OH, —CH₂OCH₃, —C(O)OH, —C(O)OCH₃, —C(O)OC(CH₃)₃, —C(O)NH₂, —CH₂NHC(O)OC(CH₃)₃, —NHC(O)OC(CH₃)₃, —N(CH₃)C(O)OC(CH₃)₃, —S(O)₂CH₃, —P(O)(OH)O(phenyl), —C(O)(phenyl), —NH(phenyl), —N(CH₃)(phenyl), methyl oxadiazolyl, thiophenyl, —O(phenyl), —O(tert-butoxycarbonyl)phenyl), —O((tert-butoxycarbonyl)amino)phenyl), or phenyl substituted with zero, 1, or 2 R₁ₐ;
each R₁ₐ is independently F, Cl, —OH, —CHF₂, —CF₃, or —S(O)₂CH₃;
each R₂ is independently F, —CN, —OH, or —CH₃;
each R₃ is independently F or C₁;
m is zero or 1;
n is zero, 1, or 2; and
p is zero, 1, or 2.

One embodiment provides a compound of Formula (I) or a salt thereof, wherein Z is CR₆R₆. Compounds of this embodiment have the structure of Formula (Ia):

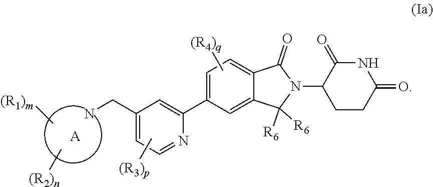

Included in this embodiment are compounds in which each R₆ is hydrogen. Also included in this embodiment are compounds in which one R₆ is hydrogen and the other R₆ is C₁₋₃ alkyl. Additionally, included in this embodiment are compounds in which one R₆ is hydrogen and the other R₆ is —CH₃.

One embodiment provides a compound of Formula (I) or a salt thereof, wherein Z is C═O. Compounds of this embodiment have the structure of Formula (Ib):

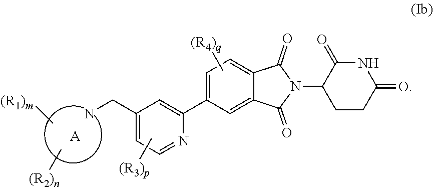

One embodiment provides a compound of Formula (I) or a compound of Formula (II) or salt thereof, wherein Ring A is azetidinyl, pyrrolyl, piperidinyl, tetrahydropyridinyl, or 1,4-azaphosphinane. Included in this embodiment are compounds in which m is 1. Also included in this embodiment are compounds in which n is zero or 1.

One embodiment provides a compound of Formula (I) or a compound of Formula (II) or salt thereof, wherein Ring A is azetidinyl, pyrrolyl, or piperidinyl. Included in this embodiment are compounds in which m is 1. Also included in this embodiment are compounds in which n is zero or 1.

One embodiment provides a compound of Formula (I) or a compound of Formula (II) or salt thereof, wherein Ring A is 2,3-dihydrospiro[indene-1,4′-piperidinyl], 2H-spiro[benzofuran-3,4′-piperidinyl], 2-oxa-6-azaspiro[3.3]heptanyl, 3-azabicyclo[3.1.0]hexanyl, 3-oxa-6-azabicyclo[3.1.1]heptanyl, 6-oxa-2,9-diazaspiro[4.5]decane, 6-oxa-2,9-diazaspiro[4.5]decanyl, 6-oxa-2-azaspiro[3.4]octanyl, 6-oxa-3-azabicyclo[3.1.1]heptanyl, 8-oxa-3-azabicyclo[3.2.1]octanyl, azaspiro[3.3]heptyl, azaspiro[3.5]nonanyl, or spiro[indoline-3,4′-piperidinyl]. Included in this embodiment are compounds in which m is 1. Also included in this embodiment are compounds in which n is zero or 1.

One embodiment provides a compound of Formula (I) or a compound of Formula (II) or salt thereof, wherein Ring A is 2-oxa-6-azaspiro[3.3]heptanyl, 6-oxa-2,9-diazaspiro[4.5]decane, 6-oxa-2,9-diazaspiro[4.5]decanyl, 6-oxa-2-azaspiro[3.4]octanyl, azaspiro[3.3]heptyl, or azaspiro[3.5]nonanyl. Included in this embodiment are compounds in which m is 1. Also included in this embodiment are compounds in which n is zero or 1.

One embodiment provides a compound of Formula (I) or a compound of Formula (II) or salt thereof, wherein Ring A is 3-azabicyclo[3.1.0]hexanyl, 3-oxa-6-azabicyclo[3.1.1]heptanyl, 6-oxa-3-azabicyclo[3.1.1]heptanyl, or 8-oxa-3-azabicyclo[3.2.1]octanyl. Included in this embodiment are compounds in which m is 1. Also included in this embodiment are compounds in which n is zero or 1.

One embodiment provides a compound of Formula (I) or salt thereof, wherein Ring A is azetidinyl, having the structure:

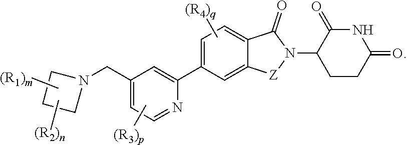

Included in this embodiment are compounds in which Z is CR₆R₆. Also included in this embodiment are compounds in which Z is C═O.

One embodiment provides a compound of Formula (II) or salt thereof, wherein Ring A is azetidinyl, having the structure:

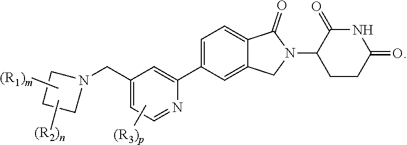

One embodiment provides a compound of Formula (I) or a compound of Formula (II) or salt thereof, wherein Ring A is pyrrolyl, having the structure:

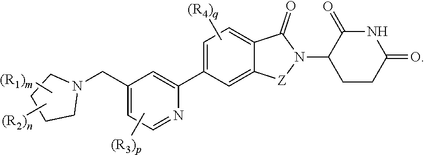

Included in this embodiment are compounds in which Z is CR₆R₆. Also included in this embodiment are compounds in which Z is C═O.

One embodiment provides a compound of Formula (II) or a compound of Formula (II) or salt thereof, wherein Ring A is pyrrolyl, having the structure:

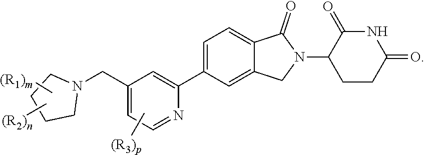

One embodiment provides a compound of Formula (I) or salt thereof, wherein Ring A is piperidinyl, having the structure:

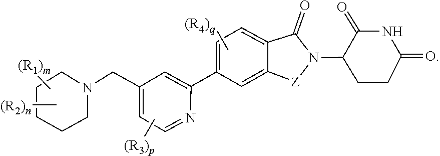

Included in this embodiment are compounds in which Z is CR₆R₆. Also included in this embodiment are compounds in which Z is C═O.

One embodiment provides a compound of Formula (II) or salt thereof, wherein Ring A is piperidinyl, having the structure:

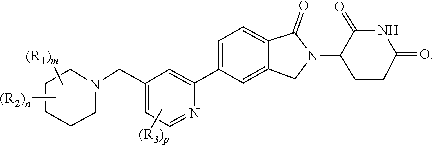

One embodiment provides a compound of Formula (I) or salt thereof, wherein Ring A is 6-oxa-2,9-diazaspiro[4.5]decanyl. Included in this embodiment are compounds having the structure:

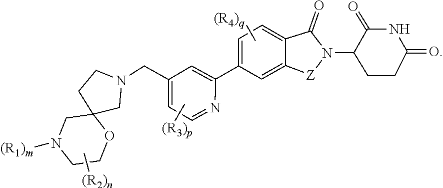

Included in this embodiment are compounds in which Z is CR₆R₆. Also included in this embodiment are compounds in which Z is C═O.

One embodiment provides a compound of Formula (II) or salt thereof, wherein Ring A is 6-oxa-2,9-diazaspiro[4.5]decanyl. Included in this embodiment are compounds having the structure:

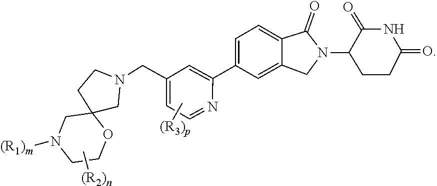

One embodiment provides a compound of Formula (I) or salt thereof, wherein Ring A is azaspiro[3.5]nonanyl. Included in this embodiment are compounds having the structure:

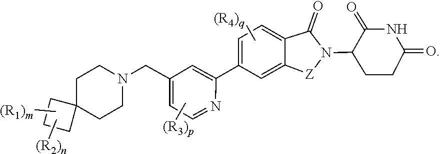

Included in this embodiment are compounds in which Z is CR₆R₆. Also included in this embodiment are compounds in which Z is C═O.

One embodiment provides a compound of Formula (II) or salt thereof, wherein Ring A is azaspiro[3.5]nonanyl. Included in this embodiment are compounds having the structure:

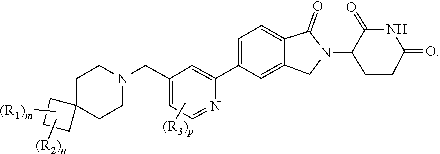

One embodiment provides a compound of Formula (I) or salt thereof, wherein Ring A is 3-azabicyclo[3.1.0]hexanyl. Included in this embodiment are compounds having the structure:

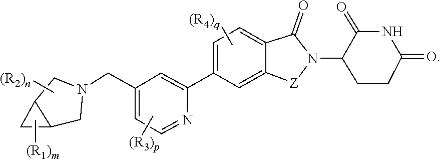

Included in this embodiment are compounds in which Z is CR₆R₆. Also included in this embodiment are compounds in which Z is C═O.

One embodiment provides a compound of Formula (II) or salt thereof, wherein Ring A is 3-azabicyclo[3.1.0]hexanyl. Included in this embodiment are compounds having the structure:

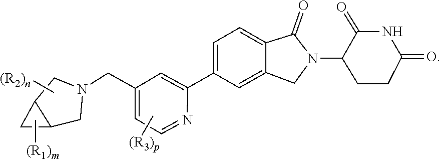

One embodiment provides a compound of Formula (I) or salt thereof, wherein Ring A is spiro[indoline-3,4′-piperidinyl]. Included in this embodiment are compounds having the structure:

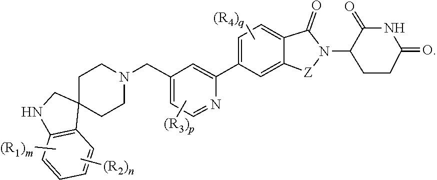

Included in this embodiment are compounds in which Z is CR₆R₆. Also included in this embodiment are compounds in which Z is C═O.

One embodiment provides a compound of Formula (II) or salt thereof, wherein Ring A is spiro[indoline-3,4′-piperidinyl]. Included in this embodiment are compounds having the structure:

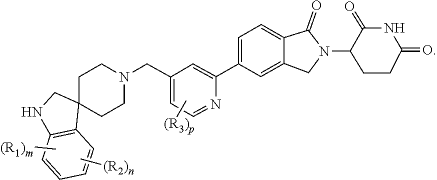

One embodiment provides a compound of Formula (I) or salt thereof, wherein Ring A is 2H-spiro[benzofuran-3,4′-piperidinyl]. Included in this embodiment are compounds having the structure:

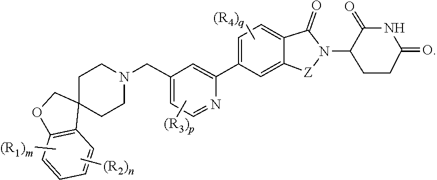

Included in this embodiment are compounds in which Z is CR₆R₆. Also included in this embodiment are compounds in which Z is C═O.

One embodiment provides a compound of Formula (II) or salt thereof, wherein Ring A is 2H-spiro[benzofuran-3,4′-piperidinyl]. Included in this embodiment are compounds having the structure:

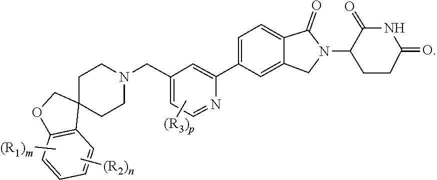

One embodiment provides a compound of Formula (I) or salt thereof, wherein Ring A is 2,3-dihydrospiro[indene-1,4′-piperidinyl]. Included in this embodiment are compounds having the structure:

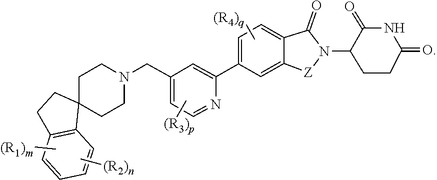

Included in this embodiment are compounds in which Z is CR₆R₆. Also included in this embodiment are compounds in which Z is C═O.

One embodiment provides a compound of Formula (II) or salt thereof, wherein Ring A is 2,3-dihydrospiro[indene-1,4′-piperidinyl]. Included in this embodiment are compounds having the structure:

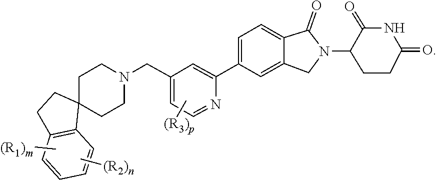

One embodiment provides a compound of Formula (I) or salt thereof, wherein Ring A is 3-oxa-6-azabicyclo[3.1.1]heptanyl. Included in this embodiment are compounds having the structure:

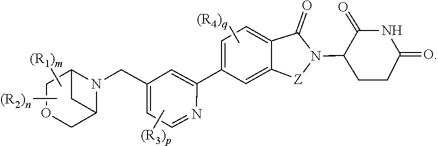

Included in this embodiment are compounds in which Z is CR₆R₆. Also included in this embodiment are compounds in which Z is C═O.

One embodiment provides a compound of Formula (II) or salt thereof, wherein Ring A is 3-oxa-6-azabicyclo[3.1.1]heptanyl. Included in this embodiment are compounds having the structure:

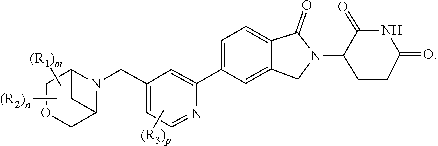

One embodiment provides a compound of Formula (I) or salt thereof, wherein Ring A is 6-oxa-3-azabicyclo[3.1.1]heptanyl. Included in this embodiment are compounds having the structure:

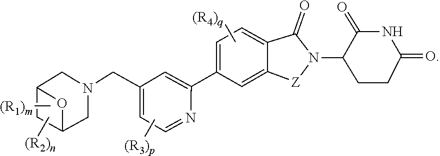

Included in this embodiment are compounds in which Z is CR₆R₆. Also included in this embodiment are compounds in which Z is C═O.

One embodiment provides a compound of Formula (II) or salt thereof, wherein Ring A is 6-oxa-3-azabicyclo[3.1.1]heptanyl. Included in this embodiment are compounds having the structure:

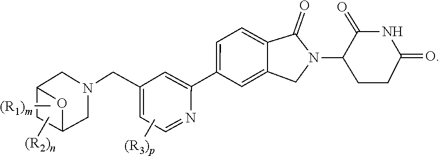

One embodiment provides a compound of Formula (I) or salt thereof, wherein Ring A is 8-oxa-3-azabicyclo[3.2.1]octanyl. Included in this embodiment are compounds having the structure:

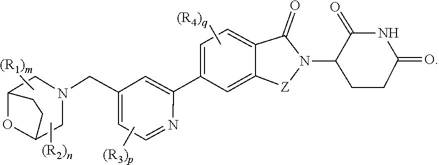

Included in this embodiment are compounds in which Z is CR₆R₆. Also included in this embodiment are compounds in which Z is C═O.

One embodiment provides a compound of Formula (II) or salt thereof, wherein Ring A is 8-oxa-3-azabicyclo[3.2.1]octanyl. Included in this embodiment are compounds having the structure:

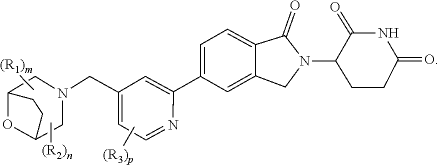

One embodiment provides a compound of Formula (I) or salt thereof, wherein Ring A is 1,2,3,6-tetrahydropyridinyl. Included in this embodiment are compounds having the structure:

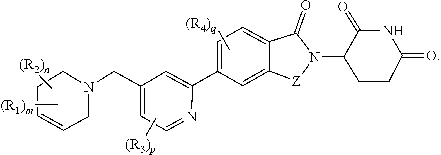

Included in this embodiment are compounds in which Z is CR₆R₆. Also included in this embodiment are compounds in which Z is C═O.

One embodiment provides a compound of Formula (II) or salt thereof, wherein Ring A is 1,2,3,6-tetrahydropyridinyl. Included in this embodiment are compounds having the structure:

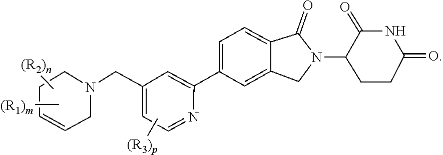

One embodiment provides a compound of Formula (I) or salt thereof, wherein Ring A is 1,4-azaphosphinane 4-oxide. Included in this embodiment are compounds having the structure:

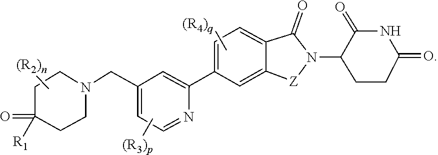

Included in this embodiment are compounds in which Z is CR₆R₆. A so included in this embodiment are compounds in which Z is C═O.

One embodiment provides a compound of Formula (II) or salt thereof, wherein Ring A is 1,4-azaphosphinane 4-oxide. Included in this embodiment are compounds having the structure:

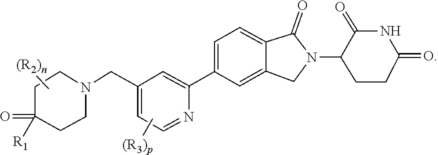

One embodiment provides a compound of Formula (I) or salt thereof, wherein Ring A is azaspiro[3.3]heptyl. Included in this embodiment are compounds having the structure:

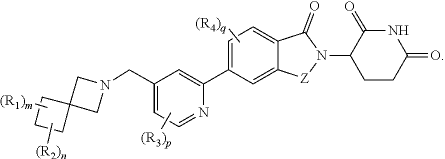

Included in this embodiment are compounds in which Z is CR₆R₆. Also included in this embodiment are compounds in which Z is C═O.

One embodiment provides a compound of Formula (II) or salt thereof, wherein Ring A is azaspiro[3.3]heptyl. Included in this embodiment are compounds having the structure:

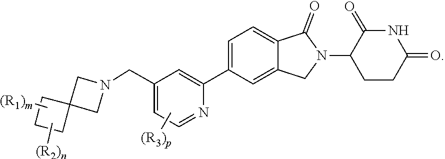

One embodiment provides a compound of Formula (I) or salt thereof, wherein Ring A is 6-oxa-2-azaspiro[3.4]octanyl. Included in this embodiment are compounds having the structure:

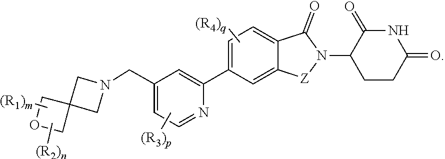

Included in this embodiment are compounds in which Z is CR₆R₆. Also included in this embodiment are compounds in which Z is C═O.

One embodiment provides a compound of Formula (II) or salt thereof, wherein Ring A is 6-oxa-2-azaspiro[3.4]octanyl. Included in this embodiment are compounds having the structure:

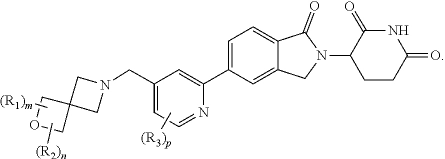

One embodiment provides a compound of Formula (I) or salt thereof, wherein Ring A is 6-oxa-2-azaspiro[3.4]octanyl. Included in this embodiment are compounds having the structure:

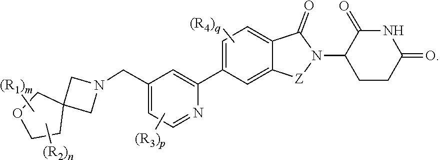

Included in this embodiment are compounds in which Z is CR₆R₆. Also included in this embodiment are compounds in which Z is C═O.

One embodiment provides a compound of Formula (II) or salt thereof, wherein Ring A is 6-oxa-2-azaspiro[3.4]octanyl. Included in this embodiment are compounds having the structure:

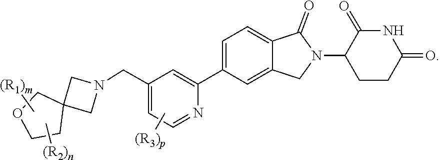

One embodiment provides a compound of Formula (I) or a compound of Formula (II) or salt thereof, wherein m is 1; and R₁ is F, —CN, —OH, —CH₃, —C(CH₃)₃, —CF₂CH₂CH₃, —CH₂OH, —CH(CH₃)OH, —C(CH₃)₂OH, —CH₂OCH₃, —C(O)CH₃, —C(O)OH, —C(O)OCH₃, —C(O)OCH₂CH₃, —C(O)OCH(CH₃)₂, —C(O)OC(CH₃)₃, —C(O)NH₂, —CH₂NHC(O)OC(CH₃)₃, —NHC(O)OCH₃, —NHC(O)OC(CH₃)₃, —N(CH₃)C(O)OCH₃, —N(CH₃)C(O)OC(CH₃)₃, —C(O)NHC(CH₃)₃, —C(O)NH(cyclohexyl), —S(O)₂CH₃, —P(O)(OH)O(phenyl), —CH₂(phenyl), —C(O)(cyclopropyl), —C(O)(phenyl), —NH(phenyl), —N(CH₃)(phenyl), —CH₂NHC(O)(phenyl), —CH₂N(CH₃)C(O)(phenyl), —N(CH₃)C(O)(phenyl), —CH₂O(fluorophenyl), —CH₂O(chloropyridinyl), —N(CH₃)S(O)₂CH₃, —N(CH₃)S(O)₂(phenyl), cyclopropyl, methyl oxadiazolyl, methylisoxazolyl, thiophenyl, —O(phenyl), —O(tert-butoxycarbonyl)phenyl), —O((tert-butoxycarbonyl)amino)phenyl), or phenyl substituted with zero, 1, or 2 R^{1a}.

One embodiment provides a compound of Formula (I) or a compound of Formula (II) or salt thereof, wherein m is 1; and R₁ is F, —CN, —OH, —CH₃, —C(CH₃)₃, —CF₂CH₂CH₃, —CH₂OH, —CH(CH₃)OH, —C(CH₃)₂OH, —CH₂OCH₃, —C(O)CH₃, —C(O)OH, —C(O)OCH₃, —C(O)OCH₂CH₃, —C(O)OCH(CH₃)₂, —C(O)OC(CH₃)₃, —C(O)NH₂, —CH₂NHC(O)OC(CH₃)₃, —NHC(O)OCH₃, —NHC(O)OC(CH₃)₃, —N(CH₃)C(O)OCH₃, —N(CH₃)C(O)OC(CH₃)₃, or —C(O)NHC(CH₃)₃.

One embodiment provides a compound of Formula (I) or a compound of Formula (II) or salt thereof, wherein m is 1; and R₁ is F, —CN, —OH, or C₁₋₄ alkyl. Included in this embodiment are compounds in which R₁ is F, —CN, —OH, —CH₃, —C(CH₃)₃, or —CF₂CH₂CH₃.

One embodiment provides a compound of Formula (I) or a compound of Formula (II) or salt thereof, wherein m is 1; and R₁ is C₁₋₃ hydroxyalkyl, —CH₂OCH₃, —C(O)(C₁₋₄ alkyl), —C(O)OH, —C(O)O(C₁₋₄ alkyl), —C(O)NR_{y}R_{y}, —CH₂NRₓC(O)OC(CH₃)₃, —NRₓC(O)OCH₃, or —NRₓC(O)OC(CH₃)₃. Included in this embodiment are compounds in which R₁ is —CH₂OH, —CH(CH₃)OH, —C(CH₃)₂OH, —CH₂OCH₃, —C(O)CH₃, —C(O)OH, —C(O)OCH₃, —C(O)OCH₂CH₃, —C(O)OCH(CH₃)₂, —C(O)OC(CH₃)₃, —C(O)NH₂, —CH₂NHC(O)OC(CH₃)₃, —NHC(O)OCH₃, —NHC(O)OC(CH₃)₃, —N(CH₃)C(O)OCH₃, —N(CH₃)C(O)OC(CH₃)₃, or —C(O)NHC(CH₃)₃. Additionally, included in this embodiment are compounds in which R₁ is —CH₂OH, —CH(CH₃)OH, —C(CH₃)₂OH, or —CH₂OCH₃.

One embodiment provides a compound of Formula (I) or a compound of Formula (II) or salt thereof, wherein R₁ is —C(CH₃)₃, —C(CH₃)₂OH, —CH₂OCH₃, —C(O)OH, —C(O)OCH₃, —C(O)OC(CH₃)₃, —C(O)NH₂, —CH₂NHC(O)OC(CH₃)₃, —NHC(O)OC(CH₃)₃, —N(CH₃)C(O)OC(CH₃)₃, or —S(O)₂CH₃.

One embodiment provides a compound of Formula (I) or a compound of Formula (II) or salt thereof, wherein R₁ is —P(O)(OH)O(phenyl).

One embodiment provides a compound of Formula (I) or a compound of Formula (II) or salt thereof, wherein R₁ is —C(O)(phenyl), —NH(phenyl), or —N(CH₃)(phenyl).

One embodiment provides a compound of Formula (I) or a compound of Formula (II) or salt thereof, wherein R₁ is methyl oxadiazolyl or thiophenyl.

One embodiment provides a compound of Formula (I) or a compound of Formula (II) or salt thereof, wherein R₁ is —O(phenyl), —O(tert-butoxycarbonyl)phenyl), —O((tert-butoxycarbonyl)amino) phenyl), or phenyl substituted with zero, 1, or 2 R^{1a}.

One embodiment provides a compound of Formula (I) or a compound of Formula (II) or salt thereof, wherein R₁ is —CH₂(phenyl), —C(O)(cyclopropyl), —C(O)(phenyl), —NRₓ(phenyl), —CH₂NRₓC(O)(phenyl), —NRₓC(O)(phenyl), —CH₂O(fluorophenyl), —CH₂O(chloropyridinyl), or —NRₓS(O)₂(phenyl).

One embodiment provides a compound of Formula (I) or a compound of Formula (II) or salt thereof, wherein R₁ is cyclopropyl, methyl oxadiazolyl, methylisoxazolyl, or thiophenyl.

One embodiment provides a compound of Formula (I) or a compound of Formula (II) or salt thereof, wherein R₁ is phenyl substituted with zero, 1, or 2 R^{1a}. Included in this embodiment are compounds in which R₁ is phenyl substituted with zero or 1 R^{1a}. Also included in this embodiment are compounds in which R₁ is phenyl substituted with 2 R^{1a}.

One embodiment provides a compound of Formula (I) or a compound of Formula (II) or salt thereof, wherein each R^{1a} is independently F, Cl, —OH, —CIF2, or —CF₃. Included in embodiment are compounds in which each R^{1a} is independently F or C₁.

One embodiment provides a compound of Formula (I) or a compound of Formula (II) or salt thereof, wherein R₁ is phenyl substituted with 1 or 2 R^{1a}. Included in this embodiment are compounds in which each R^{1a} is independently F, Cl, —OH, —CH₃, —CIF2, or —CF₃. Also included in this embodiment are compounds in which each Ria is independently F, Cl, —OH, —CHF₂, —CF₃, or —S(O)₂CH₃.

One embodiment provides a compound of Formula (I) or salt thereof, wherein each R₂ is independently F, —CN, —OH, —CH₃, —CF₃, or —C(CH₃)₂OH. Included in this embodiment are compounds in which n is 1. Also included in this embodiment are compounds in which n is 2.

One embodiment provides a compound of Formula (I) or salt thereof, wherein each R₂ is independently F, —CN, —OH, —CH₃, or —CF₃. Included in this embodiment are compounds in which n is 1. Also included in this embodiment are compounds in which n is 2.

One embodiment provides a compound of Formula (I) or salt thereof, wherein each R₂ is independently —OH or —CH₃. Included in this embodiment are compounds in which n is 1. Also included in this embodiment are compounds in which n is 2.

One embodiment provides a compound of Formula (I) or a compound of Formula (II) or salt thereof, wherein each R₂ is independently F, —OH, or —CH₃.

One embodiment provides a compound of Formula (I) or a compound of Formula (II) or salt thereof, wherein each R₂ is independently F.

One embodiment provides a compound of Formula (I) or a compound of Formula (II) or salt thereof, wherein n is 1 and R₂ is —OH.

One embodiment provides a compound of Formula (I) or a compound of Formula (II) or salt thereof, wherein n is 1 and R₂ is —CN.

One embodiment provides a compound of Formula (I) or a compound of Formula (II) or salt thereof, wherein n is 1 and R₂ is —CH₃.

One embodiment provides a compound of Formula (I) or a compound of Formula (II) or salt thereof, wherein n is 1 and R₂ is —CF₃.

One embodiment provides a compound of Formula (I) or a compound of Formula (II) or salt thereof, wherein each R₃ is independently F. Included in this embodiment are compounds in which p is 1. Also included in this embodiment are compounds in which p is 2.

One embodiment provides a compound of Formula (I) or a compound of Formula (II) or salt thereof, wherein each R₃ is independently C₁.

One embodiment provides a compound of Formula (I) or a compound of Formula (II) or salt thereof, wherein each R₃ is independently —CH₃.

One embodiment provides a compound of Formula (I) or a compound of Formula (II) or salt thereof, wherein each R₃ is independently —CF₃.

One embodiment provides a compound of Formula (I) or a compound of Formula (II) or salt thereof, wherein each R₃ is independently —OCH₃.

One embodiment provides a compound of Formula (I) or a compound of Formula (II) or salt thereof, wherein each R₃ is independently —OCF₃.

One embodiment provides a compound of Formula (I) or a compound of Formula (II) or salt thereof, wherein each R₃ is independently —NH₂.

One embodiment provides a compound of Formula (I) or a compound of Formula (II) or salt thereof, wherein p is 1; and R₃ is F.

One embodiment provides a compound of Formula (I) or a compound of Formula (II) or salt thereof, wherein p is 1; and R₃ is C₁.

One embodiment provides a compound of Formula (I) or a compound of Formula (II) or salt thereof, wherein p is 1; and R₃ is —CH₃.

One embodiment provides a compound of Formula (I) or a compound of Formula (II) or salt thereof, wherein p is 1; and R₃ is —CF₃.

One embodiment provides a compound of Formula (I) or a compound of Formula (II) or salt thereof, wherein p is 1; and R₃ is —OCH₃.

One embodiment provides a compound of Formula (I) or salt thereof, wherein each R₄ is independently F or C₁. Included in this embodiment are compounds in which q is 1 or 2. Also included in this embodiment are compounds in which q is 1.

One embodiment provides a compound of Formula (I) or salt thereof, wherein each R₄ is independently F or —CH₃. Included in this embodiment are compounds in which q is 1 or 2. Also included in this embodiment are compounds in which q is 1.

One embodiment provides a compound of Formula (I) or salt thereof, wherein each R₄ is independently F. Included in this embodiment are compounds in which q is 1 or 2. Also included in this embodiment are compounds in which q is 1.

One embodiment provides a compound of Formula (I) or salt thereof, wherein each R₄ is independently C₁. Included in this embodiment are compounds in which q is 1 or 2. Also included in this embodiment are compounds in which q is 1.

One embodiment provides a compound of Formula (I) or salt thereof, wherein each R₄ is independently —CH₃. Included in this embodiment are compounds in which q is 1 or 2. Also included in this embodiment are compounds in which q is 1.

One embodiment provides a compound of Formula (I) or salt thereof, wherein Z is CR₆R₆; each R₄ is independently F. Included in this embodiment are compounds in which q is 1. Also included in this embodiment are compounds in which one R₆ is hydrogen and the other R₆ is —CH₃. Additionally, included in this embodiment are compounds in which each R₆ is hydrogen.

One embodiment provides a compound of Formula (I) or salt thereof, wherein Z is CO; Ring A is pyrrolidinyl, piperidinyl, or oxaazaspiro[3.5]nonanyl; R₁ is —OH, —CH₂OH, —CH(CH₃)OH, —C(CH₃)₂OH, —CH₂OCH₃, or phenyl; R₂ is —OH or —CH₃; with the provisos (i) if Ring A is pyrrolidinyl or piperidinyl, then m is 1; and (ii) if Ring A is pyrrolidinyl or piperidinyl; m is 1; and R₁ is phenyl, then R₂ is —OH. Included in this embodiment are compounds in which Ring A is pyrrolidinyl or piperidinyl.

One embodiment provides a compound of Formula (I) or salt thereof, wherein Z is CR₆R₆; Ring A is pyrrolidinyl; R₁ is —OH, C₁₋₃ hydroxyalkyl, or —CH₂OCH₃; each R₂ is independently F, —CN, or —OH; R₃ is F, —CH₃, or —CF₃; R₄ is F; m is 1; n is zero or 1; p is zero or 1; and q is zero or 1. Included in this embodiment are compounds in which n is zero and p is zero. Also included in this embodiment are compounds in which n is zero; p is zero; and q is zero.

One embodiment provides a compound of Formula (I) or salt thereof, wherein Z is CR₆R₆; Ring A is piperidinyl; R₁ is —OH, C₁₋₃ hydroxyalkyl, or —CH₂OCH₃; each R₂ is independently F, —CN, or —OH; R₃ is F, —CH₃, or —CF₃; R₄ is F; m is 1; n is zero or 1; p is 1 or 2; and q is zero or 1. Included in this embodiment are compounds in which n is zero and p is 1. Also included in this embodiment are compounds in which n is zero; p is 1; and q is zero.

One embodiment provides a compound of Formula (I) or a compound of Formula (II) or salt thereof, wherein m is zero; and Ring A is azepanyl, tetrahydropyridazinyl, 1,4-azaphosphinane 4-oxide, pyrazolyl, isoindolinyl, dihydropyrrolo[3,4-c]pyridinyl, decahydroquinolinyl, tetrahydropyridinyl, tetrahydroisoquinolinyl, tetrahydronaphthyridinyl, hexahydrocyclopenta[c]pyrrolyl, hexahydrofuro[3,4-c]pyrrolyl, tetrahydropyrazolo[4,3-c]pyridinyl, tetrahydroisoxazolo[4,5-c]pyridinyl, tetrahydro[1,2,4]triazolo[4,3-a]pyrazinyl, octahydroisoindolyl, octahydropyrrolo[3,4-b]pyridinyl, octahydrocyclopenta[c]pyrrolyl, octahydropyrrolo[3,4-c]pyrrolyl, azaspiro[3.3]heptanyl, diazaspiro[3.3]heptanyl, oxaazaspiro[3.3]heptanyl, oxaazabicyclo[3.1.1]heptanyl, diazaspiro[3.4]octanyl, oxaazaspiro[3.4]octanyl, azaspiro[3.5]nonanyl, oxaazaspiro[3.5]nonanyl, diazaspiro[3.5]nonanyl, diazaspiro[4.4]nonanyl, azaspiro[4.5]decanyl, diazaspiro[4.5]decanyl, oxaazaspiro[4.5]decanyl, diazaspiro[4.5]decanonyl, oxadiazaspiro[4.5]decanyl, spiro[indoline-3,4′-piperidinyl], 2H-spiro[benzofuran-3,4′-piperidinyl], 3H-spiro[isobenzofuran-1,4′-piperidinyl], 2,3-dihydrospiro[indene-1,4′-piperidinyl], azabicyclo[3.1.0]hexanyl, azabicyclo[3.1.1]heptanyl, oxaazabicyclo[3.1.1]heptanyl, azabicyclo[3.2.1]octanyl, diazabicyclo[3.2.1]octanyl, or oxaazabicyclo[3.2.1]octanyl.

One embodiment provides a compound of Formula (I) or a compound of Formula (II) or salt thereof, wherein m is 1.

One embodiment provides a compound of Formula (I) or a compound of Formula (II) or salt thereof, wherein n is zero or 1.

One embodiment provides a compound of Formula (I) or a compound of Formula (II) or salt thereof, wherein n is 1 or 2.

One embodiment provides a compound of Formula (I) or a compound of Formula (II) or salt thereof, wherein n is zero.

One embodiment provides a compound of Formula (I) or a compound of Formula (II) or salt thereof, wherein n is 1.

One embodiment provides a compound of Formula (I) or a compound of Formula (II) or salt thereof, wherein n is 2.

One embodiment provides a compound of Formula (I) or a compound of Formula (II) or salt thereof, wherein p is zero or 1.

One embodiment provides a compound of Formula (I) or a compound of Formula (II) or salt thereof, wherein p is 1 or 2.

One embodiment provides a compound of Formula (I) or a compound of Formula (II) or salt thereof, wherein p is zero.

One embodiment provides a compound of Formula (I) or a compound of Formula (II) or salt thereof, wherein p is 1.

One embodiment provides a compound of Formula (I) or a compound of Formula (II) or salt thereof, wherein p is 2.

One embodiment provides a compound of Formula (I) or a compound of Formula (II) or salt thereof, wherein m is zero; n is zero or 1; and p is zero.

One embodiment provides a compound of Formula (I) or a compound of Formula (II) or salt thereof, wherein m is 1; n is zero or 1; and p is zero.

One embodiment provides a compound of Formula (I) or a compound of Formula (II) or salt thereof, wherein m is 1; n is zero; and p is zero.

One embodiment provides a compound of Formula (I) or a compound of Formula (II) or salt thereof, wherein m is 1; n is 1; and p is zero.

One embodiment provides a compound of Formula (I) or a compound of Formula (II) or salt thereof, wherein m is 1; n is zero or 1; and p is one.

One embodiment provides a compound of Formula (I) or a compound of Formula (II) or salt thereof, wherein m is 1; n is zero; and p is one.

One embodiment provides a compound of Formula (I) or a compound of Formula (II) or salt thereof, wherein m is 1; n is 1; and p is one.

One embodiment provides a compound of Formula (I) or a compound of Formula (II) or salt thereof, wherein m is 1; n is 2; and p is zero.

One embodiment provides a compound of Formula (I) or a compound of Formula (II) or salt thereof, wherein m is 1; n is 2; and p is one.

One embodiment provides a compound of Formula (I) or a salt thereof, wherein said compound is: 3-(5-(5-chloro-4-((3-(3,4-difluorophenyl)azetidin-1-yl)methyl)pyridin-2-yl)-1-oxoisoindolin-2-yl)piperidine-2,6-dione (1); 3-(5-(5-fluoro-4-((3-(methoxymethyl)azetidin-1-yl)methyl)pyridin-2-yl)-1-oxoisoindolin-2-yl)piperidine-2,6-dione (2); 3-(5-(4-((3-(3,4-difluorophenyl)azetidin-1-yl)methyl)-5-fluoropyridin-2-yl)-1-oxoisoindolin-2-yl)piperidine-2,6-dione (3); 3-(5-(5-fluoro-4-((3-(4-fluorophenyl) azetidin-1-yl)methyl)pyridin-2-yl)-1-oxoisoindolin-2-yl)piperidine-2,6-dione (4); 3-(5-(5-fluoro-4-((3-hydroxy-3-phenylazetidin-1-yl)methyl)pyridin-2-yl)-1-oxoisoindolin-2-yl)piperidine-2,6-dione (5); 3-(5-(4-((3-(3,4-difluorophenyl)azetidin-1-yl)methyl)-3-fluoropyridin-2-yl)-1-oxoisoindolin-2-yl)piperidine-2,6-dione (6); 3-(5-(3-fluoro-4-((3-(methoxymethyl)azetidin-1-yl)methyl)pyridin-2-yl)-1-oxoisoindolin-2-yl)piperidine-2,6-dione (7); 3-(5-(3-fluoro-4-((2-(4-fluorophenyl)azetidin-1-yl)methyl)pyridin-2-yl)-1-oxoisoindolin-2-yl)piperidine-2,6-dione (8); 3-(5-(3-fluoro-4-((3-hydroxy-3-phenylazetidin-1-yl)methyl)pyridin-2-yl)-1-oxoisoindolin-2-yl)piperidine-2,6-dione (9); 3-(5-(4-((2-(3-fluorophenyl)azetidin-1-yl)methyl)pyridin-2-yl)-1-oxoisoindolin-2-yl) piperidine-2,6-dione (10); 3-(5-(4-((3-(3-fluorophenyl)azetidin-1-yl)methyl)pyridin-2-yl)-1-oxoisoindolin-2-yl) piperidine-2,6-dione (11); 3-(5-(4-((3-(2-fluorophenyl)azetidin-1-yl)methyl)pyridin-2-yl)-1-oxoisoindolin-2-yl) piperidine-2,6-dione (12); 3-(5-(4-((3-(4-fluorophenyl)azetidin-1-yl)methyl)pyridin-2-yl)-1-oxoisoindolin-2-yl) piperidine-2,6-dione (13); 3-(5-(4-((3-(2-chlorophenyl)azetidin-1-yl)methyl)pyridin-2-yl)-1-oxoisoindolin-2-yl) piperidine-2,6-dione (14); 3-(5-(4-((3-(3-chlorophenyl)azetidin-1-yl) methyl)pyridin-2-yl)-1-oxoisoindolin-2-yl)piperidine-2,6-dione (15); 3-(5-(4-((3-(4-chlorophenyl)azetidin-1-yl)methyl)pyridin-2-yl)-1-oxoisoindolin-2-yl) piperidine-2,6-dione (16); 3-(1-oxo-5-(4-((3-(2-(trifluoromethyl)phenyl)azetidin-1-yl)methyl)pyridin-2-yl) isoindolin-2-yl)piperidine-2,6-dione (17); 3-(1-oxo-5-(4-((3-(3-(trifluoromethyl) phenyl)azetidin-1-yl)methyl)pyridin-2-yl) isoindolin-2-yl)piperidine-2,6-dione (18); 3-(1-oxo-5-(4-((3-(4-(trifluoromethyl)phenyl)azetidin-1-yl)methyl)pyridin-2-yl) isoindolin-2-yl)piperidine-2,6-dione (19); 3-(5-(4-((3-(3-(difluoromethyl)phenyl)azetidin-1-yl)methyl) pyridin-2-yl)-1-oxoisoindolin-2-yl)piperidine-2,6-dione (20); 3-(5-(4-((3-(4-(difluoromethyl)phenyl)azetidin-1-yl)methyl)pyridin-2-yl)-1-oxoisoindolin-2-yl) piperidine-2,6-dione (21); 3-(5-(4-((3-(2,4-difluorophenyl)azetidin-1-yl)methyl)pyridin-2-yl)-1-oxoisoindolin-2-yl)piperidine-2,6-dione (22); 3-(5-(4-((3-(2,3-difluorophenyl) azetidin-1-yl)methyl)pyridin-2-yl)-1-oxoisoindolin-2-yl)piperidine-2,6-dione (23); 3-(5-(4-((3-(3,5-difluorophenyl)azetidin-1-yl)methyl)pyridin-2-yl)-1-oxoisoindolin-2-yl)piperidine-2,6-dione (24); 3-(5-(4-((3-(3,4-dichlorophenyl)azetidin-1-yl)methyl) pyridin-2-yl)-1-oxoisoindolin-2-yl)piperidine-2,6-dione (25); 3-(5-(4-((3-(3,5-dichlorophenyl)azetidin-1-yl)methyl)pyridin-2-yl)-1-oxoisoindolin-2-yl)piperidine-2,6-dione (26); 3-(1-oxo-5-(4-((3-(thiophen-3-yl)azetidin-1-yl)methyl)pyridin-2-yl) isoindolin-2-yl) piperidine-2,6-dione (27); tert-butyl (1-((2-(2-(2,6-dioxopiperidin-3-yl)-1-oxoisoindolin-5-yl)pyridin-4-yl) methyl)azetidin-3-yl)carbamate (28); 3-(1-oxo-5-(4-((3-(phenylamino)azetidin-1-yl)methyl)pyridin-2-yl)isoindolin-2-yl) piperidine-2,6-dione (29); 3-(5-(4-((3-(methyl(phenyl)amino)azetidin-1-yl)methyl)pyridin-2-yl)-1-oxoisoindolin-2-yl)piperidine-2,6-dione (30); 3-(5-(4-((3-fluoro-3-phenylazetidin-1-yl) methyl)pyridin-2-yl)-1-oxoisoindolin-2-yl) piperidine-2,6-dione (31); tert-butyl (4-((7-((2-(2-(2,6-dioxopiperidin-3-yl)-1-oxoisoindolin-5-yl)pyridin-4-yl) methyl)-7-azaspiro[3.5]nonan-2-yl)oxy)phenyl)carbamate (32); 3-(5-(4-((3-hydroxyazetidin-1-yl)methyl)pyridin-2-yl)-1-oxoisoindolin-2-yl) piperidine-2,6-dione (33); 3-(5-(4-((3-hydroxy-3-methylazetidin-1-yl)methyl)pyridin-2-yl)-1-oxoisoindolin-2-yl) piperidine-2,6-dione (34); 3-(5-(4-(((2S,3R)-3-hydroxy-2-methylazetidin-1-yl)methyl)pyridin-2-yl)-1-oxoisoindolin-2-yl)piperidine-2,6-dione (35); 3-(5-(4-((3,3-difluoroazetidin-1-yl) methyl)pyridin-2-yl)-1-oxoisoindolin-2-yl) piperidine-2,6-dione (36); 3-(5-(4-((3-hydroxy-3-phenylazetidin-1-yl)methyl)pyridin-2-yl)-1-oxoisoindolin-2-yl) piperidine-2,6-dione (37); 3-(1-oxo-5-(4-((3-phenoxyazetidin-1-yl)methyl)pyridin-2-yl)isoindolin-2-yl) piperidine-2,6-dione (38); 3-(5-(4-(((S)-3-hydroxypyrrolidin-1-yl)methyl)pyridin-2-yl)-1-oxoisoindolin-2-yl) piperidine-2,6-dione (39); 3-(5-(4-(((R)-3-hydroxypyrrolidin-1-yl) methyl)pyridin-2-yl)-1-oxoisoindolin-2-yl) piperidine-2,6-dione (40); 3-(5-(4-(((S)-3-fluoropyrrolidin-1-yl)methyl)pyridin-2-yl)-1-oxoisoindolin-2-yl) piperidine-2,6-dione (41); 3-(5-(4-(((R)-3-fluoropyrrolidin-1-yl)methyl)pyridin-2-yl)-1-oxoisoindolin-2-yl) piperidine-2,6-dione (42); 3-(5-(4-((3-hydroxy-3-phenylpyrrolidin-1-yl)methyl)pyridin-2-yl)-1-oxoisoindolin-2-yl)piperidine-2,6-dione (43); 3-(1-oxo-5-(4-((1-phenyl-3-azabicyclo[3.1.0]hexan-3-yl)methyl)pyridin-2-yl) isoindolin-2-yl)piperidine-2,6-dione (44); tert-butyl 2-((2-(2-(2,6-dioxopiperidin-3-yl)-1-oxoisoindolin-5-yl)pyridin-4-yl) methyl)-6-oxa-2,9-diazaspiro[4.5]decane-9-carboxylate (45); 3-(1-oxo-5-(4-((3-phenoxypyrrolidin-1-yl)methyl)pyridin-2-yl)isoindolin-2-yl) piperidine-2,6-dione (46); 3-(5-(4-((4-(3-methyl-1,2,4-oxadiazol-5-yl)piperidin-1-yl)methyl)pyridin-2-yl)-1-oxoisoindolin-2-yl)piperidine-2,6-dione (47); 3-(5-(4-((4-hydroxy-4-phenylpiperidin-1-yl)methyl)pyridin-2-yl)-1-oxoisoindolin-2-yl)piperidine-2,6-dione (48); 1-((2-(2-(2,6-dioxopiperidin-3-yl)-1-oxoisoindolin-5-yl)pyridin-4-yl)methyl)-4-phenylpiperidine-4-carbonitrile (49); 3-(1-oxo-5-(4-((4-phenoxypiperidin-1-yl)methyl)pyridin-2-yl) isoindolin-2-yl) piperidine-2,6-dione (50); 3-(1-oxo-5-(4-(spiro[indoline-3,4′-piperidin]-1′-ylmethyl)pyridin-2-yl)isoindolin-2-yl)piperidine-2,6-dione (51); 3-(5-(4-((2H-spiro[benzofuran-3,4′-piperidin]-1′-yl)methyl)pyridin-2-yl)-1-oxoisoindolin-2-yl) piperidine-2,6-dione (52); 3-(5-(4-((2,3-dihydrospiro[indene-1,4′-piperidin]-1′-yl)methyl) pyridin-2-yl)-1-oxoisoindolin-2-yl)piperidine-2,6-dione (53); 3-(5-(4-((4-hydroxypiperidin-1-yl)methyl)pyridin-2-yl)-1-oxoisoindolin-2-yl) piperidine-2,6-dione (54); 3-(5-(4-((4-(2-hydroxypropan-2-yl)piperidin-1-yl)methyl)pyridin-2-yl)-1-oxoisoindolin-2-yl)piperidine-2,6-dione (55); 3-(5-(4-((2-hydroxy-7-azaspiro[3.5]nonan-7-yl)methyl)pyridin-2-yl)-1-oxoisoindolin-2-yl)piperidine-2,6-dione (56); 3-(5-(4-((4-(4-(methylsulfonyl)phenyl)piperidin-1-yl)methyl)pyridin-2-yl)-1-oxoisoindolin-2-yl) piperidine-2,6-dione (57); 3-(5-(4-((4-(3-hydroxyphenyl)piperidin-1-yl)methyl)pyridin-2-yl)-1-oxoisoindolin-2-yl)piperidine-2,6-dione (58); 3-(1-oxo-5-(4-((4-(2-(trifluoromethyl) phenyl)piperidin-1-yl)methyl)pyridin-2-yl) isoindolin-2-yl)piperidine-2,6-dione (59); 3-(5-(4-((4-(3-chlorophenyl)piperidin-1-yl)methyl)pyridin-2-yl)-1-oxoisoindolin-2-yl) piperidine-2,6-dione (60); 3-(5-(4-((4-(4-chlorophenyl)piperidin-1-yl)methyl)pyridin-2-yl)-1-oxoisoindolin-2-yl) piperidine-2,6-dione (61); 3-(5-(4-((4-(2-fluorophenyl) piperidin-1-yl)methyl)pyridin-2-yl)-1-oxoisoindolin-2-yl) piperidine-2,6-dione (62); 3-(5-(4-((4-(3-fluorophenyl)piperidin-1-yl)methyl)pyridin-2-yl)-1-oxoisoindolin-2-yl) piperidine-2,6-dione (63); 3-(5-(4-((3-oxa-6-azabicyclo[3.1.1]heptan-6-yl)methyl) pyridin-2-yl)-1-oxoisoindolin-2-yl)piperidine-2,6-dione (64); 3-(5-(4-((6-oxa-3-azabicyclo[3.1.1]heptan-3-yl)methyl)pyridin-2-yl)-1-oxoisoindolin-2-yl)piperidine-2,6-dione (65); 3-(5-(4-((8-oxa-3-azabicyclo[3.2.1]octan-3-yl)methyl)pyridin-2-yl)-1-oxoisoindolin-2-yl)piperidine-2,6-dione (66); methyl 4-((7-((2-(2-(2,6-dioxopiperidin-3-yl)-1-oxoisoindolin-5-yl)pyridin-4-yl) methyl)-7-azaspiro[3.5]nonan-2-yl)oxy)benzoate (67); 3-(5-(4-((6-oxa-2,9-diazaspiro[4.5]decan-2-yl)methyl)pyridin-2-yl)-1-oxoisoindolin-2-yl)piperidine-2,6-dione (68); 3-(5-(4-((9-benzoyl-6-oxa-2,9-diazaspiro[4.5]decan-2-yl)methyl)pyridin-2-yl)-1-oxoisoindolin-2-yl)piperidine-2,6-dione (69); 3-(5-(4-((2-(2-hydroxypropan-2-yl)azetidin-1-yl)methyl)pyridin-2-yl)-1-oxoisoindolin-2-yl)piperidine-2,6-dione (70); 3-(5-(4-((3-(methoxymethyl)azetidin-1-yl) methyl)pyridin-2-yl)-1-oxoisoindolin-2-yl) piperidine-2,6-dione (71); 3-(5-(4-((3,6-dihydropyridin-1(2H)-yl)methyl)pyridin-2-yl)-1-oxoisoindolin-2-yl) piperidine-2,6-dione (72); phenyl hydrogen (1-((2-(2-(2,6-dioxopiperidin-3-yl)-1-oxoisoindolin-5-yl)pyridin-4-yl)methyl)pyrrolidin-2-yl)phosphonate (73); 3-(5-(4-((4-methyl-4-oxido-1,4-azaphosphinan-1-yl)methyl)pyridin-2-yl)-1-oxoisoindolin-2-yl)piperidine-2,6-dione (74); 1-((2-(2-(2,6-dioxopiperidin-3-yl)-1-oxoisoindolin-5-yl)pyridin-4-yl)methyl) azetidine-2-carboxylic acid (75); 3-(5-(4-((3,3-difluoro-2-phenylazetidin-1-yl)methyl)pyridin-2-yl)-1-oxoisoindolin-2-yl)piperidine-2,6-dione (76); 3-(5-(4-((2-(4-fluorophenyl)azetidin-1-yl) methyl)pyridin-2-yl)-1-oxoisoindolin-2-yl) piperidine-2,6-dione (77); 3-(5-(4-((3-(3,4-difluorophenyl)azetidin-1-yl)methyl)pyridin-2-yl)-1-oxoisoindolin-2-yl)piperidine-2,6-dione (78); 1-((2-(2-(2,6-dioxopiperidin-3-yl)-1-oxoisoindolin-5-yl)pyridin-4-yl)methyl) azetidine-2-carboxamide (79); methyl 1-((2-(2-(2,6-dioxopiperidin-3-yl)-1-oxoisoindolin-5-yl)pyridin-4-yl)methyl) azetidine-2-carboxylate (80); 3-(5-(4-((2-azaspiro[3.3]heptan-2-yl)methyl)pyridin-2-yl)-1-oxoisoindolin-2-yl) piperidine-2,6-dione (81); 3-(5-(4-((2-oxa-6-azaspiro[3.3]heptan-6-yl)methyl)pyridin-2-yl)-1-oxoisoindolin-2-yl)piperidine-2,6-dione (82); 3-(5-(4-((6-oxa-2-azaspiro[3.4]octan-2-yl)methyl)pyridin-2-yl)-1-oxoisoindolin-2-yl) piperidine-2,6-dione (83); 3-(5-(4-((3-(methylsulfonyl)azetidin-1-yl) methyl)pyridin-2-yl)-1-oxoisoindolin-2-yl) piperidine-2,6-dione (84); tert-butyl ((1-((2-(2-(2,6-dioxopiperidin-3-yl)-1-oxoisoindolin-5-yl)pyridin-4-yl) methyl)azetidin-3-yl) methyl)carbamate (85); tert-butyl (1-((2-(2-(2,6-dioxopiperidin-3-yl)-1-oxoisoindolin-5-yl)pyridin-4-yl) methyl)azetidin-3-yl)(methyl)carbamate (86); or 3-(5-(3-fluoro-4-((4-(2-hydroxypropan-2-yl)piperidin-1-yl)methyl)pyridin-2-yl)-1-oxoisoindolin-2-yl)piperidine-2,6-dione (87).

One embodiment provides a compound of Formula (I) or a salt thereof, wherein said compound is: 3-(5-(4-((2-azaspiro[3.3]heptan-2-yl)methyl)-3-fluoropyridin-2-yl)-1-oxoisoindolin-2-yl)piperidine-2,6-dione (88); 3-(5-(3-fluoro-4-((3-(2-hydroxypropan-2-yl)azetidin-1-yl)methyl)pyridin-2-yl)-1-oxoisoindolin-2-yl)piperidine-2,6-dione (89); 3-(5-(3-fluoro-4-(((2S,3R)-3-hydroxy-2-methylazetidin-1-yl)methyl)pyridin-2-yl)-1-oxoisoindolin-2-yl)piperidine-2,6-dione (90); 3-(5-(3-fluoro-4-((4-hydroxy-4-phenylpiperidin-1-yl)methyl)pyridin-2-yl)-1-oxoisoindolin-2-yl)piperidine-2,6-dione (91); 3-(5-(5-fluoro-4-((4-(2-hydroxypropan-2-yl)piperidin-1-yl)methyl)pyridin-2-yl)-1-oxoisoindolin-2-yl)piperidine-2,6-dione (92); 3-(5-(4-((2-azaspiro[3.3]heptan-2-yl) methyl)-5-fluoropyridin-2-yl)-1-oxoisoindolin-2-yl) piperidine-2,6-dione (93); 3-(5-(3-chloro-4-((4-(2-hydroxypropan-2-yl)piperidin-1-yl)methyl)pyridin-2-yl)-1-oxoisoindolin-2-yl)piperidine-2,6-dione (94); 3-(5-(3-chloropyridin-2-yl)-1-oxoisoindolin-2-yl) piperidine-2,6-dione (95); 3-(5-(3-chloro-4-(((R)-2-(2-hydroxypropan-2-yl)pyrrolidin-1-yl)methyl)pyridin-2-yl)-1-oxoisoindolin-2-yl)piperidine-2,6-dione (96); 3-(5-(3-chloro-4-((3-(2-hydroxypropan-2-yl)azetidin-1-yl)methyl)pyridin-2-yl)-1-oxoisoindolin-2-yl) piperidine-2,6-dione (97); tert-butyl (4-((7-((2-(2-(2,6-dioxopiperidin-3-yl)-1-oxoisoindolin-5-yl)pyridin-4-yl) methyl)-7-azaspiro[3.5]nonan-2-yl)oxy)phenyl) carbamate (98); 3-(5-(4-((3-(methoxymethyl)piperidin-1-yl)methyl)pyridin-2-yl)-1-oxoisoindolin-2-yl)piperidine-2,6-dione (99); 3-(5-(4-((2-oxa-7-azaspiro[3.5]nonan-7-yl)methyl)pyridin-2-yl)-1-oxoisoindolin-2-yl)piperidine-2,6-dione (100); 3-(5-(4-(((1R,5S,6r)-6-(hydroxymethyl)-3-azabicyclo[3.1.0]hexan-3-yl)methyl)pyridin-2-yl)-1-oxoisoindolin-2-yl)piperidine-2,6-dione (101); 3-(5-(4-(((R)-3-(hydroxymethyl) pyrrolidin-1-yl)methyl)pyridin-2-yl)-1-oxoisoindolin-2-yl)piperidine-2,6-dione (102); 3-(5-(4-((6-chloro-1,3-dihydro-2H-pyrrolo[3,4-c]pyridin-2-yl)methyl)pyridin-2-yl)-1-oxoisoindolin-2-yl)piperidine-2,6-dione (103); 3-(5-(4-(((S)-3-(2-hydroxypropan-2-yl) pyrrolidin-1-yl)methyl)pyridin-2-yl)-1-oxoisoindolin-2-yl)piperidine-2,6-dione (104); 3-(5-(4-(((S)-3-(hydroxymethyl)pyrrolidin-1-yl)methyl)pyridin-2-yl)-1-oxoisoindolin-2-yl) piperidine-2,6-dione (105); 3-(5-(4-(((R)-3-(2-hydroxypropan-2-yl)pyrrolidin-1-yl) methyl)pyridin-2-yl)-1-oxoisoindolin-2-yl)piperidine-2,6-dione (106); 3-(5-(4-((3-(1-hydroxyethyl)pyrrolidin-1-yl)methyl)pyridin-2-yl)-1-oxoisoindolin-2-yl)piperidine-2,6-dione (107); 3-(5-(4-((4-(1-hydroxyethyl)piperidin-1-yl)methyl)pyridin-2-yl)-1-oxoisoindolin-2-yl)piperidine-2,6-dione (108); 3-(5-(3-fluoro-4-(((S)-3-(hydroxymethyl) pyrrolidin-1-yl)methyl)pyridin-2-yl)-1-oxoisoindolin-2-yl)piperidine-2,6-dione (109); 3-(5-(3-fluoro-4-(((S)-3-(2-hydroxypropan-2-yl)pyrrolidin-1-yl)methyl)pyridin-2-yl)-1-oxoisoindolin-2-yl)piperidine-2,6-dione (110); 3-(5-(3-fluoro-4-(((R)-3-(2-hydroxypropan-2-yl)pyrrolidin-1-yl)methyl)pyridin-2-yl)-1-oxoisoindolin-2-yl) piperidine-2,6-dione (111); 3-(5-(3-fluoro-4-((3-(1-hydroxyethyl)pyrrolidin-1-yl)methyl)pyridin-2-yl)-1-oxoisoindolin-2-yl)piperidine-2,6-dione (112); 3-(5-(3-fluoro-4-((4-(1-hydroxyethyl)piperidin-1-yl)methyl)pyridin-2-yl)-1-oxoisoindolin-2-yl) piperidine-2,6-dione (113); 3-(5-(3-fluoro-4-(((S)-3-hydroxypyrrolidin-1-yl)methyl) pyridin-2-yl)-1-oxoisoindolin-2-yl)piperidine-2,6-dione (114); 3-(5-(3-fluoro-4-(((R)-3-hydroxypyrrolidin-1-yl)methyl)pyridin-2-yl)-1-oxoisoindolin-2-yl)piperidine-2,6-dione (115); 2-(2,6-dioxopiperidin-3-yl)-5-(4-(((S)-3-(hydroxymethyl)pyrrolidin-1-yl)methyl) pyridin-2-yl)isoindoline-1,3-dione (116); 3-(5-(4-(((R)-3-(2-hydroxypropan-2-yl) pyrrolidin-1-yl)methyl)pyridin-2-yl)-1-oxoisoindolin-2-yl)piperidine-2,6-dione (117); 3-(5-(4-(((S)-3-(2-hydroxypropan-2-yl)pyrrolidin-1-yl)methyl)pyridin-2-yl)-1-oxoisoindolin-2-yl)piperidine-2,6-dione (118); 3-(5-(4-((3-(1-hydroxyethyl)pyrrolidin-1-yl)methyl)pyridin-2-yl)-1-oxoisoindolin-2-yl)piperidine-2,6-dione (119); 3-(5-(4-((4-(2-hydroxypropan-2-yl)piperidin-1-yl)methyl)pyridin-2-yl)-1-oxoisoindolin-2-yl)piperidine-2,6-dione (120); 3-(5-(4-((4-(1-hydroxyethyl)piperidin-1-yl)methyl)pyridin-2-yl)-1-oxoisoindolin-2-yl)piperidine-2,6-dione (121); 3-(5-(4-((2-oxa-7-azaspiro[3.5]nonan-7-yl)methyl)pyridin-2-yl)-1-oxoisoindolin-2-yl)piperidine-2,6-dione (122); 3-(5-(4-(((S)-3-hydroxypyrrolidin-1-yl)methyl)pyridin-2-yl)-1-oxoisoindolin-2-yl)piperidine-2,6-dione (123); 3-(5-(4-(((R)-3-hydroxypyrrolidin-1-yl)methyl)pyridin-2-yl)-1-oxoisoindolin-2-yl)piperidine-2,6-dione (124); 3-(5-(4-((3-hydroxy-3-phenylpyrrolidin-1-yl)methyl) pyridin-2-yl)-1-oxoisoindolin-2-yl)piperidine-2,6-dione (125); 3-(5-(4-((3-hydroxy-3-methylpyrrolidin-1-yl)methyl)pyridin-2-yl)-1-oxoisoindolin-2-yl)piperidine-2,6-dione (126); (R)-3-((R)-4-fluoro-5-(4-(((S)-3-hydroxypyrrolidin-1-yl)methyl)pyridin-2-yl)-3-methyl-1-oxoisoindolin-2-yl)piperidine-2,6-dione (127); (S)-3-((R)-4-fluoro-5-(4-(((R)-3-hydroxypyrrolidin-1-yl)methyl)pyridin-2-yl)-3-methyl-1-oxoisoindolin-2-yl)piperidine-2,6-dione (128); (S)-3-((R)-4-fluoro-5-(4-(((S)-3-(2-hydroxypropan-2-yl)pyrrolidin-1-yl) methyl)pyridin-2-yl)-3-methyl-1-oxoisoindolin-2-yl)piperidine-2,6-dione (129); (S)-3-((R)-4-fluoro-5-(4-(((R)-3-(2-hydroxypropan-2-yl)pyrrolidin-1-yl)methyl)pyridin-2-yl)-3-methyl-1-oxoisoindolin-2-yl)piperidine-2,6-dione (130); (S)-3-((R)-4-fluoro-5-(4-(((S)-3-(hydroxymethyl)pyrrolidin-1-yl)methyl)pyridin-2-yl)-3-methyl-1-oxoisoindolin-2-yl) piperidine-2,6-dione (131); (S)-3-((R)-4-fluoro-5-(4-((4-(2-hydroxypropan-2-yl) piperidin-1-yl)methyl)pyridin-2-yl)-3-methyl-1-oxoisoindolin-2-yl)piperidine-2,6-dione (132); (S)-3-((R)-4-fluoro-5-(4-(((R)-3-(hydroxymethyl)pyrrolidin-1-yl)methyl)pyridin-2-yl)-3-methyl-1-oxoisoindolin-2-yl)piperidine-2,6-dione (133); (3S)-3-((3R)-4-fluoro-5-(4-((3-(1-hydroxyethyl)pyrrolidin-1-yl)methyl)pyridin-2-yl)-3-methyl-1-oxoisoindolin-2-yl)piperidine-2,6-dione (134); (3S)-3-((3R)-4-fluoro-5-(4-((4-(1-hydroxyethyl)piperidin-1-yl)methyl)pyridin-2-yl)-3-methyl-1-oxoisoindolin-2-yl)piperidine-2,6-dione (135); (S)-3-((R)-5-(4-((2-oxa-7-azaspiro[3.5]nonan-7-yl)methyl)pyridin-2-yl)-4-fluoro-3-methyl-1-oxoisoindolin-2-yl)piperidine-2,6-dione (136); (S)-3-((R)-4-fluoro-5-(4-((3-hydroxyazetidin-1-yl)methyl)pyridin-2-yl)-3-methyl-1-oxoisoindolin-2-yl)piperidine-2,6-dione (137); (S)-3-((R)-4-fluoro-5-(4-((3-hydroxy-3-methylazetidin-1-yl)methyl)pyridin-2-yl)-3-methyl-1-oxoisoindolin-2-yl)piperidine-2,6-dione (138); (3S)-3-((3R)-4-fluoro-5-(4-((3-hydroxy-3-phenylpyrrolidin-1-yl)methyl)pyridin-2-yl)-3-methyl-1-oxoisoindolin-2-yl)piperidine-2,6-dione (139); (3S)-3-((3R)-4-fluoro-5-(4-((3-hydroxy-3-methylpyrrolidin-1-yl)methyl)pyridin-2-yl)-3-methyl-1-oxoisoindolin-2-yl)piperidine-2,6-dione (140); (S)-3-((R)-4-fluoro-3-methyl-1-oxo-5-(4-(((R)-3-phenoxypyrrolidin-1-yl)methyl)pyridin-2-yl)isoindolin-2-yl)piperidine-2,6-dione (141); S)-3-((R)-4-fluoro-5-(4-(((S)-3-fluoropyrrolidin-1-yl)methyl)pyridin-2-yl)-3-methyl-1-oxoisoindolin-2-yl) piperidine-2,6-dione (142); 3-((R)-4-fluoro-5-(3-fluoro-4-((4-(2-hydroxypropan-2-yl) piperidin-1-yl)methyl)pyridin-2-yl)-3-methyl-1-oxoisoindolin-2-yl)piperidine-2,6-dione (143); (S)-3-((R)-4-fluoro-5-(3-fluoro-4-(((S)-3-hydroxypyrrolidin-1-yl)methyl)pyridin-2-yl)-3-methyl-1-oxoisoindolin-2-yl)piperidine-2,6-dione (144); (S)-3-(5-(4-((6-hydroxy-2-azaspiro[3.3]heptan-2-yl)methyl)pyridin-2-yl)-1-oxoisoindolin-2-yl)piperidine-2,6-dione (145); (3S)-3-(5-(4-((5-hydroxyhexahydrocyclopenta[c]pyrrol-2(1H)-yl)methyl) pyridin-2-yl)-1-oxoisoindolin-2-yl)piperidine-2,6-dione (146); (3S)-3-(5-(4-((5-hydroxy-5-methylhexahydrocyclopenta[c]pyrrol-2(1H)-yl)methyl) pyridin-2-yl)-1-oxoisoindolin-2-yl)piperidine-2,6-dione (147); (3S)-3-(5-(4-((5-methyl-3,3a,4,6a-tetrahydrocyclopenta[c]pyrrol-2(1H)-yl)methyl) pyridin-2-yl)-1-oxoisoindolin-2-yl) piperidine-2,6-dione (148); (3S)-3-(5-(3-fluoro-4-(((3aR,7aS)-5-hydroxyoctahydro-2H-isoindol-2-yl)methyl)pyridin-2-yl)-1-oxoisoindolin-2-yl)piperidine-2,6-dione (149); (S)-3-(5-(3-fluoro-4-((1-oxo-2,8-diazaspiro[4.5]decan-8-yl)methyl)pyridin-2-yl)-1-oxoisoindolin-2-yl)piperidine-2,6-dione (150); (S)-3-(5-(3-fluoro-4-(((3aR,6aS)-tetrahydro-1H-furo[3,4-c]pyrrol-5(3H)-yl)methyl) pyridin-2-yl)-1-oxoisoindolin-2-yl) piperidine-2,6-dione (151); (S)-3-(1-oxo-5-(4-(((3aR,6aS)-tetrahydro-1H-furo[3,4-c]pyrrol-5(3H)-yl)methyl)pyridin-2-yl)isoindolin-2-yl)piperidine-2,6-dione (152); (S)-3-(5-(3-fluoro-4-((6-hydroxy-2-azaspiro[3.3]heptan-2-yl)methyl)pyridin-2-yl)-1-oxoisoindolin-2-yl)piperidine-2,6-dione (153); tert-butyl (3aS,6aS)-5-((2-(2-((S)-2,6-dioxopiperidin-3-yl)-1-oxoisoindolin-5-yl)-3-fluoropyridin-4-yl)methyl) hexahydropyrrolo[3,4-c]pyrrole-2(1H)-carboxylate (154); (3S)-3-(5-(3-fluoro-4-((hexahydrocyclopenta[c]pyrrol-2(1H)-yl)methyl)pyridin-2-yl)-1-oxoisoindolin-2-yl) piperidine-2,6-dione (155); tert-butyl (3aR,6aS)-5-((2-(2-((S)-2,6-dioxopiperidin-3-yl)-1-oxoisoindolin-5-yl)-3-fluoropyridin-4-yl)methyl)hexahydropyrrolo[3,4-c]pyrrole-2(1H)-carboxylate (156); 3-(5-(4-((2-azaspiro[3.5]nonan-2-yl)methyl)pyridin-2-yl)-1-oxoisoindolin-2-yl) piperidine-2,6-dione (157); (S)-3-(5-(4-((7-oxa-2-azaspiro[3.5]nonan-2-yl)methyl)pyridin-2-yl)-1-oxoisoindolin-2-yl)piperidine-2,6-dione (158); N-((1-((2-(2-(2,6-dioxopiperidin-3-yl)-1-oxoisoindolin-5-yl)pyridin-4-yl)methyl)azetidin-3-yl)methyl) benzamide (159); (S)—N-(1-((2-(2-(2,6-dioxopiperidin-3-yl)-1-oxoisoindolin-5-yl)pyridin-4-yl)methyl)azetidin-3-yl)-N-methylbenzamide (160); (S)—N-(1-((2-(2-(2,6-dioxopiperidin-3-yl)-1-oxoisoindolin-5-yl)pyridin-4-yl)methyl)azetidin-3-yl)-N-methylmethanesulfonamide (161); (S)—N-(1-((2-(2-(2,6-dioxopiperidin-3-yl)-1-oxoisoindolin-5-yl)pyridin-4-yl)methyl)azetidin-3-yl)-N-methylbenzenesulfonamide (162); methyl (S)-(1-((2-(2-(2,6-dioxopiperidin-3-yl)-1-oxoisoindolin-5-yl)pyridin-4-yl) methyl)azetidin-3-yl)(methyl)carbamate (163); (S)—N-((1-((2-(2-(2,6-dioxopiperidin-3-yl)-1-oxoisoindolin-5-yl)pyridin-4-yl)methyl)azetidin-3-yl)methyl)-N-methylbenzamide (164); 3-(5-(4-((1H-pyrazol-1-yl)methyl)pyridin-2-yl)-1-oxoisoindolin-2-yl)piperidine-2,6-dione (165); (S)-3-(5-(4-((4-methyl-1H-pyrazol-1-yl)methyl)pyridin-2-yl)-1-oxoisoindolin-2-yl)piperidine-2,6-dione (166); 3-(5-(4-((4-(2-hydroxypropan-2-yl) piperidin-1-yl)methyl)-5-methylpyridin-2-yl)-1-oxoisoindolin-2-yl)piperidine-2,6-dione (167); 3-(5-(4-((2-azaspiro[3.3]heptan-2-yl)methyl)-5-methylpyridin-2-yl)-1-oxoisoindolin-2-yl)piperidine-2,6-dione (168); 3-(5-(4-((3-cyclopropylpyrrolidin-1-yl) methyl)-5-methylpyridin-2-yl)-1-oxoisoindolin-2-yl)piperidine-2,6-dione (169); 3-(5-(4-((2-azaspiro[3.3]heptan-2-yl)methyl)-3-methylpyridin-2-yl)-1-oxoisoindolin-2-yl) piperidine-2,6-dione (170); 3-(5-(4-((4-(2-hydroxypropan-2-yl)piperidin-1-yl)methyl)-3-methylpyridin-2-yl)-1-oxoisoindolin-2-yl)piperidine-2,6-dione (171); 3-(5-(4-((4-(2-hydroxypropan-2-yl)piperidin-1-yl)methyl)-3-methoxypyridin-2-yl)-1-oxoisoindolin-2-yl)piperidine-2,6-dione (172); 3-(5-(4-((3-cyclopropylpyrrolidin-1-yl)methyl)-3-methoxypyridin-2-yl)-1-oxoisoindolin-2-yl)piperidine-2,6-dione (173); 3-(5-(4-((2-azaspiro[3.3]heptan-2-yl)methyl)-6-methoxypyridin-2-yl)-1-oxoisoindolin-2-yl) piperidine-2,6-dione (174); 3-(5-(4-((3-cyclopropylpyrrolidin-1-yl)methyl)-6-methoxypyridin-2-yl)-1-oxoisoindolin-2-yl)piperidine-2,6-dione (175); 3-(5-(4-((4-(2-hydroxypropan-2-yl)piperidin-1-yl)methyl)-3-(trifluoromethyl)pyridin-2-yl)-1-oxoisoindolin-2-yl)piperidine-2,6-dione (176); 3-(5-(4-((3-(methoxymethyl)azetidin-1-yl) methyl)-3-(trifluoromethyl)pyridin-2-yl)-1-oxoisoindolin-2-yl)piperidine-2,6-dione (177); 3-(5-(4-((2-azaspiro[3.3]heptan-2-yl)methyl)-3-(trifluoromethyl)pyridin-2-yl)-1-oxoisoindolin-2-yl)piperidine-2,6-dione (178); 3-(5-(4-((4-(2-hydroxypropan-2-yl) piperidin-1-yl)methyl)-5-(trifluoromethyl)pyridin-2-yl)-1-oxoisoindolin-2-yl)piperidine-2,6-dione (179); 3-(5-(4-((2-azaspiro[3.3]heptan-2-yl)methyl)-5-(trifluoromethyl)pyridin-2-yl)-1-oxoisoindolin-2-yl)piperidine-2,6-dione (180); 3-(5-(4-((3-(methoxymethyl) azetidin-1-yl)methyl)-5-(trifluoromethyl)pyridin-2-yl)-1-oxoisoindolin-2-yl)piperidine-2,6-dione (181); 3-(5-(3-fluoro-4-((4-(trifluoromethoxy)piperidin-1-yl)methyl)pyridin-2-yl)-1-oxoisoindolin-2-yl)piperidine-2,6-dione (182); 3-(5-(3-fluoro-4-((3-(trifluoromethoxy)pyrrolidin-1-yl)methyl)pyridin-2-yl)-1-oxoisoindolin-2-yl)piperidine-2,6-dione (183); 3-(5-(3-fluoro-4-(((S)-3-fluoropyrrolidin-1-yl)methyl)pyridin-2-yl)-1-oxoisoindolin-2-yl)piperidine-2,6-dione (184); 3-(5-(3-fluoro-4-((3-hydroxy-3-(trifluoromethyl)pyrrolidin-1-yl)methyl)pyridin-2-yl)-1-oxoisoindolin-2-yl)piperidine-2,6-dione (185); 3-(5-(3-fluoro-4-((4-fluoroisoindolin-2-yl)methyl)pyridin-2-yl)-1-oxoisoindolin-2-yl)piperidine-2,6-dione (186); 3-(5-(4-((5-chloroisoindolin-2-yl)methyl)-3-fluoropyridin-2-yl)-1-oxoisoindolin-2-yl)piperidine-2,6-dione (187); N-(tert-butyl)-1-((2-(2-(2,6-dioxopiperidin-3-yl)-1-oxoisoindolin-5-yl)pyridin-4-yl) methyl)pyrrolidine-2-carboxamide (188); 3-(5-(4-((1-(methoxymethyl)-2-azaspiro[3.3]heptan-2-yl)methyl) pyridin-2-yl)-1-oxoisoindolin-2-yl)piperidine-2,6-dione (189); (2S)—N-cyclohexyl-1-((2-(2-(2,6-dioxopiperidin-3-yl)-1-oxoisoindolin-5-yl)pyridin-4-yl)methyl)pyrrolidine-2-carboxamide (190); 3-(5-(4-(((R)-2-(((6-chloropyridin-3-yl)oxy)methyl)azetidin-1-yl) methyl)pyridin-2-yl)-1-oxoisoindolin-2-yl)piperidine-2,6-dione (191); 3-(5-(4-((2-((4-fluorophenoxy)methyl)azetidin-1-yl)methyl)pyridin-2-yl)-1-oxoisoindolin-2-yl) piperidine-2,6-dione (192); ethyl 3-((2-(2-(2,6-dioxopiperidin-3-yl)-1-oxoisoindolin-5-yl)pyridin-4-yl)methyl)-3-azabicyclo[3.1.0]hexane-2-carboxylate (193); 3-(5-(4-((2-azabicyclo[3.1.0]hexan-2-yl)methyl)pyridin-2-yl)-1-oxoisoindolin-2-yl)piperidine-2,6-dione (194); tert-butyl (((2S)-1-((2-(2-(2,6-dioxopiperidin-3-yl)-1-oxoisoindolin-5-yl) pyridin-4-yl)methyl)pyrrolidin-2-yl)methyl)carbamate (195); 3-(5-(4-((2-(isopropoxymethyl)azetidin-1-yl)methyl)pyridin-2-yl)-1-oxoisoindolin-2-yl)piperidine-2,6-dione (196); 3-(5-(4-((3-fluoro-3-phenylpiperidin-1-yl)methyl)pyridin-2-yl)-1-oxoisoindolin-2-yl)piperidine-2,6-dione (197); 3-(5-(3-fluoro-4-((4-fluoro-4-methylpiperidin-1-yl)methyl)pyridin-2-yl)-1-oxoisoindolin-2-yl)piperidine-2,6-dione (198); 3-(5-(4-((2-acetyltetrahydropyridazin-1(2H)-yl)methyl)-3-fluoropyridin-2-yl)-1-oxoisoindolin-2-yl)piperidine-2,6-dione (199); 3-(5-(4-((4-(1,1-difluoropropyl)piperidin-1-yl)methyl)-3-fluoropyridin-2-yl)-1-oxoisoindolin-2-yl)piperidine-2,6-dione (200); tert-butyl 2-((2-(2-(2,6-dioxopiperidin-3-yl)-1-oxoisoindolin-5-yl)-3-fluoropyridin-4-yl) methyl)-2,5-diazaspiro[3.5]nonane-5-carboxylate (201); 3-(5-(4-((1-acetyl-1,8-diazaspiro[4.5]decan-8-yl)methyl)-3-fluoropyridin-2-yl)-1-oxoisoindolin-2-yl)piperidine-2,6-dione (202); methyl 8-((2-(2-(2,6-dioxopiperidin-3-yl)-1-oxoisoindolin-5-yl)-3-fluoropyridin-4-yl) methyl)-1,8-diazaspiro[4.5]decane-1-carboxylate (203); tert-butyl 1-((2-(2-(2,6-dioxopiperidin-3-yl)-1-oxoisoindolin-5-yl)-3-fluoropyridin-4-yl)methyl) octahydro-6H-pyrrolo[3,4-b]pyridine-6-carboxylate (204); 3-(5-(3-fluoro-4-((3-fluoropiperidin-1-yl)methyl)pyridin-2-yl)-1-oxoisoindolin-2-yl)piperidine-2,6-dione (205); 3-(5-(4-((6,6-dimethyl-3-azabicyclo[3.1.0]hexan-3-yl)methyl)-3-fluoropyridin-2-yl)-1-oxoisoindolin-2-yl)piperidine-2,6-dione (206); tert-butyl 2-((2-(2-(2,6-dioxopiperidin-3-yl)-1-oxoisoindolin-5-yl)-3-fluoropyridin-4-yl)methyl)-2,6-diazaspiro[3.4]octane-6-carboxylate (207); tert-butyl 2-((2-(2-(2,6-dioxopiperidin-3-yl)-1-oxoisoindolin-5-yl)-3-fluoropyridin-4-yl)methyl)-2,6-diazaspiro[3.5]nonane-6-carboxylate (208); 3-(5-(3-fluoro-4-((4-hydroxy-4-(trifluoromethyl)piperidin-1-yl) methyl)pyridin-2-yl)-1-oxoisoindolin-2-yl)piperidine-2,6-dione (209); 3-(5-(4-((8-azaspiro[4.5]decan-8-yl)methyl)-3-fluoropyridin-2-yl)-1-oxoisoindolin-2-yl)piperidine-2,6-dione (210); 3-(5-(3-fluoro-4-((4-hydroxy-2-methylpiperidin-1-yl)methyl)pyridin-2-yl)-1-oxoisoindolin-2-yl)piperidine-2,6-dione (211-212); tert-butyl 7-((2-(2-(2,6-dioxopiperidin-3-yl)-1-oxoisoindolin-5-yl)-3-fluoropyridin-4-yl)methyl)-1,7-diazaspiro[4.4]nonane-1-carboxylate (213); tert-butyl (2S,4R)-4-(tert-butoxy)-1-((2-(2-(2,6-dioxopiperidin-3-yl)-1-oxoisoindolin-5-yl)-3-fluoropyridin-4-yl)methyl)pyrrolidine-2-carboxylate (214-215); tert-butyl 6-((2-(2-(2,6-dioxopiperidin-3-yl)-1-oxoisoindolin-5-yl)-3-fluoropyridin-4-yl)methyl)-1,6-diazaspiro[3.5]nonane-1-carboxylate (216); 3-(5-(3-fluoro-4-(((R)-2-((R)-1-hydroxy-1-phenylethyl)pyrrolidin-1-yl)methyl)pyridin-2-yl)-1-oxoisoindolin-2-yl)piperidine-2,6-dione (217); 3-(5-(3-fluoro-4-(((R)-2-((S)-1-hydroxy-1-phenylethyl)pyrrolidin-1-yl)methyl)pyridin-2-yl)-1-oxoisoindolin-2-yl)piperidine-2,6-dione (218); 3-(5-(3-fluoro-4-((6-hydroxy-3-azabicyclo[3.1.1]heptan-3-yl)methyl) pyridin-2-yl)-1-oxoisoindolin-2-yl)piperidine-2,6-dione (219); 3-(5-(3-fluoro-4-((4-hydroxy-4-methylazepan-1-yl)methyl)pyridin-2-yl)-1-oxoisoindolin-2-yl)piperidine-2,6-dione (220); 3-(5-(3-fluoro-4-((4-hydroxy-3,3-dimethylpiperidin-1-yl)methyl)pyridin-2-yl)-1-oxoisoindolin-2-yl)piperidine-2,6-dione (221); 3-(5-(3-fluoro-4-((3-hydroxy-1-oxa-8-azaspiro[4.5]decan-8-yl)methyl)pyridin-2-yl)-1-oxoisoindolin-2-yl)piperidine-2,6-dione (222); 3-(5-(3-fluoro-4-((3-hydroxy-2-methylazetidin-1-yl)methyl)pyridin-2-yl)-1-oxoisoindolin-2-yl)piperidine-2,6-dione (223); 3-(5-(3-fluoro-4-((3-hydroxy-3-methylazetidin-1-yl)methyl)pyridin-2-yl)-1-oxoisoindolin-2-yl)piperidine-2,6-dione (224); 3-(5-(3-fluoro-4-((2-hydroxy-7-azaspiro[3.5]nonan-7-yl)methyl)pyridin-2-yl)-1-oxoisoindolin-2-yl)piperidine-2,6-dione (225); 3-(5-(3-fluoro-4-((4-hydroxypiperidin-1-yl)methyl)pyridin-2-yl)-1-oxoisoindolin-2-yl)piperidine-2,6-dione (226); 3-(5-(4-((3-acetyl-3,8-diazabicyclo[3.2.1]octan-8-yl)methyl)-3-fluoropyridin-2-yl)-1-oxoisoindolin-2-yl)piperidine-2,6-dione (227); methyl 8-((2-(2-(2,6-dioxopiperidin-3-yl)-1-oxoisoindolin-5-yl)-3-fluoropyridin-4-yl)methyl)-3,8-diazabicyclo[3.2.1]octane-3-carboxylate (228); 3-(5-(4-((3-benzoyl-3,8-diazabicyclo[3.2.1]octan-8-yl)methyl)-3-fluoropyridin-2-yl)-1-oxoisoindolin-2-yl)piperidine-2,6-dione (229); 3-(5-(3-fluoro-4-((3-hydroxy-3,8-diazabicyclo[3.2.1]octan-8-yl)methyl)pyridin-2-yl)-1-oxoisoindolin-2-yl)piperidine-2,6-dione (230); 3-(5-(4-((1-(cyclopropanecarbonyl)-1,8-diazaspiro[4.5]decan-8-yl)methyl)-3-fluoropyridin-2-yl)-1-oxoisoindolin-2-yl)piperidine-2,6-dione (231); 3-(5-(3-fluoro-4-(((3S,4R)-3-hydroxy-4-methylpyrrolidin-1-yl)methyl) pyridin-2-yl)-1-oxoisoindolin-2-yl)piperidine-2,6-dione (232); 3-(5-(4-((5-acetyl-2,5-diazaspiro[3.5]nonan-2-yl)methyl)-3-fluoropyridin-2-yl)-1-oxoisoindolin-2-yl)piperidine-2,6-dione (233); 3-(5-(3-fluoro-4-((3-(4-fluorophenyl)-3-hydroxypyrrolidin-1-yl)methyl) pyridin-2-yl)-1-oxoisoindolin-2-yl)piperidine-2,6-dione (234); 3-(5-(4-((4-benzyl-4-hydroxypiperidin-1-yl)methyl)-3-fluoropyridin-2-yl)-1-oxoisoindolin-2-yl)piperidine-2,6-dione (235); tert-butyl ((3S,5S)-1-((2-(2-(2,6-dioxopiperidin-3-yl)-1-oxoisoindolin-5-yl)-3-fluoropyridin-4-yl)methyl)-5-fluoropiperidin-3-yl)carbamate (236); 3-(5-(3-fluoro-4-((4-(4-fluorophenyl)-4-hydroxy-2-methylpyrrolidin-1-yl)methyl)pyridin-2-yl)-1-oxoisoindolin-2-yl)piperidine-2,6-dione (237); 3-(5-(4-((3-(2-chlorophenyl)-3-hydroxypyrrolidin-1-yl)methyl)-3-fluoropyridin-2-yl)-1-oxoisoindolin-2-yl)piperidine-2,6-dione (238); 3-(5-(3-fluoro-4-(((4aR,8R,8aR)-8-hydroxyoctahydroquinolin-1(2H)-yl) methyl)pyridin-2-yl)-1-oxoisoindolin-2-yl)piperidine-2,6-dione (239); methyl (2S,3R)-1-((2-(2-(2,6-dioxopiperidin-3-yl)-1-oxoisoindolin-5-yl)-3-fluoropyridin-4-yl)methyl)-3-hydroxypyrrolidine-2-carboxylate (240); 3-(5-(3-fluoro-4-((3-hydroxy-3-(p-tolyl) pyrrolidin-1-yl)methyl)pyridin-2-yl)-1-oxoisoindolin-2-yl)piperidine-2,6-dione (241); 3-(5-(4-((3-(3-chlorophenyl)-3-hydroxypyrrolidin-1-yl)methyl)-3-fluoropyridin-2-yl)-1-oxoisoindolin-2-yl)piperidine-2,6-dione (242); 3-(5-(3-fluoro-4-((4-fluoro-4-phenylpiperidin-1-yl)methyl)pyridin-2-yl)-1-oxoisoindolin-2-yl)piperidine-2,6-dione (243); 3-(5-(3-fluoro-4-((2-(3-methylisoxazol-5-yl)pyrrolidin-1-yl)methyl)pyridin-2-yl)-1-oxoisoindolin-2-yl)piperidine-2,6-dione (244); 3-(5-(3-fluoro-4-(((S)-2-(3-methylisoxazol-5-yl)pyrrolidin-1-yl)methyl)pyridin-2-yl)-1-oxoisoindolin-2-yl) piperidine-2,6-dione (245); tert-butyl 7-((2-(2-(2,6-dioxopiperidin-3-yl)-1-oxoisoindolin-5-yl)-3-fluoropyridin-4-yl)methyl)-2,7-diazaspiro[3.5]nonane-2-carboxylate (246); 3-(5-(3-fluoro-4-((4-hydroxy-3-methylpiperidin-1-yl)methyl)pyridin-2-yl)-1-oxoisoindolin-2-yl)piperidine-2,6-dione (247); methyl ((3S,4S)-1-((2-(2-(2,6-dioxopiperidin-3-yl)-1-oxoisoindolin-5-yl)-3-fluoropyridin-4-yl)methyl)-4-hydroxypyrrolidin-3-yl)carbamate (248); 3-(5-(4-((3H-spiro[isobenzofuran-1,4′-piperidin]-1′-yl)methyl)-3-fluoropyridin-2-yl)-1-oxoisoindolin-2-yl)piperidine-2,6-dione (249); 3-(5-(3-fluoro-4-((3-hydroxy-3-phenylpiperidin-1-yl)methyl)pyridin-2-yl)-1-oxoisoindolin-2-yl)piperidine-2,6-dione (250); 3-(5-(4-((2-acetyl-2,7-diazaspiro[3.5]nonan-7-yl)methyl)-3-fluoropyridin-2-yl)-1-oxoisoindolin-2-yl)piperidine-2,6-dione (251); methyl 7-((2-(2-(2,6-dioxopiperidin-3-yl)-1-oxoisoindolin-5-yl)-3-fluoropyridin-4-yl)methyl)-2,7-diazaspiro[3.5]nonane-2-carboxylate (252); 3-(5-(3-fluoro-4-((3-fluoro-3-phenylpiperidin-1-yl)methyl)pyridin-2-yl)-1-oxoisoindolin-2-yl)piperidine-2,6-dione (253); 3-(5-(3-fluoro-4-((1,4,6,7-tetrahydro-5H-pyrazolo[4,3-c]pyridin-5-yl)methyl)pyridin-2-yl)-1-oxoisoindolin-2-yl) piperidine-2,6-dione (254); 3-(5-(3-fluoro-4-((5-hydroxy-5-methylhexahydrocyclopenta[c]pyrrol-2(1H)-yl)methyl)pyridin-2-yl)-1-oxoisoindolin-2-yl)piperidine-2,6-dione (255); 3-(5-(4-((3-(2,5-dimethylphenyl)-3-hydroxypyrrolidin-1-yl)methyl)-3-fluoropyridin-2-yl)-1-oxoisoindolin-2-yl)piperidine-2,6-dione (256); 3-(5-(3-fluoro-4-((3-hydroxy-3-phenylpyrrolidin-1-yl)methyl)pyridin-2-yl)-1-oxoisoindolin-2-yl)piperidine-2,6-dione (257); 3-(5-(3-fluoro-4-(((R)-3-fluoropyrrolidin-1-yl)methyl) pyridin-2-yl)-1-oxoisoindolin-2-yl)piperidine-2,6-dione (258); 3-(5-(4-((6,7-dihydroisoxazolo[4,5-c]pyridin-5(4H)-yl)methyl)-3-fluoropyridin-2-yl)-1-oxoisoindolin-2-yl)piperidine-2,6-dione (259); 3-(5-(3-fluoro-4-((4-fluoropiperidin-1-yl)methyl)pyridin-2-yl)-1-oxoisoindolin-2-yl)piperidine-2,6-dione (260); 3-(5-(4-((4,4-difluoropiperidin-1-yl)methyl)-3-fluoropyridin-2-yl)-1-oxoisoindolin-2-yl)piperidine-2,6-dione (261); 3-(5-(3-fluoro-4-((3-methyl-5,6-dihydro-[1,2,4]triazolo[4,3-a]pyrazin-7(8H)-yl)methyl) pyridin-2-yl)-1-oxoisoindolin-2-yl)piperidine-2,6-dione (262); 3-(5-(4-((5,6-dihydro-[1,2,4]triazolo[4,3-a]pyrazin-7(8H)-yl)methyl)-3-fluoropyridin-2-yl)-1-oxoisoindolin-2-yl)piperidine-2,6-dione (263); 3-(5-(4-((1,3-dimethyl-1,4,6,7-tetrahydro-5H-pyrazolo[4,3-c]pyridin-5-yl)methyl)-3-fluoropyridin-2-yl)-1-oxoisoindolin-2-yl)piperidine-2,6-dione (264); 3-(5-(4-((2,3-dimethyl-2,4,6,7-tetrahydro-5H-pyrazolo[4,3-c]pyridin-5-yl)methyl)-3-fluoropyridin-2-yl)-1-oxoisoindolin-2-yl)piperidine-2,6-dione (265); 3-(5-(3-fluoro-4-((3-(trifluoromethyl)-5,6-dihydro-[1,2,4]triazolo[4,3-a]pyrazin-7(8H)-yl)methyl)pyridin-2-yl)-1-oxoisoindolin-2-yl)piperidine-2,6-dione (266); 3-(5-(4-((5-chloro-3,4-dihydro-2,6-naphthyridin-2(1H)-yl)methyl)-3-fluoropyridin-2-yl)-1-oxoisoindolin-2-yl)piperidine-2,6-dione (267); 3-(5-(4-((8-chloro-3,4-dihydroisoquinolin-2(1H)-yl)methyl)-3-fluoropyridin-2-yl)-1-oxoisoindolin-2-yl)piperidine-2,6-dione (268); 3-(5-(4-((6-acetyl-2,6-diazaspiro[3.3]heptan-2-yl)methyl)-3-fluoropyridin-2-yl)-1-oxoisoindolin-2-yl) piperidine-2,6-dione (269); 3-(5-(4-((1-acetyl-1,7-diazaspiro[4.4]nonan-7-yl)methyl)-3-fluoropyridin-2-yl)-1-oxoisoindolin-2-yl)piperidine-2,6-dione (270); methyl 7-((2-(2-(2,6-dioxopiperidin-3-yl)-1-oxoisoindolin-5-yl)-3-fluoropyridin-4-yl)methyl)-1,7-diazaspiro[4.4]nonane-1-carboxylate (271); 3-(5-(4-((6-acetyl-2,6-diazaspiro[3.4]octan-2-yl)methyl)-3-fluoropyridin-2-yl)-1-oxoisoindolin-2-yl)piperidine-2,6-dione (272); methyl 2-((2-(2-(2,6-dioxopiperidin-3-yl)-1-oxoisoindolin-5-yl)-3-fluoropyridin-4-yl)methyl)-2,6-diazaspiro[3.4]octane-6-carboxylate (273); 3-(5-(4-((6-acetyloctahydro-1H-pyrrolo[3,4-b]pyridin-1-yl)methyl)-3-fluoropyridin-2-yl)-1-oxoisoindolin-2-yl)piperidine-2,6-dione (274); methyl 1-((2-(2-(2,6-dioxopiperidin-3-yl)-1-oxoisoindolin-5-yl)-3-fluoropyridin-4-yl)methyl)octahydro-6H-pyrrolo[3,4-b]pyridine-6-carboxylate (275); 3-(4-fluoro-5-(3-fluoro-4-((4-(2-hydroxypropan-2-yl)piperidin-1-yl)methyl)pyridin-2-yl)-1-oxoisoindolin-2-yl)piperidine-2,6-dione (276); 3-(4-fluoro-5-(3-fluoro-4-(((S)-3-(hydroxymethyl)pyrrolidin-1-yl)methyl)pyridin-2-yl)-1-oxoisoindolin-2-yl)piperidine-2,6-dione (277); 3-(4-fluoro-5-(3-fluoro-4-(((R)-3-(2-hydroxypropan-2-yl)pyrrolidin-1-yl)methyl)pyridin-2-yl)-1-oxoisoindolin-2-yl)piperidine-2,6-dione (278); 3-(4-fluoro-5-(3-fluoro-4-(((S)-3-(2-hydroxypropan-2-yl)pyrrolidin-1-yl)methyl)pyridin-2-yl)-1-oxoisoindolin-2-yl)piperidine-2,6-dione (279); 3-(4-fluoro-5-(3-fluoro-4-((4-hydroxy-4-phenylpiperidin-1-yl)methyl)pyridin-2-yl)-1-oxoisoindolin-2-yl)piperidine-2,6-dione (280); 3-(4-fluoro-5-(3-fluoro-4-((3-(2-hydroxypropan-2-yl)azetidin-1-yl)methyl)pyridin-2-yl)-1-oxoisoindolin-2-yl)piperidine-2,6-dione (281); 3-(4-fluoro-5-(3-fluoro-4-((3-(trifluoromethyl)-5,6-dihydro-[1,2,4]triazolo[4,3-a]pyrazin-7(8H)-yl)methyl)pyridin-2-yl)-1-oxoisoindolin-2-yl)piperidine-2,6-dione (282); 3-(5-(4-((5-chloro-3,4-dihydro-2,6-naphthyridin-2(1H)-yl)methyl)-3-fluoropyridin-2-yl)-4-fluoro-1-oxoisoindolin-2-yl) piperidine-2,6-dione (283); 3-(5-(4-((6-acetyl-2,6-diazaspiro[3.3]heptan-2-yl)methyl)-3-fluoropyridin-2-yl)-4-fluoro-1-oxoisoindolin-2-yl)piperidine-2,6-dione (284); 3-(5-(6-amino-5-((8-benzoyl-3,8-diazabicyclo[3.2.1]octan-3-yl)methyl)pyridin-2-yl)-1-oxoisoindolin-2-yl)piperidine-2,6-dione (285); 3-(5-(5-((2-azaspiro[3.3]heptan-2-yl) methyl)-6-aminopyridin-2-yl)-1-oxoisoindo-lin-2-yl) piperidine-2,6-dione (286); 3-(5-(3,5-difluoro-4-((4-(2-hydroxypropan-2-yl)piperidin-1-yl)methyl)pyridin-2-yl)-1-oxoisoindolin-2-yl)piperidine-2,6-dione (287); 3-(5-(3,5-difluoro-4-((3-(methoxymethyl) azetidin-1-yl)methyl)pyridin-2-yl)-4-fluoro-1-oxoisoindolin-2-yl)piperidine-2,6-dione (288); or 3-(5-(4-((2-azaspiro[3.3]heptan-2-yl)methyl)-3,5-difluoropyridin-2-yl)-4-fluoro-1-oxoisoindolin-2-yl)piperidine-2,6-dione (289).

The present invention may be embodied in other specific forms without departing from the spirit or essential attributes thereof. This invention encompasses all combinations of the aspects and/or embodiments of the invention noted herein. It is understood that any and all embodiments of the present invention may be taken in conjunction with any other embodiment or embodiments to describe additional embodiments. It is also to be understood that each individual element of the embodiments is meant to be combined with any and all other elements from any embodiment to describe an additional embodiment.

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof. Embodiments identified herein as exemplary or preferred are intended to be illustrative and not limiting.

Unless specifically stated otherwise herein, references made in the singular may also include the plural. For example, “a” and “an” may refer to either one, or one or more.

As used herein, the phrase “compounds and/or salts thereof” refers to at least one compound, at least one salt of the compounds, or a combination thereof. For example, compounds of Formula (I) and/or salts thereof includes a compound of Formula (I); two compounds of Formula (I); a salt of a compound of Formula (I); a compound of Formula (I) and one or more salts of the compound of Formula (I); and two or more salts of a compound of Formula (I).

Unless otherwise indicated, any atom with unsatisfied valences is assumed to have hydrogen atoms sufficient to satisfy the valences.

The definitions set forth herein take precedence over definitions set forth in any patent, patent application, and/or patent application publication incorporated herein by reference.

Listed below are definitions of various terms used to describe the present invention. These definitions apply to the terms as they are used throughout the specification (unless they are otherwise limited in specific instances) either individually or as part of a larger group.

Throughout the specification, groups and substituents thereof may be chosen by one skilled in the field to provide stable moieties and compounds.

In accordance with a convention used in the art,

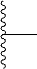

is used in structural formulas herein to depict the bond that is the point of attachment of the moiety or substituent to the core or backbone structure.

The terms “halo” and “halogen,” as used herein, refer to F, Cl, Br, and I.

The term “cyano” refers to the group —CN.

The term “amino” refers to the group —NH₂.

The term “oxo” refers to the group ═O.

The term “alkyl” as used herein, refers to both branched and straight-chain saturated aliphatic hydrocarbon groups containing, for example, from 1 to 12 carbon atoms, from 1 to 6 carbon atoms, and from 1 to 4 carbon atoms. Examples of alkyl groups include, but are not limited to, methyl (Me), ethyl (Et), propyl (e.g., n-propyl and i-propyl), butyl (e.g., n-butyl, i-butyl, sec-butyl, and t-butyl), and pentyl (e.g., n-pentyl, isopentyl, neopentyl), n-hexyl, 2-methylpentyl, 2-ethylbutyl, 3-methylpentyl, and 4-methylpentyl. When numbers appear in a subscript after the symbol “C”, the subscript defines with more specificity the number of carbon atoms that a particular group may contain. For example, “C₁₋₄ alkyl” denotes straight and branched chain alkyl groups with one to four carbon atoms.

The term “fluoroalkyl” as used herein is intended to include both branched and straight-chain saturated aliphatic hydrocarbon groups substituted with one or more fluorine atoms. For example, “C₁₋₄ fluoroalkyl” is intended to include C₁, C₂, C₃, and C₄ alkyl groups substituted with one or more fluorine atoms. Representative examples of fluoroalkyl groups include, but are not limited to, —CF₃ and —CH₂CF₃.

The term “alkoxy,” as used herein, refers to an alkyl group attached to the parent molecular moiety through an oxygen atom, for example, methoxy group (—OCH₃). For example, “C₁₋₃ alkoxy” denotes alkoxy groups with one to three carbon atoms.

The terms “fluoroalkoxy” and “—O(fluoroalkyl)” represent a fluoroalkyl group as defined above attached through an oxygen linkage (—O—). For example, “C₁₋₄ fluoroalkoxy” is intended to include C₁, C₂, C₃, and C₄ fluoroalkoxy groups.

The term “cycloalkyl,” as used herein, refers to a group derived from a non-aromatic monocyclic or polycyclic hydrocarbon molecule by removal of one hydrogen atom from a saturated ring carbon atom. Representative examples of cycloalkyl groups include, but are not limited to, cyclopropyl, cyclopentyl, and cyclohexyl. When numbers appear in a subscript after the symbol “C”, the subscript defines with more specificity the number of carbon atoms that a particular cycloalkyl group may contain. For example, “C₃-C₆ cycloalkyl” denotes cycloalkyl groups with three to six carbon atoms.

The compounds of the present invention include all isotopes of atoms occurring in the present compounds. Isotopes include those atoms having the same atomic number but different mass numbers. By way of general example and without limitation, isotopes of hydrogen include deuterium (D) and tritium (T). Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds of Formula (I) can form salts which are also within the scope of this invention. Unless otherwise indicated, reference to an inventive compound is understood to include reference to one or more salts thereof. The term “salt(s)” denotes acidic and/or basic salt(s) formed with inorganic and/or organic acids and bases. In addition, the term “salt(s) may include zwitterions (inner salts), e.g., when a compound of Formula (I) contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated within the scope of the invention. Salts of the compounds of the formula (I) may be formed, for example, by reacting a compound of the Formula (I) with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, maleates (formed with maleic acid), 2-hydroxyethanesulfonates, lactates, methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts; alkaline earth metal salts such as calcium and magnesium salts; barium, zinc, and aluminum salts; salts with organic bases (for example, organic amines) such as trialkylamines such as triethylamine, procaine, dibenzylamine, N-benzyl-β-phenethylamine, 1-ephenamine, N,N′-dibenzylethylene-diamine, dehydroabietylamine, N-ethylpiperidine, benzylamine, dicyclohexylamine or similar pharmaceutically acceptable amines and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

The compounds of Formula (I) can be provided as amorphous solids or crystalline solids. Lyophilization can be employed to provide the compounds of Formula (I) as a solid.

It should further be understood that solvates (e.g., hydrates) of the Compounds of Formula (I) are also within the scope of the present invention. The term “solvate” means a physical association of a compound of Formula (I) with one or more solvent molecules, whether organic or inorganic. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, isopropanolates, acetonitrile solvates, and ethyl acetate solvates. Methods of solvation are known in the art.

Various forms of prodrugs are well known in the art and are described in Rautio, J. et al., Nature Review DrugDiscovery, 17, 559-587 (2018).

In addition, compounds of Formula (I), subsequent to their preparation, can be isolated and purified to obtain a composition containing an amount by weight equal to or greater than 99% of a compound of Formula (I) (“substantially pure”), which is then used or formulated as described herein. Such “substantially pure” compounds of Formula (I) are also contemplated herein as part of the present invention.

“Stable compound” and “stable structure” are meant to indicate a compound that is sufficiently robust to survive isolation to a useful degree of purity from a reaction mixture, and formulation into an efficacious therapeutic agent. The present invention is intended to embody stable compounds.

The term “Helios inhibitor” refers to an agent capable of decreasing Helios protein levels, decreasing Helios activity level and/or inhibiting Helios expression level in the cells to control Treg differentiation. The Helios inhibitor may be a reversible or irreversible inhibitor.

As used herein, “Helios” protein refers a protein that is a member of the Ikaros family of zinc finger proteins. In humans, Helios is encoded by the IKZF2 gene. Helios is also known as IKAROS family zinc finger 2, ANF1A2, ZNF1A2, ZNFN1A2, zinc finger protein, subfamily 1A, 2, and Ikaros family zinc finger protein 2. The members of this protein family include Ikaros, Helios, Aiolos, Eos, and Pegasus. As used herein Helios protein includes various isoform, which includes the isoforms 1-5 listed below.

Isoform 1 (UniProt Q9UKS7-1)
(SEQ ID NO: 1)
METEAIDGYITCDNELSPEREHSNMAIDLTSSTPNGQHASPSHMTSTNSV
KLEMQSDEECDRKPLSREDEIRGHDEGSSLEEPLIESSEVADNRKVQELQ
GEGGIRLPNGKLKCDVCGMVCIGPNVLMVHKRSHTGERPFHCNQCGASFT
QKGNLLRHIKLHSGEKPFKCPFCSYACRRRDALTGHLRTHSVGKPHKCNY
CGRSYKQRSSLEEHKERCHNYLQNVSMEAAGQVMSHHVPPMEDCKEQEPI
MDNNISLVPFERPAVIEKLTGNMGKRKSSTPQKFVGEKLMRFSYPDIHFD
MNLTYEKEAELMQSHMMDQAINNAITYLGAEALHPLMQHPPSTIAEVAPV
ISSAYSQVYHPNRIERPISRETADSHENNMDGPISLIRPKSRPQEREASP
SNSCLDSTDSESSHDDHQSYQGHPALNPKRKQSPAYMKEDVKALDTTKAP
KGSLKDIYKVENGEGEQIRAFKCEHCRVLFLDHVMYTIHMGCHGYRDPLE
CNICGYRSQDRYEFSSHIVRGEHTFH
Isoform 2 (UniProt Q9UKS7-2)
(SEQ ID NO: 2)
METEAIDGYITCDNELSPEREHSNMAIDLTSSTPNGQHASPSHMTSTNSV
KLEMQSDEECDRKPLSREDEIRGHDEGSSLEEPLIESSEVADNRKVQELQ
GEGGIRLPNGERPFHCNQCGASFTQKGNLLRHIKLHSGEKPFKCPFCSYA
CRRRDALTGHLRTHSVGKPHKCNYCGRSYKQRSSLEEHKERCHNYLQNVS
MEAAGQVMSHHVPPMEDCKEQEPIMDNNISLVPFERPAVIEKLTGNMGKR
KSSTPQKFVGEKLMRFSYPDIHEDMNLTYEKEAELMQSHMMDQAINNAIT
YLGAEALHPLMQHPPSTIAEVAPVISSAYSQVYHPNRIERPISRETADSH
ENNMDGPISLIRPKSRPQEREASPSNSCLDSTDSESSHDDHQSYQGHPAL
NPKRKQSPAYMKEDVKALDTTKAPKGSLKDIYKVENGEGEQIRAFKCEHC
RVLFLDHVMYTIHMGCHGYRDPLECNICGYRSQDRYEFSSHIVRGEHTFH
Isoform 4 (UniProt Q9UKS7-4)
(SEQ ID NO: 3)
METEAIDGYITCDNELSPEREHSNMAIDLTSSTPNGQHASPSHMTSTNSV
KLEMQSDEECDRKPLSREDEIRGHDEGSSLEEPLIESSEVADNRKVQELQ
GEGGIRLPNGERPFHCNQCGASFTQKGNLLRHIKLHSGEKPFKCPFCSYA
CRRRDALTGHLRTHSVGKPHKCNYCGRSYKQRSSLEEHKERCHNYLQNVS
MEAAGQVMSHHGEKLMRFSYPDIHFDMNLTYEKEAELMQSHMMDQAINNA
ITYLGAEALHPLMQHPPSTIAEVAPVISSAYSQVYHPNRIERPISRETAD
SHENNMDGPISLIRPKSRPQEREASPSNSCLDSTDSESSHDDHQSYQGHP
ALNPKRKQSPAYMKEDVKALDTTKAPKGSLKDIYKVFNGEGEQIRAFKCE
HCRVLFLDHVMYTIHMGCHGYRDPLECNICGYRSQDRYEFSSHIVRGEHT
FH
Isoform 6 (UniProt Q9UKS7-6)
(SEQ ID NO: 4)
METEAIDGYITCDNELSPEREHSNMAIDLTSSTPNGQHASPSHMTSTNSV
KLEMQSDEECDRKPLSREDEIRGHDEGSSLEEPLIESSEVADNRKVQELQ
GEGGIRLPNGKLKCDVCGMVCIGPNVLMVHKRSHTGERPFHCNQCGASFT
QKGNLLRHIKLHSGEKPFKCPFCSYACRRRDALTGHLRTHSVGKPHKCNY
CGRSYKQRSSLEEHKERCHNYLQNVSMEAAGQVMSHHDS
Isoform 7 (UniProt Q9UKS7-7)
(SEQ ID NO: 5)
METEAIDGYITCDNELSPEREHSNMAIDLTSSTPNGQHASPSHMTSTNSV
KLEMQSDEECDRKPLSREDEIRGHDEGSSLEEPLIESSEVADNRKVQELQ
GEGGIRLPNGERPFHCNQCGASFTQKGNLLRHIKLHSGEKPFKCPFCSYA
CRRRDALTGHLRTHSVPPMEDCKEQEPIMDNNISLVPFERPAVIEKLTGN
MGKRKSSTPQKFVGEKLMRFSYPDIHFDMNLTYEKEAELMQSHMMDQAIN
NAITYLGAEALHPLMQHPPSTIAEVAPVISSAYSQVYHPNRIERPISRET
ADSHENNMDGPISLIRPKSRPQEREASPSNSCLDSTDSESSHDDHQSYQG
HPALNPKRKQSPAYMKEDVKALDTTKAPKGSLKDIYKVFNGEGEQIRAFK
CEHCRVLFLDHVMYTIHMGCHGYRDPLECNICGYRSQDRYEFSSHIVRGE
HTFH

The “Helios” isoforms 1, 2, 4, 6, and 7 listed above includes the degron FHCNQCGASFTQKGNLLRHIKLH (SEQ ID NO: 6)(bold and underlined). A degron is a portion of a protein that plays a role in regulating protein degradation rates.

As used herein, “Eos” protein is encoded by the IKZF4 gene, and is also known as IKAROS family zinc finger 4, ZNFN1A4, zinc finger protein, subfamily 1A, 4, Ikaros family zinc finger protein 4, and KIAA1782. “Eos” protein includes isoforms encoded by the following two human isoforms 1 (Q9H2S9-1) and 2 (Q9H2S9-2):

Isoform 1 (UniProt Q9H2S9-1)
(SEQ ID NO: 7)
MHTPPALPRRFQGGGRVRTPGSHRQGKDNLERDPSGGCVPDFLPQAQDSN
HFIMESLFCESSGDSSLEKEFLGAPVGPSVSTPNSQHSSPSRSLSANSIK
VEMYSDEESSRLLGPDERLLEKDDSVIVEDSLSEPLGYCDGSGPEPHSPG
GIRLPNGKLKCDVCGMVCIGPNVLMVHKRSHTGERPFHCNQCGASFTQKG
NLLRHIKLHSGEKPFKCPFCNYACRRRDALTGHLRTHSVSSPTVGKPYKC
NYCGRSYKQQSTLEEHKERCHNYLQSLSTEAQALAGQPGDEIRDLEMVPD
SMLHSSSERPTFIDRLANSLTKRKRSTPQKFVGEKQMRFSLSDLPYDVNS
GGYEKDVELVAHHSLEPGFGSSLAFVGAEHLRPLRLPPTNCISELTPVIS
SVYTQMQPLPGRLELPGSREAGEGPEDLADGGPLLYRPRGPLTDPGASPS
NGCQDSTDTESNHEDRVAGVVSLPQGPPPQPPPTIVVGRHSPAYAKEDPK
PQEGLLRGTPGPSKEVLRVVGESGEPVKAFKCEHCRILFLDHVMFTIHMG
CHGFRDPFECNICGYHSQDRYEFSSHIVRGEHKVG
Isoform 2 (UniProt Q9H2S9-2)
(SEQ ID NO: 8)
MDSRYLQLQLYLPSCSLLQGSGDSSLEKEFLGAPVGPSVSTPNSQHSSPS
RSLSANSIKVEMYSDEESSRLLGPDERLLEKDDSVIVEDSLSEPLGYCDG
SGPEPHSPGGIRLPNGKLKCDVCGMVCIGPNVLMVHKRSHTGERPFHCNQ
CGASFTQKGNLLRHIKLHSGEKPFKCPFCNYACRRRDALTGHLRTHSVSS
PTVGKPYKCNYCGRSYKQQSTLEEHKERCHNYLQSLSTEAQALAGQPGDE
IRDLEMVPDSMLHSSSERPTFIDRLANSLTKRKRSTPQKFVGEKQMRFSL
SDLPYDVNSGGYEKDVELVAHHSLEPGFGSSLAFVGAEHLRPLRLPPTNC
ISELTPVISSVYTQMQPLPGRLELPGSREAGEGPEDLADGGPLLYRPRGP
LTDPGASPSNGCQDSTDTESNHEDRVAGVVSLPQGPPPQPPPTIVVGRHS
PAYAKEDPKPQEGLLRGTPGPSKEVLRVVGESGEPVKAFKCEHCRILFLD
HVMFTIHMGCHGERDPFECNICGYHSQDRYEFSSHIVRGEHKVG

The “Eos” protein isoforms 1 and 2 listed above includes the degron FHCNQCGASFTQKGNLLRHIKLH (SEQ ID NO: 6) (bold and underlined), which is the same as the degron for the “Helios” protein.

As used herein, “Ikaros” protein is encoded by the IKZF1 gene. Ikaros is also known as IKAROS family zinc finger 1, ZNFN1A1, zinc finger protein, subfamily 1A, 1, Ikaros family zinc finger protein 1, IK1, lymphoid transcription factor LyF-1, Hs.54452, PPP1R92, protein phosphatase 1, regulatory subunit 92, PR00758, CVID13, and CLL-associated antigen KW-6. Ikaros protein includes isoforms encoded by amino acid sequences Q13422-1, Q13422-2, Q13422-3, Q13422-4, Q13422-7, and Q13422-8. Ikaros protein also includes isoforms encoded by amino acid sequences Q13422-5 and Q13422-6.

As used herein, “Aiolos” protein is encoded by the IKZF3 gene. Aiolos protein is also known as IKAROS family zinc finger 3, ZNFN1A3, zinc finger protein, subfamily 1A, 3, Ikaros family zinc finger protein 3, and AIO. Aiolos protein includes isoforms encoded by amino acid sequences Q9UKT9-1, Q9UKT9-3, Q9UKT9-4, Q9UKT9-6, Q9UKT9-7, Q9UKT9-8, Q9UKT9-9, and Q9UKT9-14. Aiolos protein also includes isoforms encoded by amino acid sequences Q9UKT9-2, Q9UKT9-5, Q9UKT9-10, Q9UKT9-11, Q9UKT9-12, and Q9UKT9-13, Q9UKT9-15, and Q9UKT9-16.

As used herein, “Pegasus” protein is also known as IKAROS family zinc finger 5, ZNFN1A5, zinc finger protein, subfamily 1A, 5, and Ikaros family zinc finger protein 5. Pegasus is encoded by the IKZF5 gene.

As used herein, the term “contacting” refers to the bringing together of indicated moieties in an in vitro system or an in vivo system. For example, “contacting” Helios protein with a compound of Formula (I) includes the administration of a compound of the present invention to an individual or patient, such as a human, having Helios protein, as well as, for example, introducing a compound of Formula (I) into a sample containing a cellular or purified preparation containing Helios protein.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease. In contrast, “prophylaxis” or “prevention” refers to administration to a subject who does not have a disease to prevent the disease from occurring. “Treat,” “treating,” and “treatment” does not encompass prophylaxis or prevention.

“Therapeutically effective amount” is intended to include an amount of a compound of the present invention alone or an amount of the combination of compounds claimed or an amount of a compound of the present invention in combination with other active ingredients effective to decrease Helios protein levels, decrease Helios activity levels and/or inhibit Helios expression levels in the cells, or effective to treat or prevent viral infections and proliferative disorders, such as cancer.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

The term “patient” includes human and other mammalian subjects that receive either therapeutic or prophylactic treatment.

The term “subject” includes any human or non-human animal. For example, the methods and compositions herein disclosed can be used to treat a subject having cancer.

A non-human animal includes all vertebrates, e.g., mammals and non-mammals, including non-human primates, sheep, dogs, cows, chickens, amphibians, reptiles, etc. In one embodiment, the subject is a human subject.

The phrase “pharmaceutically acceptable carrier” as used herein means a pharmaceutically acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation, including, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms; and not injurious to the patient.

The term “pharmaceutical composition” means a composition comprising a compound of the invention in combination with at least one additional pharmaceutically acceptable carrier.

### Utility

The compounds of Formula (I) are useful for the treatment of cancer.

In one embodiment, the present invention provides a combined preparation of a compound of Formula (I), and/or a pharmaceutically acceptable salt thereof, a stereoisomer thereof or a tautomer thereof, and additional therapeutic agent(s) for simultaneous, separate or sequential use in the treatment and/or prophylaxis of multiple diseases or disorders associated with the activity of Helios protein. The combined preparation can be used to decrease Helios protein level, Helios activity level and/or Helios expression level in the cells to control Treg differentiation.

The compounds for Formula (I) and pharmaceutical compositions comprising at least one compound of Formula (I) are useful in treating or preventing any diseases or conditions that are associated with the activity of Helios protein. These include viral and other infections (e.g., skin infections, GI infection, urinary tract infections, genito-urinary infections, systemic infections), and proliferative diseases (e.g., cancer). The compounds of Formula (I) and pharmaceutical compositions comprising in at least one compound of Formula (I) may be administered to animals, preferably mammals (e.g., domesticated animals, cats, dogs, mice, rats), and more preferably humans. Any method of administration may be used to deliver the compound or pharmaceutical composition to the patient. In certain embodiments, the compound of Formula (I) or pharmaceutical composition comprising at least compound of Formula (I) is administered orally. In other embodiments, the Formula (I) or pharmaceutical composition comprising at least compound of Formula (I) is administered parenterally.

The compounds of Formula (I) can selectively decrease Helios protein levels, decrease Helios activity levels and/or inhibit Helios expression levels in the cells to control Treg differentiation. For example, the compounds of Formula (I) can be used to selectively decrease Helios activity levels and/or inhibit Helios expression levels in the cells to control Treg differentiation in a cell or in an individual in need of a decrease in Helios protein levels, decrease in Helios activity levels and/or inhibition of Helios expression level by administering an inhibiting amount of a compound of Formula (I) or a salt thereof.

In one aspect, the compound(s) of Formula (I) are sequentially administered prior to administration of the immuno-oncology agent. In another aspect, compound(s) of Formula (I) are administered concurrently with the immuno-oncology agent. In yet another aspect, compound(s) of Formula (I) are sequentially administered after administration of the immuno-oncology agent.

In another aspect, compounds of Formula (I) may be co-formulated with an immuno-oncology agent.

Immuno-oncology agents include, for example, a small molecule drug, antibody, or other biologic or small molecule. Examples of biologic immuno-oncology agents include, but are not limited to, cancer vaccines, antibodies, and cytokines. In one aspect, the antibody is a monoclonal antibody. In another aspect, the monoclonal antibody is humanized or human.

In one aspect, the immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators).

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fni4, TWEAK, BAFFR, EDAR, XEDAR, TACI, APRIL, BCMA, LTOR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α 1β2, FAS, FASL, RELT, DR6, TROY, NGFR.

In one aspect, T cell responses can be stimulated by a combination of a compound of Formula (I) and one or more of (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

Other agents that can be combined with compounds of Formula (I) for the treatment of cancer include antagonists of inhibitory receptors on NK cells or agonists of activating receptors on NK cells. For example, compounds of Formula (I) can be combined with antagonists of KIR, such as lirilumab.

Yet other agents for combination therapies include agents that inhibit or deplete macrophages or monocytes, including but not limited to CSF-1R antagonists such as CSF-1R antagonist antibodies including RG7155 (WO11/70024, WO11/107553, WO 11/131407, WO13/87699, WO13/119716, WO13/132044) or FPA-008 (WO11/140249; WO13169264; WO14/036357).

In another aspect, compounds of Formula (I) can be used with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites.

In one aspect, the immuno-oncology agent is a CTLA-4 antagonist, such as an antagonistic CTLA-4 antibody. Suitable CTLA-4 antibodies include, for example, YERVOY (ipilimumab) or tremelimumab.

In another aspect, the immuno-oncology agent is a PD-1 antagonist, such as an antagonistic PD-1 antibody. Suitable PD-1 antibodies include, for example, OPDIVO (nivolumab), KEYTRUDA (pembrolizumab), or MEDI-0680 (AMP-514; WO2012/145493). The immuno-oncology agent may also include pidilizumab (CT-011), though its specificity for PD-1 binding has been questioned. Another approach to target the PD-1 receptor is the recombinant protein composed of the extracellular domain of PD-L2 (B7-DC) fused to the Fc portion of IgG1, called AMP-224.

In another aspect, the immuno-oncology agent is a PD-L1 antagonist, such as an antagonistic PD-L1 antibody. Suitable PD-L1 antibodies include, for example, MPDL3280A (RG7446; WO2010/077634), durvalumab (MEDI4736), BMS-936559 (WO207/005874), and MSB0010718C (WO2013/79174).

In another aspect, the immuno-oncology agent is a LAG-3 antagonist, such as an antagonistic LAG-3 antibody. Suitable LAG3 antibodies include, for example, BMS-986016 (WO10/19570, WO14/08218), or IMP-731 or IMP-321 (WO08/132601, WO09/44273).

In another aspect, the immuno-oncology agent is a CD137 (4-1BB) agonist, such as an agonistic CD137 antibody. Suitable CD137 antibodies include, for example, urelumab and PF-05082566 (WO12/32433).

In another aspect, the immuno-oncology agent is a GITR agonist, such as an agonistic GITR antibody. Suitable GITR antibodies include, for example, BMS-986153, BMS-986156, TRX-518 (WO06/105021, WO09/009116) and MK-4166 (WO11/028683).

In another aspect, the immuno-oncology agent is an IDO antagonist. Suitable IDO antagonists include, for example, INCB-024360 (WO206/122150, WO07/75598, WO08/36653, WO08/36642), indoximod, or NLG-919 (WO09/73620, WO09/1156652, WO11/56652, WO12/142237).

In another aspect, the immuno-oncology agent is an OX40 agonist, such as an agonistic OX40 antibody. Suitable OX40 antibodies include, for example, MEDI-6383 or MEDI-6469.

In another aspect, the immuno-oncology agent is an OX40L antagonist, such as an antagonistic OX40 antibody. Suitable OX40L antagonists include, for example, RG-7888 (WO06/029879).

In another aspect, the immuno-oncology agent is a CD40 agonist, such as an agonistic CD40 antibody. In yet another embodiment, the immuno-oncology agent is a CD40 antagonist, such as an antagonistic CD40 antibody. Suitable CD40 antibodies include, for example, lucatumumab or dacetuzumab.

In another aspect, the immuno-oncology agent is a CD27 agonist, such as an agonistic CD27 antibody. Suitable CD27 antibodies include, for example, varlilumab.

In another aspect, the immuno-oncology agent is MGA271 (to B7H3) (WO11/109400).

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Combination therapy also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Types of cancers that may be treated with the compound of Formula (I) include, but are not limited to, brain cancers, skin cancers, bladder cancers, ovarian cancers, breast cancers, gastric cancers, pancreatic cancers, prostate cancers, colon cancers, blood cancers, lung cancers and bone cancers. Examples of such cancer types include neuroblastoma, intestine carcinoma such as rectum carcinoma, colon carcinoma, familiar adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroid carcinoma, papillary thyroid carcinoma, renal carcinoma, kidney parenchymal carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, diffuse large B-cell lymphoma (DLBCL), hepatocellular carcinoma, gall bladder carcinoma, bronchial carcinoma, small cell lung carcinoma, non-small cell lung carcinoma, multiple myeloma, basalioma, teratoma, retinoblastoma, choroid melanoma, seminoma, rhabdomyosarcoma, craniopharyngioma, osteosarcoma, chondrosarcoma, myosarcoma, liposarcoma, fibrosarcoma, Ewing sarcoma and plasmocytoma.

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2 and GM-CSF), and/or tyrosine kinase inhibitors can be optionally used in combination with the compounds of Formula (I) for treatment of Helios protein associated diseases, disorders or conditions. The agents can be combined with the present compounds in a single dosage form, or the agents can be administered simultaneously or sequentially as separate dosage forms.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (CYTOXAN®), ifosfamide, melphalan, chlorambucil, pipobroman, triethylene-melamine, triethylenethiophosphoramine, busulfan, carmustine, lomustine, streptozocin, dacarbazine, and temozolomide.

In the treatment of melanoma, suitable agents for use in combination with the compounds of Formula (I) include: dacarbazine (DTIC), optionally, along with other chemotherapy drugs such as carmustine (BCNU) and cisplatin; the “Dartmouth regimen”, which consists of DTIC, BCNU, cisplatin and tamoxifen; a combination of cisplatin, vinblastine, and DTIC, temozolomide or YERVOY™. Compounds of Formula (I) may also be combined with immunotherapy drugs, including cytokines such as interferon alpha, interleukin 2, and tumor necrosis factor (TNF) in the treatment of melanoma.

Compounds of Formula (I) may also be used in combination with vaccine therapy in the treatment of melanoma. Antimelanoma vaccines are, in some ways, similar to the anti-virus vaccines which are used to prevent diseases caused by viruses such as polio, measles, and mumps. Weakened melanoma cells or parts of melanoma cells called antigens may be injected into a patient to stimulate the body's immune system to destroy melanoma cells.

Melanomas that are confined to the arms or legs may also be treated with a combination of agents including one or more compounds of Formula (I), using a hyperthermic isolated limb perfusion technique. This treatment protocol temporarily separates the circulation of the involved limb from the rest of the body and injects high doses of chemotherapy into the artery feeding the limb, thus providing high doses to the area of the tumor without exposing internal organs to these doses that might otherwise cause severe side effects. Usually the fluid is warmed to 38.9° C. to 40° C. Melphalan is the drug most often used in this chemotherapy procedure. This can be given with another agent called tumor necrosis factor (TNF).

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (Taxol), mithramycin, deoxyco-formycin, mitomycin-C, L-asparaginase, interferons (especially IFN-α), etoposide, and teniposide.

Other cytotoxic agents include navelbene, CPT-11, anastrazole, letrazole, capecitabine, reloxafine, and droloxafine.

Also suitable are cytotoxic agents such as epidophyllotoxin; an antineoplastic enzyme; a topoisomerase inhibitor; procarbazine; mitoxantrone; platinum coordination complexes such as cisplatin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (HERCEPTIN®), antibodies to costimulatory molecules such as CTLA-4, 4-1BB and PD-1, or antibodies to cytokines (IL-10 or TGF-β).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses.

The pharmaceutical composition of the invention may optionally include at least one signal transduction inhibitor (STI). A “signal transduction inhibitor” is an agent that selectively inhibits one or more vital steps in signaling pathways, in the normal function of cancer cells, thereby leading to apoptosis. Suitable STIs include, but are not limited to: (i) bcr/abl kinase inhibitors such as, for example, STI 571 (GLEEVEC®); (ii) epidermal growth factor (EGF) receptor inhibitors such as, for example, kinase inhibitors (IRESSA®, SSI-774) and antibodies (Imclone: C225 [Goldstein et al., Clin. Cancer Res., 1:1311-1318 (1995)], and Abgenix: ABX-EGF); (iii) her-2/neu receptor inhibitors such as farnesyl transferase inhibitors (FTI) such as, for example, L-744,832 (Kohl et al., Nat. Med., 1(8):792-797 (1995)); (iv) inhibitors of Akt family kinases or the Akt pathway, such as, for example, rapamycin (see, for example, Sekulic et al., Cancer Res., 60:3504-3513 (200)); (v) cell cycle kinase inhibitors such as, for example, flavopiridol and UCN-01 (see, for example, Sausville, Curr. Med. Chem. Anti-Canc. Agents, 3:47-56 (203)); and (vi) phosphatidyl inositol kinase inhibitors such as, for example, LY294002 (see, for example, Vlahos et al., J. Biol. Chem., 269:5241-5248 (1994)). Alternatively, at least one STI and at least one compound of Formula (I) may be in separate pharmaceutical compositions. In a specific embodiment of the present invention, at least one compound of Formula (I) and at least one STI may be administered to the patient concurrently or sequentially. In other words, at least one compound of Formula (I) may be administered first, at least one STI may be administered first, or at least one compound of Formula (I) and at least one STI may be administered at the same time. Additionally, when more than one compound of Formula (I) and/or STI is used, the compounds may be administered in any order.

The present invention further provides a pharmaceutical composition for the treatment of a chronic viral infection in a patient comprising at least one compound of Formula (I), optionally, at least one chemotherapeutic drug, and, optionally, at least one antiviral agent, in a pharmaceutically acceptable carrier.

Also provided is a method for treating a chronic viral infection in a patient by administering an effective amount of the above pharmaceutical composition.

In a specific embodiment of the present invention, at least one compound of Formula (I) and at least one chemotherapeutic agent are administered to the patient concurrently or sequentially. In other words, at least one compound of Formula (I) may be administered first, at least one chemotherapeutic agent may be administered first, or at least one compound of Formula (I) and the at least one STI may be administered at the same time. Additionally, when more than one compound of Formula (I) and/or chemotherapeutic agent is used, the compounds may be administered in any order. Similarly, any antiviral agent or STI may also be administered at any point in comparison to the administration of the compound of Formula (I).

Chronic viral infections that may be treated using the present combinatorial treatment include, but are not limited to, diseases caused by: hepatitis C virus (HCV), human papilloma virus (HPV), cytomegalovirus (CMV), herpes simplex virus (HSV), Epstein-Barr virus (EBV), varicella zoster virus, coxsackie virus, human immunodeficiency virus (HIV).

Suitable antiviral agents contemplated for use in combination with the compound of Formula (I) can comprise nucleoside and nucleotide reverse transcriptase inhibitors (NRTIs), non-nucleoside reverse transcriptase inhibitors (NNRTIs), protease inhibitors and other antiviral drugs.

Examples of suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH—I0652; emitricitabine [(−)-FTC]; beta-L-FD4 (also called beta-L-D4C and named beta-L-2′,3′-dicleoxy-5-fluoro-cytidene); DAPD, ((−)-beta-D-2,6-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1549. Other antiviral agents include hydroxyurea, ribavirin, IL-2, IL-12, pentafuside and Yissum Project No. 11607.

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Combination therapy also can embrace the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

### Pharmaceutical Compositions

The invention also provides pharmaceutically compositions which comprise a therapeutically effective amount of one or more of the compounds of Formula (I), formulated together with one or more pharmaceutically acceptable carriers (additives) and/or diluents, and optionally, one or more additional therapeutic agents described above.

The compounds of Formula (I) may be administered by any suitable route, preferably in the form of a pharmaceutical composition adapted to such a route, and in a dose effective for the treatment intended. The compounds and compositions of the compound of Formula (I) can be administered for any of the uses described herein by any suitable means, for example, orally, such as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories. They can be administered alone, but generally will be administered with a pharmaceutical carrier selected on the basis of the chosen route of administration and standard pharmaceutical practice.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg, preferably from about 0.25 to 250 mg, and more preferably from about 0.5 to 100 mg. A suitable daily dose for a human or other mammal may vary widely depending on the condition of the patient and other factors, but, can be determined using routine methods.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the invention can contain at least one agent selected from sweetening agents, flavoring agents, coloring agents, demulcents, antioxidants, and preserving agents.

A tablet can, for example, be prepared by admixing at least one compound of Formula (I) and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can, for example, be prepared by mixing at least one compound of Formula (I) and/or at least one salt thereof with at least one inert solid diluent, such as, for example, calcium carbonate; calcium phosphate; and kaolin.

Soft gelatin capsules can, for example, be prepared by mixing at least one compound of Formula (I) and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of Formula (I) and/or at least one pharmaceutically acceptable salt thereof with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) and/or at least one pharmaceutically acceptable salt thereof in either a vegetable oil, such as, for example, arachis oil; olive oil; sesame oil; and coconut oil; or in mineral oil, such as, for example, liquid paraffin. An oily suspension can also contain at least one thickening agent, such as, for example, beeswax; hard paraffin; and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of Formula (I) and/or at least one pharmaceutically acceptable salt thereof with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound of Formula (I) and/or at least one pharmaceutically acceptable salt thereof can, for example, be prepared as an oil-in-water emulsion. The oily phase of the emulsions comprising compounds of Formula (I) may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The compounds of Formula (I) and/or at least one pharmaceutically acceptable salt thereof can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions; and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound of Formula (I) in an oily phase, such as, for example, a mixture of soybean oil and lecithin; 2) combining the Formula (I) containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Allen, L. V. Jr. et al. Remington: The Science and Practice of Pharmacy (2 Volumes), 22nd Edition (2012), Pharmaceutical Press.

Pharmaceutically acceptable carriers, adjuvants, and vehicles that may be used in the pharmaceutical compositions of this invention include, but are not limited to, ion exchangers, alumina, aluminum stearate, lecithin, self-emulsifying drug delivery systems (SEDDS) such as d-alpha-tocopherol polyethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this invention can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

The amounts of compounds that are administered and the dosage regimen for treating a disease condition with the compounds and/or compositions of this invention depends on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.0025 and about 50 mg/kg body weight and most preferably between about 0.005 to 10 mg/kg body weight, may be appropriate. The daily dose can be administered in one to four doses per day. Other dosing schedules include one dose per week and one dose per two day cycle.

Pharmaceutical compositions of this invention comprise at least one compound of Formula (I) and/or at least one pharmaceutically acceptable salt thereof, and optionally an additional agent selected from any pharmaceutically acceptable carrier, adjuvant, and vehicle. Alternate compositions of this invention comprise a compound of the Formula (I) described herein, or a prodrug thereof, and a pharmaceutically acceptable carrier, adjuvant, or vehicle.

The present invention also includes pharmaceutical kits useful, for example, in the treatment or prevention of Helios protein-associated diseases or disorders, and other diseases referred to herein which include one or more containers containing a pharmaceutical composition comprising a therapeutically effective amount of a compound of Formula (I). Such kits can further include, if desired, one or more of various conventional pharmaceutical kit components, such as, for example, containers with one or more pharmaceutically acceptable carriers, additional containers, as will be readily apparent to those skilled in the art. Instructions, either as inserts or as labels, indicating quantities of the components to be administered, guidelines for administration, and/or guidelines for mixing the components, can also be included in the kit.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired.

By way of general guidance, the daily oral dosage of each active ingredient, when used for the indicated effects, will range between about 0.001 to about 5000 mg per day, preferably between about 0.01 to about 1000 mg per day, and most preferably between about 0.1 to about 250 mg per day. Intravenously, the most preferred doses will range from about 0.01 to about 10 mg/kg/minute during a constant rate infusion. Compounds of Formula (I) may be administered in a single daily dose, or the total daily dosage may be administered in divided doses of two, three, or four times daily.

The compounds are typically administered in admixture with suitable pharmaceutical diluents, excipients, or carriers (collectively referred to herein as pharmaceutical carriers) suitably selected with respect to the intended form of administration, e.g., oral tablets, capsules, elixirs, and syrups, and consistent with conventional pharmaceutical practices.

Dosage forms (pharmaceutical compositions) suitable for administration may contain from about 1 milligram to about 200 milligrams of active ingredient per dosage unit. In these pharmaceutical compositions the active ingredient will ordinarily be present in an amount of about 0.1-95% by weight based on the total weight of the composition.

A typical capsule for oral administration contains at least one of the compounds of Formula (I) (250 mg), lactose (75 mg), and magnesium stearate (15 mg). The mixture is passed through a 60 mesh sieve and packed into a No. 1 gelatin capsule.

A typical injectable preparation is produced by aseptically placing at least one of the compounds of Formula (I) (250 mg) into a vial, aseptically freeze-drying and sealing. For use, the contents of the vial are mixed with 2 mL of physiological saline, to produce an injectable preparation.

The present invention includes within its scope pharmaceutical compositions comprising, as an active ingredient, a therapeutically effective amount of at least one of the compounds of Formula (I), alone or in combination with a pharmaceutical carrier. Optionally, compounds of Formula (I) can be used alone, in combination with other compounds of Formula (I), or in combination with one or more other therapeutic agent(s), e.g., an anticancer agent or other pharmaceutically active material.

Regardless of the route of administration selected, the compounds of Formula (I), which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the therapeutic response for a particular patient, composition, and mode of administration, without being toxic to the patient.

The selected dosage level will depend upon a variety of factors including the activity of the particular compound of Formula (I) employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of Formula (I) employed in the pharmaceutical composition at levels lower than that required in order to achieve the therapeutic effect and gradually increase the dosage until the effect is achieved.

In general, a suitable daily dose of a compound of Formula (I) will be that amount of the compound which is the lowest dose effective to produce a therapeutic effect. Such an effective dose will generally depend upon the factors described above. Generally, oral, intravenous, intracerebroventricular and subcutaneous doses of the compounds of Formula (I) for a patient will range from about 0.01 to about 50 mg per kilogram of body weight per day.

If desired, the effective daily dose of the active compound may be administered as two, three, four, five, six or more sub-doses administered separately at appropriate intervals throughout the day, optionally, in unit dosage forms. In certain aspects of the invention, dosing is one administration per day.

While it is possible for a compound of Formula (I) to be administered alone, it is preferable to administer the compound as a pharmaceutical formulation (composition).

The above other therapeutic agents, when employed in combination with the compounds of Formula (I), may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds.

### Methods of Preparation

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Preferred methods include, but are not limited to, those described below. All references cited herein are hereby incorporated by reference in their entirety.

The compounds of this invention may be prepared using the reactions and techniques described in this section. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents that are compatible with the reaction conditions will be readily apparent to one skilled in the art and alternate methods must then be used. This will sometimes require a judgment to modify the order of the synthetic steps or to select one particular process scheme over another in order to obtain a compound of the invention. It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene and Wuts (Protective Groups In Organic Synthesis, Fourth Edition, Wiley and Sons, 207).

Compounds of Formula (I) may be prepared by reference to the methods illustrated in the following Schemes. As shown therein the end product is a compound having the same structural formula as Formula (I). It will be understood that any compound of Formula (I) may be produced by the schemes by the suitable selection of reagents with appropriate substitution. Solvents, temperatures, pressures, and other reaction conditions may readily be selected by one of ordinary skill in the art. Starting materials are commercially available or readily prepared by one of ordinary skill in the art. Constituents of compounds are as defined herein or elsewhere in the specification.

General routes to compounds described in the invention are illustrated in Schemes 1-2, where the R₁, R₂, R₄ and R₆ are defined previously in the text or a functional group that can be converted to the final substituent. The substituent X is a leaving group such as a halide (preferably I, Br, or Cl) or a triflate. The substituent M is a suitable coupling partner, such as boronic acid, boronic ester or stannane. The substituent R is a carboxylic acid protecting group such as tert-butyl, methyl, ethyl, or benzyl. As shown in Scheme 1, a general procedure for the preparation of compounds of the invention involves starting with a suitably substituted aryl fluoride 1. When treated with a suitable nucleophile, such as intermediate 2 where M can be MgBr or Li, substituted aryl halides such as 3 can be produced. Halogenation, preferably bromination, can be accomplished by treating 3 with reagents such as N-bromosuccinimide, to afford the bromide 4. Treatment of 4 with an amine 5 under basic conditions such as potassium carbonate, will result in initial displacement of the secondary bromide followed by cyclization to afford the lactam 6.

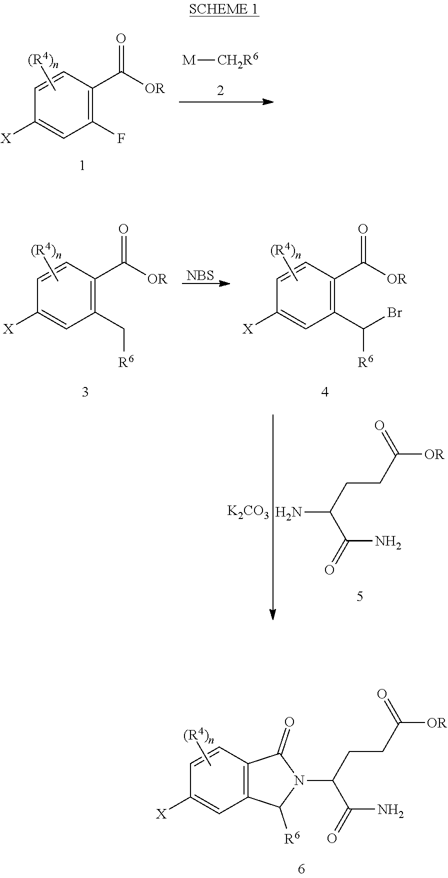

When M is a stannane, 6 can be united with a suitably substituted heterocycle 7 in a Stille coupling reaction using a suitable catalyst system (e.g. Pd(PPh₃)₄ or bis(triphenylphosphine)dichloropalladium(II)/CuI) to give 8. Alternatively, 6 can be converted to the boronic acid or boronic ester 9 by conditions well-known to one skilled in the art. The boronic acid or boronate ester, 9 can be united with a suitably substituted heterocycle 10 in a Suzuki-Miyaura coupling reaction using a suitable palladium catalyst (e.g. Pd(PPh₃)₄ or 1,1′-bis(diphenylphosphino)ferrocene]dichloropalladium(II)) in the presence of a suitable base (e.g. cesium carbonate, potassium phosphate, or sodium bicarbonate) to give 8.

Depending on the specific selection of acid protecting group R in intermediate 8, different conditions may be required to convert it into compound 11 (Scheme 2). For instance where R=methyl, ethyl, or benzyl, base-induced cyclization of 8 may be preferred for the direct conversion 8 to 11 using a suitable base (e.g. LiHMDS) in a suitable solvent (e.g. tetrahydrofuran). Where R=tert-butyl, acid-induced cyclization of 12 may be preferred for direct conversion of 8 to 11 using a suitable acid (e.g. benzenesulfonic acid) in a suitable solvent (e.g. acetonitrile). In some cases, it may be preferable to use a twostep procedure, first liberating free carboxylic acid corresponding to 8 using conditions which are appropriate to the specific acid protecting group R. Such methods are well known to one of ordinary skill in the art of organic synthesis. For instance where R=tert-butyl, acid hydrolysis using a suitable acid (e.g. trifluoroacetic acid or hydrochloric acid) may be preferred. Where R=methyl, ethyl, or benzyl, basic hydrolysis using a suitable base (e.g. LiOH) may be preferred. In other cases, where R=benzyl, it may be advantageous to deprotect by the action of palladium-catalyzed hydrogenolysis. Once liberated, the carboxylic acid can be activated toward intramolecular attack by the pendant primary amide by the action of thionyl chloride/dimethylformamide or carbonyldiimidazole/dimethylaminopyridine to afford 11.

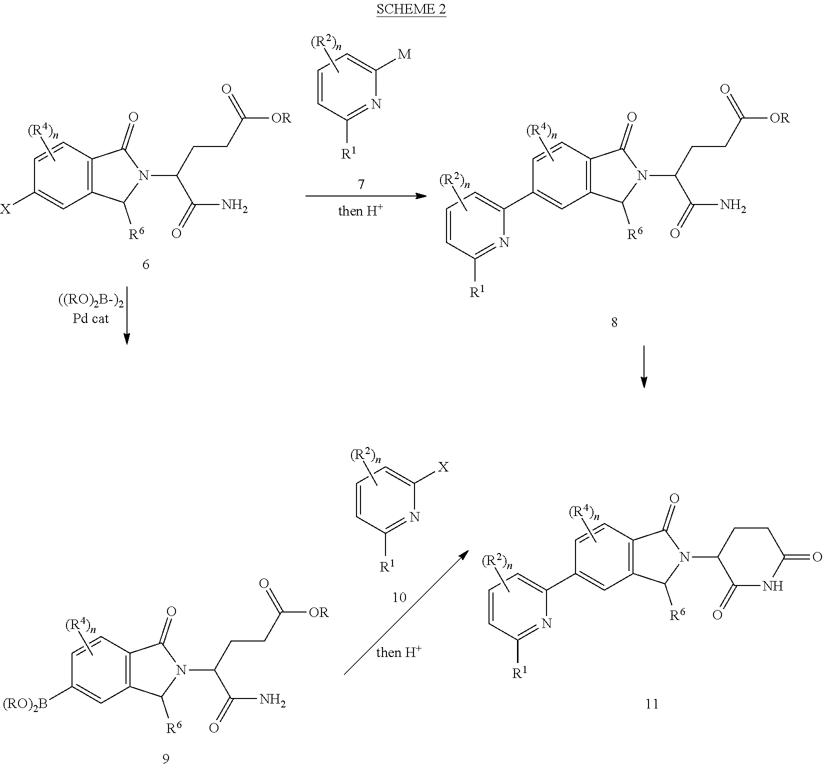

### EXAMPLES

The following examples illustrate the particular embodiments of the present invention and do not limit the scope of the present invention. Chemical abbreviations and symbols as well as scientific abbreviations and symbols have their usual and customary meanings unless otherwise specified. Additional abbreviations employed in the Examples and elsewhere in this application are defined above. Common intermediates are generally useful for the preparation of more than one Example. Compounds of the Examples are identified by the example and step in which they were prepared (e.g., “1-A” denotes the Example 1, step A), or by the example only where the compound is the title compound of the example (for example, “1” denotes the title compound of Example 1). In some instances alternate preparations of intermediates or examples are described. Frequently chemists skilled in the art of synthesis may devise alternative preparations which may be desirable based on one or more considerations such as shorter reaction time, less expensive starting materials, ease of operation or isolation, improved yield, amenable to catalysis, avoidance of toxic reagents, accessibility of specialized instrumentation, and decreased number of linear steps, etc. The intent of describing alternative preparations is to further enable the preparation of the examples of this invention. In some instances some functional groups in the outlined examples and claims may be replaced by well-known bioisosteric replacements known in the art, for example, replacement of a carboxylic acid group with a tetrazole or a phosphate moiety.

### Abbreviations

ACN acetonitrile
AcOH acetic acid
AIBN 2,2-azobisiosbutyronitrile
BisPin bis(pinacolato)diboron
n-BuLi n-butyl lithium
DCE dichloroethane
DCM dichloromethane
DIPEA N,N-diisopropylethylamine
DME dimethyl ether
DMF dimethylformamide
DMSO dimethyl sulfoxide
dppf bis(diphenylphosphino)ferrocene
EtOH ethanol
EtOAc ethyl acetate
HATU O-(7-azabenzotriazol-1-yl)-N, N, N′, N′-tetramethyluronium hexafluorophosphate
Hex hexanes
H-Glu(OtBu)-NH₂ HCl tert-butyl (4S)-4,5-diamino-5-oxopentanoate hydrochloride
HPLC High Performance Liquid Chromatography
Hunig's base N,N-diisopropylethylamine
LiHMDS lithium bis(trimethylsilyl)amide
MeCN acetonitrile
min minute(s)
mL milliliter(s)
NBS n-bromosuccinimide
Pd(dppf)₂Cl₂ [1,1′-bis(diphenylphosphino)ferrocene]dichloropalladium(II)
Pd(dtbpf)Cl₂ [1,1′-Bis(di-tert-butylphosphino)ferrocene]dichloropalladium(II)
Pd(OAc)₂ palladium(II) acetate
Pd(PPh₃)₄ tetrakis(triphenylphosphine)palladium
PhSO₃H benzenesulfonic acid
PTSOH para-toluenesulfonic acid
TEA triethylamine
THE tetrahydrofuran
XPhos Pd G2 chloro(2-dicyclohexylphosphino-2′,4′,6′-triisopropyl-1,1′-biphenyl)[2-(2′-amino-1,1′-biphenyl)]palladium(II)

Preparative HPLC Method 1: XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-min hold at 15% B, 15-50% B over 25 min, then a 6-min hold at 100% B; Flow Rate: 20 mL/min; Column Temperature: 25° C. Fraction collection was triggered by MS signals.

UHPLC-MS method: Column: Waters Acquity BEH Phenyl 1.7 μm 2.1×50 mm; Mobile Phase A: 5:95 acetonitrile: water with 10-mM ammonium acetate; Mobile Phase B: 95:5 acetonitrile: water with 10-mM ammonium acetate; Gradient: Start B, 0% B, Final B, 100% B over 3 min, then a 0.5 min hold at 100% B; Flow Rate: 1 mL/min; Fraction collection was triggered by MS signals.

### Example 1

### 3-(5-(5-chloro-4-((3-(3,4-difluorophenyl)azetidin-1-yl)methyl)pyridin-2-yl)-1-oxoisoindolin-2-yl)piperidine-2,6-dione

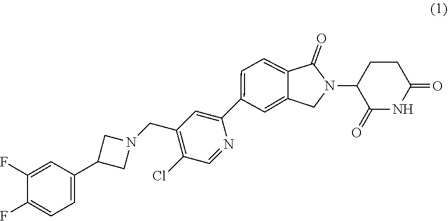 [SMILES: O=C1CCC(N2Cc3cc(-c4cc(CN5CC(c6ccc(F)c(F)c6)C5)c(Cl)cn4)ccc3C2=O)C(=O)N1]

### Intermediate 1A: tert-butyl (S)-5-amino-4-(5-(5-chloro-4-(hydroxymethyl)pyridin-2-yl)-1-oxoisoindolin-2-yl)-5-oxopentanoate

A 20 mL vial was charged with (2,5-dichloropyridin-4-yl)methanol (0.2 g, 1.124 mmol), tert-butyl (S)-5-amino-5-oxo-4-(1-oxo-5-(4,4,5,5-tetramethyl-1,3,2-dioxaborolan-2-yl)isoindolin-2-yl)pentanoate (0.749 g, 1.685 mmol), Pd(dtbpf)Cl₂ (0.022 g, 0.034 mmol), 1,4-dioxane (5.62 mL) and 3 M aqueous K₃PO₄ (1.873 mL, 5.62 mmol). The vial was sealed and the air was replaced with nitrogen. The reaction mixture was heated at 60° C. for 16 hrs. The reaction mixture was cooled to room temperature, diluted with EtOAc (25 mL), washed with brine, and the layers separated. The organics were dried over sodium sulfate, filtered, and concentrated. The crude product was purified using silica gel column by ISCO column chromatography (40 g Gold column, eluting with 0-100% of 20% methanolic ammonia in DCM-DCM) to give tert-butyl-5-amino-4-(5-(5-chloro-4-(hydroxymethyl)pyridin-2-yl)-1-oxoisoindolin-2-yl)-5-oxopentanoate (320 mg, 0.668 mmol, 59.4% yield) as a light brown solid. The enantiomeric excess of this material and subsequent intermediates were not determined. MS (ES): m/z=460.05 [M+H]⁺.

### Intermediate 1B: 3-(5-(5-chloro-4-(chloromethyl)pyridin-2-yl)-1-oxoisoindolin-2-yl) piperidine-2,6-dione

Thionyl chloride (0.132 mL, 1.826 mmol) was added dropwise to a cooled (0° C.) solution of tert-butyl-5-amino-4-(5-(5-chloro-4-(hydroxymethyl)pyridin-2-yl)-1-oxoisoindolin-2-yl)-5-oxopentanoate (0.280 g, 0.609 mmol) in DCM (3.00 mL). After 10 minutes, the ice-bath was removed, and the reaction mixture was allowed to warm to room temperature. After 30 minutes, the reaction mixture was concentrated to dryness. The residue was dissolved in acetic acid (3 mL) and benzenesulfonic acid (0.212 g, 1.339 mmol) was added and the reaction mixture was heated at 120° C. in microwave oven for 25 minutes. The reaction mixture was concentrated to dryness, and 2 mL of 3 M HCl in MeOH was added and stirred at 0° C. (ice-water bath) until there was complete dissolution. Then 8 mL of EtOAc was added, and the reaction mixture was stirred. After 5 minutes, the reaction mixture was allowed to stay still in the ice-water bath for 30 minutes. The precipitate was filtered, washed with EtOAc, and then air-dried to give 3-(5-(5-chloro-4-(chloromethyl)pyridin-2-yl)-1-oxoisoindolin-2-yl)piperidine-2,6-dione (180 mg, 0.401 mmol, 65.8% yield) as a HCl salt. MS (ES): m/z=404.3, 406.3 [M+H]⁺.

### Example 1

To a solution of 3-(5-(5-chloro-4-(chloromethyl)pyridin-2-yl)-1-oxoisoindolin-2-yl)piperidine-2,6-dione, HCl (40 mg, 0.091 mmol) in DMF (0.5 mL) was added 3-(3,4-difluorophenyl)azetidine, HCl (21.5 mg, 0.104 mmol) followed by Hunig's base (95 μL, 0.545 mmol). The resulting mixture was heated to 80° C. with stirring for 3 hours. It was then cooled and diluted further with DMF (0.5 mL). The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 35% B, 35-75% B over 22 minutes, then a 0-minute hold at 100% B; Flow Rate: 20 mL/min; Column Temperature: 25° C. Fraction collection was triggered by MS signals. Fractions containing the product were combined and dried via centrifugal evaporation to obtain 3-(5-(5-chloro-4-((3-(3,4-difluorophenyl)azetidin-1-yl)methyl) pyridin-2-yl)-1-oxoisoindolin-2-yl)piperidine-2,6-dione (25.2 mg, 0.046 mmol, 51% yield). LCMS (Method A): Retention Time 1.97 min; MS (ES): m/z=537.17 [M+H]⁺; ¹H NMR (400 MHz, DMSO-d6) δ 8.89 (s, 1H), 8.82 (s, 1H), 8.33 (s, 1H), 8.24 (m, m 1H), 7.99 (s, 1H), 7.84 (d, J=8.0 Hz, 1H), 7.41-7.31 (m, 5H), 7.26-7.20 (m, 1H), 5.16 (m, 1H), 4.56 (d, J=17.4 Hz, 1H), 4.44 (d, J=17.4 Hz, 1H), 3.80-3.66 (m, 5H), 3.26-3.18 (m, 2H), 3.00-2.88 (m, 1H), 2.49-2.37 (m, 1H), 2.10-2.01 (m, 1H).

### Examples 2-5

The compounds in Table 1 were prepared according to the procedures described for Example 1, replacing 3-phenylazetidine with the appropriate amine.

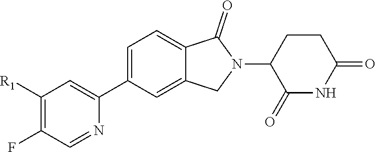

TABLE 1

| Ex. | | LC/MS | Retention | HPLC |
|---|---|---|---|---|
| No. | R₁ | [M + H]⁺ | Time (min) | Method |
| 2 | 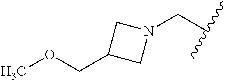 [SMILES: *CN1CC(COC)C1] | 453.30 | 1.32 | A |
| 3 | 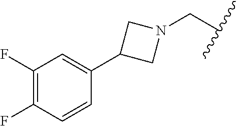 [SMILES: *CN1CC(c2ccc(F)c(F)c2)C1] | 521.12 | 1.88 | A |
| 4 | 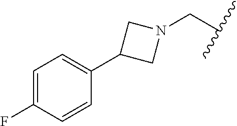 [SMILES: *CN1CC(c2ccc(F)cc2)C1] | 503.08 | 1.85 | A |
| 5 | 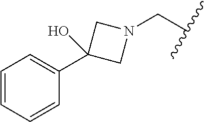 [SMILES: *CN1CC(O)(c2ccccc2)C1] | 501.01 | 1.58 | A |

### Examples 6-9

The compounds in Table 2 were prepared according to the general procedures described for Example 1, replacing 3-phenylazetidine with the appropriate amine.

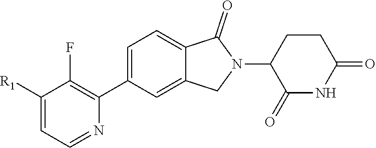

TABLE 2

| Ex. | | LC/MS | Retention | HPLC |
|---|---|---|---|---|
| No. | R₁ | [M + H]⁺ | Time (min) | Method |
| 6 | 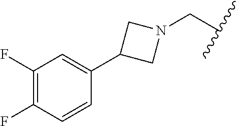 [SMILES: *CN1CC(c2ccc(F)c(F)c2)C1] | 521.18 | 1.70 | A |
| 7 | 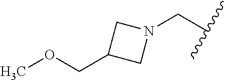 [SMILES: *CN1CC(COC)C1] | 453.21 | 1.23 | A |
| 8 | 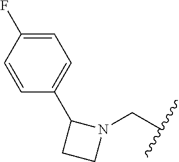 [SMILES: *CN1CCC1c1ccc(F)cc1] | 503.33 | 1.73 | A |
| 9 | 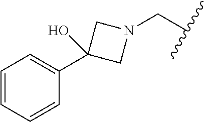 [SMILES: *CN1CC(O)(c2ccccc2)C1] | 501.18 | 1.41 | A |

### Example 10

### 3-(5-(4-((2-(3-fluorophenyl)azetidin-1-yl)methyl)pyridin-2-yl)-1-oxoisoindolin-2-yl) piperidine-2,6-dione

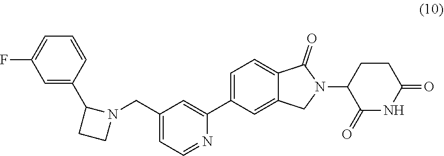 [SMILES: O=C1CCC(N2Cc3cc(-c4cc(CN5CCC5c5cccc(F)c5)ccn4)ccc3C2=O)C(=O)N1]

### Intermediate 10A: 1-cyclopropyl-3-fluorobenzene

A 20 mL scintillation vial was charged with 1-bromo-3-fluorobenzene (500 mg, 2.86 mmol), cyclopropylboronic acid (258 mg, 3.00 mmol), Pd(dtbpf)Cl₂ (55.9 mg, 0.086 mmol), THE (10 mL) and aqueous K₃PO₄ (2.86 mL, 8.57 mmol). The air was replaced with nitrogen, and the reaction mixture was heated for 2 h at 80° C. The reaction mixture was cooled to room temperature. The reaction was quenched with brine. The reaction mixture was diluted with ether. The organic layer was concentrated, and purified using a 40 gram silica gel column by ISCO, eluting with 0-5% EtOAc/hexanes. Appropriate fractions were combined and the solvents evaporated to obtain 261 mg (67% yield) of the title compound. ¹H NMR (400 MHz, CHLOROFORM-d) δ 7.26-7.19 (m, 1H), 6.91-6.81 (m, 2H), 6.76 (dt, J=10.4, 2.1 Hz, 1H), 1.95-1.87 (m, 1H), 1.04-0.98 (m, 2H), 0.74-0.69 (m, 2H).

### Intermediate 10B: 1-(1,3-dibromopropyl)-3-fluorobenzene

To a solution of 1-cyclopropyl-3-fluorobenzene (180 mg, 1.322 mmol) in DCM (10 mL) was added potassium bromide (346 mg, 2.91 mmol) and a solution of cerium ammonium nitrate (1667 mg, 3.04 mmol) in water (10 mL) at room temperature. The reaction mixture was stirred. After 0.75 h, the organic layer was separated, washed with brine and concentrated. The residue on purification by column chromatography on silica gel (40 gram) using hexane as eluent afforded the title product in 64% yield. ¹H NMR (400 MHz, CHLOROFORM-d) δ 7.35 (td, J=8.0, 5.9 Hz, 1H), 7.21 (d, J=7.8 Hz, 1H), 7.16 (dt, J=9.6, 2.1 Hz, 1H), 7.04 (t, J=8.2 Hz, 1H), 5.18 (dd, J=8.9, 5.8 Hz, 1H), 3.58 (ddd, J=10.4, 7.9, 5.4 Hz, 1H), 3.44 (dt, J=10.4, 5.8 Hz, 1H), 2.76 (ddt, J=14.8, 9.0, 5.8 Hz, 1H), 2.55 (ddt, J=15.0, 7.9, 5.7 Hz, 1H).

### Example 10

To 1-(1,3-dibromopropyl)-3-fluorobenzene (25 mg, 0.084 mmol) dissolved in DMF (1 mL) was added 3-(5-(4-(aminomethyl)pyridin-2-yl)-1-oxoisoindolin-2-yl) piperidine-2,6-dione, 2 HCl (35.8 mg, 0.084 mmol) followed by Hunig's base (0.118 mL, 0.676 mmol). The reaction mixture was heated to 100° C. for 1 h, cooled to room temperature, and purified by preparative HPLC method 1 to obtain 3.2 mg of the title compound. Analytical HPLC Method B retention time 1.68 min, [M+H]⁺ 485.2.

### Example 11

### 3-(5-(4-((3-(3-fluorophenyl)azetidin-1-yl)methyl)pyridin-2-yl)-1-oxoisoindolin-2-yl) piperidine-2,6-dione

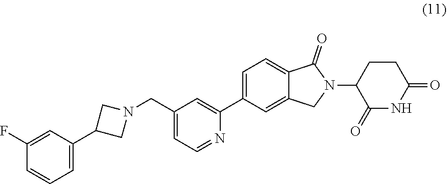 [SMILES: O=C1CCC(N2Cc3cc(-c4cc(CN5CC(c6cccc(F)c6)C5)ccn4)ccc3C2=O)C(=O)N1]

### Intermediate 11A: tert-butyl 3-(2-tosylhydrazineylidene)azetidine-1-carboxylate

To tert-butyl 3-oxoazetidine-1-carboxylate (4 g, 23.4 mmol) was added 4-methylbenzenesulfonohydrazide (4.35 g, 23.4 mmol) followed by toluene (8 mL). The reaction mixture was heated at reflux for 40 min. The reaction mixture was cooled down. The solid was collected by filtration to give tert-butyl 3-(2-tosylhydrazineylidene) azetidine-1-carboxylate (7.133 g, 21 mmol, 90% yield). MS (ES): m/z=337.9 [M−H]⁻. ¹H NMR (400 MHz, DMSO-d6) δ 10.85 (br s, 1H), 7.71 (d, J=8.1 Hz, 2H), 7.42 (d, J=8.1 Hz, 2H), 4.53-4.44 (m, 4H), 3.32 (s, 3H), 2.40 (s, 3H), 1.39 (s, 9H). References: 1) Allwood, D. M. J. Org. Chem. 2014, 79, 328-338; 2) Barluenga, J. Nat. Chem., 2009, 1, 494-499.

### Intermediate 11B: tert-butyl 3-(3-fluorophenyl)azetidine-1-carboxylate

tert-Butyl 3-(2-tosylhydrazineylidene)azetidine-1-carboxylate (200 mg, 0.589 mmol), (3-fluorophenyl)boronic acid (124 mg, 0.884 mmol), and cesium carbonate (288 mg, 0.884 mmol) were placed in a pressure-relief vial in vacuo for 30 min. The vial was back filled with nitrogen. Dry 1,4-dioxane (3 mL) was added under nitrogen. The vial was sealed and heated to 110° C. for 18 h before being cooled to room temperature. The reaction was quenched by aqueous saturated NaHCO₃ solution. The mixture was extracted with CH₂Cl₂. The organic phase was dried over Na₂SO₄, and solvent was removed to give a residue, which was purified by flash column chromatography (0-40% EtOAc/hexanes) to give tert-butyl 3-(3-fluorophenyl)azetidine-1-carboxylate (65 mg, 0.26 mmol, 43.9% yield). ¹H NMR (400 MHz, METHANOL-d₄) δ 7.47-7.30 (m, 1H), 7.21-7.15 (m, 1H), 7.13-7.07 (m, 1H), 7.04-6.97 (m, 1H), 4.41-4.33 (m, 2H), 3.98-3.90 (m, 2H), 3.89-3.80 (m, 1H), 1.49 (s, 9H).

### Example 11

To tert-butyl 3-(3-fluorophenyl)azetidine-1-carboxylate (61 mg, 0.243 mmol) was added TFA/DCM (1:1, 1 mL). The reaction mixture was stirred at room temperature for 2 h. The solvent was evaporated to give 3-(3-fluorophenyl)azetidine, TFA salt. To 3-(5-(4-(chloromethyl)pyridin-2-yl)-1-oxoisoindolin-2-yl)piperidine-2,6-dione, HCl salt (25 mg, 0.062 mmol), 3-(3-fluorophenyl)azetidine, TFA salt (20 mg, 0.075 mmol) was added DMF (2 mL) followed by N-ethyl-N-isopropylpropan-2-amine (0.107 mL, 0.615 mmol). The reaction mixture was heated at 80° C. for 2 h. The reaction mixture was cooled to room temperature, filtered and purified by preparative HPLC to afford 3-(5-(4-((3-(3-fluorophenyl)azetidin-1-yl)methyl)pyridin-2-yl)-1-oxoisoindolin-2-yl)piperidine-2,6-dione (13.8 mg, 0.028 mmol, 46.3% yield). Preparative HPLC condition: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: 20-60% B over 25 minutes; Flow rate: 20 mL/min; Column temperature: 25° C. The enantiomeric excess of 3-(5-(4-((3-(3-fluorophenyl)azetidin-1-yl)methyl)pyridin-2-yl)-1-oxoisoindolin-2-yl)piperidine-2,6-dione was not determined. LCMS (Method A): retention time 1.16 min. MS (ES): m/z=484.9 [M+H]⁺. ¹H NMR (500 MHz, DMSO-d6) δ 8.64 (d, J=4.6 Hz, 1H), 8.32 (br s, 1H), 8.23 (br d, J=7.8 Hz, 1H), 7.97 (s, 1H), 7.84 (d, J=8.0 Hz, 1H), 7.41-7.34 (m, 2H), 7.26-7.17 (m, 2H), 7.05 (br t, J=8.2 Hz, 1H), 5.21-5.11 (m, 1H), 4.56 (br d, J=17.2 Hz, 1H), 4.43 (br d, J=17.3 Hz, 1H), 3.77 (s, 2H), 3.74-3.69 (m, 3H), 3.22 (br s, 2H), 2.96-2.82 (m, 1H), 2.63 (br d, J=17.3 Hz, 1H), 2.44 (br dd, J=12.4, 4.0 Hz, 1H), 2.19-1.99 (m, 1H).

### Examples 12-27

The compounds in Table 3 were prepared according to the procedures described for Example 1.

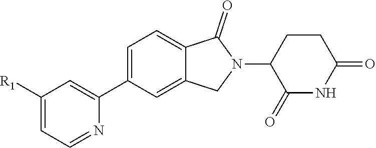

TABLE 3

| Ex. | | LC/MS | Retention | HPLC |
|---|---|---|---|---|
| No. | R₁ | [M + H]⁺ | Time (min) | Method |
| 12 | 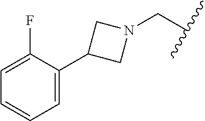 [SMILES: *CN1CC(c2ccccc2F)C1] | 485.2 | 1.05 | A |
| 13 | 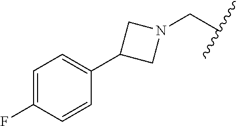 [SMILES: *CN1CC(c2ccc(F)cc2)C1] | 484.9 | 1.16 | A |
| 14 | 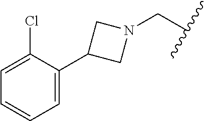 [SMILES: *CN1CC(c2ccccc2Cl)C1] | 501.2 | 1.22 | A |
| 15 | 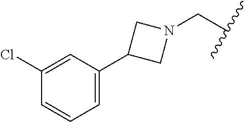 [SMILES: *CN1CC(c2cccc(Cl)c2)C1] | 500.9 | 1.28 | A |
| 16 | 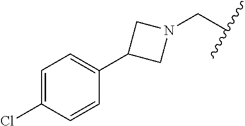 [SMILES: *CN1CC(c2ccc(Cl)cc2)C1] | 501.3 | 1.24 | A |
| 17 | 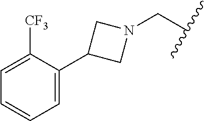 [SMILES: *CN1CC(c2ccccc2C(F)(F)F)C1] | 535.2 | 1.20 | A |
| 18 | 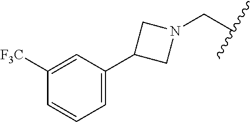 [SMILES: *CN1CC(c2cccc(C(F)(F)F)c2)C1] | 534.9 | 1.28 | A |
| 19 | 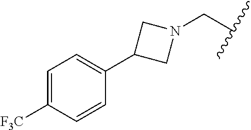 [SMILES: *CN1CC(c2ccc(C(F)(F)F)cc2)C1] | 535.2 | 1.25 | A |
| 20 | 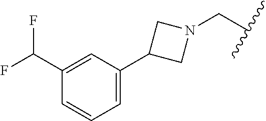 [SMILES: *CN1CC(c2cccc(C(F)F)c2)C1] | 517.2 | 1.13 | A |
| 21 | 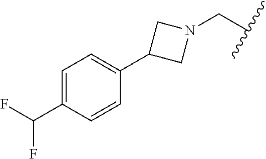 [SMILES: *CN1CC(c2ccc(C(F)F)cc2)C1] | 517.0 | 1.25 | A |
| 22 | 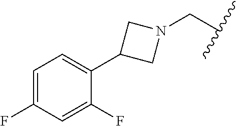 [SMILES: *CN1CC(c2ccc(F)cc2F)C1] | 503.2 | 1.62 | B |
| 23 | 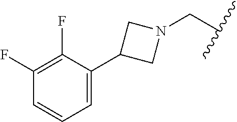 [SMILES: *CN1CC(c2cccc(F)c2F)C1] | 503.2 | 1.09 | A |
| 24 | 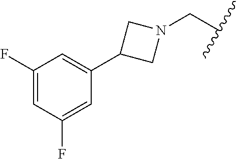 [SMILES: *CN1CC(c2cc(F)cc(F)c2)C1] | 503.2 | 1.09 | A |
| 25 | 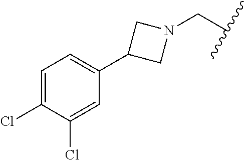 [SMILES: *CN1CC(c2ccc(Cl)c(Cl)c2)C1] | 535.1 | 1.41 | A |
| 26 | 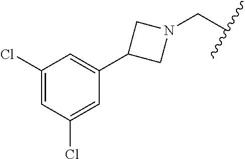 [SMILES: *CN1CC(c2cc(Cl)cc(Cl)c2)C1] | 535.1 | 1.28 | A |
| 27 | 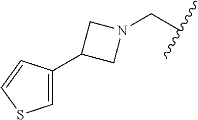 [SMILES: *CN1CC(c2ccsc2)C1] | 473.1 | 1.01 | A |

### Examples 28-30

The compounds in Table 4 were prepared according to the general procedures described for Example 1 using commercial available amines.

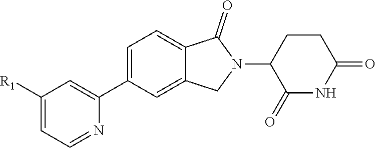

TABLE 4

| | | | Retention | |
|---|---|---|---|---|
| Ex. | | LC/MS | Time | HPLC |
| No. | R₁ | [M + H]⁺ | (min) | Method |
| 28 | 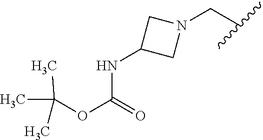 [SMILES: *CN1CC(NC(=O)OC(C)(C)C)C1] | 505.9 | 1.14 | A |
| 29 | 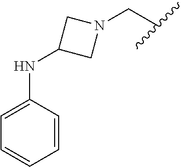 [SMILES: *CN1CC(Nc2ccccc2)C1] | 482.2 | 1.07 | A |
| 30 | 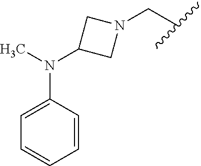 [SMILES: *CN1CC(N(C)c2ccccc2)C1] | 496.1 | 1.17 | A |
| 31 | 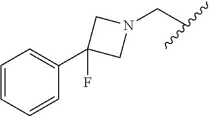 [SMILES: *CN1CC(F)(c2ccccc2)C1] | 485.2 | 1.07 | A |

### Example 32

### tert-butyl (4-((7-((2-(2-(2,6-dioxopiperidin-3-yl)-1-oxoisoindolin-5-yl)pyridin-4-yl) methyl)-7-azaspiro[3.5]nonan-2-yl)oxy)phenyl)carbamate

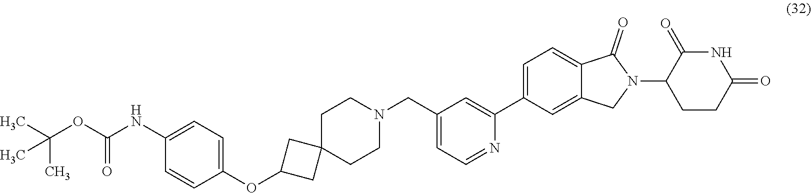 [SMILES: CC(C)(C)OC(=O)Nc1ccc(OC2CC3(CCN(Cc4ccnc(-c5ccc6c(c5)CN(C5CCC(=O)NC5=O)C6=O)c4)CC3)C2)cc1]

To a suspension of tert-butyl (4-((7-azaspiro[3.5]nonan-2-yl)oxy)phenyl) carbamate (17.53 mg, 0.053 mmol) and 3-(5-(4-(chloromethyl)pyridin-2-yl)-1-oxoisoindolin-2-yl)piperidine-2,6-dione (15 mg, 0.041 mmol) in DMF (1 mL) at room temperature was added Hunig's base (0.071 mL, 0.406 mmol). The reaction mixture became a clear solution. The vial was sealed and heated at 80° C. for 1.5 h. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 38% B, 38-78% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The yield of the product was 5.2 mg, (19%). ¹H NMR (500 MHz, DMSO-d₆) δ 9.11-9.01 (m, 1H), 8.68-8.58 (m, 1H), 8.33-8.29 (m, 1H), 8.24-8.12 (m, 1H), 7.99-7.92 (m, 1H), 7.89-7.79 (m, 1H), 7.40-7.18 (m, 3H), 6.75-6.64 (m, 2H), 5.19-5.06 (m, 1H), 4.66-4.61 (m, 1H), 4.58-4.32 (m, 2H), 3.62-3.51 (m, 5H), 2.97-2.84 (m, 1H), 2.71-2.60 (m, 1H), 2.47-2.18 (m, 7H), 2.13-1.99 (m, 1H), 1.77-1.65 (m, 2H), 1.67-1.55 (m, 5H), 1.4.

### Examples 33-67

The compounds in Table 5 were prepared according to the procedures described for Example 1, replacing tert-butyl (4-((7-azaspiro[3.5]nonan-2-yl)oxy)phenyl)carbamate with the appropriate primary or secondary amine.

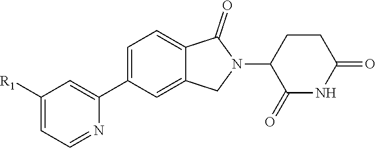

TABLE 5

| | | | Retention | |
|---|---|---|---|---|
| Ex. | | LC/MS | Time | HPLC |
| No. | R₁ | [M + H]⁺ | (min) | Method |
| 33 | 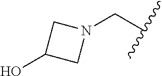 [SMILES: *CN1CC(O)C1] | 407.2 | 0.83 | A |
| 34 | 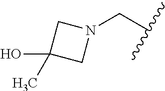 [SMILES: *CN1CC(C)(O)C1] | 421.2 | 0.87 | A |
| 35 | 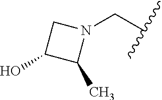 [SMILES: *CN1C[C@@H](O)[C@@H]1C] | 421.2 | 0.86 | A |
| 36 | 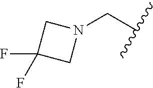 [SMILES: *CN1CC(F)(F)C1] | 427.0 | 1.00 | A |
| 37 | 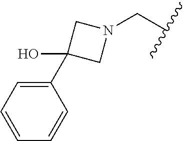 [SMILES: *CN1CC(O)(c2ccccc2)C1] | 483.1 | 1.10 | A |
| 38 | 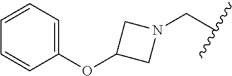 [SMILES: *CN1CC(Oc2ccccc2)C1] | 483.3 | 1.19 | A |
| 39 | 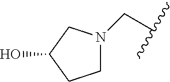 [SMILES: *CN1CC[C@H](O)C1] | 421.2 | 0.84 | A |
| 40 | 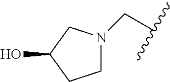 [SMILES: *CN1CC[C@@H](O)C1] | 421.3 | 0.89 | A |
| 41 | 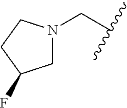 [SMILES: *CN1CC[C@H](F)C1] | 423.19 | 0.94 | A |
| 42 | 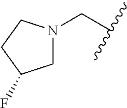 [SMILES: *CN1CC[C@@H](F)C1] | 423.19 | 0.94 | A |
| 43 | 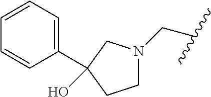 [SMILES: *CN1CCC(O)(c2ccccc2)C1] | 497.1 | 1.06 | A |
| 44 | 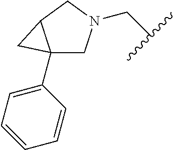 [SMILES: *CN1CC2CC2(c2ccccc2)C1] | 493.4 | 1.15 | A |
| 45 | 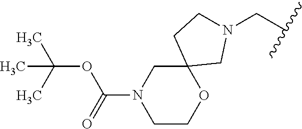 [SMILES: *CN1CCC2(C1)CN(C(=O)OC(C)(C)C)CCO2] | 575.9 | 1.17 | A |
| 46 | 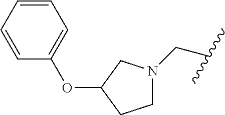 [SMILES: *CN1CCC(Oc2ccccc2)C1] | 497.1 | 1.19 | A |
| 47 | 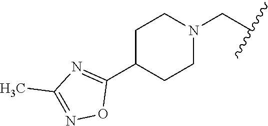 [SMILES: *CN1CCC(c2nc(C)no2)CC1] | 501.2 | 0.93 | A |
| 48 | 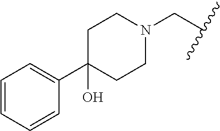 [SMILES: *CN1CCC(O)(c2ccccc2)CC1] | 511.2 | 1.1 | A |
| 49 | 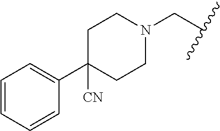 [SMILES: *CN1CCC(C#N)(c2ccccc2)CC1] | 520.4 | 1.26 | A |
| 50 | 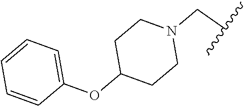 [SMILES: *CN1CCC(Oc2ccccc2)CC1] | 511.1 | 1.23 | A |
| 51 | 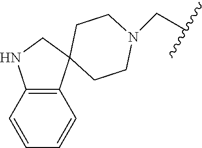 [SMILES: *CN1CCC2(CC1)CNc1ccccc12] | 522.3 | 0.91 | A |
| 52 | 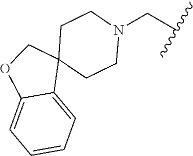 [SMILES: *CN1CCC2(CC1)COc1ccccc12] | 523.2 | 1.30 | A |
| 53 | 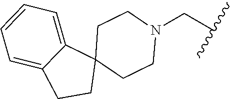 [SMILES: *CN1CCC2(CCc3ccccc32)CC1] | 521.1 | 1.38 | A |
| 54 | 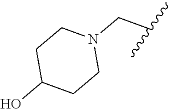 [SMILES: *CN1CCC(O)CC1] | 435.2 | 0.84 | A |
| 55 | 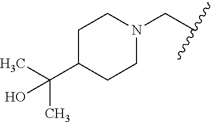 [SMILES: *CN1CCC(C(C)(C)O)CC1] | 477.1 | 0.97 | A |
| 56 | 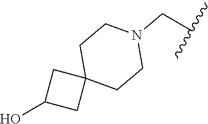 [SMILES: *CN1CCC2(CC1)CC(O)C2] | 474.9 | 0.91 | A |
| 57 | 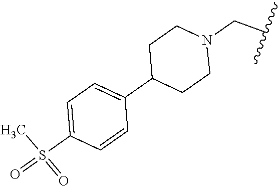 [SMILES: *CN1CCC(c2ccc(S(C)(=O)=O)cc2)CC1] | 573.2 | 0.99 | A |
| 58 | 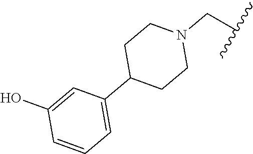 [SMILES: *CN1CCC(c2cccc(O)c2)CC1] | 511.2 | 1.0 | A |
| 59 | 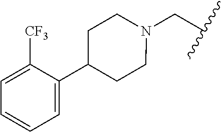 [SMILES: *CN1CCC(c2ccccc2C(F)(F)F)CC1] | 563.1 | 1.38 | A |
| 60 | 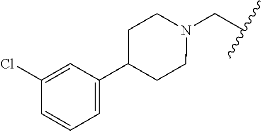 [SMILES: *CN1CCC(c2cccc(Cl)c2)CC1] | 529.2 | 1.27 | A |
| 61 | 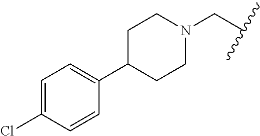 [SMILES: *CN1CCC(c2ccc(Cl)cc2)CC1] | 529.10 | 1.42 | A |
| 62 | 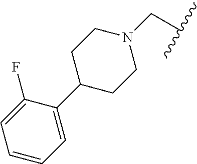 [SMILES: *CN1CCC(c2ccccc2F)CC1] | 513.1 | 1.23 | A |
| 63 | 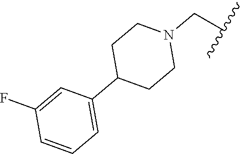 [SMILES: *CN1CCC(c2cccc(F)c2)CC1] | 513.2 | 1.18 | A |
| 64 | 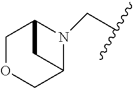 [SMILES: *CN1C2COC[C@H]1C2] | 432.9 | 0.85 | A |
| 65 | 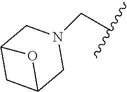 [SMILES: *CN1CC2CC(C1)O2] | 433.1 | 0.92 | A |
| 66 | 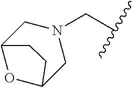 [SMILES: *CN1CC2CCC(C1)O2] | 447.2 | 0.98 | A |
| 67 | 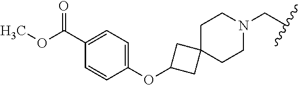 [SMILES: *CN1CCC2(CC1)CC(Oc1ccc(C(=O)OC)cc1)C2] | 609.0 | 1.39 | A |

### Example 68

### 3-(5-(4-((6-oxa-2,9-diazaspiro[4.5]decan-2-yl)methyl)pyridin-2-yl)-1-oxoisoindolin-2-yl) piperidine-2,6-dione

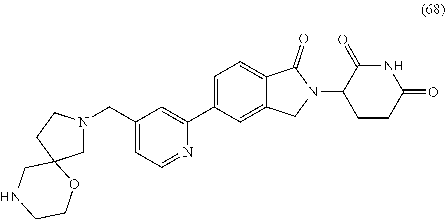 [SMILES: O=C1CCC(N2Cc3cc(-c4cc(CN5CCC6(CNCCO6)C5)ccn4)ccc3C2=O)C(=O)N1]

To tert-butyl 2-((2-(2-(2,6-dioxopiperidin-3-yl)-1-oxoisoindolin-5-yl)pyridin-4-yl) methyl)-6-oxa-2,9-di azaspiro[4.5]decane-9-carboxylate (9.3 mg, 0.012 mmol) in a 2 dram vial was added 20% TFA/CH₂Cl₂ (1 mL, 0.012 mmol). The reaction mixture was stirred at room temperature for 1 h. The reaction mixture was concentrated and dissolved in DMF. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 0% B, 0-40% B over 20 minutes, then a 0-minute hold at 100% B; Flow Rate: 20 mL/min; Column Temperature: 25° C. Fraction collection was triggered by MS signals. Fractions containing the product were combined and dried via centrifugal evaporation. The yield of the product was 4.0 mg. ¹H NMR (500 MHz, DMSO-d₆) δ 8.70-8.61 (m, 1H), 8.34-8.27 (m, 1H), 8.25-8.14 (m, 1H), 8.00-7.94 (m, 1H), 7.89-7.81 (m, 1H), 7.37 (br d, J=4.3 Hz, 1H), 5.13 (br dd, J=13.3, 5.3 Hz, 1H), 4.65-4.35 (m, 2H), 3.58-3.42 (m, 2H), 2.98-2.86 (m, 1H), 2.81-2.73 (m, 1H), 2.69-2.32 (m, 14H), 2.11-1.98 (m, 1H), 1.88-1.82 (m, 2H), 1.81-1.70 (m, 2H). LC MS: M+H=476.1 tr=0.88 min. (Method A).

### Example 69

### 3-(5-(4-((9-benzoyl-6-oxa-2,9-diazaspiro[4.5]decan-2-yl)methyl)pyridin-2-yl)-1-oxoisoindolin-2-yl)piperidine-2,6-dione

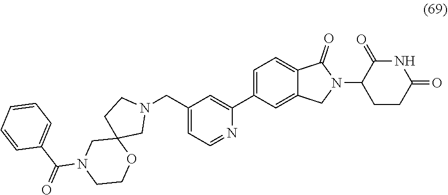 [SMILES: O=C1CCC(N2Cc3cc(-c4cc(CN5CCC6(C5)CN(C(=O)c5ccccc5)CCO6)ccn4)ccc3C2=O)C(=O)N1]

To a solution of 3-(5-(4-((6-oxa-2,9-diazaspiro[4.5]decan-2-yl)methyl)pyridin-2-yl)-1-oxoisoindolin-2-yl)piperidine-2,6-dione (10 mg, 0.021 mmol) in DMF (1 mL) was added benzoic acid (3.85 mg, 0.032 mmol), followed by HATU (11.99 mg, 0.032 mmol) and Hunig's base (0.037 mL, 0.210 mmol). The reaction mixture was stirred at room temperature for 5 h. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 1% B, 1-41% B over 20 minutes, then a 0-minute hold at 100% B; Flow Rate: 20 mL/min; Column Temperature: 25° C. Fraction collection was triggered by MS signals. Fractions containing the product were combined and dried via centrifugal evaporation. The yield of the product was 22.5 mg. ¹H NMR (500 MHz, DMSO-d₆) δ 11.12-10.89 (m, 1H), 8.89-8.74 (m, 1H), 8.39-8.17 (m, 3H), 7.96-7.84 (m, 1H), 7.61-7.39 (m, 5H), 5.24-5.02 (m, 1H), 4.67-4.40 (m, 3H), 3.84-3.62 (m, 1H), 3.03-2.84 (m, 1H), 2.72-2.59 (m, 1H), 2.58-2.49 (m, 13H), 2.48-2.31 (m, 2H), 2.15-1.93 (in, 2H). LC MS: M+H=580.2 tr=1.01 min. (Method A).

### Examples 70-88

The compounds in Table 6 were prepared according to the general procedures described for Example 1.

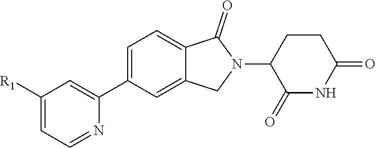

TABLE 6

| | | | Retention | |
|---|---|---|---|---|
| Ex | | LC/MS | Time | HPLC |
| No. | R₁ | [M + H]⁺ | (min) | Method |
| 70 | 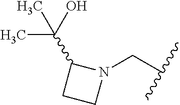 [SMILES: *CN1CCC1C(C)(C)O] | 449.2 | 1.06 | a |
| 71 | 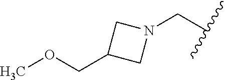 [SMILES: *CN1CC(COC)C1] | 435.12 | 1.17 | A |
| 72 | 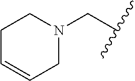 [SMILES: *CN1CC=CCC1] | 417.2 | 1.07 | A |
| 73 | 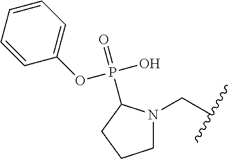 [SMILES: *CN1CCCC1P(=O)(O)Oc1ccccc1] | 561.0 | 1.06 | A |
| 74 | 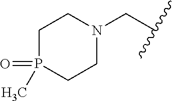 [SMILES: *CN1CCP(C)(=O)CC1] | 467.3 | 0.89 | A |
| 75 | 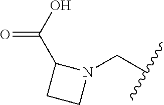 [SMILES: *CN1CCC1C(=O)O] | 435.0 | 0.94 | A |
| 76 | 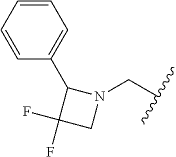 [SMILES: *CN1CC(F)(F)C1c1ccccc1] | 503.1 | 1.27 | A |
| 77 | 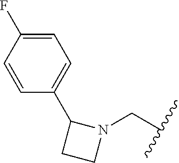 [SMILES: *CN1CCC1c1ccc(F)cc1] | 485.2 | 1.09 | A |
| 78 | 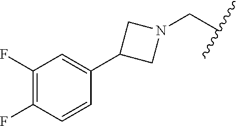 [SMILES: *CN1CC(c2ccc(F)c(F)c2)C1] | 503.1 | 1.19 | A |
| 79 | 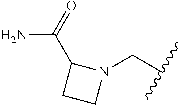 [SMILES: *CN1CCC1C(N)=O] | 434.1 | 0.82 | A |
| 80 | 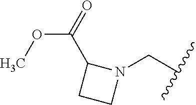 [SMILES: *CN1CCC1C(=O)OC] | 449.0 | 0.96 | A |
| 81 | 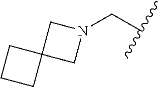 [SMILES: *CN1CC2(CCC2)C1] | 431.2 | 0.93 | A |
| 82 | 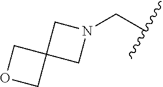 [SMILES: *CN1CC2(COC2)C1] | 433.3 | 0.87 | A |
| 83 | 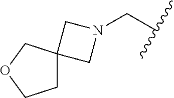 [SMILES: *CN1CC2(CCOC2)C1] | 447.1 | 0.92 | A |
| 84 | 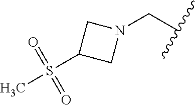 [SMILES: *CN1CC(S(C)(=O)=O)C1] | 469.2 | 1.05 | B |
| 85 | 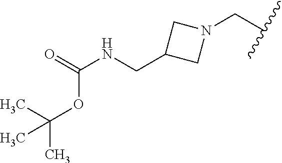 [SMILES: *CN1CC(CNC(=O)OC(C)(C)C)C1] | 520.2 | 1.53 | B |
| 86 | 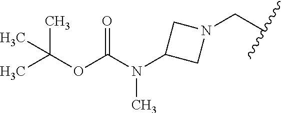 [SMILES: *CN1CC(N(C)C(=O)OC(C)(C)C)C1] | 520.2 | 1.34 | B |

*The absolute stereochemistry was not determined.

### Example 87

### 3-(5-(3-fluoro-4-((4-(2-hydroxypropan-2-yl)piperidin-1-yl)methyl)pyridin-2-yl)-1-oxoisoindolin-2-yl)piperidine-2,6-dione

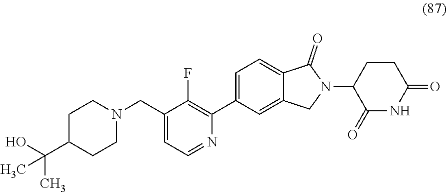 [SMILES: CC(C)(O)C1CCN(Cc2ccnc(-c3ccc4c(c3)CN(C3CCC(=O)NC3=O)C4=O)c2F)CC1]

### Intermediate 87A: tert-butyl-5-amino-4-(5-(3-fluoro-4-(hydroxymethyl)pyridin-2-yl)-1-oxoisoindolin-2-yl)-5-oxopentanoate

A 100 mL round bottom flask was charged with (2-chloro-3-fluoropyridin-4-yl) methanol (1.00 g, 6.19 mmol), tert-butyl (S)-5-amino-5-oxo-4-(1-oxo-5-(4,4,5,5-tetramethyl-1,3,2-dioxaborolan-2-yl)isoindolin-2-yl)pentanoate (3.16 g, 7.12 mmol), Pd(OAc)₂ (0.069 g, 0.309 mmol), di-tert-butyl(methyl)phosphonium tetrafluoroborate (0.154 g, 0.619 mmol), dioxane (50 mL) and aqueous K₂CO₃ (10.32 mL, 30.9 mmol). The reaction vessel was sealed and the air was replaced with nitrogen. The reaction mixture was heated for 24 hours at 80° C. while stirring. It was cooled to room temperature, diluted with EtOAc, washed with brine, and the organic layer separated, dried over MgSO₄ and concentrated. The crude mixture was purified using a 120 gram silica gel column by ISCO eluting with 0-10% DCM/MeOH, to obtain 2.37 g (86%) of the title compound. ¹H NMR (400 MHz, CHLOROFORM-d) δ ppm 8.56 (d, J=4.68 Hz, 1H) 8.08 (d, J=7.86 Hz, 2H) 8.07 (s, 1H) 7.94 (d, J=7.70 Hz, 1H) 7.54 (t, J=4.98 Hz, 1H) 6.41 (br s, 1H) 5.38 (br s, 1H) 4.92-4.99 (m, 3H) 4.51-4.64 (m, 2H) 2.16-2.45 (m, 5H) 1.45 (s, 9H).

### Intermediate 87B: 3-(5-(4-(chloromethyl)-3-fluoropyridin-2-yl)-1-oxoisoindolin-2-yl) piperidine-2,6-dione, HCl

To tert-butyl 5-amino-4-(5-(3-fluoro-4-(hydroxymethyl)pyridin-2-yl)-1-oxoisoindolin-2-yl)-5-oxopentanoate (2.38 g, 5.37 mmol) dissolved in DCM (50 mL) and cooled in an ice-water bath was added thionyl chloride (1.168 mL, 16.10 mmol), dropwise. After 10 minutes, the ice-bath was removed, and the reaction mixture was allowed to warm to room temperature. After 20 min., the reaction mixture was concentrated to dryness, and the residue obtained was dissolved in acetic acid (60 mL) and then PhSO₃H (1.867 g, 11.81 mmol) was added. The reaction mixture was refluxed (118° C.). After 3 h, the reaction mixture was concentrated to dryness, and 30 mL of 3 M HCl in MeOH was added and stirred at 0° C. for about 0.25 h. Next, 70 mL of EtOAc was added, and the reaction mixture was stirred. After 5 min., the mixture was allowed to remain in the ice-water bath for about 0.5 hour. The title compound precipitated out, and was filtered, washed with EtOAc, and then air-dried (74% yield). ¹H NMR (400 MHz, DMSO-d₆) δ ppm 11.02 (s, 1H) 8.61 (d, J=4.68 Hz, 1H) 8.14 (s, 1H) 8.05 (d, J=8.00 Hz, 1H) 7.89 (d, J=8.00 Hz, 1H) 7.68 (t, J=5.07 Hz, 1H) 5.17 (dd, J=13.32, 5.12 Hz, 1H) 4.94 (s, 2H) 4.53-4.62 (m, 1H) 4.41-4.50 (m, 1H) 2.88-3.00 (m, 1H) 2.58-2.69 (m, 1H) 2.36-2.48 (m, 1H) 2.02-2.11 (m, 1H).

### Example 87

A 40 mL vial was charged with 3-(5-(4-(chloromethyl)-3-fluoropyridin-2-yl)-1-oxoisoindolin-2-yl)piperidine-2,6-dione, HCl (700 mg, 1.650 mmol), N-ethyl-N-isopropylpropan-2-amine (2.87 mL, 16.50 mmol), DMF (20 mL) and N-ethyl-N-isopropylpropan-2-amine (2.87 mL, 16.50 mmol) and then heated for 1.5 hour at 80° C. The reaction mixture was cooled to room temperature, diluted with EtOAc, washed twice with 10% aqueous LiCl and brine, dried over MgSO₄, and then concentrated. The mixture was purified using a 24 gram silica gel column by ISCO, eluting with 0-90% B/DCM, where B=15% EtOH in EtOAc+0.1% triethylamine, to obtain the title compound in 58.4% yield (486 mg) as a free base. MS (ES): m/z=495.2 [M+H]⁺, (calculated for C₂₇H₃₁FN₄O₄ 494.2); UHPLC-MS retention time was 1.16 min; ¹H NMR (400 MHz, DMSO-d₆) δ ppm 11.01 (s, 1H) 8.53 (d, J=4.68 Hz, 1H) 8.12 (s, 1H) 8.04 (d, J=8.00 Hz, 1H) 7.87 (d, J=8.39 Hz, 1H) 7.53 (t, J=4.93 Hz, 1H) 5.16 (dd, J=13.32, 5.12 Hz, 1H) 4.56 (d, J=17.56 Hz, 1H) 4.44 (d, J=17.46 Hz, 1H) 4.05 (s, 1H) 3.64 (s, 2H) 2.88-2.99 (m, 3H) 2.59-2.67 (m, 1H) 2.36-2.48 (m, 1H) 2.02-2.09 (m, 1H) 1.98 (br t, J=10.78 Hz, 2H) 1.67 (br d, J=12.10 Hz, 2H) 1.29 (qd, J=12.21, 3.37 Hz, 2H) 1.12-1.22 (m, 1H) 1.04 (s, 6H).

Analytical HPLC Method A: Column: Waters XBridge C18, 2.1 mm×50 mm, 1.7 m particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm).

Analytical HPLC method B: Column: Waters XBridge C18, 2.1 mm×50 mm, 1.7 m particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm).

### Examples 88-91

The compounds in Table 7 were prepared according to the general procedures described for Example 87.

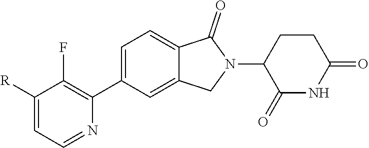

TABLE 7

| | | | Retention | |
|---|---|---|---|---|
| Ex. | | LC/MS | Time | HPLC |
| No. | R | [M + H]⁺ | (min) | Method |
| 88 | 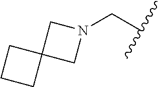 [SMILES: *CN1CC2(CCC2)C1] | 449.0 | 1.04 | B |
| 89 | 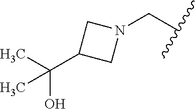 [SMILES: *CN1CC(C(C)(C)O)C1] | 467.20 | 1.11 | B |
| 90 | 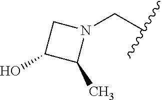 [SMILES: *CN1C[C@@H](O)[C@@H]1C] | 439.2 | 0.90 | B |
| 91 | 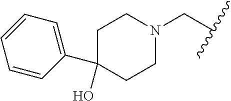 [SMILES: *CN1CCC(O)(c2ccccc2)CC1] | 529.2 | 1.01 | B |

### Example 92

### 3-(5-(5-fluoro-4-((4-(2-hydroxypropan-2-yl)piperidin-1-yl)methyl)pyridin-2-yl)-1-oxoisoindolin-2-yl)piperidine-2,6-dione

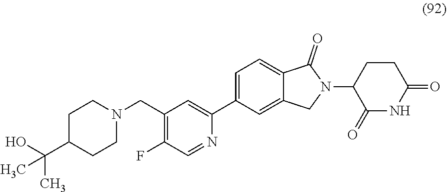 [SMILES: CC(C)(O)C1CCN(Cc2cc(-c3ccc4c(c3)CN(C3CCC(=O)NC3=O)C4=O)ncc2F)CC1]

### Intermediate 92A: tert-butyl 5-amino-4-(5-(5-fluoro-4-(hydroxymethyl)pyridin-2-yl)-1-oxoisoindolin-2-yl)-5-oxopentanoate

A 20 mL vial was charged with (2-chloro-5-fluoropyridin-4-yl)methanol (0.4 g, 2.476 mmol), tert-butyl (S)-5-amino-5-oxo-4-(1-oxo-5-(4,4,5,5-tetramethyl-1,3,2-dioxaborolan-2-yl)iso indolin-2-yl)pentanoate (prepared by methods shown in WO2018102725) (1.650 g, 3.71 mmol), Pd(dtbpf)Cl₂ (0.048 g, 0.074 mmol), 1,4-dioxane (12.38 mL) and 3 M aqueous K₃PO₄ (4.13 mL, 12.38 mmol). The vial was sealed and the air was replaced with nitrogen. The reaction mixture was heated at 60° C. for 16 hours. The reaction mixture was cooled to room temperature and diluted with ethyl acetate-water (25:5 mL). The organic layer was separated, washed with brine, dried over sodium sulfate, filtered, and concentrated. The crude product was purified using ISCO silica gel column chromatography (40 g Gold column, eluting with 0-100% of 20% methanolic ammonia in DCM-DCM) to give tert-butyl-5-amino-4-(5-(5-fluoro-4-(hydroxymethyl) pyridin-2-yl)-1-oxoisoindolin-2-yl)-5-oxopentanoate (650 mg, 1.466 mmol, 59.2% yield) as a pale brown solid. The enantiomeric excess of this material and subsequent intermediates were not determined. MS (ES): m/z=444.04 [M+H]⁺. ¹H NMR (400 MHz, CHLOROFORM-d) δ ppm 8.62 (d, J=4.68 Hz, 1H) 8.08 (d, J=7.86 Hz, 2H) 8.07 (s, 1H) 7.94 (d, J=7.70 Hz, 1H) 7.54 (t, J=4.98 Hz, 1H) 6.41 (br s, 1H) 5.38 (br s, 1H) 4.92-4.99 (m, 3H) 4.51-4.64 (m, 2H) 2.16-2.45 (m, 5H) 1.45 (s, 9H).

### Intermediate 92B: 3-(5-(5-fluoro-4-(chloromethyl)pyridin-2-yl)-1-oxoisoindolin-2-yl) piperidine-2,6-dione, HCl salt

Thionyl chloride (0.319 mL, 4.40 mmol) was added dropwise to a cooled (0° C.) solution of tert-butyl 5-amino-4-(5-(5-fluoro-4-(hydroxymethyl)pyridin-2-yl)-1-oxoisoindolin-2-yl)-5-oxopentanoate (0.65 g, 1.466 mmol) in DCM (7.5 mL). After 10 minutes, the ice-bath was removed, and the reaction mixture was allowed to warm to room temperature. After 30 minutes, the reaction mixture was concentrated to dryness. The residue was dissolved in acetic acid (3 mL) and benzenesulfonic acid (0.510 g, 3.22 mmol) was added. The reaction mixture was heated at 120° C. in a microwave oven for 25 minutes. The reaction mixture was concentrated to dryness, and 4 mL of 3 M HCl in MeOH was added followed by stirring at 0° C. (ice-water bath) until there was complete dissolution. To this solution was added 12 mL of ethyl acetate. The reaction mixture was stirred for 5 minutes. The reaction mixture was allowed to stay still in the ice-water bath for 30 minutes. The precipitate was filtered, washed with EtOAc, and then air-dried to give 3-(5-(4-(chloromethyl)-5-fluoropyridin-2-yl)-1-oxoisoindolin-2-yl)piperidine-2,6-dione (480 mg, 1.238 mmol, 84% yield) as a HCl salt. MS (ES): m/z=388.2 [M+H]⁺.

### Example 92

To 3-(5-(3-(chloromethyl)-4-fluorophenyl)-1-oxoisoindolin-2-yl)piperidine-2,6-dione, HCl (55 mg, 0.130 mmol) dissolved in DMF (650 μL) was added 2-(piperidin-4-yl)propan-2-ol, HCl (50 mg, 0.280 mmol) followed by Hunig's base (136 μL, 0.780 mmol). The resulting mixture was heated to 80° C. with stirring for 1 hour. The reaction mixture was then cooled and diluted further with DMF (0.5 mL). The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 35% B, 35-75% B over 22 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to obtain 3-(5-(5-fluoro-4-((4-(2-hydroxypropan-2-yl) piperidin-1-yl)methyl)pyridin-2-yl)-1-oxoisoindolin-2-yl)piperidine-2,6-dione (13.6 mg, 0.027 mmol, 21.21% yield). LCMS (Method A): Retention Time 1.02 min; MS (ES): m/z=495.10 [M+H]⁺; ¹H NMR (400 MHz, DMSO-d₆) δ ppm 11.01 (s, 1H) 8.53 (d, J=4.68 Hz, 1H) 8.12 (s, 1H) 8.04 (d, J=8.00 Hz, 1H) 7.87 (d, J=8.39 Hz, 1H) 7.53 (t, J=4.93 Hz, 1H) 5.16 (dd, J=13.32, 5.12 Hz, 1H) 4.56 (d, J=17.56 Hz, 1H) 4.44 (d, J=17.46 Hz, 1H) 4.05 (s, 1H) 3.64 (s, 2H) 2.88-2.99 (m, 3H) 2.59-2.67 (m, 1H) 2.36-2.48 (m, 1H) 2.02-2.09 (m, 1H) 1.98 (br t, J=10.78 Hz, 2H) 1.67 (br d, J=12.10 Hz, 2H) 1.29 (qd, J=12.21, 3.37 Hz, 2H) 1.12-1.22 (m, 1H) 1.04 (s, 6H).

### Example 93

### 3-(5-(4-((2-azaspiro[3.3]heptan-2-yl)methyl)-5-fluoropyridin-2-yl)-1-oxoisoindolin-2-yl) piperidine-2,6-dione

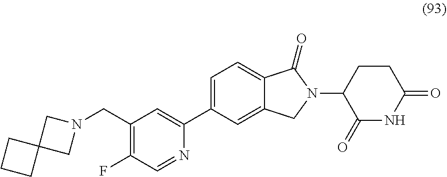 [SMILES: O=C1CCC(N2Cc3cc(-c4cc(CN5CC6(CCC6)C5)c(F)cn4)ccc3C2=O)C(=O)N1]

Example 93 was prepared according to the general procedure described in Example 92. m/z=449.00 [M+H]⁺; Retention time (Method B): 1.06 min.

### Example 94

### 3-(5-(3-chloro-4-((4-(2-hydroxypropan-2-yl)piperidin-1-yl)methyl)pyridin-2-yl)-1-oxoisoindolin-2-yl)piperidine-2,6-dione

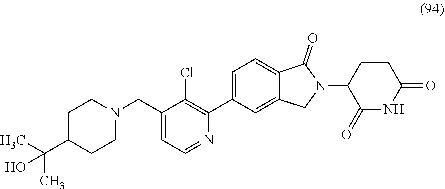 [SMILES: CC(C)(O)C1CCN(Cc2ccnc(-c3ccc4c(c3)CN(C3CCC(=O)NC3=O)C4=O)c2Cl)CC1]

### Intermediate 94A: tert-butyl 5-amino-4-(5-(3-chloro-4-(hydroxymethyl)pyridin-2-yl)-1-oxoisoindolin-2-yl)-5-oxopentanoate

A 20 mL vial was charged with (2,3-dichloropyridin-4-yl)methanol (400 mg, 2.247 mmol), tert-butyl (S)-5-amino-5-oxo-4-(1-oxo-5-(4,4,5,5-tetramethyl-1,3,2-dioxaborolan-2-yl)isoindolin-2-yl)pentanoate (prepared by methods disclosed in WO2018102725) (1498 mg, 3.37 mmol), Pd(dtbpf)Cl₂ (43.9 mg, 0.067 mmol), 1,4-dioxane (12 mL) and 3 M aqueous K₃PO₄ (3.75 mL, 11.24 mmol). The vial was sealed and the air was replaced with nitrogen. The reaction mixture was heated at 60° C. for 16 hours. The reaction mixture was cooled and diluted with ethyl acetate-water (25:5 mL). The organic layer was separated, washed with brine, dried over sodium sulfate, filtered, and concentrated. The crude product was purified using silica gel column by ISCO column chromatography (40 g Gold column, eluting with 0-100% of 20% methanolic ammonia in DCM-DCM) to give tert-butyl 5-amino-4-(5-(3-chloro-4-(hydroxymethyl) pyridin-2-yl)-1-oxoisoindolin-2-yl)-5-oxopentanoate (700 mg, 1.400 mmol, 62.3% yield) as a light brown solid. The enantiomeric excess of this material and subsequent intermediates were not determined. MS (ES): m/z=460.03 [M+H]⁺. ¹H NMR (400 MHz, CHLOROFORM-d) δ ppm 8.62 (d, J=4.68 Hz, 1H) 7.85-7.62 (m, 6H), 5.71 (br s, 1H) 5.38 (br s, 1H) 4.92-4.99 (m, 3H) 4.51-4.64 (m, 2H) 2.16-2.45 (m, 5H) 1.45 (s, 9H).

### Intermediate 94B: 3-(5-(3-chloro-4-(chloromethyl)pyridin-2-yl)-1-oxoisoindolin-2-yl) piperidine-2,6-dione HCl salt

Thionyl chloride (0.33 mL, 4.52 mmol) was added dropwise to a cooled (0° C.) solution of tert-butyl 5-amino-4-(5-(3-chloro-4-(hydroxymethyl)pyridin-2-yl)-1-oxoisoindolin-2-yl)-5-oxopentanoate (0.7 g, 1.52 mmol) in DCM (7.5 mL). After 10 minutes, the ice-bath was removed, and the reaction mixture was allowed to warm to room temperature. After 30 minutes, the reaction mixture was concentrated to dryness. The residue was dissolved in acetic acid (3 mL) and benzenesulfonic acid (0.53 g, 3.35 mmol) was added. The reaction mixture was heated at 120° C. in microwave oven for 25 minutes. The reaction was concentrated to dryness. Next, 4 mL of 3 M HCl in MeOH was added to the residue and the reaction mixture was stirred at 0° C. (ice-water bath) until there was complete dissolution. Then 12 mL of ethyl acetate was added, and the reaction mixture was stirred. After 5 minutes, the reaction mixture was allowed to stay still in the ice-water bath for 30 minutes. The precipitate was filtered, washed with ethyl acetate, and then air-dried to give 3-(5-(3-chloro-4-(chloromethyl)pyridin-2-yl)-1-oxoisoindolin-2-yl)piperidine-2,6-dione (400 mg, 0.940 mmol, 61.8% yield) as a HCl salt. MS (ES): m/z=404.1, 406.1 [M+H]⁺ and [M+2H]⁺, respectively.

### Example 94

To 3-(5-(3-chloro-4-(chloromethyl)pyridin-2-yl)-1-oxoisoindolin-2-yl)piperidine-2,6-dione, HCl (25 mg, 0.06 mmol) dissolved in DMF (650 μL) was added 2-(piperidin-4-yl)propan-2-ol, HCl (15.3 mg, 0.09 mmol) followed by Hunig's base (60 μL, 0.30 mmol). The resulting mixture was heated to 80° C. with stirring for 1 hour. The reaction mixture was cooled and diluted further with DMF (0.5 mL). The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 35% B, 35-75% B over 22 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to obtain 3-(5-(3-chloro-4-((4-(2-hydroxypropan-2-yl) piperidin-1-yl)methyl)pyridin-2-yl)-1-oxoisoindolin-2-yl)piperidine-2,6-dione (15.8 mg, 0.030 mmol, 52.3% yield). LCMS (Method A): Retention Time 1.03 min; MS (ES): m/z=511.20 [M+H]⁺; ¹H NMR (400 MHz, DMSO-d₆) δ ppm 11.01 (s, 1H) 8.21 (d, J=4.68 Hz, 1H) 8.04 (s, 1H) 7.81 (m, 2H) 7.41 (t, J=4.93 Hz, 1H) 5.16 (dd, J=13.32, 5.12 Hz, 1H) 4.56 (d, J=17.56 Hz, 1H) 4.44 (d, J=17.46 Hz, 1H) 4.05 (s, 1H) 3.64 (s, 2H) 2.88-2.99 (m, 3H) 2.59-2.67 (m, 1H) 2.36-2.48 (m, 1H) 2.02-2.09 (m, 1H) 1.98 (br t, J=10.78 Hz, 2H) 1.67 (br d, J=12.10 Hz, 2H) 1.29 (qd, J=12.21, 3.37 Hz, 2H) 1.12-1.22 (m, 1H) 1.04 (s, 6H).

### Examples 95-97

The compounds in Table 8 were prepared according to the general procedures described for Example 93.

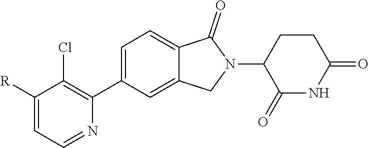

TABLE 8

| | | | Retention | |
|---|---|---|---|---|
| Ex. | | LC/MS | Time | HPLC |
| No. | R₁ | [M + H]⁺ | (min) | Method |
| 95 | 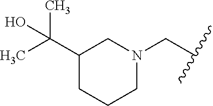 [SMILES: *CN1CCCC(C(C)(C)O)C1] | 511.20 | 1.05 | B |
| 96 | 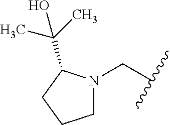 [SMILES: *CN1CCC[C@@H]1C(C)(C)O] | 497.20 | 0.96 | B |
| 97 | 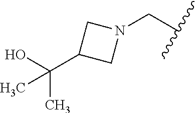 [SMILES: *CN1CC(C(C)(C)O)C1] | 483.10 | 1.01 | B |

### Example 98

### tert-butyl (4-((7-((2-(2-(2,6-dioxopiperidin-3-yl)-1-oxoisoindolin-5-yl)pyridin-4-yl) methyl)-7-azaspiro[3.5]nonan-2-yl)oxy)phenyl)carbamate

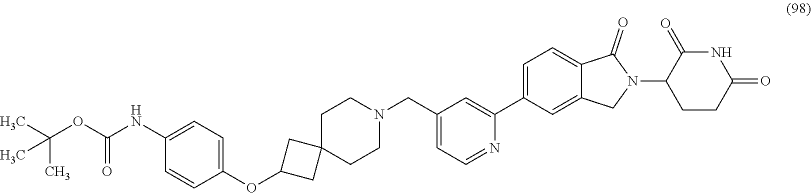 [SMILES: CC(C)(C)OC(=O)Nc1ccc(OC2CC3(CCN(Cc4ccnc(-c5ccc6c(c5)CN(C5CCC(=O)NC5=O)C6=O)c4)CC3)C2)cc1]

### Intermediate 98A: tert-butyl (S)-5-amino-4-(5-(4-(hydroxymethyl)pyridin-2-yl)-1-oxoisoindolin-2-yl)-5-oxopentanoate

A 100 mL flask was charged with (2-chloropyridin-4-yl)methanol (1.50 g, 10.45 mmol), tert-butyl (S)-5-amino-5-oxo-4-(1-oxo-5-(4,4,5,5-tetramethyl-1,3,2-dioxaborolan-2-yl)isoindolin-2-yl)pentanoate (6.96 g, 15.67 mmol), Pd(dtbpf)Cl₂ (0.204 g, 0.313 mmol), dioxane (50 mL) and aqueous K₃PO₄ (17.41 mL, 52.2 mmol). The flask was sealed and the air was replaced with nitrogen, and then heated overnight at 60° C. with stirring. The reaction mixture was cooled to room temperature, diluted with EtOAc, washed with brine, and the organic layer dried over MgSO₄, and then concentrated. The solid material was purified using silica gel column by ISCO eluting with 0-85% DCM/B [B=15% EtOH/EtOAc+0.1% triethylamine] to afford 3.01 g (68% yield) of the titled compound. MS (ES): m/z=426.2 [M+H]⁺.

### Intermediate 98B: (S)-3-(5-(4-(chloromethyl)pyridin-2-yl)-1-oxoisoindolin-2-yl) piperidine-2,6-dione hydrochloride

To tert-butyl (S)-5-amino-4-(5-(4-(hydroxymethyl)pyridin-2-yl)-1-oxoisoindolin-2-yl)-5-oxopentanoate (7.1 g, 16.69 mmol) dissolved in DCM (110 mL) and cooled in an ice-water bath was added thionyl chloride (3.63 mL, 50.1 mmol) dropwise. After 10 min, the ice-water bath was removed, and the reaction mixture was allowed to warm to room temperature. After 0.5 h, the reaction mixture was concentrated to dryness. The residue obtained was dissolved in acetic acid (110 mL) and then PhSO₃H (5.81 g, 36.7 mmol) was added. The reaction mixture was refluxed at 118° C. for 3 h. The reaction mixture was concentrated to dryness. Next, 70 mL of 3 M HCl in MeOH was added and the mixture was stirred at 0° C. (ice-water bath) until there was complete dissolution. Then, 150 mL of EtOAc was added, and the mixture was stirred. After about 5 min, the mixture was allowed to stay still in the ice-water bath for about 0.5 h. The precipitates were filtered, washed with cold EtOAc, and then air-dried to 89% yield of the titled compound. ¹H NMR (400 MHz, DMSO-d₆) δ 11.05-10.98 (m, 1H), 8.76 (d, J=5.0 Hz, 1H), 8.34 (s, 1H), 8.24 (dd, J=8.0, 1.4 Hz, 1H), 8.19 (s, 1H), 7.87 (d, J=8.0 Hz, 1H), 7.56 (dd, J=5.1, 1.2 Hz, 1H), 5.16 (dd, J=13.3, 5.2 Hz, 1H), 4.90 (s, 2H), 4.56 (d, J=1.0 Hz, 1H), 4.45 (d, J=1.0 Hz, 1H), 3.00-2.88 (m, 1H), 2.67-2.59 (m, 1H), 2.48-2.38 (m, 1H), 2.11-1.99 (m, 1H).

### Example 98

To a suspension of tert-butyl (4-((7-azaspiro[3.5]nonan-2-yl)oxy)phenyl) carbamate (17.53 mg, 0.053 mmol) and 3-(5-(4-(chloromethyl)pyridin-2-yl)-1-oxoisoindolin-2-yl)piperidine-2,6-dione (15 mg, 0.041 mmol) in DMF (1 mL) at room temperature was added Hunig's base (0.071 mL, 0.406 mmol). The reaction mixture became a clear solution. The vial was sealed and heated at 80° C. for 1.5 h. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 38% B, 38-78% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The yield of the product was 5.2 mg, (19%). ¹H NMR (500 MHz, DMSO-d₆) δ 9.11-9.01 (m, 1H), 8.68-8.58 (m, 1H), 8.33-8.29 (m, 1H), 8.24-8.12 (m, 1H), 7.99-7.92 (m, 1H), 7.89-7.79 (m, 1H), 7.40-7.18 (m, 3H), 6.75-6.64 (m, 2H), 5.19-5.06 (m, 1H), 4.66-4.61 (m, 1H), 4.58-4.32 (m, 2H), 3.62-3.51 (m, 5H), 2.97-2.84 (m, 1H), 2.71-2.60 (m, 1H), 2.47-2.18 (m, 7H), 2.13-1.99 (m, 1H), 1.77-1.65 (m, 2H), 1.67-1.55 (m, 5H), 1.48-1.40 (m, 9H) LCMS (Method B): retention time 1.56 min, [M+H]⁺660.2.

### Examples 99-108

The compounds in Table 9 were prepared according to the general procedures described for Example 98, replacing tert-butyl (4-((7-azaspiro[3.5]nonan-2-yl)oxy) phenyl)carbamate with the appropriate primary or secondary amine.

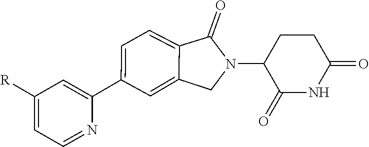

TABLE 9

| | | | Retention | |
|---|---|---|---|---|
| | | LC/MS | Time | HPLC |
| Ex. No. | R | [M + H]⁺ | (min) | Method |
| 99 | 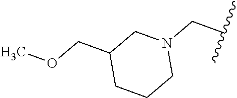 [SMILES: *CN1CCCC(COC)C1] | 462.7 | 0.93 | B |
| 100 | 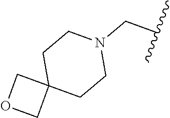 [SMILES: *CN1CCC2(CC1)COC2] | 461.4 | 0.86 | B |
| 101 | 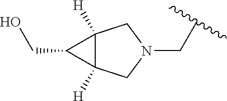 [SMILES: *CN1C[C@@H]2[C@@H](CO)[C@@H]2C1] | 447.2 | 0.88 | B |
| 102 | 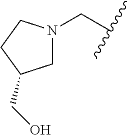 [SMILES: *CN1CC[C@@H](CO)C1] | 435.1 | 0.87 | B |
| 103 | 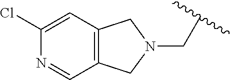 [SMILES: *CN1Cc2cnc(Cl)cc2C1] | 488.2 | 0.99 | B |
| 104 | 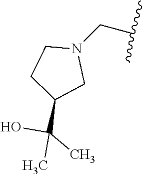 [SMILES: *CN1CC[C@H](C(C)(C)O)C1] | 463.2 | 0.90 | B |
| 105 | 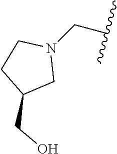 [SMILES: *CN1CC[C@H](CO)C1] | 435.2 | 0.87 | B |
| 106 | 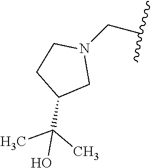 [SMILES: *CN1CC[C@@H](C(C)(C)O)C1] | 463.2 | 0.93 | B |
| 107 | 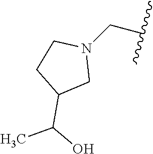 [SMILES: *CN1CCC(C(C)O)C1] | 449.2 | 0.92 | B |
| 108 | 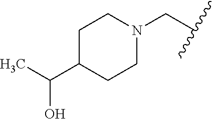 [SMILES: *CN1CCC(C(C)O)CC1] | 463.2 | 0.89 | B |

### Examples 109-115

The compounds in Table 10 were prepared according to the general procedures described for Example, 149 using the appropriate primary or secondary amine.

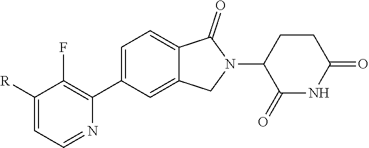

TABLE 10

| | | | Retention | |
|---|---|---|---|---|
| | | LC/MS | Time | HPLC |
| Ex. No. | R | [M + H]⁺ | (min) | Method |
| 109 | 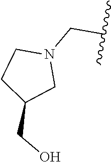 [SMILES: *CN1CC[C@H](CO)C1] | 453.0 | 0.91 | B |
| 110 | 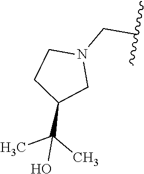 [SMILES: *CN1CC[C@H](C(C)(C)O)C1] | 481.1 | 0.92 | B |
| 111 | 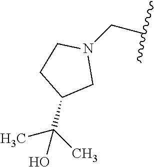 [SMILES: *CN1CC[C@@H](C(C)(C)O)C1] | 481.2 | 1.02 | B |
| 112 | 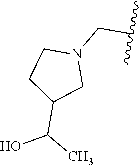 [SMILES: *CN1CCC(C(C)O)C1] | 467.2 | 0.95 | B |
| 113 | 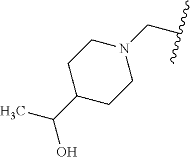 [SMILES: *CN1CCC(C(C)O)CC1] | 481.2 | 0.94 | B |
| 114 | 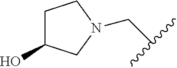 [SMILES: *CN1CC[C@H](O)C1] | 439.2 | 0.90 | B |
| 115 | 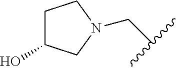 [SMILES: *CN1CC[C@@H](O)C1] | 439.2 | 0.90 | B |

### Example 116

### 2-(2,6-dioxopiperidin-3-yl)-5-(4-(((S)-3-(hydroxymethyl)pyrrolidin-1l-yl)methyl)pyridin-2-yl)isoindoline-1,3-dione

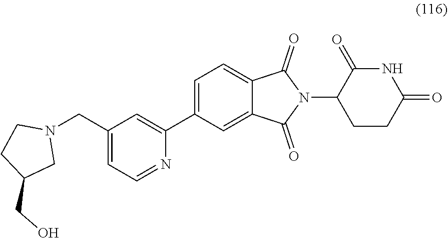 [SMILES: O=C1CCC(N2C(=O)c3ccc(-c4cc(CN5CC[C@H](CO)C5)ccn4)cc3C2=O)C(=O)N1]

### Intermediate 116A: 5-bromo-2-(2,6-dioxopiperidin-3-yl)isoindoline-1,3-dione

To 5-bromoisobenzofuran-1,3-dione (5 g, 22.03 mmol), tert-butyl 4,5-diamino-5-oxopentanoate (4.45 g, 22.03 mmol) and sodium acetate (1.807 g, 22.03 mmol) in a 250 mL bottle was added acetic acid (50 mL). The bottle was sealed and heated at 150° C. for 16 h. The reaction mixture was cooled to room temperature and a precipitate formed. The solid was collected by filtration and dried under vacuum for 6 h to afford 5-bromo-2-(2,6-dioxopiperidin-3-yl)isoindoline-1,3-dione (2 g, 25% yield) as an off-white solid. MS: M−H=335.

### Intermediate 116B: 2-(2,6-dioxopiperidin-3-yl)-5-(4-(hydroxymethyl)pyridin-2-yl) isoindoline-1,3-dione

To a stirred solution of (2-chloropyridin-4-yl)methanol (500 mg, 3.48 mmol) in dioxane (20 mL) was added hexamethylditin, 97% (1.083 mL, 5.22 mmol). The mixture was purged with N₂ for 5 min and then [1,1′-bis(di-tert-butylphosphino)ferrocene]dichloropalladium(II) (227 mg, 0.348 mmol) was added. The reaction vessel was sealed and heated at 100° C. for 2 h. The reaction mixture was cooled to room temperature. The reaction mixture was filtered through a celite pad and washed with dioxane. The combined filtrate solution was transferred into a 250 mL of sealed bottle. Next, 5-bromo-2-(2,6-dioxopiperidin-3-yl)isoindoline-1,3-dione (1 g, 2.97 mmol) was added. The mixture was purged with N₂ for 5 min. Then bis(triphenylphosphine)palladium(II) dichloride (0.312 g, 0.445 mmol) was added. The reaction vessel was sealed and heated at 110° C. for 3 h. The reaction mixture was filtered through celite. The filtrate was concentrated and purified with ISCO 80 g column, 65 mL/min, 0-15% MeOH/CH₂Cl₂. The desired product was eluted with 10% MeOH/CH₂Cl₂. After concentration, 2-(2,6-dioxopiperidin-3-yl)-5-(4-(hydroxymethyl)pyridin-2-yl)isoindoline-1,3-dione (370 mg, 0.962 mmol, 32.4% yield) was obtained as an off-white solid. MS: M+H=366; ¹H NMR (500 MHz, DMSO-d₆) δ 11.12 (s, 1H), 8.67 (d, J=4.9 Hz, 1H), 8.57 (d, J=7.9 Hz, 1H), 8.53 (s, 1H), 8.10 (s, 1H), 8.02 (d, J=7.9 Hz, 1H), 7.42 (d, J=4.6 Hz, 1H), 5.17 (dd, J=12.8, 5.2 Hz, 1H), 4.64 (s, 2H), 2.97-2.75 (m, 1H), 2.62-2.50 (m, 9H), 2.18-2.01 (m, 1H).

### Intermediate 116C: 5-(4-(chloromethyl)pyridin-2-yl)-2-(2,6-dioxopiperidin-3-yl) isoindoline-1,3-dione

To a solution of 2-(2,6-dioxopiperidin-3-yl)-5-(4-(hydroxymethyl)pyridin-2-yl) isoindoline-1,3-dione (370 mg, 1.013 mmol) in CH₂Cl₂ (3 mL) at 0° C. was added thionyl chloride (0.220 mL, 3.04 mmol) dropwise. The reaction mixture was stirred at room temperature for 1 h. The reaction mixture was concentrated to give 5-(4-(chloromethyl) pyridin-2-yl)-2-(2,6-dioxopiperidin-3-yl)isoindoline-1,3-dione (389 mg, 0.912 mmol, 90% yield) as off-white solid. MS: M+H=384.2.

### Example 116

To a solution of 3-(5-(4-(chloromethyl)-3-fluoropyridin-2-yl)-1-oxoisoindolin-2-yl)piperidine-2,6-dione (15 mg, 0.039 mmol) in DMF (1 mL) at room temperature was added 1-(pyrrolidin-3-yl)ethan-1-ol (8.91 mg, 0.077 mmol). The reaction vessel was sealed and heated at 80° C. for 3 h. The reaction mixture was cooled to room temperature. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 3% B, 3-43% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to afford 2-(2,6-dioxopiperidin-3-yl)-5-(4-(((S)-3-(hydroxymethyl)pyrrolidin-1-yl)methyl)pyridin-2-yl)isoindoline-1,3-dione (1.1 mg, 2.453 μmol, 6.28% yield). M+H=449.1 tr=0.95 min. (Method B). ¹H NMR (500 MHz, DMSO-d₆) δ 11.15 (s, 1H), 8.83 (br s, 2H), 8.62 (br d, J=7.6 Hz, 2H), 8.56 (s, 1H), 8.35 (br s, 2H), 8.09 (br d, J=7.9 Hz, 2H), 7.59 (br s, 1H), 5.20 (br dd, J=13.0, 5.6 Hz, 2H), 3.89 (s, 1H), 3.40 (br d, J=12.8 Hz, 2H), 3.16 (s, 2H), 3.00-2.78 (m, 4H), 2.75-2.56 (m, 3H), 2.56-2.52 (m, 4H), 2.21-2.00 (m, 3H), 1.90 (s, 1H), 1.16 (t, J=7.3 Hz, 3H).

### Examples 117-126

The compounds in Table 11 were prepared according to the general procedures described for Example 116, displacing 5-(4-(chloromethyl)pyridin-2-yl)-2-(2,6-dioxopiperidin-3-yl)isoindoline-1,3-dione with the appropriate primary or secondary amine.

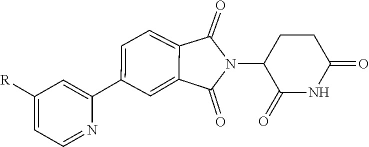

TABLE 11

| | | | Retention | |
|---|---|---|---|---|
| | | LC/MS | Time | HPLC |
| Ex. No. | R | [M + H]⁺ | (min) | Method |
| 117 | 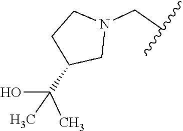 [SMILES: *CN1CC[C@@H](C(C)(C)O)C1] | 477.2 | 1.01 | B |
| 118 | 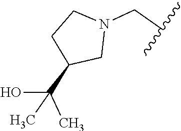 [SMILES: *CN1CC[C@H](C(C)(C)O)C1] | 477.2 | 0.99 | B |
| 119 | 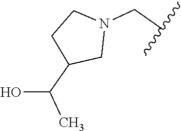 [SMILES: *CN1CCC(C(C)O)C1] | 463.1 | 0.98 | B |
| 120 | 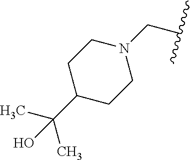 [SMILES: *CN1CCC(C(C)(C)O)CC1] | 491.2 | 1.05 | B |
| 121 | 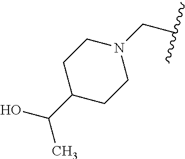 [SMILES: *CN1CCC(C(C)O)CC1] | 477.1 | 1.19 | B |
| 122 | 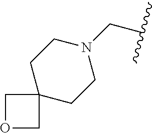 [SMILES: *CN1CCC2(CC1)COC2] | 475.2 | 1.02 | B |
| 123 | 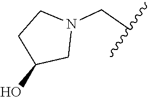 [SMILES: *CN1CC[C@H](O)C1] | 435.1 | 0.98 | B |
| 124 | 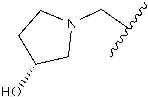 [SMILES: *CN1CC[C@@H](O)C1] | 425.1 | 0.90 | B |
| 125 | 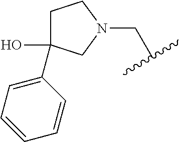 [SMILES: *CN1CCC(O)(c2ccccc2)C1] | 511.0 | 1.20 | B |
| 126 | 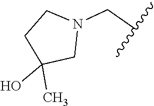 [SMILES: *CN1CCC(C)(O)C1] | 449.1 | 1.0 | B |

### Example 127

### (R)-3-((R)-4-fluoro-5-(4-(((S)-3-hydroxypyrrolidin-1-yl)methyl)pyridin-2-yl)-3-methyl-1-oxoisoindolin-2-yl)piperidine-2,6-dione

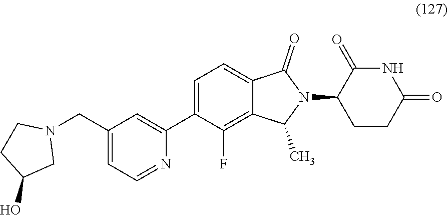 [SMILES: C[C@@H]1c2c(ccc(-c3cc(CN4CC[C@H](O)C4)ccn3)c2F)C(=O)N1[C@@H]1CCC(=O)NC1=O]

### Intermediate 127A: 4-Bromo-2-ethyl-3-fluorobenzoic acid

A solution of 4-bromo 2,3-difluorobenzoic acid (2.0 g, 8.44 mmol) in tetrahydrofuran (40 mL) was cooled to −78° C. and 1 M solution of ethyl magnesium bromide in THE (8.44 mL, 25.3 mmol) was added dropwise. The reaction mixture was allowed to warm to room temperature and stirred under nitrogen atmosphere for 12 h. The reaction was quenched by the addition of MeOH (15 mL) dropwise at 0° C. Volatiles were removed under reduced pressure and the residue was partitioned between EtOAc and 2 M aqueous HCl. The layers were separated, and the aqueous layer was extracted with EtOAc. The combined organic layer was washed with brine, dried over anhydrous Na₂SO₄, filtered and concentrated under reduced pressure to give crude product. The crude product was purified by flash chromatography (SiO₂, 40 g column, 0-80% EtOAc/Pet-ether) to afford 4-bromo-2-ethyl-3-fluorobenzoic acid (1 g, 48% yield). LCMS (Method A): retention time 0.69 min, [M−H]⁺245.1, 247.1; ¹H NMR (300 MHz, DMSO-d₆) δ 13.36 (s, ¹H), 7.68-7.53 (m, 2H), 2.95 (qd, J=7.4, 2.6 Hz, 2H), 1.24-1.06 (m, 3H).

### Intermediate 127B: Methyl 4-bromo-2-ethyl-3-fluorobenzoate

To a stirred mixture of 4-bromo-2-ethyl-3-fluorobenzoic acid (0.7 g, 2.83 mmol) and K₂CO₃ (0.783 g, 5.67 mmol) in acetone (15 mL) was added dimethyl sulfate (0.541 mL, 5.67 mmol) dropwise at room temperature. The reaction mixture was stirred at 50° C. for 14 h and filtered through celite pad. The filtrate was concentrated under vacuum and purified by flash chromatography (SiO₂, 24 g column, 0-50% EtOAc/Pet-ether) to afford methyl 4-bromo-2-ethyl-3-fluorobenzoate (0.51 g, 69% yield) as a color-less oil. ¹H NMR (300 MHz, CHLOROFORM-d) δ 7.58-7.51 (m, 1H), 7.49-7.37 (m, 1H), 3.90 (s, 3H), 3.01 (qd, J=7.4, 2.6 Hz, 2H), 1.23 (t, J 7.4 Hz, 3H).

### Intermediate 127C: Methyl 4-bromo-2-(1-bromoethyl)-3-fluorobenzoate

To a stirred solution of methyl 4-bromo-2-ethyl-3-fluorobenzoate (0.515 g, 1.972 mmol) in DCE (10 mL) was added NBS (0.386 g, 2.170 mmol) followed by AIBN (0.065 g, 0.394 mmol). The reaction mixture was heated at 85° C. for 15 h. The reaction mixture was diluted with ethyl acetate, washed with saturated 10% sodium thiosulfate solution and brine solution. The organic layer was dried over anhydrous sodium sulphate, filtered and concentrated under vacuum. The resulting residue was purified by flash chromatography (SiO₂, 24 g column, 0-30% EtOAc/Pet-ether) to afford methyl 4-bromo-2-(1-bromoethyl)-3-fluorobenzoate (0.6 g, 89% yield) as a white solid. ¹H NMR (300 MHz, CHLOROFORM-d) δ 7.70-7.53 (m, 1H), 7.52-7.44 (m, 1H), 6.16-5.87 (m, 1H), 3.93 (s, 3H), 1.95 (dd, J=7.0, 1.3 Hz, 3H).

### Intermediate 127D: tert-Butyl (4S)-5-amino-4-(5-bromo-4-fluoro-3-methyl-1-oxoisoindolin-2-yl)-5-oxopentanoate

To a stirred solution of methyl 4-bromo-2-(1-bromoethyl)-3-fluorobenzoate (0.86 g, 2.53 mmol) and H-GLU(OTBU)—NH₂ HCL (0.845 g, 3.54 mmol) in acetonitrile (15 mL) was added DIPEA (1.325 mL, 7.59 mmol). The reaction mixture was heated at 85° C. for 15 h. Volatiles were removed under reduced pressure and purified by flash chromatography (SiO₂, 40 g column, 0-10% MeOH/DCM) to afford tert-butyl (4S)-5-amino-4-(5-bromo-4-fluoro-3-methyl-1-oxoisoindolin-2-yl)-5-oxopentanoate (0.23 g, 21% yield) as a pale yellow solid. LCMS (Method A): retention time 1.19 min, [M+23H]⁺451.3, 453.4.

### Intermediate 127 E: tert-butyl (S)-5-amino-4-((R)-5-bromo-4-fluoro-3-methyl-1-oxoisoindolin-2-yl)-5-oxopentanoate

Intermediate 127D was resolved by preparative SFC using the following conditions: Chiralpak IG column (250×30 mm (5 m), 65% CO₂, eluting with 40% 5 nM NH₄OAc in ACN/MeOH (1:1), 100 bar, flow rate of 160 g/min, 40° C., monitoring at 220 nM. Intermediate 127E eluted at 5.5 minutes.

Analytical SFC conditions: Chiralpak IG column (250×4.6 mm (5 m), 65% CO₂, eluting with 35% 5 nM NH₄OAc in ACN/MeOH (1:1), 100 bar, flow rate of 3.0 g/min, 35° C., monitoring at 220 nM. (RT=7.5 minutes).

### Intermediate 127F: ((R)-2-((S)-1-amino-5-(tert-butoxy)-1,5-dioxopentan-2-yl)-4-fluoro-3-methyl-1-oxoisoindolin-5-yl)boronic acid

To a stirred solution of Intermediate 127 E (3.5 g, 8.15 mmol), potassium acetate (1.200 g, 12.23 mmol), BisPin (3.11 g, 12.23 mmol) in DME (150 mL) was purged with argon for 5 min and added PdCl₂(dppf)₂-CH₂Cl₂ adduct (0.666 g, 0.815 mmol) at 25° C. The reaction mixture was sealed and heated to 90° C. and continued to stir for 16 h. The reaction mixture was filtered through the celite pad and washed with EtOAC (20 mL). The filtrate was evaporated under reduced pressure to give crude compound. The crude compound was redissolved in diethyl ether (150 mL), filtered through the celite pad and washed with EtOAC (20 ml), the filtrate was evaporated under reduced pressure to give tert-butyl (S)-5-amino-4-((R)-4-fluoro-3-methyl-1-oxo-5-(4,4,5,5-tetramethyl-1,3,2-dioxaborolan-2-yl)isoindolin-2-yl)-5-oxopentanoate crude compound as a black liquid. MS: M+H=477.

### Intermediate 127G: tert-butyl (S)-5-amino-4-((R)-4-fluoro-5-(4-(hydroxymethyl)pyridin-2-yl)-3-methyl-1-oxoisoindolin-2-yl)-5-oxopentanoate

A 250 mL round bottom flask was charged with (2-chloropyridin-4-yl)methanol (1.754 g, 12.22 mmol), tert-butyl (S)-5-amino-4-((R)-4-fluoro-3-methyl-1-oxo-5-(4,4,5,5-tetramethyl-1,3,2-dioxaborolan-2-yl)isoindolin-2-yl)-5-oxopentanoate (3.88 g, 8.15 mmol), dioxane (80 mL) and 2 M aqueous K₃PO₄ (12.22 mL, 24.44 mmol). The flask was purged with N₂ for 5 min., then Pd(dtbpf)Cl₂ (0.265 g, 0.407 mmol) was added. The reaction flask was sealed and heated overnight at 60° C. The reaction mixture was diluted with EtOAc, washed with brine, and the organic layer separated and concentrated. The crude material was purified with ISCO 80 g column, 60 mL/min. 0-20% MeOH/CH₂Cl₂ in 40 min. The desired product was eluted with 10% MeOH/CH₂Cl₂. Fractions containing the desired product were pooled and concentrated to give tert-butyl (S)-5-amino-4-((R)-4-fluoro-5-(4-(hydroxymethyl)pyridin-2-yl)-3-methyl-1-oxoisoindolin-2-yl)-5-oxopentanoate (700 mg, 1.530 mmol, 18.78% yield) as a light yellow solid. LC MS: tr=0.76 min. M+H=458.1.

### Intermediate 127H: tert-butyl (S)-5-amino-4-((R)-5-(4-(chloromethyl)pyridin-2-yl)-4-fluoro-3-methyl-1-oxoisoindolin-2-yl)-5-oxopentanoate

To a solution of Intermediate 127G (700 mg, 1.530 mmol) in CH₂Cl₂ (10 mL) at 0° C. was added thionyl chloride (0.222 mL, 3.06 mmol). The reaction mixture was stirred at 0° C. for 3 h. The mixture was rotovapped to remove the solvent to afford tert-butyl (S)-5-amino-4-((R)-5-(4-(chloromethyl)pyridin-2-yl)-4-fluoro-3-methyl-1-oxoisoindolin-2-yl)-5-oxopentanoate (700 mg, 1.471 mmol, 96% yield). MS: M+H=476.4.

### Intermediate 1271: (R)-3-((R)-5-(4-(chloromethyl)pyridin-2-yl)-4-fluoro-3-methyl-1-oxoisoindolin-2-yl)piperidine-2,6-dione

To Intermediate 127H (525 mg, 1.103 mmol) in a vial was added AcOH (5 mL), followed by benzenesulfonic acid (174 mg, 1.103 mmol). The reaction vessel was sealed and heated at 120° C. for 3 hours. The reaction mixture was cooled to room temperature and the solvent was removed. (R)-3-((R)-5-(4-(chloromethyl)pyridin-2-yl)-4-fluoro-3-methyl-1-oxoisoindolin-2-yl)piperidine-2,6-dione (443 mg, 0.937 mmol, 85% yield) was obtained as a black solid. MS: M+H=402.3.

### Example 127

To a solution of 5-(4-(chloromethyl)pyridin-2-yl)-2-(2,6-dioxopiperidin-3-yl) isoindoline-1,3-dione (15 mg, 0.039 mmol) in DMF (1 mL) at room temperature was added (S)-pyrrolidin-3-ol (6.50 mg, 0.075 mmol) and Hunig's base (0.033 mL, 0.187 mmol). The reaction vessel was sealed and heated at 80° C. for 3 h. The reaction mixture was cooled to room temperature. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 1% B, 1-41% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. (R)-3-((R)-4-fluoro-5-(4-(((S)-3-hydroxypyrrolidin-1-yl)methyl)pyridin-2-yl)-3-methyl-1-oxoisoindolin-2-yl)piperidine-2,6-dione (12.6 mg, 0.027 mmol, 73.3% yield) was obtained. LC MS: M+H=453.2 tr=0.90 min (Method B). ¹H NMR (500 MHz, DMSO-d₆) δ 8.67 (d, J=4.9 Hz, 2H), 8.04 (br t, J=7.2 Hz, 2H), 7.79 (s, 2H), 7.71-7.57 (m, 2H), 7.39 (d, J=4.6 Hz, 2H), 5.05 (br d, J=6.7 Hz, 1H), 4.94 (q, J=6.5 Hz, 1H), 4.82 (br dd, J=12.8, 5.2 Hz, 1H), 4.73 (br s, 1H), 4.22 (br s, 2H), 3.76-3.64 (m, 3H), 2.94-2.78 (m, 2H), 2.77-2.56 (m, 8H), 2.54 (s, 13H), 2.48-2.43 (m, 2H), 2.41-2.20 (m, 3H), 2.10-1.94 (m, 4H), 1.90 (s, 4H), 1.63-1.47 (m, 8H).

### Examples 128-142

The compounds in Table 12 were prepared according to the procedures described for Example 127, displacing the chloride in Intermediate 127H with the appropriate primary or secondary amine.

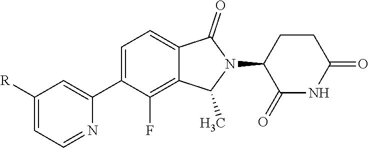

TABLE 12

| | | | Retention | |
|---|---|---|---|---|
| | | LC/MS | Time | HPLC |
| Ex. No. | R | [M + H]⁺ | (min) | Method |
| 128 | 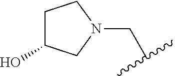 [SMILES: *CN1CC[C@@H](O)C1] | 453.0 | 0.94 | B |
| 129 | 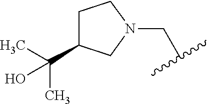 [SMILES: *CN1CC[C@H](C(C)(C)O)C1] | 495.2 | 0.96 | B |
| 130 | 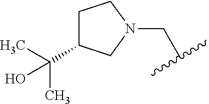 [SMILES: *CN1CC[C@@H](C(C)(C)O)C1] | 495.2 | 0.96 | B |
| 131 | 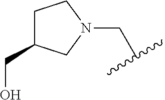 [SMILES: *CN1CC[C@H](CO)C1] | 467.1 | 0.94 | B |
| 132 | 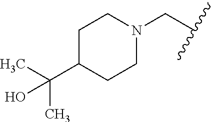 [SMILES: *CN1CCC(C(C)(C)O)CC1] | 509.1 | 0.97 | B |
| 133 | 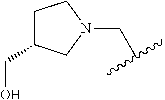 [SMILES: *CN1CC[C@@H](CO)C1] | 467.1 | 0.94 | B |
| 134 | 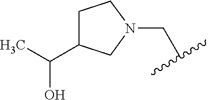 [SMILES: *CN1CCC(C(C)O)C1] | 481.1 | 0.97 | B |
| 135 | 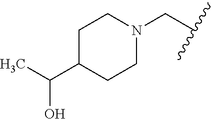 [SMILES: *CN1CCC(C(C)O)CC1] | 495.1 | 0.98 | B |
| 136 | 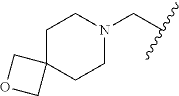 [SMILES: *CN1CCC2(CC1)COC2] | 493.1 | 0.96 | B |
| 137 | 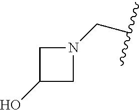 [SMILES: *CN1CC(O)C1] | 439.1 | 0.94 | B |
| 138 | 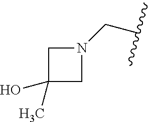 [SMILES: *CN1CC(C)(O)C1] | 453.0 | 0.91 | B |
| 139 | 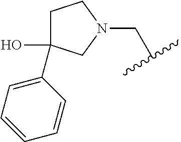 [SMILES: *CN1CCC(O)(c2ccccc2)C1] | 529.2 | 1.07 | B |
| 140 | 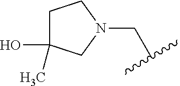 [SMILES: *CN1CCC(C)(O)C1] | 467.1 | 0.97 | B |
| 141 | 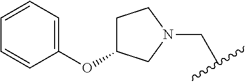 [SMILES: *CN1CC[C@@H](Oc2ccccc2)C1] | 529.1 | 1.23 | B |
| 142 | 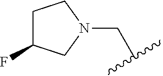 [SMILES: *CN1CC[C@H](F)C1] | 455.1 | 0.90 | B |

### Example 143

### 3-((R)-4-fluoro-5-(3-fluoro-4-((4-(2-hydroxypropan-2-yl)piperidin-1-yl)methyl) pyridin-2-yl)-3-methyl-1-oxoisoindolin-2-yl)piperidine-2,6-dione

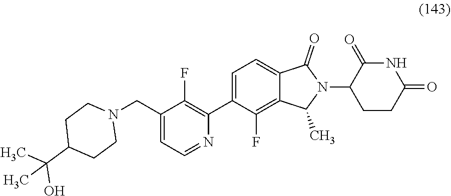 [SMILES: C[C@@H]1c2c(ccc(-c3nccc(CN4CCC(C(C)(C)O)CC4)c3F)c2F)C(=O)N1C1CCC(=O)NC1=O]

### Intermediate 143A: tert-butyl (S)-5-amino-4-((R)-4-fluoro-5-(3-fluoro-4-(hydroxymethyl) pyridin-2-yl)-3-methyl-1-oxoisoindolin-2-yl)-5-oxopentanoate

To a solution of (2-chloro-3-fluoropyridin-4-yl)methanol (100 mg, 0.619 mmol) in dioxane (3 mL) at room temperature was added hexamethylditin (243 mg, 0.743 mmol). The mixture was purged with N₂ for 2 min. Then [1,1′-bis(di-tert-butylphosphino)ferrocene]dichloropalladium(II) (40.3 mg, 0.062 mmol) was added. The reaction vessel was sealed and heated at 100° C. for 80 min. The reaction mixture was filtered through celite. M+H=292.1.

To this filtrate was added Intermediate 130E (50 mg, 0.116 mmol). The mixture was purged with N₂ for 2 min. Then bis(triphenylphosphine)palladium(II) dichloride (8.18 mg, 0.012 mmol) was added and the reaction vessel was sealed and heated at 110° C. for 2 h. The reaction mixture was cooled to room temperature and then filtered through celite. The filtrate was concentrated and purified with ISCO 40 g column, 40 mL/min. 0-10% MeOH/CH₂Cl₂ in 30 min. The desired product was eluted with 10% MeOH/CH₂Cl₂. Fractions containing desired product were combined and concentrated to give tert-butyl (S)-5-amino-4-((R)-4-fluoro-5-(3-fluoro-4-(hydroxymethyl)pyridin-2-yl)-3-methyl-1-oxoisoindolin-2-yl)-5-oxopentanoate (26 mg, 0.049 mmol, 42.3% yield) as a light brown solid. LC MS: M+H=476 tr=0.86 min (Method A). ¹H NMR (400 MHz, CHLOROFORM-d) δ 8.57 (d, J=4.8 Hz, 1H), 7.84-7.70 (m, 2H), 7.60 (t, J=5.0 Hz, 1H), 7.38-7.17 (m, 2H), 6.73 (br s, 1H), 5.41-5.28 (m, 1H), 5.00-4.85 (m, 3H), 4.75 (t, J=7.7 Hz, 1H), 2.63-2.50 (m, 1H), 2.44-2.18 (m, 4H), 1.69-1.58 (m, 5H), 1.49-1.40 (m, 10H), 1.37-1.22 (m, 2H), 1.05-0.79 (m, 1H).

### Intermediate 143B: tert-butyl (S)-5-amino-4-((R)-5-(4-(chloromethyl)-3-fluoropyridin-2-yl)-4-fluoro-3-methyl-1-oxoisoindolin-2-yl)-5-oxopentanoate

To tert-butyl (S)-5-amino-4-((R)-4-fluoro-5-(3-fluoro-4-(hydroxymethyl) pyridin-2-yl)-3-methyl-1-oxoisoindolin-2-yl)-5-oxopentanoate (26 mg, 0.055 mmol) in CH₂Cl₂ (1 mL) at 0° C. was added thionyl chloride (7.93 μL, 0.109 mmol) dropwise. The reaction mixture was stirred at 0° C. for 2 h. The solvent was removed to afford tert-butyl (S)-5-amino-4-((R)-5-(4-(chloromethyl)-3-fluoropyridin-2-yl)-4-fluoro-3-methyl-1-oxoisoindolin-2-yl)-5-oxopentanoate (27 mg, 0.049 mmol, 90% yield) as a light brown solid. LC MS: MS: M+H=494.3.

### Intermediate 143C: (R)-3-((R)-5-(4-(chloromethyl)-3-fluoropyridin-2-yl)-4-fluoro-3-methyl-1-oxoisoindolin-2-yl) piperidine-2,6-dione

To tert-butyl (S)-5-amino-4-((R)-5-(4-(chloromethyl)-3-fluoropyridin-2-yl)-4-fluoro-3-methyl-1-oxoisoindolin-2-yl)-5-oxopentanoate (27 mg, 0.055 mmol) in AcOH (2 mL) at room temperature was added benzenesulfonic acid (8.65 mg, 0.055 mmol). The reaction vessel was sealed and heated at 130° C. for 3 h. The solvent was removed to afford (R)-3-((R)-5-(4-(chloromethyl)-3-fluoropyridin-2-yl)-4-fluoro-3-methyl-1-oxoisoindolin-2-yl) piperidine-2,6-dione (22 mg, 0.047 mmol, 86% yield) as a brown solid. LC MS: M+H=420.3.

### Example 143

To a solution of 3-(5-(4-(chloromethyl)-3-fluoropyridin-2-yl)-1-oxoisoindolin-2-yl)piperidine-2,6-dione (15 mg, 0.039 mmol) in DMF (1 mL) at room temperature was added 2-(piperidin-4-yl)propan-2-ol (6.82 mg, 0.048 mmol), followed by Hunig's base (0.021 mL, 0.119 mmol). The reaction vessel was sealed and heated at 80° C. for 3 h. The reaction mixture was cooled to room temperature. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 8% B, 8-48% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to afford 3-((R)-4-fluoro-5-(3-fluoro-4-((4-(2-hydroxypropan-2-yl)piperidin-1-yl)methyl)pyridin-2-yl)-3-methyl-1-oxoisoindolin-2-yl) piperidine-2,6-dione (7.8 mg, 0.014 mmol, 59.1% yield). LC MS: tr=0.968 min (Method B), M+H=527.2. 1H NMR (500 MHz, DMSO-d₆) δ 10.98 (br d, J=19.7 Hz, 1H), 8.54 (d, J=4.7 Hz, 2H), 7.78-7.64 (m, 4H), 7.59 (t, J=5.0 Hz, 2H), 5.06 (q, J=6.5 Hz, 1H), 4.95 (q, J=6.6 Hz, 1H), 4.82 (br dd, J=12.4, 5.2 Hz, 1H), 4.72 (br s, 1H), 4.09 (s, 1H), 3.62 (s, 3H), 3.52-3.31 (m, 2H), 2.99 (s, 1H), 2.94-2.79 (m, 5H), 2.78-2.70 (m, 1H), 2.69-2.57 (m, 4H), 2.54 (s, 16H), 2.48-2.28 (m, 1H), 2.20-2.01 (m, 2H), 1.96 (br t, J=11.0 Hz, 4H), 1.66 (br d, J=11.8 Hz, 4H), 1.53 (br d, J=6.7 Hz, 3H), 1.51 (br d, J=6.8 Hz, 3H), 1.38-1.21 (m, 4H), 1.21-1.09 (m, 2H), 1.03 (s, 11H).

### Example 144

The compound in Table 13 was prepared according to the general procedures described for Example 14, displacing the benzyl chloride in Intermediate 143C with the appropriate primary or secondary amine.

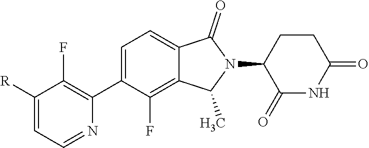

TABLE 13

| | | | Retention | |
|---|---|---|---|---|
| Ex. | | LC/MS | Time | HPLC |
| No. | R | [M + H]⁺ | (min) | Method |
| 144 | 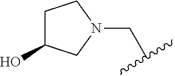 [SMILES: *CN1CC[C@H](O)C1] | 471.2 | 0.91 | A |

### Example 145

### (S)-3-(5-(4-((6-hydroxy-2-azaspiro[3.3]heptan-2-yl)methyl)pyridin-2-yl)-1-oxoisoindolin-2-yl)piperidine-2,6-dione

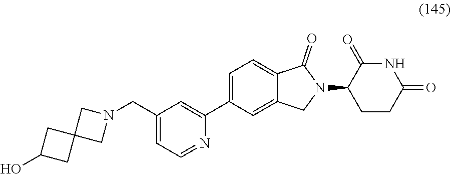 [SMILES: O=C1CC[C@@H](N2Cc3cc(-c4cc(CN5CC6(CC(O)C6)C5)ccn4)ccc3C2=O)C(=O)N1]

To the solution of tert-butyl 6-hydroxy-2-azaspiro[3.3]heptane-2-carboxylate (43 mg, 0.202 mmol) in CH₂Cl₂ (0.5 mL) was added 3 M hydrogen chloride (0.504 mL, 2.016 mmol) in methyl t-butyl ether. The resulting solution was stirred for 2 hours. The volatiles were removed, the residue was re-dissolved in DMF (1 mL). To this solution was added Intermediate 101B (74.6 mg, 0.202 mmol) followed by Hunig's base (0.070 mL, 0.403 mmol). The reaction mixture became a clear solution. The vial was sealed and heated at 70° C. for 4.5 h. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 2-minute hold at 0% B, 0-40% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 3.7 mg, (3.9%). MS (ES): m/z=447.10 [M+H]⁺. ¹H NMR (500 MHz, DMSO-d₆) δ 11.02 (s, 1H), 8.79 (br d, J=4.6 Hz, 1H), 8.32 (s, 1H), 8.23 (br d, J=7.6 Hz, 1H), 8.13 (s, 1H), 7.89 (d, J=7.9 Hz, 1H), 7.47 (br d, J=4.6 Hz, 1H), 5.16 (dd, J=13.0, 5.0 Hz, 1H), 4.63-4.51 (m, 1H), 4.47 (s, 2H), 4.50-4.41 (m, 1H), 4.23-4.07 (m, 4H), 3.98 (br t, J=7.0 Hz, 1H), 2.93 (br s, 1H), 2.63 (br d, J=16.5 Hz, 1H), 2.48-2.28 (m, 2H), 2.10-1.98 (m, 4H).

### Example 146

### (3S)-3-(5-(4-((5-hydroxyhexahydrocyclopenta[c]pyrrol-2(1H)-yl)methyl)pyridin-2-yl)-1-oxoisoindolin-2-yl)piperidine-2,6-dione

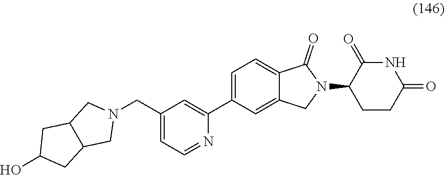 [SMILES: O=C1CC[C@@H](N2Cc3cc(-c4cc(CN5CC6CC(O)CC6C5)ccn4)ccc3C2=O)C(=O)N1]

To the solution of tert-butyl 5-hydroxyhexahydrocyclopenta[c]pyrrole-2(1H)-carboxylate (30 mg, 0.132 mmol) in CH₂Cl₂ (1 mL) was added TFA (10.17 μL, 0.132 mmol). The resulting solution was stirred for 2 hours. The volatiles were removed and the residue was re-dissolved in DMF (1 mL). To this solution was added Intermediate 101B (48.8 mg, 0.132 mmol) followed by Hunig's base (0.046 mL, 0.264 mmol). The reaction mixture became a clear solution. The vial was sealed and heated at 70° C. for 4.5 h. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 6% B, 6-47% B over 23 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 40.5 mg, (64.4%). MS (ES): m/z=461.20 [M+H]⁺. ¹H N-MR (500 MHz, DMSO-d₆) δ 8.67 (d, J=4.9 Hz, 1H), 8.31 (s, 1H), 8.22 (d, J=8.2 Hz, 1H), 7.96 (s, 1H), 7.85 (d, J=7.9 Hz, 1H), 7.34 (d, J=4.6 Hz, 1H), 5.15 (dd, J=13.4, 5.2 Hz, 1H), 4.56 (br d, J=17.1 Hz, 1H), 4.43 (d, J=17.4 Hz, 1H), 4.03-3.81 (m, 1H), 3.73 (s, 2H), 3.01-2.78 (m, 1H), 2.66 (br d, J=9.5 Hz, 2H), 2.61 (br d, J=4.6 Hz, 1H), 2.59-2.55 (m, 2H), 2.50-2.38 (m, 1H), 2.37-2.21 (m, 2H), 2.17-1.99 (m, 1H), 1.97-1.84 (m, 2H), 1.42 (br d, J=12.8 Hz, 2H).

### Example 147

### (3S)-3-(5-(4-((5-hydroxy-5-methylhexahydrocyclopenta[c]pyrrol-2(1H)-yl)methyl) pyridin-2-yl)-1-oxoisoindolin-2-yl)piperidine-2,6-dione

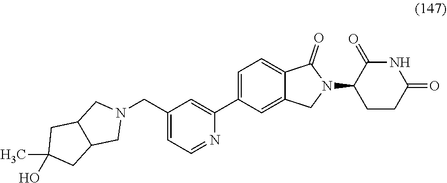 [SMILES: CC1(O)CC2CN(Cc3ccnc(-c4ccc5c(c4)CN([C@@H]4CCC(=O)NC4=O)C5=O)c3)CC2C1]

### Intermediate 147A: tert-butyl 5-hydroxy-5 methylhexahydrocyclopenta[c]pyrrole-2(1H)-carboxylate

To the solution of tert-butyl 5-oxohexahydrocyclopenta[c]pyrrole-2(1H)-carboxylate (56 mg, 0.249 mmol) in THE was added methylmagnesium bromide (746 μL, 0.746 mmol) at 0° C. The reaction mixture was stirred at room temperature for 3 hours. The reaction was quenched with ammonium acetate. The reaction mixture was extracted with ethyl acetate (3×10 mL). The organic was combined and dried over sodium sulfate and concentrated. ¹H NMR (400 MHz, CHLOROFORM-d) δ 3.55-3.42 (m, 2H), 3.35 (dd, J=11.2, 3.5 Hz, 2H), 2.68 (br d, J=4.2 Hz, 2H), 1.93 (br dd, J=13.4, 8.4 Hz, 2H), 1.67 (br dd, J=13.4, 5.0 Hz, 2H), 1.48-1.43 (m, 9H), 1.32 (s, 3H).

### Example 147

To the solution of tert-butyl 5-hydroxy-5-methylhexahydrocyclopenta[c]pyrrole-2(1H)-carboxylate (30 mg, 0.124 mmol) in CH₂Cl₂ (1 mL) was added TFA (9.58 μL, 0.124 mmol). The resulting solution was stirred for 2 hours. The volatiles were removed and the residue was redissolved in DMF (1 mL). To this solution was added Intermediate 101B (46.0 mg, 0.124 mmol) followed by Hunig's base (0.043 mL, 0.249 mmol). The reaction mixture became a clear solution. The vial was sealed and heated at 70° C. for 4.5 h. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 9% B, 9-49% B over 23 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 12% B, 12-52% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 20.2 mg, (32%). MS (ES): m/z=475.30 [M+H]⁺. ¹H NMR (500 MHz, DMSO-d₆) δ 8.68 (d, J=4.9 Hz, 1H), 8.32 (s, 1H), 8.23 (br d, J=7.9 Hz, 1H), 7.97 (s, 1H), 7.85 (d, J=7.9 Hz, 1H), 7.34 (br d, J=5.2 Hz, 1H), 5.16 (br dd, J=13.3, 5.0 Hz, 1H), 4.56 (br d, J=17.4 Hz, 1H), 4.44 (br d, J=17.4 Hz, 1H), 3.73 (s, 2H), 3.04-2.82 (m, 1H), 2.72 (br d, J=9.2 Hz, 2H), 2.68-2.58 (m, 3H), 2.49-2.37 (m, 1H), 2.30 (br t, J=7.6 Hz, 2H), 2.17-1.99 (m, 1H), 1.81 (br dd, J=12.8, 8.5 Hz, 2H), 1.50 (br d, J=12.8 Hz, 2H), 1.16 (s, 3H).

### Example 148

### (3S)-3-(5-(4-((5-methyl-3,3a,4,6a-tetrahydrocyclopenta[c]pyrrol-2(1H)-yl)methyl) pyridin-2-yl)-1-oxoisoindolin-2-yl)piperidine-2,6-dione

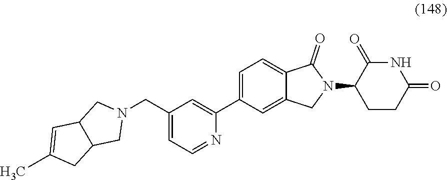 [SMILES: CC1=CC2CN(Cc3ccnc(-c4ccc5c(c4)CN([C@@H]4CCC(=O)NC4=O)C5=O)c3)CC2C1]

To the solution of tert-butyl 5-hydroxy-5-methylhexahydrocyclopenta[c]pyrrole-2(1H)-carboxylate (30 mg, 0.124 mmol) in CH₂Cl₂ (1 mL) was added TFA (9.58 μL, 0.124 mmol). The resulting solution was stirred for 2 hours. The volatiles were removed and the residue was redissolved in DMF (1 mL). To this solution was added Intermediate 101B (46.0 mg, 0.124 mmol) followed by Hunig's base (0.043 mL, 0.249 mmol). The reaction mixture became a clear solution. The vial was sealed and heated at 70° C. for 4.5 h. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 9% B, 9-49% B over 23 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 0% B, 0-40% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 8.1 mg (14.3%). MS (ES): m/z=457.20 [M+H]⁺.

### Example 149

### (3S)-3-(5-(3-fluoro-4-(((3aR,7aS)-5-hydroxyoctahydro-2H-isoindol-2-yl)methyl)pyridin-2-yl)-1-oxoisoindolin-2-yl)piperidine-2,6-dione

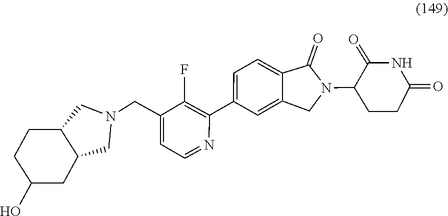 [SMILES: O=C1CCC(N2Cc3cc(-c4nccc(CN5C[C@H]6CCC(O)C[C@H]6C5)c4F)ccc3C2=O)C(=O)N1]

### Intermediate 149A: tert-butyl (S)-5-amino-4-(5-(3-fluoro-4-(hydroxymethyl)pyridin-2-yl)-1-oxoisoindolin-2-yl)-5-oxopentanoate

A 200 mL flask was charged with (2-chloro-3-fluoropyridin-4-yl)methanol (2.55 g, 15.78 mmol), tert-butyl (S)-5-amino-5-oxo-4-(1-oxo-5-(4,4,5,5-tetramethyl-1,3,2-dioxaborolan-2-yl)isoindolin-2-yl)pentanoate (8.00 g, 18.00 mmol), Pd(OAc)₂ (0.202 g, 0.900 mmol), di-tert-butyl(methyl)phosphonium tetrafluoroborate (0.447 g, 1.800 mmol), dioxane (90 mL) and aqueous 3 M K₂CO₃ solution (30.0 mL, 90 mmol). The flask was sealed and the air was replaced with nitrogen. The reaction mixture was heated for 20 h at 80° C. The reaction mixture was cooled to room temperature, diluted with EtOAc, washed with brine, and the organic layer separated, dried over MgSO₄ and concentrated. The mixture was purified using a 220 g silica gel column by ISCO eluting with 0-10% DCM/MeOH to obtain 5.45 g (68% yield) of the titled compound. MS (ES): m/z=444.3 [M+H]⁺.

### Intermediate 149B: (S)-3-(5-(4-(chloromethyl)-3-fluoropyridin-2-yl)-1-oxoisoindolin-2-yl)piperidine-2,6-dione hydrochloride

To tert-butyl 5-amino-4-(5-(3-fluoro-4-(hydroxymethyl)pyridin-2-yl)-1-oxoisoindolin-2-yl)-5-oxopentanoate (5.30 g, 11.95 mmol) dissolved in DCM (120 mL) and cooled in an ice-water bath was added thionyl chloride (2.60 mL, 35.9 mmol), (dropwise). After 10 min., the ice-bath was removed and the reaction mixture was allowed to warm to room temperature. After 30 min, the reaction mixture was concentrated to dryness. The residue obtained was dissolved in acetic acid (60 mL) and then PhSO₃H (4.16 g, 26.3 mmol) was added. The mixture was refluxed (118° C.) for 3 h. The mixture was concentrated to dryness and 30 mL of 3 M HCl in MeOH was added. The mixture was stirred at 0° C. until there was complete dissolution. Then, 70 mL of EtOAc was added, and the mixture was stirred. After about 5 minutes, the mixture was allowed to stay still in the ice-water bath for about 0.5 h. The precipitates were filtered, washed with cold EtOAc, and then air-dried to obtain 87% yield of the titled compound. ¹H NMR (400 MHz, DMSO-d₆) δ ppm 11.02 (s, 1H) 8.61 (d, J=4.68 Hz, 1H) 8.14 (s, 1H) 8.05 (d, J=8.00 Hz, 1H) 7.89 (d, J=8.00 Hz, 1H) 7.68 (t, J=5.07 Hz, 1H) 5.17 (dd, J=13.27, 5.07 Hz, 1H) 4.94 (s, 2H) 4.58 (d, J=17.46 Hz, 1H) 4.45 (d, J=17.56 Hz, 1H) 2.89-2.99 (m, 1H) 2.57-2.67 (m, 1H) 2.38-2.48 (m, 1H) 2.01-2.10 (m, 1H).

### Example 149

To the solution of (S)-3-(5-(4-(chloromethyl)-3-fluoropyridin-2-yl)-1-oxoisoindolin-2-yl)piperidine-2,6-dione (55 mg, 0.142 mmol) was added (3aR,7aS)-octahydro-1H-isoindol-5-ol hydrochloride (25.2 mg, 0.142 mmol) followed by Hunig's base (0.050 mL, 0.284 mmol). The reaction mixture became a clear solution. The vial was sealed and heated at 70° C. for 4.5 h. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 4% B, 4-44% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 35.1 mg, (50.2%). MS (ES): m/z=493.20 [M+H]⁺. ¹H NMR (500 MHz, DMSO-d₆) δ 8.58-8.50 (m, 1H), 8.12 (s, 1H), 8.04 (br d, J=7.9 Hz, 1H), 7.87 (d, J=7.9 Hz, 1H), 7.63-7.52 (m, 1H), 5.16 (dd, J=13.3, 5.0 Hz, 1H), 4.57 (br d, J=17.4 Hz, 1H), 4.44 (br d, J=17.4 Hz, 1H), 3.95-3.79 (m, 2H), 3.75-3.57 (m, 1H), 2.99-2.90 (m, 1H), 2.90-2.79 (m, 1H), 2.73-2.66 (m, 1H), 2.66-2.58 (m, 2H), 2.49-2.31 (m, 2H), 2.30-1.10 (m, 9H).

### Example 150

### (S)-3-(5-(3-fluoro-4-((1-oxo-2,8-diazaspiro[4.5]decan-8-yl)methyl)pyridin-2-yl)-1-oxoisoindolin-2-yl)piperidine-2,6-dione

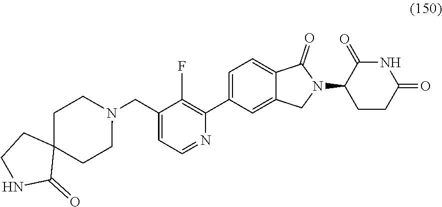 [SMILES: O=C1CC[C@@H](N2Cc3cc(-c4nccc(CN5CCC6(CCNC6=O)CC5)c4F)ccc3C2=O)C(=O)N1]

To the solution of Intermediate 149B (55 mg, 0.142 mmol) was added 2,8-diazaspiro[4.5]decan-1-one hydrochloride (27.0 mg, 0.142 mmol) followed by Hunig's base (0.074 mL, 0.425 mmol). The reaction mixture became a clear solution. The vial was sealed and heated at 70° C. for 4.5 h. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 5% B, 5-45% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 28.7 mg, (40.2%). MS (ES): m/z=506.20 [M+H]⁺. ¹H NMR (500 MHz, DMSO-d₆) δ 8.54 (d, J=4.6 Hz, 1H), 8.12 (s, 1H), 8.04 (br d, J=8.2 Hz, 1H), 7.87 (d, J=8.2 Hz, 1H), 7.62-7.47 (m, 2H), 5.16 (dd, J=13.1, 5.2 Hz, 1H), 4.57 (d, J=17.4 Hz, 1H), 4.44 (d, J=17.4 Hz, 1H), 3.68 (s, 2H), 3.16 (t, J=6.7 Hz, 2H), 3.06-2.86 (m, 1H), 2.80 (br d, J=11.6 Hz, 2H), 2.63 (br d, J=17.4 Hz, 1H), 2.50-2.31 (m, 1H), 2.16 (br t, J=11.0 Hz, 2H), 2.10-2.00 (m, 1H), 1.92 (t, J=6.7 Hz, 2H), 1.74 (td, J=12.5, 3.7 Hz, 2H), 1.35 (br d, J=11.9 Hz, 2H).

### Example 151

### (S)-3-(5-(3-fluoro-4-(((3aR,6aS)-tetrahydro-1H-furo[3,4-c]pyrrol-5(3H)-yl)methyl) pyridin-2-yl)-1-oxoisoindolin-2-yl)piperidine-2,6-dione

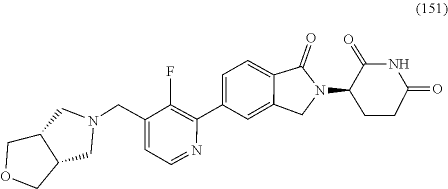 [SMILES: O=C1CC[C@@H](N2Cc3cc(-c4nccc(CN5C[C@H]6COC[C@H]6C5)c4F)ccc3C2=O)C(=O)N1]

To the solution of Intermediate 149B (30 mg, 0.077 mmol) was added (3aR,6aS)-hexahydro-1H-furo[3,4-c]pyrrole (8.75 mg, 0.077 mmol) followed by Hunig's base (0.027 mL, 0.155 mmol). The reaction mixture became a clear solution. The vial was sealed and heated at 70° C. for 4.5 h. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 4% B, 4-44% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 20.0 mg, (54.8%). MS (ES): m/z=465.20 [M+H]⁺. ¹H NMR (500 MHz, DMSO-d₆) δ 11.01 (s, 1H), 8.54 (d, J=4.6 Hz, 1H), 8.11 (s, 1H), 8.03 (br d, J=7.9 Hz, 1H), 7.87 (d, J=7.9 Hz, 1H), 7.54 (t, J=4.9 Hz, 1H), 5.15 (dd, J=13.4, 5.2 Hz, 1H), 4.57 (d, J=17.4 Hz, 1H), 4.44 (d, J=17.4 Hz, 1H), 3.73-3.68 (m, 4H), 3.44 (m 1H), 3.07-2.86 (m, 1H), 2.79-2.69 (m, 2H), 2.68-2.59 (m, 3H), 2.49-2.34 (m, 3H), 2.15-1.96 (m, 1H).

### Example 152

### (S)-3-(1-oxo-5-(4-(((3aR,6aS)-tetrahydro-1H-furo[3,4-c]pyrrol-5(3H)-yl)methyl)pyridin-2-yl)isoindolin-2-yl)piperidine-2,6-dione

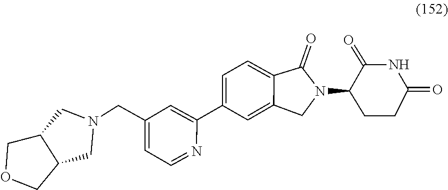 [SMILES: O=C1CC[C@@H](N2Cc3cc(-c4cc(CN5C[C@H]6COC[C@H]6C5)ccn4)ccc3C2=O)C(=O)N1]

To the solution of Intermediate 98B (30 mg, 0.081 mmol) was added (3aR,6aS)-hexahydro-1H-furo[3,4-c]pyrrole (9.18 mg, 0.081 mmol) followed by Hunig's base (0.028 mL, 0.162 mmol). The reaction mixture became a clear solution. The vial was sealed and heated at 70° C. for 4.5 h. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 2% B, 2-42% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 36.6 mg, (99.0%). MS (ES): m/z=447.20 [M+H]⁺. ¹H NMR (500 MHz, DMSO-d₆) δ 8.65 (d, J=4.9 Hz, 1H), 8.31 (s, 1H), 8.23 (d, J=7.9 Hz, 1H), 7.97 (s, 1H), 7.85 (d, J=7.9 Hz, 1H), 7.38 (d, J=4.9 Hz, 1H), 5.15 (dd, J=13.1, 5.2 Hz, 1H), 4.56 (d, J=17.4 Hz, 1H), 4.44 (d, J=17.4 Hz, 1H), 3.81-3.70 (m, 2H), 3.68 (s, 2H), 3.44 (br s, 1H), 3.02-2.83 (m, 1H), 2.74 (br s, 2H), 2.65-2.57 (m, 3H), 2.49-2.39 (m, 1H), 2.37 (dd, J=9.0, 2.0 Hz, 2H), 2.18-1.96 (m, 1H).

### Example 153

### (S)-3-(5-(3-fluoro-4-((6-hydroxy-2-azaspiro[3.3]heptan-2-yl)methyl)pyridin-2-yl)-1-oxoisoindolin-2-yl)piperidine-2,6-dione

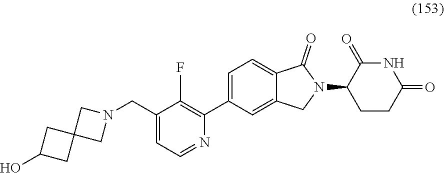 [SMILES: O=C1CC[C@@H](N2Cc3cc(-c4nccc(CN5CC6(CC(O)C6)C5)c4F)ccc3C2=O)C(=O)N1]

To the solution of tert-butyl 6-hydroxy-2-azaspiro[3.3]heptane-2-carboxylate (50 mg, 0.234 mmol) in CH₂Cl₂ (0.5 mL) was added 3 M hydrogen chloride (0.584 mL, 2.344 mmol) in methyl t-butyl ether. The resulting solution was stirred for 2 hours. The volatiles were removed and the residue was redissolved in DMF (1 mL). To this solution was added Intermediate 153B (91 mg, 0.234 mmol) followed by Hunig's base (0.082 mL, 0.469 mmol). The reaction mixture became a clear solution. The vial was sealed and heated at 70° C. for 4.5 h. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 0% B, 0-40% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 16.4 mg, (14.4%). MS (ES): m/z=465.10 [M+H]⁺. ¹H NMR (500 MHz, DMSO-d₆) δ 11.02 (s, 1H), 8.55 (br d, J=4.3 Hz, 1H), 8.11 (s, 1H), 8.03 (br d, J=7.9 Hz, 1H), 7.88 (d, J=7.9 Hz, 1H), 7.49 (br t, J=4.6 Hz, 1H), 5.16 (dd, J=13.4, 4.9 Hz, 1H), 4.57 (d, J=17.7 Hz, 1H), 4.44 (d, J=17.1 Hz, 1H), 4.00-3.81 (m, 1H), 3.06-2.87 (m, 1H), 2.69-2.60 (m, 1H), 2.49-2.37 (m, 3H), 2.23-2.00 (m, 1H), 2.00-1.87 (m, 2H).

### Example 154

### tert-butyl (3aS,6aS)-5-((2-(2-((S)-2,6-dioxopiperidin-3-yl)-1-oxoisoindolin-5-yl)-3-fluoropyridin-4-yl)methyl)hexahydropyrrolo[3,4-c]pyrrole-2(1H)-carboxylate

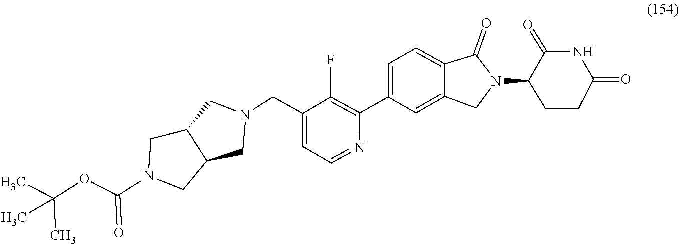 [SMILES: CC(C)(C)OC(=O)N1C[C@@H]2CN(Cc3ccnc(-c4ccc5c(c4)CN([C@@H]4CCC(=O)NC4=O)C5=O)c3F)C[C@H]2C1]

To the solution of Intermediate 153B (10 mg, 0.026 mmol) was added tert-butyl (3aS,6aS)-hexahydropyrrolo[3,4-c]pyrrole-2(1H)-carboxylate (5.47 mg, 0.026 mmol) followed by Hunig's base (0.009 mL, 0.052 mmol). The reaction mixture became a clear solution. The vial was sealed and heated at 70° C. for 4.5 h. The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 17% B, 17-57% B over 20 minutes, then a 4-minute hold at 100% B; Flow Rate: 20 mL/min; Column Temperature: 25° C. Fraction collection was triggered by MS signals. Fractions containing the desired product were combined and dried via centrifugal evaporation. The yield of the product was 5.2 mg, (34.7%). MS (ES): m/z=564.40 [M+H]⁺. ¹H NMR (500 MHz, DMSO-d₆) δ 8.53 (d, J=4.9 Hz, 1H), 8.11 (s, 1H), 8.03 (br d, J=7.6 Hz, 1H), 7.87 (d, J=7.9 Hz, 1H), 7.56 (t, J=4.7 Hz, 1H), 5.15 (dd, J=13.3, 5.0 Hz, 1H), 4.56 (d, J=17.4 Hz, 1H), 4.44 (d, J=17.4 Hz, 1H), 4.03 (s, 2H), 2.98-2.80 (m, 5H), 2.69-2.58 (m, 3H), 2.43 (br dd, J=13.1, 4.9 Hz, 1H), 2.32-2.15 (m, 2H), 2.14-1.93 (m, 1H), 1.40 (s, 9H).

### Example 155

### (3S)-3-(5-(3-fluoro-4-((hexahydrocyclopenta[c]pyrrol-2(1H)-yl)methyl)pyridin-2-yl)-1-oxoisoindolin-2-yl)piperidine-2,6-dione

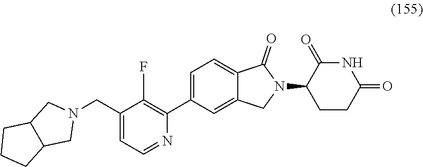 [SMILES: O=C1CC[C@@H](N2Cc3cc(-c4nccc(CN5CC6CCCC6C5)c4F)ccc3C2=O)C(=O)N1]

To the solution of tert-butyl hexahydrocyclopenta[c]pyrrole-2(1H)-carboxylate (30 mg, 0.142 mmol) in CH₂Cl₂ (0.5 mL) was added 3 M hydrogen chloride (0.292 mL, 1.17 mmol) in methyl t-butyl ether. The resulting solution was stirred for 2 hours. The volatiles were removed and the residue was re-dissolved in DMF (1 mL). To this solution was added Intermediate 153B (30 mg, 0.077 mmol) followed by Hunig's base (0.054 mL, 0.311 mmol). The reaction mixture became a clear solution. The vial was sealed and heated at 70° C. for 4.5 h. The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 21% B, 21-61% B over 20 minutes, then a 4-minute hold at 100% B; Flow Rate: 20 mL/min; Column Temperature: 25° C. Fraction collection was triggered by MS signals. Fractions containing the desired product were combined and dried via centrifugal evaporation. The yield of the product was 10.2 mg, (27.9%). MS (ES): m/z=463.20 [M+H]⁺. ¹H NMR (500 MHz, DMSO-d₆) δ 11.01 (s, 1H), 8.53 (d, J=4.7 Hz, 1H), 8.11 (s, 1H), 8.03 (br d, J=8.2 Hz, 1H), 7.87 (d, J=8.0 Hz, 1H), 7.53 (t, J=4.8 Hz, 1H), 5.16 (dd, J=13.2, 5.1 Hz, 1H), 4.56 (d, J=17.4 Hz, 1H), 4.44 (d, J=17.5 Hz, 1H), 3.70 (s, 2H), 3.00-2.87 (m, 1H), 2.69-2.58 (m, 3H), 2.49-2.36 (m, 1H), 2.31-2.21 (m, 2H), 2.14-2.01 (m, 1H), 1.71-1.59 (m, 4H), 1.51-1.34 (m, 4H).

### Example 156

### tert-butyl (3aR,6aS)-5-((2-(2-((S)-2,6-dioxopiperidin-3-yl)-1-oxoisoindolin-5-yl)-3-fluoropyridin-4-yl)methyl)hexahydropyrrolo[3,4-c]pyrrole-2(1H)-carboxylate

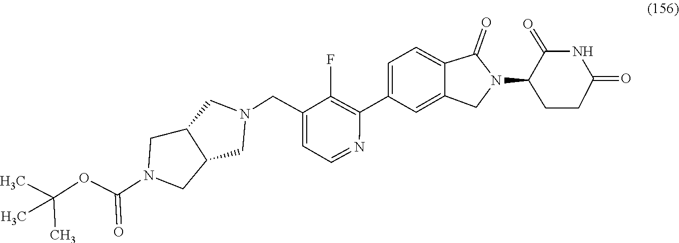 [SMILES: CC(C)(C)OC(=O)N1C[C@H]2CN(Cc3ccnc(-c4ccc5c(c4)CN([C@@H]4CCC(=O)NC4=O)C5=O)c3F)C[C@H]2C1]

To the solution Intermediate 149B (42 mg, 0.108 mmol) was added tert-butyl (3aR,6aS)-hexahydropyrrolo[3,4-c]pyrrole-2(1H)-carboxylate (22.9 mg, 0.108 mmol) followed by Hunig's base (0.038 mL, 0.226 mmol). The reaction mixture became a clear solution. The vial was sealed and heated at 70° C. for 4.5 h. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 20% B, 20-60% B over 20 minutes, then a 0-minute hold at 17% B, 17-57% B over 20 minutes, then a 4-minute hold at 100% B; Flow Rate: 20 mL/min; Column Temperature: 25° C. Fraction collection was triggered by MS signals. Fractions containing the desired product were combined and dried via centrifugal evaporation. The yield of the product was 41.1 mg, (64.3%). MS (ES): m/z=564.40 [M+H]⁺. ¹H NMR (500 MHz, DMSO-d₆) δ 8.53 (d, J=4.6 Hz, 1H), 8.11 (s, 1H), 8.03 (br d, J=7.9 Hz, 1H), 7.87 (d, J=7.9 Hz, 1H), 7.53 (t, J=4.6 Hz, 1H), 5.15 (dd, J=13.4, 5.2 Hz, 1H), 4.56 (d, J=17.4 Hz, 1H), 4.43 (d, J=17.4 Hz, 1H), 3.76 (s, 2H), 3.15 (br d, J=8.9 Hz, 2H), 3.03-2.83 (m, 1H), 2.77 (br s, 2H), 2.65-2.58 (m, 3H), 2.50-2.29 (m, 3H), 2.13-2.00 (m, 1H), 1.43-1.35 (m, 9H).

### Example 157

### 3-(5-(4-((2-azaspiro[3.5]nonan-2-yl)methyl)pyridin-2-yl)-1-oxoisoindolin-2-yl) piperidine-2,6-dione

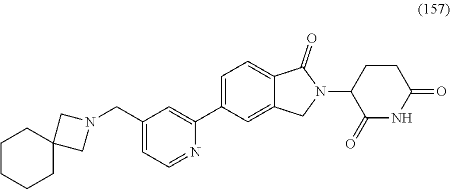 [SMILES: O=C1CCC(N2Cc3cc(-c4cc(CN5CC6(CCCCC6)C5)ccn4)ccc3C2=O)C(=O)N1]

To Intermediate 98B (20 mg, 0.049 mmol), 2-aza-spiro[3.5]nonane hydrochloride (9.55 mg, 0.059 mmol) was added DMF (2 mL) followed by N-ethyl-N-isopropylpropan-2-amine (0.069 mL, 0.394 mmol). The reaction mixture was heated at 80° C. for 2 h. The reaction mixture was cooled to room temperature, filtered and purified by preparative HPLC to afford 3-(5-(4-((2-azaspiro[3.5]nonan-2-yl)methyl)pyridin-2-yl)-1-oxoisoindolin-2-yl)piperidine-2,6-dione (10.2 mg, 0.022 mmol, 45.2% yield). Preparative HPLC condition: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: 13-53% B over 20 minutes; Flow rate: 20 mL/min; Column temperature: 25° C. The enantiomeric excess of 3-(5-(4-((2-azaspiro[3.5]nonan-2-yl)methyl)pyridin-2-yl)-1-oxoisoindolin-2-yl)piperidine-2,6-dione was not determined. LCMS (Method B): retention time 1.08 min. MS (ES): m/z=459.2 [M+H]⁺. ¹H NMR (500 MHz, DMSO-d₆) δ 8.63 (d, J=4.6 Hz, 1H), 8.31 (s, 1H), 8.23 (br d, J=8.2 Hz, 1H), 7.92 (s, 1H), 7.84 (d, J=7.9 Hz, 1H), 7.32 (br d, J=4.6 Hz, 1H), 5.16 (br dd, J=13.4, 4.9 Hz, 1H), 4.56 (br d, J=17.7 Hz, 1H), 4.43 (br d, J=17.4 Hz, 1H), 3.70 (s, 2H), 3.01-2.91 (m, 4H), 2.63 (br d, J=18.0 Hz, 1H), 2.44 (br dd, J=13.3, 4.4 Hz, 1H), 2.16-1.99 (m, 1H), 1.92 (s, 1H), 1.60 (br s, 4H), 1.43-1.24 (m, 6H).

### Example 158

The compounds in Table 14 were prepared according to the general procedures described for Example 157.

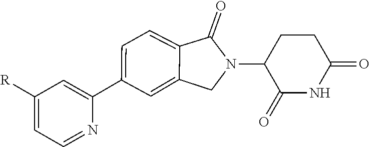

TABLE 14

| | | | Retention | |
|---|---|---|---|---|
| Ex. | | LC/MS | Time | HPLC |
| No. | R | [M + H]⁺ | (min) | Method |
| 158 | 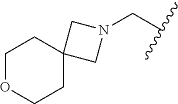 [SMILES: *CN1CC2(CCOCC2)C1] | 461.2 | 0.90 | B |

### Example 159

### N-((1-((2-(2-(2,6-dioxopiperidin-3-yl)-1-oxoisoindolin-5-yl)pyridin-4-yl)methyl)azetidin-3-yl)methyl)benzamide

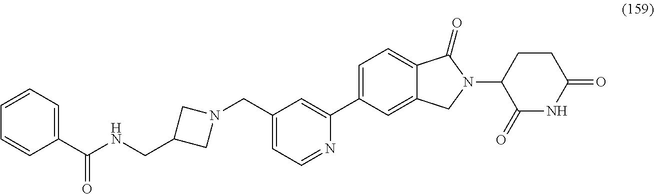 [SMILES: O=C1CCC(N2Cc3cc(-c4cc(CN5CC(CNC(=O)c6ccccc6)C5)ccn4)ccc3C2=O)C(=O)N1]

### Intermediate 159A: 3-(5-(4-((3-(aminomethyl)azetidin-1-yl)methyl)pyridin-2-yl)-1-oxoisoindolin-2-yl)piperidine-2,6-dione, HCl salt

To tert-butyl (azetidin-3-ylmethyl)carbamate (44.0 mg, 0.236 mmol) in DMF (2 mL) was added Intermediate 98B (80 mg, 0.197 mmol) followed by N-ethyl-N-isopropylpropan-2-amine (0.275 mL, 1.575 mmol). The reaction mixture was heated at 80° C. for 2 h. The reaction was quenched by water. The reaction mixture was extracted with ethyl acetate. The organic phase was combined and the solvent was evaporated. To the residue was added 4 N HCl in dioxane (1 mL). The reaction mixture was stirred at room temperature for 2 h. The solvent was evaporated to give 3-(5-(4-((3-(aminomethyl) azetidin-1-yl)methyl)pyridin-2-yl)-1-oxoisoindolin-2-yl)piperidine-2,6-dione, HCl salt (86 mg, 83% yield). MS (ES): m/z=420.1 [M+H]⁺.

### Example 159

To 3-(5-(4-((3-(aminomethyl)azetidin-1-yl)methyl)pyridin-2-yl)-1-oxoisoindolin-2-yl)piperidine-2,6-dione, HCl salt (19 mg, 0.036 mmol), benzoic acid (11.06 mg, 0.091 mmol) in DMF (2 mL) was added HATU (17.22 mg, 0.045 mmol) followed by N-ethyl-N-isopropylpropan-2-amine (0.063 mL, 0.362 mmol). The reaction mixture was stirred at room temperature for 2 h. The reaction mixture was filtered and purified by preparative HPLC to afford N-((1-((2-(2-(2,6-dioxopiperidin-3-yl)-1-oxoisoindolin-5-yl)pyridin-4-yl) methyl)azetidin-3-yl)methyl)benzamide (5.2 mg, 27.8% yield). Preparative HPLC condition: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: 5-45% B over 20 minutes; Flow rate: 20 mL/min; Column temperature: 25° C. The enantiomeric excess of N-((1-((2-(2-(2,6-dioxopiperidin-3-yl)-1-oxoisoindolin-5-yl)pyridin-4-yl)methyl)azetidin-3-yl)methyl) benzamide was not determined. LCMS (Method B): retention time 1.05 min. MS (ES): m/z=524.1 [M+H]⁺. ¹H NMR (500 MHz, DMSO-d₆) δ 8.63 (d, J=5.2 Hz, 1H), 8.56 (br t, J=5.2 Hz, 1H), 8.31 (s, 1H), 8.22 (br d, J=7.9 Hz, 1H), 7.93 (s, 1H), 7.84 (d, J=7.6 Hz, 2H), 7.54-7.43 (m, 3H), 7.32 (br d, J=4.6 Hz, 1H), 5.15 (br dd, J=13.1, 4.6 Hz, 1H), 4.55 (br d, J=17.4 Hz, 1H), 4.43 (br d, J=17.4 Hz, 1H), 3.68 (s, 2H), 3.50-3.32 (m, 2H), 3.18 (s, 2H), 3.03 (br t, J=6.3 Hz, 2H), 2.97-2.90 (m, 1H), 2.80-2.58 (m, 2H), 2.49-2.40 (m, 1H), 2.12-1.98 (m, 1H).

### Examples 160-164

The compounds in Table 15 were prepared according to the general procedures described for Example 159.

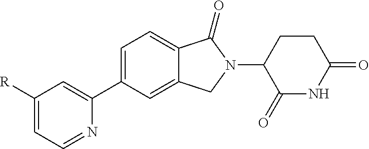

TABLE 15

| | | | Retention | |
|---|---|---|---|---|
| | | LC/MS | Time | HPLC |
| Ex. No. | R | [M + H]⁺ | (min) | Method |
| 160 | 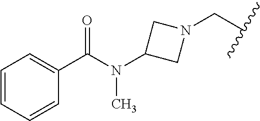 [SMILES: *CN1CC(N(C)C(=O)c2ccccc2)C1] | 524.2 | 1.09 | B |
| 161 | 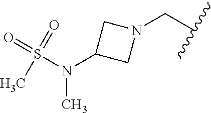 [SMILES: *CN1CC(N(C)S(C)(=O)=O)C1] | 498.1 | 0.94 | B |
| 162 | 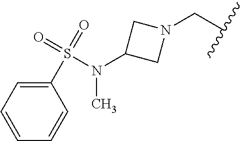 [SMILES: *CN1CC(N(C)S(=O)(=O)c2ccccc2)C1] | 560.5 | 1.09 | B |
| 163 | 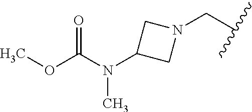 [SMILES: *CN1CC(N(C)C(=O)OC)C1] | 478.3 | 0.87 | B |
| 164 | 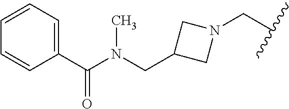 [SMILES: *CN1CC(CN(C)C(=O)c2ccccc2)C1] | 538.5 | 1.01 | B |

### Example 165

### 3-(5-(4-((1H-pyrazol-1-yl)methyl)pyridin-2-yl)-1-oxoisoindolin-2-yl)piperidine-2,6-dione

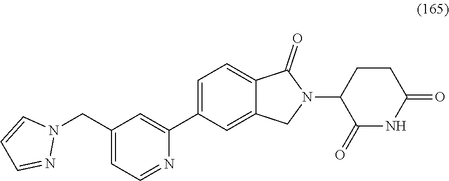 [SMILES: O=C1CCC(N2Cc3cc(-c4cc(Cn5cccn5)ccn4)ccc3C2=O)C(=O)N1]

To 1H-pyrazole (6.70 mg, 0.098 mmol) in DMF (2 mL) was added NaH (2.95 mg, 0.074 mmol). The mixture was stirred at room temperature for 10 min. Intermediate 101B (20 mg, 0.049 mmol) was added. The reaction mixture was heated at 80° C. for 2 h. The reaction mixture was cooled to room temperature and the reaction was quenched by water. The reaction mixture was filtered and purified by preparative HPLC to afford 3-(5-(4-((1H-pyrazol-1-yl)methyl)pyridin-2-yl)-1-oxoisoindolin-2-yl)piperidine-2,6-dione (6.6 mg, 32.6% yield). Preparative HPLC condition: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: 3-43% B over 20 minutes; Flow rate: 20 mL/min; Column temperature: 25° C. The enantiomeric excess of 3-(5-(4-((1H-pyrazol-1-yl)methyl)pyridin-2-yl)-1-oxoisoindolin-2-yl)piperidine-2,6-dione was not determined. LCMS (Method B): retention time 0.97 min. MS (ES): m/z=402.2 [M+H]⁺. ¹H NMR (500 MHz, DMSO-d₆) δ 8.65 (d, J=5.2 Hz, 1H), 8.26 (s, 1H), 8.16 (br d, J=8.2 Hz, 1H), 7.95 (d, J=2.1 Hz, 1H), 7.91-7.81 (m, 2H), 7.64-7.47 (m, 1H), 7.09 (d, J=4.3 Hz, 1H), 6.36 (t, J=2.0 Hz, 1H), 5.50 (s, 2H), 5.27-5.06 (m, 1H), 4.56 (br d, J=17.4 Hz, 1H), 4.43 (br d, J=17.4 Hz, 1H), 3.01-2.85 (m, 1H), 2.63 (br d, J=15.6 Hz, 1H), 2.43 (br dd, J=13.0, 4.1 Hz, 1H), 2.23-2.01 (m, 1H).

### Example 166

The compounds in Table 16 were prepared according to the general procedures described for Example 165.

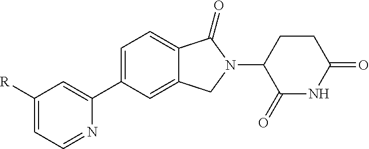

TABLE 16

| | | LC/MS | Retention | HPLC |
|---|---|---|---|---|
| Ex. No. | R | [M + H]+ | Time (min) | Method |
| 166 | 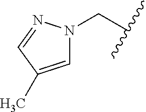 [SMILES: *Cn1cc(C)cn1] | 416.1 | 1.10 | B |

### Example 167

### 3-(5-(4-((4-(2-hydroxypropan-2-yl)piperidin-1-yl)methyl)-5-methylpyridin-2-yl)-1-oxoisoindolin-2-yl)piperidine-2,6-dione

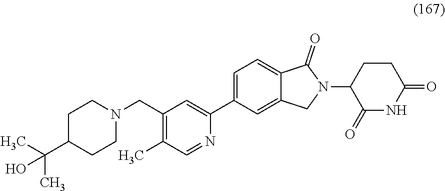 [SMILES: Cc1cnc(-c2ccc3c(c2)CN(C2CCC(=O)NC2=O)C3=O)cc1CN1CCC(C(C)(C)O)CC1]

### Intermediate 167A: (2-chloro-5-methylpyridin-4-yl)methanol

To a solution of 2-chloro-5-methyl-isonicotinic acid methyl ester (0.115 g, 0.620 mmol) in THE (1.5 mL) was added sodium borohydride (0.070 g, 1.859 mmol) followed by MeOH (0.6 mL). The reaction mixture was stirred at room temperature for 2 h. Water was added to quench the reaction. The solvent was evaporated and the residue was dissolved in EtOAc and washed by brine. The solvent was evaporated to give (2-chloro-5-methylpyridin-4-yl)methanol (0.095 g, 0.603 mmol, 97% yield). MS (ES): m/z=158.2 [M+H]⁺. ¹H NMR (400 MHz, DMSO-d₆) δ 8.13 (s, 1H), 7.42 (s, 1H), 5.51 (t, J=5.5 Hz, 1H), 4.55-4.50 (m, 2H), 2.16 (s, 3H).

### Intermediate 167B: tert-butyl 5-amino-4-(5-(4-(hydroxymethyl)-5-methylpyridin-2-yl)-1-oxoisoindolin-2-yl)-5-oxopentanoate

To a pressure-relief vial was added (2-chloro-5-methylpyridin-4-yl)methanol (0.092 g, 0.584 mmol), tert-butyl (S)-5-amino-5-oxo-4-(1-oxo-5-(4,4,5,5-tetramethyl-1,3,2-dioxaborolan-2-yl)isoindolin-2-yl)pentanoate (prepared by methods shown in WO2018/102725) (0.285 g, 0.642 mmol), Pd(dtbpf)Cl₂ (0.011 g, 0.018 mmol), dioxane (2 mL) and 2 M K₃PO₄ (0.876 mL, 1.751 mmol). The reaction mixture was bubbled with nitrogen for 5 min. The vial was sealed and the reaction mixture was heated to 70° C. for 2 h. The reaction mixture was cooled down to room temperature, diluted by ethyl acetate and washed with brine. The organic phase was dried over Na₂SO₄, and the solvent was evaporated to give a residue, which was purified by flash column chromatography (0-20% MeOH/DCM) to give tert-butyl 5-amino-4-(5-(4-(hydroxymethyl)-5-methylpyridin-2-yl)-1-oxoisoindolin-2-yl)-5-oxopentanoate (0.140 g, 54.6% yield). The enantiomeric excess of tert-butyl 5-amino-4-(5-(4-(hydroxymethyl)-5-methylpyridin-2-yl)-1-oxoisoindolin-2-yl)-5-oxopentanoate and subsequent intermediates were not determined. MS (ES): m/z=440.0 [M+H]⁺. ¹H NMR (400 MHz, DMSO-d₆) δ 8.44 (s, 1H), 8.28 (s, 1H), 8.17 (dd, J=8.0, 1.2 Hz, 1H), 8.03 (s, 1H), 7.80 (d, J=7.9 Hz, 1H), 7.58 (s, 1H), 7.20 (s, 1H), 5.47 (t, J=5.4 Hz, 1H), 4.79-4.52 (m, 5H), 2.28-2.16 (m, 6H), 2.08-1.97 (m, 1H), 1.34 (s, 9H).

### Intermediate 167C: 3-(5-(4-(chloromethyl)-5-methylpyridin-2-yl)-1-oxoisoindolin-2-yl) piperidine-2,6-dione

To tert-butyl 5-amino-4-(5-(4-(hydroxymethyl)-5-methylpyridin-2-yl)-1-oxoisoindolin-2-yl)-5-oxopentanoate (0.14 g, 0.319 mmol) in DCM (2 mL) at 0° C. was added thionyl chloride (0.069 mL, 0.956 mmol) in DCM (1 mL) dropwise. The reaction mixture was warmed up to room temperature and stirred at room temperature for 30 min. The solvent was evaporated to give tert-butyl 5-amino-4-(5-(4-(chloromethyl)-5-methylpyridin-2-yl)-1-oxoisoindolin-2-yl)-5-oxopentanoate. MS (ES): m/z=458.0 [M+H]⁺. To tert-butyl 5-amino-4-(5-(4-(chloromethyl)-5-methylpyridin-2-yl)-1-oxoisoindolin-2-yl)-5-oxopentanoate was added benzenesulfonic acid (0.111 g, 0.701 mmol) in acetic acid (1.1 mL) and the reaction mixture was heated at reflux for 2 h. The reaction mixture was cooled down and the solvent was evaporated. To the residue was added water. Solids precipitated out. The solids was filtered and washed by water to give 3-(5-(4-(chloromethyl)-5-methylpyridin-2-yl)-1-oxoisoindolin-2-yl)piperidine-2,6-dione (0.107 g, 88% yield). MS (ES): m/z=384.0 [M+H]⁺. ¹H NMR (400 MHz, DMSO-d₆) δ 11.01 (s, 1H), 8.59 (s, 1H), 8.32 (s, 1H), 8.23 (d, J=7.7 Hz, 1H), 8.15 (s, 1H), 7.85 (d, J=7.9 Hz, 1H), 5.19-5.13 (m, 1H), 4.89 (s, 2H), 4.59-4.42 (m, 2H), 2.99-2.89 (m, 1H), 2.69-2.64 (m, 1H), 2.53-2.40 (m, 4H), 2.10-2.01 (m, 1H).

### Example 167

To 3-(5-(4-(chloromethyl)-5-methylpyridin-2-yl)-1-oxoisoindolin-2-yl)piperidine-2,6-dione (15 mg, 0.036 mmol) 2-(4-piperidyl)-2-propanol (7.67 mg, 0.054 mmol) was added DMF (2 mL) followed by N-ethyl-N-isopropylpropan-2-amine (0.05 mL, 0.286 mmol). The reaction mixture was heated at 80° C. for 2 h. The reaction mixture was cooled down to room temperature, filtered and purified by preparative HPLC to afford 3-(5-(4-((4-(2-hydroxypropan-2-yl)piperidin-1-yl)methyl)-5-methylpyridin-2-yl)-1-oxoisoindolin-2-yl)piperidine-2,6-dione (9.6 mg, 54.4% yield). Preparative HPLC condition: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: 12-52% B over 20 minutes; Flow rate: 20 mL/min; Column temperature: 25° C. The enantiomeric excess of 3-(5-(4-((4-(2-hydroxypropan-2-yl)piperidin-1-yl)methyl)-5-methylpyridin-2-yl)-1-oxoisoindolin-2-yl)piperidine-2,6-dione was not determined. LCMS (Method B): retention time 0.96 min. MS (ES): m/z=491.3 [M+H]⁺. ¹H NMR (500 MHz, DMSO-d₆) δ 11.02 (s, 1H), 8.66 (s, 1H), 8.30 (s, 1H), 8.26-8.15 (m, 2H), 7.89 (d, J=8.2 Hz, 1H), 5.14 (dd, J=13.3, 5.0 Hz, 1H), 4.63-4.50 (m, 1H), 4.50-4.34 (m, 3H), 3.24-3.04 (m, 2H), 3.02-2.86 (m, 2H), 2.64 (br d, J=18.3 Hz, 1H), 2.49-2.39 (m, 4H), 2.20-1.99 (m, 1H), 1.89 (br d, J=13.4 Hz, 2H), 1.63-1.43 (m, 3H), 1.17 (t, J=7.3 Hz, 1H), 1.06 (s, 6H).

### Examples 168-169

The compounds in Table 17 were prepared according to the procedures described for Example 167.

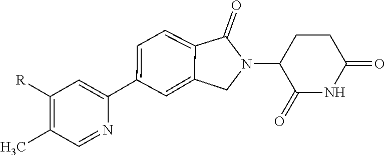

TABLE 17

| Ex. | | LC/MS | Retention | HPLC |
|---|---|---|---|---|
| No. | R | [M + H]⁺ | Time (min) | Method |
| 168 | 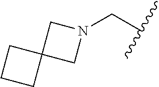 [SMILES: *CN1CC2(CCC2)C1] | 445.1 | 1.00 | B |
| 169 | 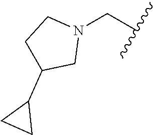 [SMILES: *CN1CCC(C2CC2)C1] | 459.3 | 1.12 | B |

### Example 170

### 3-(5-(4-((2-azaspiro[3.3]heptan-2-yl)methyl)-3-methylpyridin-2-yl)-1-oxoisoindolin-2-yl)piperidine-2,6-dione

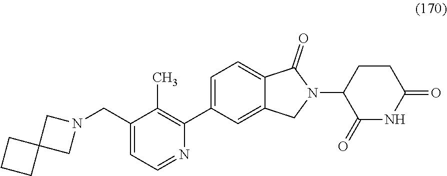 [SMILES: Cc1c(CN2CC3(CCC3)C2)ccnc1-c1ccc2c(c1)CN(C1CCC(=O)NC1=O)C2=O]

### Intermediate 170A: (2-chloro-3-methylpyridin-4-yl)methanol

To a solution of methyl 2-chloro-3-methylpyridine-4-carboxylate (0.157 g, 0.846 mmol) in THE (1.5 mL) was added sodium borohydride (0.096 g, 2.54 mmol) followed by MeOH (0.6 mL). The reaction mixture was stirred at room temperature for 2 h. Water was added to quench the reaction. The solvent was evaporated and the residue was dissolved in EtOAc and washed by brine. The solvent was evaporated to give (2-chloro-3-methylpyridin-4-yl)methanol (0.12 g, 90% yield). MS (ES): m/z=158.1 [M+H]⁺. ¹H NMR (400 MHz, DMSO-d₆) δ 8.23 (d, J=4.8 Hz, 1H), 7.45 (d, J=4.8 Hz, 1H), 5.52 (t, J=5.4 Hz, 1H), 4.59-4.53 (m, 2H), 2.25 (s, 3H).

### Intermediate 170B: tert-butyl 5-amino-4-(5-(4-(hydroxymethyl)-3-methylpyridin-2-yl)-1-oxoisoindolin-2-yl)-5-oxopentanoate

To a pressure-relief vial was added (2-chloro-3-methylpyridin-4-yl)methanol (0.117 g, 0.742 mmol), tert-butyl (S)-5-amino-5-oxo-4-(1-oxo-5-(4,4,5,5-tetramethyl-1,3,2-dioxaborolan-2-yl)isoindolin-2-yl)pentanoate (prepared by methods shown in WO2018/102725) (0.363 g, 0.817 mmol), Pd(dtbpf)Cl₂ (0.014 g, 0.022 mmol), dioxane (2 mL) and 2 M K₃PO₄ (1.114 mL, 2.227 mmol). The reaction mixture was bubbled with nitrogen for 5 min. The vial was sealed and the reaction mixture was heated to 70° C. for 2 h. The reaction mixture was cooled down to room temperature, diluted by ethyl acetate and washed with brine. The organic phase was dried over Na₂SO₄, and the solvent was evaporated to give a residue. To this residue was added ether. Solids precipitated out. The solids was filtered and washed by ether to give tert-butyl 5-amino-4-(5-(4-(hydroxymethyl)-3-methylpyridin-2-yl)-1-oxoisoindolin-2-yl)-5-oxopentanoate (0.22 g, 67.4% yield). The enantiomeric excess of tert-butyl 5-amino-4-(5-(4-(hydroxymethyl)-3-methylpyridin-2-yl)-1-oxoisoindolin-2-yl)-5-oxopentanoate and subsequent intermediates were not determined. MS (ES): m/z=440.1 [M+H]⁺. ¹H NMR (400 MHz, DMSO-d₆) δ 8.49 (d, J=4.8 Hz, 1H), 7.77 (d, J=7.7 Hz, 1H), 7.69 (s, 1H), 7.63-7.54 (m, 2H), 7.48 (d, J=4.8 Hz, 1H), 7.20 (br s, 1H), 5.47 (br s, 1H), 4.81-4.73 (m, 1H), 4.70-4.50 (m, 4H), 2.24-2.13 (m, 6H), 2.10-1.90 (m, 1H), 1.34 (s, 9H).

### Intermediate 170C: 3-(5-(4-(chloromethyl)-3-methylpyridin-2-yl)-1-oxoisoindolin-2-yl) piperidine-2,6-dione

To tert-butyl 5-amino-4-(5-(4-(hydroxymethyl)-3-methylpyridin-2-yl)-1-oxoisoindolin-2-yl)-5-oxopentanoate (0.185 g, 0.421 mmol) in DCM (2 mL) at 0° C. was added thionyl chloride (0.092 mL, 1.263 mmol) in DCM (1 mL) dropwise. The reaction mixture was warmed up to room temperature and stirred at room temperature for 30 min. The solvent was evaporated to give tert-butyl 5-amino-4-(5-(4-(chloromethyl)-3-methylpyridin-2-yl)-1-oxoisoindolin-2-yl)-5-oxopentanoate. MS (ES): m/z=458.0 [M+H]⁺. To tert-butyl 5-amino-4-(5-(4-(chloromethyl)-3-methylpyridin-2-yl)-1-oxoisoindolin-2-yl)-5-oxopentanoate was added benzenesulfonic acid (0.146 g, 0.92 mmol) in acetic acid (0.8 mL) and the reaction mixture was heated at reflux for 2 h. The reaction mixture was cooled down and the solvent was evaporated to give 3-(5-(4-(chloromethyl)-3-methylpyridin-2-yl)-1-oxoisoindolin-2-yl)piperidine-2,6-dione (0.105 g, 65% yield). MS (ES): m/z=384.0 [M+H]⁺.

### Example 170

To 3-(5-(4-(chloromethyl)-3-methylpyridin-2-yl)-1-oxoisoindolin-2-yl)piperidine-2,6-dione (15 mg, 0.036 mmol), 2-azaspiro[3.3]heptane (5.20 mg, 0.054 mmol) was added DMF (2 mL) followed by N-ethyl-N-isopropylpropan-2-amine (0.05 mL, 0.286 mmol). The reaction mixture was heated at 80° C. for 2 h. The reaction mixture was cooled down to room temperature, filtered and purified by preparative HPLC to afford 3-(5-(4-((2-azaspiro[3.3]heptan-2-yl)methyl)-3-methylpyridin-2-yl)-1-oxoisoindolin-2-yl) piperidine-2,6-dione (3.9 mg, 23.4% yield). Preparative HPLC condition: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: 9-49% B over 20 minutes; Flow rate: 20 mL/min; Column temperature: 25° C. The enantiomeric excess of 3-(5-(4-((2-azaspiro[3.3]heptan-2-yl)methyl)-3-methylpyridin-2-yl)-1-oxoisoindolin-2-yl)piperidine-2,6-dione was not determined. LCMS (Method B): retention time 0.89 min. MS (ES): m/z=445.2 [M+H]⁺. ¹H NMR (500 MHz, DMSO-d₆) δ 8.45 (br d, J=3.7 Hz, 1H), 7.81 (d, J=7.9 Hz, 1H), 7.69 (s, 1H), 7.59 (d, J=7.6 Hz, 1H), 7.32 (br d, J=4.9 Hz, 1H), 5.16 (br dd, J=13.4, 5.2 Hz, 1H), 4.54 (br d, J=17.4 Hz, 1H), 4.41 (br d, J=17.7 Hz, 1H), 3.64 (s, 2H), 3.31 (s, 4H), 3.03-2.88 (m, 1H), 2.63 (br d, J=17.4 Hz, 1H), 2.47-2.39 (m, 1H), 2.21 (s, 3H), 2.16-2.01 (m, 5H), 1.81-1.75 (m, 2H), 1.92 (s, 1H), 1.78 (quin, J=7.5 Hz, 2H), 1.17 (t, J=7.3 Hz, 1H).

### Example 171

The compound in Table 18 was prepared according to the general procedures described for Example 170.

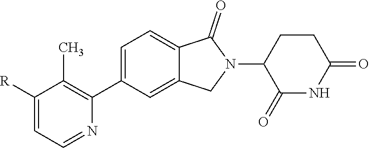

TABLE 18

| | | | Re- | |
|---|---|---|---|---|
| | | | tention | HPLC |
| Ex. | | LC/MS | Time | Meth- |
| No. | R | [M + H]⁺ | (min) | od |
| 171 | 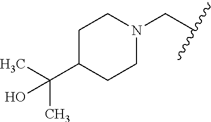 [SMILES: *CN1CCC(C(C)(C)O)CC1] | 491.3 | 0.87 | B |

### Example 172

### 3-(5-(4-((4-(2-hydroxypropan-2-yl)piperidin-1-yl)methyl)-3-methoxypyridin-2-yl)-1-oxoisoindolin-2-yl)piperidine-2,6-dione

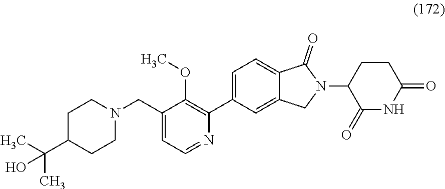 [SMILES: COc1c(CN2CCC(C(C)(C)O)CC2)ccnc1-c1ccc2c(c1)CN(C1CCC(=O)NC1=O)C2=O]

### Intermediate 172A: (2-chloro-3-methoxypyridin-4-yl)methanol

To a solution of 2-chloro-3-methoxypyridine-4-carbaldehyde (0.035 g, 0.204 mmol) in THE (1.5 mL) was added sodium borohydride (0.015 g, 0.408 mmol) followed by MeOH (0.6 mL). The reaction mixture was stirred at room temperature for 2 h. Water was added to quench the reaction. The solvent was evaporated and the residue was dissolved in EtOAc and washed by brine. The solvent was evaporated to give (2-chloro-3-methoxypyridin-4-yl)methanol (0.033 g, 0.19 mmol, 93% yield). MS (ES): m/z=174.1 [M+H]⁺. ¹H NMR (400 MHz, DMSO-d₆) δ 8.18 (d, J=4.8 Hz, 1H), 7.49 (d, J=4.8 Hz, 1H), 5.52 (t, J=5.6 Hz, 1H), 4.63 (d, J=5.7 Hz, 2H), 3.80 (s, 3H).

### Intermediate 172B: tert-butyl 5-amino-4-(5-(4-(hydroxymethyl)-3-methoxypyridin-2-yl)-1-oxoisoindolin-2-yl)-5-oxopentanoate

To a pressure-relief vial was added (2-chloro-3-methoxypyridin-4-yl)methanol (0.139 g, 0.801 mmol), tert-butyl (S)-5-amino-5-oxo-4-(1-oxo-5-(4,4,5,5-tetramethyl-1,3,2-dioxaborolan-2-yl)isoindolin-2-yl)pentanoate (prepared by methods shown in WO2018102725) (0.391 g, 0.881 mmol), Pd(dtbpf)Cl₂ (0.016 mg, 0.024 mmol), dioxane (2 mL) and 2 M K₃PO₄ (1.2 mL, 2.4 mmol). The reaction mixture was bubbled with nitrogen for 5 min. The vial was sealed and the reaction mixture was heated to 70° C. for 2 h. The reaction mixture was cooled down to room temperature, diluted by ethyl acetate and washed with brine. The organic phase was dried over Na₂SO₄, and the solvent was evaporated to give a residue, which was purified by flash column chromatography (0-20% MeOH/DCM) to give tert-butyl 5-amino-4-(5-(4-(hydroxymethyl)-3-methoxypyridin-2-yl)-1-oxoisoindolin-2-yl)-5-oxopentanoate (0.264 g, 72.4% yield). The enantiomeric excess of tert-butyl 5-amino-4-(5-(4-(hydroxymethyl)-3-methoxypyridin-2-yl)-1-oxoisoindolin-2-yl)-5-oxopentanoate and subsequent intermediates were not determined. MS (ES): m/z=456.1 [M+H]⁺. ¹H NMR (400 MHz, DMSO-d₆) δ 8.47 (d, J=4.8 Hz, 1H), 8.08 (s, 1H), 8.00 (d, J=7.3 Hz, 1H), 7.79 (d, J=7.9 Hz, 1H), 7.59 (s, 1H), 7.52 (d, J=4.6 Hz, 1H), 7.20 (s, 1H), 5.46 (t, J=5.6 Hz, 1H), 4.81-4.75 (m, 1H), 4.72-4.65 (m, 3H), 4.58-4.51 (m, 1H), 3.46 (s, 3H), 2.24-2.14 (m, 3H), 2.06-1.98 (m, 1H), 1.34 (s, 9H).

### Intermediate 172C: 3-(5-(4-(chloromethyl)-3-methoxypyridin-2-yl)-1-oxoisoindolin-2-yl) piperidine-2,6-dione

To tert-butyl 5-amino-4-(5-(4-(hydroxymethyl)-3-methoxypyridin-2-yl)-1-oxoisoindolin-2-yl)-5-oxopentanoate (0.15 g, 0.329 mmol) in DCM (2 mL) at 0° C. was added thionyl chloride (0.072 mL, 0.99 mmol) in DCM (1 mL) dropwise. The reaction mixture was warmed up to room temperature and stirred at room temperature for 30 min. The solvent was evaporated to give tert-butyl 5-amino-4-(5-(4-(chloromethyl)-3-methoxypyridin-2-yl)-1-oxoisoindolin-2-yl)-5-oxopentanoate. MS (ES): m/z=474.1 [M+H]⁺. To tert-butyl 5-amino-4-(5-(4-(chloromethyl)-3-methoxypyridin-2-yl)-1-oxoisoindolin-2-yl)-5-oxopentanoate was added benzenesulfonic acid (0.115 g, 0.724 mmol) in acetic acid (1.2 mL) and the reaction mixture was heated at reflux for 2 h. The reaction mixture was cooled down and the solvent was evaporated. The residue was dissolved in EtOAc and washed by water. The organic phase was combined and the solvent was evaporated to give 3-(5-(4-(chloromethyl)-3-methoxypyridin-2-yl)-1-oxoisoindolin-2-yl)piperidine-2,6-dione (0.075 g, 57% yield). MS (ES): m/z=400.0 [M+H]⁺.

### Example 172

To Intermediate 98B (12 mg, 0.028 mmol), 2-(4-piperidyl)-2-propanol (5.91 mg, 0.042 mmol) was added DMF (2 mL) followed by N-ethyl-N-isopropylpropan-2-amine (0.038 mL, 0.22 mmol). The reaction mixture was heated at 80° C. for 2 h. The reaction mixture was cooled down to room temperature, filtered and purified by preparative HPLC to afford 3-(5-(4-((4-(2-hydroxypropan-2-yl)piperidin-1-yl)methyl)-3-methoxypyridin-2-yl)-1-oxoisoindolin-2-yl)piperidine-2,6-dione (9.1 mg, 62% yield). Preparative HPLC condition: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: 1-41% B over 20 minutes; Flow rate: 20 mL/min; Column temperature: 25° C. The enantiomeric excess of 3-(5-(4-((4-(2-hydroxypropan-2-yl)piperidin-1-yl)methyl)-3-methoxypyridin-2-yl)-1-oxoisoindolin-2-yl)piperidine-2,6-dione was not determined. LCMS (Method B): retention time 0.93 min. MS (ES): m/z=507.2 [M+H]⁺. ¹H NMR (500 MHz, DMSO-d₆) δ 11.01 (s, 1H), 8.44 (d, J=4.7 Hz, 1H), 8.09 (s, 1H), 8.03 (d, J=7.9 Hz, 1H), 7.83 (d, J=8.0 Hz, 1H), 7.47 (d, J=4.7 Hz, 1H), 5.15 (dd, J=13.2, 5.0 Hz, 1H), 4.55 (d, J=17.4 Hz, 1H), 4.42 (d, J=17.2 Hz, 1H), 4.08 (s, 3H), 3.60-3.46 (m, 4H), 3.00-2.85 (m, 3H), 2.63 (br dd, J=16.0, 2.1 Hz, 1H), 2.47-2.37 (m, 1H), 2.06-1.94 (m, 2H), 1.68 (br d, J=12.4 Hz, 2H), 1.35-1.14 (m, 2H), 1.04 (s, 6H).

### Example 173

The compound in Table 19 was prepared according to the general procedures described for Example 172.

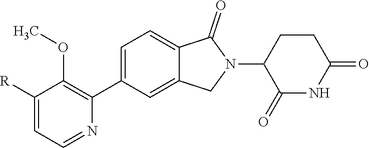

TABLE 19

| | | LC/MS | Retention | HPLC |
|---|---|---|---|---|
| Ex. No. | R | [M + H]⁺ | Time (min) | Method |
| 173 | 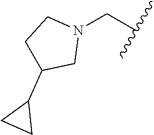 [SMILES: *CN1CCC(C2CC2)C1] | 475.3 | 1.10 | B |

### Example 174

### 3-(5-(4-((2-azaspiro[3.3]heptan-2-yl)methyl)-6-methoxypyridin-2-yl)-1-oxoisoindolin-2-yl)piperidine-2,6-dione

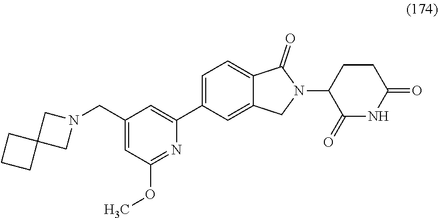 [SMILES: COc1cc(CN2CC3(CCC3)C2)cc(-c2ccc3c(c2)CN(C2CCC(=O)NC2=O)C3=O)n1]

### Intermediate 174A: (2-chloro-6-methoxypyridin-4-yl)methanol

To a solution of methyl 2-chloro-6-methoxyisonicotinate (0.057 g, 0.283 mmol) in THE (1.5 mL) was added sodium borohydride (0.032 g, 0.848 mmol) followed by MeOH (0.6 mL). The reaction mixture was stirred at room temperature for 2 h. Water was added to quench the reaction. The solvent was evaporated and the residue was dissolved in EtOAc and washed by brine. The solvent was evaporated to give (2-chloro-6-methoxypyridin-4-yl)methanol (0.048 g, 98% yield). MS (ES): m/z=174.1, [M+H]⁺. ¹H NMR (400 MHz, DMSO-d₆) δ 7.01 (s, 1H), 6.75 (s, 1H), 5.50 (t, J=5.8 Hz, 1H), 4.51 (d, 2H), 3.85 (s, 3H).

### Intermediate 174B: tert-butyl 5-amino-4-(5-(4-(hydroxymethyl)-6-methoxypyridin-2-yl)-1-oxoisoindolin-2-yl)-5-oxopentanoate

To a pressure-relief vial was added (2-chloro-6-methoxypyridin-4-yl)methanol (0.046 g, 0.265 mmol), tert-butyl (S)-5-amino-5-oxo-4-(1-oxo-5-(4,4,5,5-tetramethyl-1,3,2-dioxaborolan-2-yl)isoindolin-2-yl)pentanoate (prepared by methods shown in WO2018102725) (0.13 g, 0.291 mmol), Pd(dtbpf)Cl₂ (0.005 g, 0.008 mmol), dioxane (2 mL) and 2 M K₃PO₄ (0.4 mL, 0.8 mmol). The reaction mixture was bubbled with nitrogen for 5 min. The vial was sealed and the reaction mixture was heated to 60° C. for 16 h. The reaction mixture was cooled down to room temperature, diluted by ethyl acetate and washed with brine. The organic phase was dried over Na₂SO₄, and the solvent was evaporated to give a residue, which was purified by flash column chromatography (0-20% MeOH/DCM) to give tert-butyl 5-amino-4-(5-(4-(hydroxymethyl)-6-methoxypyridin-2-yl)-1-oxoisoindolin-2-yl)-5-oxopentanoate (0.052 g, 43.1% yield). The enantiomeric excess of tert-butyl 5-amino-4-(5-(4-(hydroxymethyl)-6-methoxypyridin-2-yl)-1-oxoisoindolin-2-yl)-5-oxopentanoate and subsequent intermediates were not determined. MS (ES): m/z=456.1 [M+H]⁺. ¹H NMR (400 MHz, DMSO-d₆) δ 8.31 (s, 1H), 8.22 (d, J=8.1 Hz, 1H), 7.80 (d, J=7.9 Hz, 1H), 7.64-7.56 (m, 2H), 7.20 (br s, 1H), 6.79 (s, 1H), 5.47 (t, J=5.7 Hz, 1H), 4.80-4.74 (m, 1H), 4.71-4.65 (m, 1H), 4.61-4.54 (m, 3H), 3.99 (s, 3H), 2.23-1.97 (m, 3H), 2.08-1.97 (m, 1H), 1.35 (s, 9H).

### Intermediate 174C: 3-(5-(4-(chloromethyl)-6-methoxypyridin-2-yl)-1-oxoisoindolin-2-yl) piperidine-2,6-dione

To tert-butyl 5-amino-4-(5-(4-(hydroxymethyl)-6-methoxypyridin-2-yl)-1-oxoisoindolin-2-yl)-5-oxopentanoate (0.050 g, 0.110 mmol) in DCM (2 mL) at 0° C. was added thionyl chloride (0.024 mL, 0.329 mmol) in DCM (1 mL) dropwise. The reaction mixture was warmed up to room temperature and stirred at room temperature for 30 min. The solvent was evaporated to give tert-butyl 5-amino-4-(5-(4-(chloromethyl)-6-methoxypyridin-2-yl)-1-oxoisoindolin-2-yl)-5-oxopentanoate. MS (ES): m/z=474.1 [M+H]⁺. To tert-butyl 5-amino-4-(5-(4-(chloromethyl)-6-methoxypyridin-2-yl)-1-oxoisoindolin-2-yl)-5-oxopentanoate was added p-toluenesulfonic acid (0.046 g, 0.241 mmol) in acetic acid (0.8 mL) and the reaction mixture was heated at reflux for 2 h. The reaction mixture was cooled down and the solvent was evaporated. The residue was dissolved in ethyl acetate and washed by water. The organic phase was combined and the solvent was evaporated to give 3-(5-(4-(chloromethyl)-6-methoxypyridin-2-yl)-1-oxoisoindolin-2-yl)piperidine-2,6-dione (0.036 g, 82% yield) without further purification. MS (ES): m/z=400.0 [M+H]⁺.

### Example 174

To 3-(5-(4-(chloromethyl)-6-methoxypyridin-2-yl)-1-oxoisoindolin-2-yl)piperidine-2,6-dione (12 mg, 0.028 mmol), 2-azaspiro[3.3]heptane (4 mg, 0.042 mmol) was added DMF (2 mL) followed by N-ethyl-N-isopropylpropan-2-amine (0.038 mL, 0.220 mmol). The reaction mixture was heated at 80° C. for 2 h. The reaction mixture was cooled down to room temperature, filtered and purified by preparative HPLC to afford 3-(5-(4-((2-azaspiro[3.3]heptan-2-yl)methyl)-6-methoxypyridin-2-yl)-1-oxoisoindolin-2-yl) piperidine-2,6-dione (5.6 mg, 43% yield). Preparative HPLC condition: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: 11-51% B over 20 minutes; Flow rate: 20 mL/min; Column temperature: 25° C. The enantiomeric excess of 3-(5-(4-((2-azaspiro[3.3]heptan-2-yl) methyl)-6-methoxypyridin-2-yl)-1-oxoisoindolin-2-yl)piperidine-2,6-dione was not determined. LCMS (Method B): retention time 1.09 min. MS (ES): m/z=461.2 [M+H]⁺. ¹H NMR (500 MHz, DMSO-d₆) δ 8.31 (s, 1H), 8.24 (d, J=8.3 Hz, 1H), 7.83 (d, J=8.0 Hz, 1H), 7.53 (s, 1H), 6.71 (s, 1H), 5.15 (dd, J=13.1, 5.1 Hz, 1H), 4.55 (d, J=17.3 Hz, 1H), 4.42 (d, J=17.2 Hz, 1H), 3.97 (s, 3H), 3.57 (s, 2H), 3.17 (s, 4H), 2.98-2.78 (m, 1H), 2.76-2.58 (m, 1H), 2.43 (br dd, J=13.1, 4.0 Hz, 1H), 2.10-2.02 (m, 5H), 1.79-1.73 (m, 2H).

### Example 175

The compound in Table 20 was prepared according to the general procedures described for Example 174.

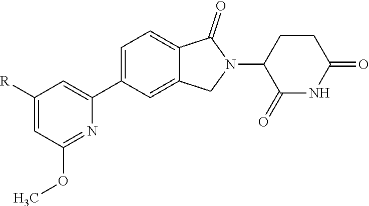

TABLE 20

| Ex. | | LC/MS | Retention | HPLC |
|---|---|---|---|---|
| No. | R | [M + H]⁺ | Time (min) | Method |
| 175 | 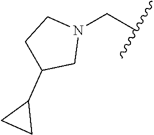 [SMILES: *CN1CCC(C2CC2)C1] | 475.1 | 1.21 | B |

### Example 176

### 3-(5-(4-((4-(2-hydroxypropan-2-yl)piperidin-1-yl)methyl)-3-(trifluoromethyl)pyridin-2-yl)-1-oxoisoindolin-2-yl)piperidine-2,6-dione

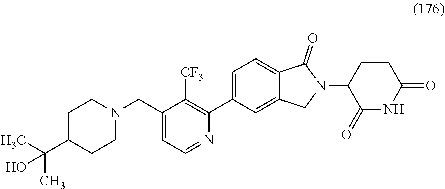 [SMILES: CC(C)(O)C1CCN(Cc2ccnc(-c3ccc4c(c3)CN(C3CCC(=O)NC3=O)C4=O)c2C(F)(F)F)CC1]

### Intermediate 176A: (2-chloro-3-(trifluoromethyl)pyridin-4-yl)methanol

To a solution of methyl 2-chloro-3-(trifluoromethyl)isonicotinate (0.250 g, 1.044 mmol) in THE (10 mL) was added sodium borohydride (0.118 g, 3.13 mmol) followed by MeOH (1 mL). The reaction mixture was stirred at room temperature for 2 h. the reaction was not complete as monitored by LCMS. To the reaction mixture was added sodium borohydride (0.1 g, 2.63 mmol). The reaction mixture was stirred at room temperature for 18 h. The reaction was not complete as monitored by LCMS. To the reaction mixture was added sodium borohydride (0.08 g, 2.11 mmol). The reaction mixture was stirred at room temperature for 2 h. The reaction was complete as monitored by LCMS. Saturated ammonium chloride solution (1 mL) was added to quench the reaction. The solvent was evaporated and the residue was dissolved in EtOAc and washed by brine. The organic phase was dried by sodium sulfate and the solvent was evaporated to give (2-chloro-3-(trifluoromethyl)pyridin-4-yl)methanol (0.094 g, 43% yield). MS (ES): m/z=212.0 [M+H]⁺.

### Intermediate 176B: tert-butyl 5-amino-4-(5-(4-(hydroxymethyl)-3-(trifluoromethyl) pyridin-2-yl)-1-oxoisoindolin-2-yl)-5-oxopentanoate

To a pressure-relief vial was added (2-chloro-3-(trifluoromethyl)pyridin-4-yl) methanol (0.094 g, 0.444 mmol), tert-butyl (S)-5-amino-5-oxo-4-(1-oxo-5-(4,4,5,5-tetramethyl-1,3,2-dioxaborolan-2-yl)isoindolin-2-yl)pentanoate (prepared by methods shown in WO2018102725) (0.217 g, 0.489 mmol), Pd(dtbpf)Cl₂ (0.009 g, 0.013 mmol), dioxane (3 mL) and 2 M K₃PO₄ (0.666 mL, 1.333 mmol). The reaction mixture was bubbled with nitrogen for 5 min. The vial was sealed and the reaction mixture was heated to 70° C. for 2 h. The reaction mixture was cooled down to room temperature, diluted by ethyl acetate and washed with brine. The organic phase was dried over Na₂SO₄, and the solvent was evaporated to give a residue, which was purified by flash column chromatography (0-20% MeOH/DCM) to give tert-butyl 5-amino-4-(5-(4-(hydroxymethyl)-3-(trifluoromethyl)pyridin-2-yl)-1-oxoisoindolin-2-yl)-5-oxopentanoate (0.09 g, 41% yield). The enantiomeric excess of tert-butyl 5-amino-4-(5-(4-(hydroxymethyl)-3-(trifluoromethyl)pyridin-2-yl)-1-oxoisoindolin-2-yl)-5-oxopentanoate and subsequent intermediates were not determined. MS (ES): m/z=494.1 [M+H]⁺.

### Intermediate 176C: 3-(5-(4-(chloromethyl)-3-(trifluoromethyl)pyridin-2-yl)-1-oxoisoindolin-2-yl) piperidine-2,6-dione

To tert-butyl 5-amino-4-(5-(4-(hydroxymethyl)-3-(trifluoromethyl)pyridin-2-yl)-1-oxoisoindolin-2-yl)-5-oxopentanoate (0.09 g, 0.182 mmol) in DCM (2 mL) at 0° C. was added thionyl chloride (0.04 mL, 0.547 mmol) in DCM (1 mL) dropwise. The reaction was warmed up to room temperature and stirred at room temperature for 30 min. The solvent was evaporated to give tert-butyl 5-amino-4-(5-(4-(chloromethyl)-3-(trifluoromethyl)pyridin-2-yl)-1-oxoisoindolin-2-yl)-5-oxopentanoate. MS (ES): m/z=550.1 [M+K]⁺. To tert-butyl 5-amino-4-(5-(4-(chloromethyl)-3-(trifluoromethyl)pyridin-2-yl)-1-oxoisoindolin-2-yl)-5-oxopentanoate was added benzenesulfonic acid (0.064 g, 0.401 mmol) in acetic acid (2 mL) and the reaction mixture was heated at reflux for 2 h. The reaction mixture was cooled down and the solvent was evaporated to give 3-(5-(4-(chloromethyl)-3-(trifluoromethyl)pyridin-2-yl)-1-oxoisoindolin-2-yl)piperidine-2,6-dione (0.075 g, 94% yield). MS (ES): m/z=438.1 [M+H]⁺.

### Example 176

To 3-(5-(4-(chloromethyl)-3-(trifluoromethyl)pyridin-2-yl)-1-oxoisoindolin-2-yl) piperidine-2,6-dione (15 mg, 0.032 mmol), 2-(4-piperidyl)-2-propanol (6.80 mg, 0.047 mmol) was added DMF (2 mL) followed by N-ethyl-N-isopropylpropan-2-amine (0.044 mL, 0.253 mmol). The reaction mixture was heated at 80° C. for 2 h. The reaction mixture was cooled down to room temperature, filtered and purified by preparative HPLC to afford 3-(5-(4-((4-(2-hydroxypropan-2-yl)piperidin-1-yl)methyl)-3-(trifluoromethyl)-pyridin-2-yl)-1-oxoisoindolin-2-yl)piperidine-2,6-dione (8.3 mg, 46.1% yield). Preparative HPLC condition: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: 16-56% B over 20 minutes; Flow rate: 20 mL/min; Column temperature: 25° C. The enantiomeric excess of 3-(5-(4-((4-(2-hydroxypropan-2-yl)piperidin-1-yl)methyl)-3-(trifluoromethyl)pyridin-2-yl)-1-oxoisoindolin-2-yl)piperidine-2,6-dione was not determined. LCMS (Method B): retention time 0.96 min. MS (ES): m/z=545.2 [M+H]⁺. ¹H NMR (500 MHz, DMSO-d₆) δ 11.02 (br s, 1H), 9.10-8.95 (m, 1H), 8.09-7.92 (m, 1H), 7.85 (br d, J=7.3 Hz, 1H), 7.77-7.64 (m, 1H), 7.57 (br d, J=7.3 Hz, 1H), 5.23-5.02 (m, 1H), 4.62-4.54 (m, 3H), 4.42 (d, J=17.2 Hz, 1H), 3.56-3.34 (m, 2H), 3.14-2.90 (m, 3H), 2.68-2.60 (m, 1H), 2.49-2.32 (m, 1H), 2.14-1.98 (m, 1H), 1.88 (br d, J=12.8 Hz, 2H), 1.67-1.45 (m, 3H), 1.07 (s, 6H).

### Examples 177-178

The compounds in Table 21 were prepared according to the general procedures described for Example 176.

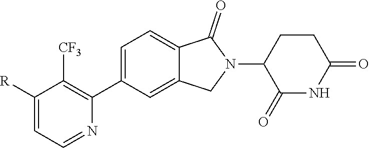

TABLE 21

| | | LC/MS | Retention | |
|---|---|---|---|---|
| Ex. No. | R | [M + H]⁺ | Time (min) | HPLC Method |
| 177 | 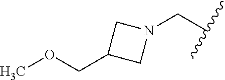 [SMILES: *CN1CC(COC)C1] | 503.2 | 1.05 | B |
| 178 | 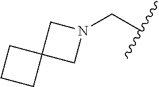 [SMILES: *CN1CC2(CCC2)C1] | 499.1 | 1.10 | B |

### Example 179

### 3-(5-(4-((4-(2-hydroxypropan-2-yl)piperidin-1-yl)methyl)-5-(trifluoromethyl)pyridin-2-yl)-1-oxoisoindolin-2-yl)piperidine-2,6-dione

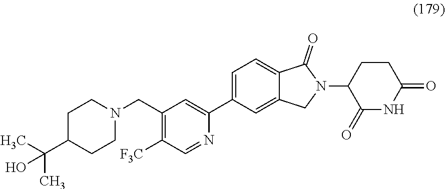 [SMILES: CC(C)(O)C1CCN(Cc2cc(-c3ccc4c(c3)CN(C3CCC(=O)NC3=O)C4=O)ncc2C(F)(F)F)CC1]

### Intermediate 179A: (2-chloro-5-(trifluoromethyl)pyridin-4-yl)methanol

To a solution of 2-chloro-5-(trifluoromethyl)-pyridine-4-carboxaldehyde (0.174 g, 0.830 mmol) in THE (1.5 mL) was added sodium borohydride (0.063 g, 1.66 mmol) followed by MeOH (0.6 mL). The reaction mixture was stirred at room temperature for 2 h. Water was added to quench the reaction. The solvent was evaporated and the residue was dissolved in EtOAc and washed by brine. The solvent was evaporated to give (2-chloro-5-(trifluoromethyl)pyridin-4-yl)methanol (0.160 g, 91% yield). MS (ES): m/z=212.0 [M+H]⁺.

### Intermediate 179B: tert-butyl 5-amino-4-(5-(4-(hydroxymethyl)-5-(trifluoromethyl)pyridin-2-yl)-1-oxoisoindolin-2-yl)-5-oxopentanoate

To a pressure-relief vial was added (2-chloro-5-(trifluoromethyl)pyridin-4-yl) methanol (0.16 g, 0.756 mmol), tert-butyl (S)-5-amino-5-oxo-4-(1-oxo-5-(4,4,5,5-tetramethyl-1,3,2-dioxaborolan-2-yl)isoindolin-2-yl)pentanoate (prepared by methods shown in WO2018102725) (0.37 g, 0.832 mmol), Pd(dtbpf)Cl₂ (0.015 g, 0.023 mmol), dioxane (2 mL) and 2 M K₃PO₄ (1.134 mL, 2.268 mmol). The reaction mixture was bubbled with nitrogen for 5 min. The vial was sealed and the reaction mixture was heated to 70° C. for 2 h. The reaction mixture was cooled down to room temperature, diluted by ethyl acetate and washed with brine. The organic phase was dried over Na₂SO₄, and the solvent was evaporated to give a residue, which was purified by flash column chromatography (0-20% MeOH/DCM) to give tert-butyl 5-amino-4-(5-(4-(hydroxymethyl)-5-(trifluoromethyl)pyridin-2-yl)-1-oxoisoindolin-2-yl)-5-oxopentanoate (0.188 g, 50.4% yield). The enantiomeric excess of tert-butyl 5-amino-4-(5-(4-(hydroxymethyl)-5-(trifluoromethyl)pyridin-2-yl)-1-oxoisoindolin-2-yl)-5-oxopentanoate and subsequent intermediates were not determined. MS (ES): m/z=494.1 [M+H]⁺.

### Intermediate 179C: 3-(5-(4-(chloromethyl)-5-(trifluoromethyl)pyridin-2-yl)-1-oxoisoindolin-2-yl) piperidine-2,6-dione

To tert-butyl 5-amino-4-(5-(4-(hydroxymethyl)-5-(trifluoromethyl)pyridin-2-yl)-1-oxoisoindolin-2-yl)-5-oxopentanoate (0.14 g, 0.284 mmol) in DCM (2 mL) at 0° C. was added thionyl chloride (0.062 mL, 0.851 mmol) in DCM (1 mL) dropwise. The reaction was warmed up to room temperature and stirred at room temperature for 30 min. The solvent was evaporated to give tert-butyl 5-amino-4-(5-(4-(chloromethyl)-5-(trifluoromethyl)pyridin-2-yl)-1-oxoisoindolin-2-yl)-5-oxopentanoate. MS (ES): m/z=550.0 [M+K]⁺. To tert-butyl 5-amino-4-(5-(4-(chloromethyl)-5-(trifluoromethyl)pyridin-2-yl)-1-oxoisoindolin-2-yl)-5-oxopentanoate was added benzenesulfonic acid (0.099 g, 0.624 mmol) in acetic acid (1.2 mL) and the reaction mixture was heated at reflux for 2 h. The reaction mixture was cooled down and the solvent was evaporated to give 3-(5-(4-(chloromethyl)-5-(trifluoromethyl)pyridin-2-yl)-1-oxoisoindolin-2-yl)piperidine-2,6-dione (0.12 g, 97% yield) without further purification. MS (ES): m/z=438.1 [M+H]⁺.

### Example 179

To 3-(5-(4-(chloromethyl)-5-(trifluoromethyl)pyridin-2-yl)-1-oxoisoindolin-2-yl) piperidine-2,6-dione (15 mg, 0.032 mmol), 2-(4-piperidyl)-2-propanol (6.80 mg, 0.047 mmol) was added DMF (2 mL) followed by N-ethyl-N-isopropylpropan-2-amine (0.044 mL, 0.253 mmol). The reaction mixture was heated at 80° C. for 2 h. The reaction mixture was cooled down to room temperature, filtered and purified by preparative HPLC to afford 3-(5-(4-((4-(2-hydroxypropan-2-yl)piperidin-1-yl)methyl)-5-(trifluoromethyl)-pyridin-2-yl)-1-oxoisoindolin-2-yl)piperidine-2,6-dione (12.8 mg, 73% yield). Preparative HPLC condition: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: 25-65% B over 20 minutes; Flow rate: 20 mL/min; Column temperature: 25° C. The enantiomeric excess of 3-(5-(4-((4-(2-hydroxypropan-2-yl)piperidin-1-yl)methyl)-5-(trifluoromethyl)pyridin-2-yl)-1-oxoisoindolin-2-yl)piperidine-2,6-dione was not determined. LCMS (Method B): retention time 1.18 min. MS (ES): m/z=545.1 [M+H]⁺. ¹H NMR (500 MHz, DMSO-d₆) δ 11.02 (br s, 1H), 8.99 (br s, 1H), 8.35 (s, 1H), 8.32 (br s, 1H), 8.25 (br d, J=8.2 Hz, 1H), 7.92 (br d, J=7.6 Hz, 1H), 5.16 (br dd, J=12.8, 4.3 Hz, 1H), 4.60 (br d, J=17.4 Hz, 1H), 4.47 (br d, J=17.4 Hz, 1H), 3.50-3.32 (m, 3H), 2.93-2.82 (m, 2H), 2.64 (br d, J=15.9 Hz, 1H), 2.48-2.41 (m, 1H), 2.11-1.91 (m, 3H), 1.68 (br d, J=11.9 Hz, 2H), 1.47-1.26 (m, 2H), 1.26-1.12 (m, 1H), 1.05 (s, 6H).

### Examples 180-182

The compounds in Table 22 were prepared according to the general procedures described for Example 184.

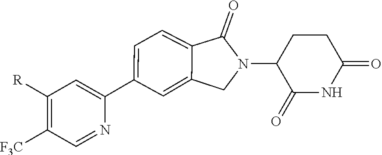

TABLE 22

| | | LC/MS | Retention | |
|---|---|---|---|---|
| Ex. No. | R | [M + H]⁺ | Time (min) | HPLC Method |
| 180 | 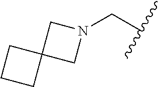 [SMILES: *CN1CC2(CCC2)C1] | 499.1 | 1.27 | B |
| 181 | 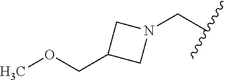 [SMILES: *CN1CC(COC)C1] | 503.0 | 1.21 | B |

### Examples 182

### 3-(5-(3-fluoro-4-((4-(trifluoromethoxy)piperidin-1-yl)methyl)pyridin-2-yl)-1-oxoisoindolin-2-yl)piperidine-2,6-dione

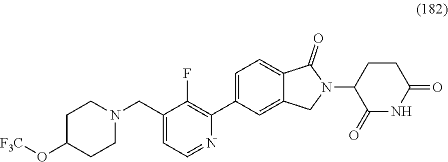 [SMILES: O=C1CCC(N2Cc3cc(-c4nccc(CN5CCC(OC(F)(F)F)CC5)c4F)ccc3C2=O)C(=O)N1]

To Intermediate 149B (19 mg, 0.045 mmol), 4-trifluoromethoxy-piperidine (11.36 mg, 0.067 mmol) was added DMF (2 mL) followed by N-ethyl-N-isopropylpropan-2-amine (0.063 mL, 0.358 mmol). The reaction mixture was heated at 80° C. for 2 h. The reaction mixture was cooled down to room temperature, filtered and purified by preparative HPLC to afford 3-(5-(3-fluoro-4-((4-(trifluoromethoxy)piperidin-1-yl)methyl) pyridin-2-yl)-1-oxoisoindolin-2-yl)piperidine-2,6-dione (14 mg, 59.2% yield). Preparative HPLC condition: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: 24-64% B over 20 minutes; Flow rate: 20 mL/min; Column temperature: 25° C. The enantiomeric excess of 3-(5-(3-fluoro-4-((4-(trifluoromethoxy)piperidin-1-yl)methyl)pyridin-2-yl)-1-oxoisoindolin-2-yl) piperidine-2,6-dione was not determined. LCMS (Method B): retention time 1.14 min. MS (ES): m/z=521.1 [M+H]⁺. ¹H NMR (500 MHz, DMSO-d₆) δ 8.54 (br s, 1H), 8.11 (br s, 1H), 8.07-7.97 (m, 1H), 7.95-7.80 (m, 1H), 7.55 (br s, 1H), 5.27-5.07 (m, 1H), 4.70-4.51 (m, 1H), 4.51-4.34 (m, 2H), 3.59-3.46 (m, 1H), 3.46-3.26 (m, 1H), 3.01-2.81 (m, 1H), 2.79-2.68 (m, 2H), 2.63 (br d, J=15.3 Hz, 1H), 2.48-2.35 (m, 3H), 2.21-2.01 (m, 1H), 2.01-1.88 (m, 2H), 1.76 (br s, 2H).

### Examples 188-197

The compounds in Table 23 were prepared according to the general procedures described for Example 182.

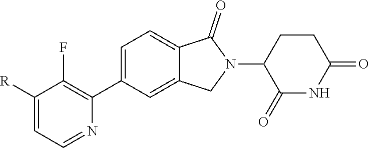

TABLE 23

| | | LC/MS | Retention | |
|---|---|---|---|---|
| Ex. No. | R | [M + H]⁺ | Time (min) | HPLC Method |
| 183 | 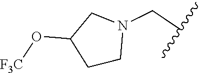 [SMILES: *CN1CCC(OC(F)(F)F)C1] | 507.0 | 1.09 | B |
| 184 | 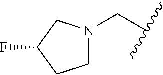 [SMILES: *CN1CC[C@H](F)C1] | 441.0 | 0.94 | B |
| 185 | 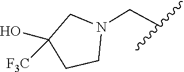 [SMILES: *CN1CCC(O)(C(F)(F)F)C1] | 507.1 | 1.00 | B |
| 186 | 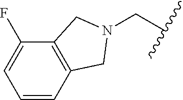 [SMILES: *CN1Cc2cccc(F)c2C1] | 489.0 | 1.09 | B |
| 187 | 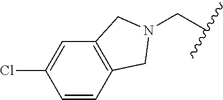 [SMILES: *CN1Cc2ccc(Cl)cc2C1] | 505.0 | 1.17 | B |

### Example 188

### N-(tert-butyl)-1-((2-(2-(2,6-dioxopiperidin-3-yl)-1-oxoisoindolin-5-yl)pyridin-4-yl) methyl)pyrrolidine-2-carboxamide

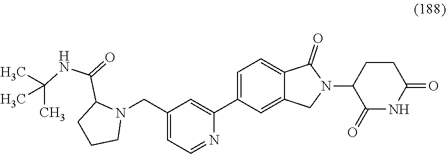 [SMILES: CC(C)(C)NC(=O)C1CCCN1Cc1ccnc(-c2ccc3c(c2)CN(C2CCC(=O)NC2=O)C3=O)c1]

A 1-dram vial was charged with Intermediate 98B (25 mg, 0.062 mmol), N-(tert-butyl)pyrrolidine-2-carboxamide (10.48 mg, 0.062 mmol), DMF (1 mL) and N-ethyl-N-isopropylpropan-2-amine (0.107 mL, 0.615 mmol) and then heated for 2 h at 80° C. The reaction mixture was cooled to room temperature, and 0.5 mL of AcOH was added. The crude material was purified using Preparative HPLC Method 1, described above. The yield of the product was 23.7 mg, and its estimated purity by LCMS analysis was 95%. Analytical HPLC was used to determine the final purity. HPLC method A conditions: Column: Waters XBridge C18, 2.1 mm×50 mm, 1.7 m particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). HPLC method A results: Purity: 98.3%; Observed Mass: 504.30; Retention Time: 1.44 min. ¹H NMR (500 MHz, DMSO-d₆) δ ppm 11.02 (s, 1H) 8.77 (d, J=4.88 Hz, 1H) 8.30 (s, 1H) 8.22 (br d, J=7.93 Hz, 1H) 8.13 (s, 1H) 8.09 (br s, 1H) 7.91 (d, J=7.93 Hz, 1H) 7.53 (d, J=4.88 Hz, 1H) 5.15 (br dd, J=13.58, 5.04 Hz, 1H) 4.53-4.62 (m, 2H) 4.40-4.49 (m, 2H) 4.14 (br t, J=7.17 Hz, 1H) 2.89-2.98 (m, 1H) 2.64 (br d, J=16.17 Hz, 1H) 2.39-2.49 (m, 2H) 2.04-2.16 (m, 2H) 1.85-1.94 (m, 1H) 1.76-1.84 (m, 1H) 0.95-1.00 (m, 9H).

### Examples 189-197

The compounds in Table 24 were prepared according to the general procedures described for Example 188, replacing N-(tert-butyl)pyrrolidine-2-carboxamide with the appropriate amine.

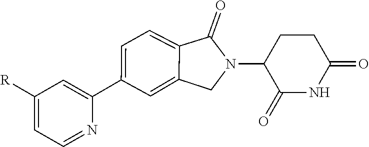

TABLE 24

| | | LC/MS | Retention | |
|---|---|---|---|---|
| Ex. No. | R | [M + H]⁺ | Time (min) | HPLC Method |
| 189 | 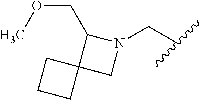 [SMILES: *CN1CC2(CCC2)C1COC] | 475.20 | 1.50 | A |
| 190 | 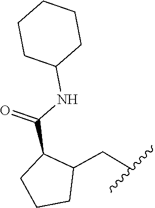 [SMILES: *CC1CCC[C@H]1C(=O)NC1CCCCC1] | 530.30 | 1.50 | A |
| 191 | 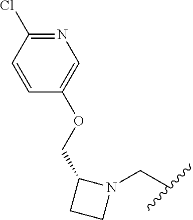 [SMILES: *CN1CC[C@@H]1COc1ccc(Cl)nc1] | 532.10 | 1.42 | a |
| 192 | 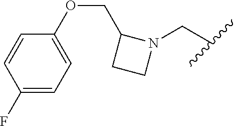 [SMILES: *CN1CCC1COc1ccc(F)cc1] | 515.20 | 1.71 | a |
| 193 | 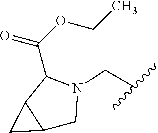 [SMILES: *CN1CC2CC2C1C(=O)OCC] | 515.20 | 1.71 | a |
| 194 | 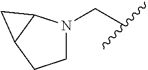 [SMILES: *CN1CCC2CC21] | 489.10 | 1.08 | a |
| 195 | 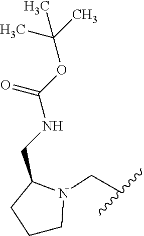 [SMILES: *CN1CCC[C@H]1CNC(=O)OC(C)(C)C] | 534.20 | 1.71 | a |
| 196 | 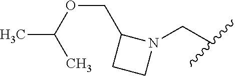 [SMILES: *CN1CCC1COC(C)C] | 463.40 | 1.31 | a |
| 197 | 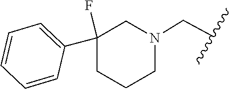 [SMILES: *CN1CCCC(F)(c2ccccc2)C1] | 510.30 | 1.18 | a |

### Example 198

### 3-(5-(3-fluoro-4-((4-fluoro-4-methylpiperidin-1-yl)methyl)pyridin-2-yl)-1-oxoisoindolin-2-yl)piperidine-2,6-dione

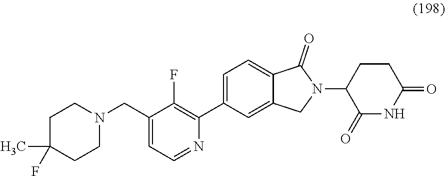 [SMILES: CC1(F)CCN(Cc2ccnc(-c3ccc4c(c3)CN(C3CCC(=O)NC3=O)C4=O)c2F)CC1]

A 1-dram vial was charged with Intermediate 149B (20 mg, 0.047 mmol), 4-fluoro-4-methylpiperidine, HCl (10.86 mg, 0.071 mmol), DMF (1 mL) and N-ethyl-N-isopropylpropan-2-amine (0.082 mL, 0.471 mmol) and then heated for 2 h at 80° C. The reaction mixture was cooled to room temperature and 0.5 mL of AcOH. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 16% B, 16-56% B over 25 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The yield of the product was 16.2 mg, and its estimated purity by LCMS analysis was 100%. Analytical HPLC was used to determine the final purity. HPLC method conditions: Column: Waters XBridge C18, 2.1 mm×50 mm, 1.7 m particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). HPLC results: Purity: 100%; Observed Mass: 469.10; Retention Time: 1.54 min. ¹H NMR (500 MHz, DMSO-d₆) δ ppm 11.01 (s, 1H) 8.54 (d, J=4.56 Hz, 1H) 8.12 (s, 1H) 8.04 (br d, J=7.80 Hz, 1 H) 7.87 (d, J=7.88 Hz, 1H) 7.53-7.57 (m, 1H) 5.16 (dd, J=13.39, 5.10 Hz, 1H) 4.56 (d, J=17.42 Hz, 1H) 4.44 (d, J=17.25 Hz, 1H) 3.70 (br s, 1H) 2.89-3.00 (m, 1H) 2.59-2.67 (m, 2H) 2.51 (br d, J=1.33 Hz, 3H) 2.32-2.48 (m, 3H) 2.02-2.09 (m, 1H) 1.73-1.80 (m, 3H) 1.64-1.72 (m, 1H) 1.28-1.38 (m, 3H).

### Example 199

### 3-(5-(4-((2-acetyltetrahydropyridazin-1(2H)-yl)methyl)-3-fluoropyridin-2-yl)-1-oxoisoindolin-2-yl)piperidine-2,6-dione

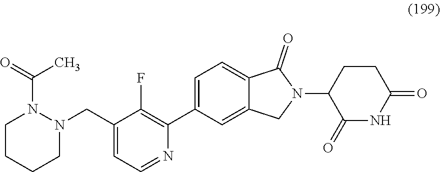 [SMILES: CC(=O)N1CCCCN1Cc1ccnc(-c2ccc3c(c2)CN(C2CCC(=O)NC2=O)C3=O)c1F]

A 1-dram vial was charged with Intermediate 149B (20 mg, 0.047 mmol), hexahydropyridazine (5.68 mg, 0.066 mmol) dissolved in DMF (1 mL), and N-ethyl-N-isopropylpropan-2-amine (0.082 mL, 0.471 mmol) and then stirred at 80° C. for 90 min. The reaction mixture was cooled to room temperature. Acetic anhydride (9.63 mg, 0.094 mmol) was added, the reaction mixture stirred at room temperature for 30 min., and acidified with a few drops of AcOH. The mixture was purified by Preparative HPLC Method 1 to obtain 34% yield of the titled compound. HPLC Method A results: Purity: 100%; Observed Mass: 480.20; Retention Time: 1.21 min. ¹H NMR (500 MHz, DMSO-d₆) δ ppm 11.02 (br s, 1H) 8.52-8.61 (m, 1H) 8.08-8.16 (m, 1H) 8.02 (br d, J=7.63 Hz, 1H) 7.86-7.93 (m, 1H) 7.54 (br s, 1H) 5.12-5.20 (m, 1H) 4.54-4.64 (m, 1H) 4.40-4.49 (m, 1H) 4.16-4.31 (m, 3H) 3.08-3.18 (m, 1H) 2.88-3.07 (m, 3H) 2.63 (br d, J=16.78 Hz, 1H) 2.37-2.47 (m, 1H) 1.99-2.10 (m, 2H) 1.76-1.83 (m, 3H) 1.73 (br d, J=11.90 Hz, 1H) 1.33-1.51 (m, 2H).

### Example 200

### 3-(5-(4-((4-(1,1-difluoropropyl)piperidin-1-yl)methyl)-3-fluoropyridin-2-yl)-1-oxoisoindolin-2-yl)piperidine-2,6-dione

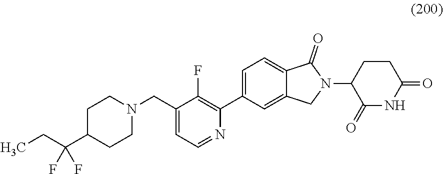 [SMILES: CCC(F)(F)C1CCN(Cc2ccnc(-c3ccc4c(c3)CN(C3CCC(=O)NC3=O)C4=O)c2F)CC1]

### Intermediate 200A: 4-(1,1-difluoropropyl)piperidine

To a solution of 1-(pyridin-4-yl)propan-1-one (1. g, 7.40 mmol) dissolved in DCM (30 mL) at 0° C. was added N,N-diethyl-1,1,1-trifluoro-14-sulfanamine (2.172 mL, 14.80 mmol). The reaction mixture was warmed to room temperature and stirred for 4 days. A 5% aqueous solution of sodium bicarbonate was added, the layers were separated. The organic layer was washed with saturated sodium bicarbonate, and brine then dried over sodium sulfate, filtered and concentrated to provide a 5:1 mixture of 4-(1,1-difluoropropyl)pyridine and 1-(pyridin-4-yl)propan-1-one. The mixture obtained was dissolved in methanol (10 mL) and then 1 M HCl (10 mL) was added, followed by catalytic platinum oxide (50 mg). The resulting suspension was hydrogenated in a Parr shaker at about 40 psi for 4 days. The mixture was filtered through celite and concentrated to obtain a mixture containing about 20% of the titled compound as an HCl salt.

### Example 200

To 4-(1,1-difluoropropyl)piperidine, HCl (8.47 mg, 0.042 mmol) was added Intermediate 149B (18 mg, 0.042 mmol), DMF (1 mL) and N-ethyl-N-isopropylpropan-2-amine (0.074 mL, 0.424 mmol) and then stirred at 90° C. for 1 hour. The mixture was purified by Preparative HPLC Method 1 to obtain 14% yield of the titled compound. HPLC method A results: Purity: 100%; Observed Mass: 515.20; Retention Time: 1.84 min. ¹H NMR (500 MHz, DMSO-d₆) δ ppm 11.02 (br s, 1H) 8.54 (br s, 1H) 8.12 (br s, 1H) 8.04 (br d, J=5.80 Hz, 1H) 7.87 (br d, J=6.71 Hz, 1H) 7.54 (br s, 1H) 5.12-5.19 (m, 1H) 4.53-4.61 (m, 1H) 4.41-4.48 (m, 1H) 2.90-2.97 (m, 2H) 2.60-2.67 (m, 1H) 2.37-2.47 (m, 1H) 2.06 (br t, J=9.31 Hz, 3H) 1.77-1.93 (m, 3H) 1.67-1.76 (m, 2H) 1.39-1.51 (m, 2H) 0.94 (br t, J=7.02 Hz, 3H).

### Example 201

### tert-butyl 2-((2-(2-(2,6-dioxopiperidin-3-yl)-1-oxoisoindolin-5-yl)-3-fluoropyridin-4-yl) methyl)-2,5-diazaspiro[3.5]nonane-5-carboxylate

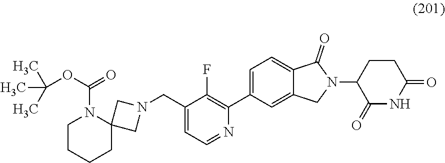 [SMILES: CC(C)(C)OC(=O)N1CCCCC12CN(Cc1ccnc(-c3ccc4c(c3)CN(C3CCC(=O)NC3=O)C4=O)c1F)C2]

A 2-dram vial was charged with Intermediate 149B (60 mg, 0.141 mmol), tert-butyl 2,5-diazaspiro[3.5]nonane-5-carboxylate, 0.5 equivalent oxalic acid salt (46.0 mg, 0.170 mmol), acetonitrile (1 mL) and N-ethyl-N-isopropylpropan-2-amine (0.246 mL, 1.414 mmol) and then stirred at 75° C. for 2 h. The mixture was purified by Preparative HPLC Method 1 to obtain 67% yield of the titled compound. HPLC method A results: Purity: 97.9%; Observed Mass: 578.20; Retention Time: 1.8 min. ¹H NMR (500 MHz, DMSO-d₆) δ ppm 8.53 (d, J=4.73 Hz, 1H) 8.11 (s, 1H) 8.03 (br d, J=7.78 Hz, 1H) 7.87 (d, J=7.93 Hz, 1H) 7.50 (t, J=4.69 Hz, 1H) 5.15 (dd, J=13.31, 4.84 Hz, 1H) 4.56 (d, J=17.32 Hz, 1H) 4.43 (d, J=17.32 Hz, 1H) 3.01-3.10 (m, 2H) 2.87-2.99 (m, 1H) 2.63 (br d, J=15.87 Hz, 1H) 2.37-2.48 (m, 1H) 2.01-2.09 (m, 1H) 1.76-1.85 (m, 2H) 1.68 (br s, 2H) 1.38 (s, 9H) 1.28-1.34 (m, 2H).

### Example 202

### 3-(5-(4-((1-acetyl-1,8-diazaspiro[4.5]decan-8-yl)methyl)-3-fluoropyridin-2-yl)-1-oxoisoindolin-2-yl)piperidine-2,6-dione

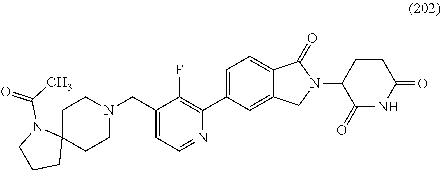 [SMILES: CC(=O)N1CCCC12CCN(Cc1ccnc(-c3ccc4c(c3)CN(C3CCC(=O)NC3=O)C4=O)c1F)CC2]

### Intermediate 202A: 3-(5-(4-((1,8-diazaspiro[4.5]decan-8-yl)methyl)-3-fluoropyridin-2-yl)-1-oxoisoindolin-2-yl)piperidine-2,6-dione

A 2-dram vial was charged with Intermediate 149B (65 mg, 0.153 mmol), tert-butyl 1,8-diazaspiro[4.5]decane-1-carboxylate (44.2 mg, 0.184 mmol), acetonitrile (5 mL) and N-ethyl-N-isopropylpropan-2-amine (0.267 mL, 1.532 mmol) and then heated for 2 h at 75° C. The mixture was concentrated to dryness, and then 5 mL of 1:1 TFA in DCM was added. The mixture was stirred at room temperature. After 1 h, the mixture was concentrated to dryness to obtain a TFA-salt of the titled compound.

### Example 202

A 1-dram vial was charged with 3-(5-(4-((1,8-diazaspiro[4.5]decan-8-yl)methyl)-3-fluoropyridin-2-yl)-1-oxoisoindolin-2-yl)piperidine-2,6-dione, TFA (25 mg, 0.041 mmol), acetic anhydride (12.64 mg, 0.124 mmol), DMF (1.5 mL) and N-ethyl-N-isopropylpropan-2-amine (0.072 mL, 0.413 mmol) and then stirred for 1 h at room temperature. The mixture was purified by Preparative HPLC Method 1 to obtain 63% yield of the titled compound. HPLC method A results: Purity: 99.2%; Observed Mass: 534.20; Retention Time: 1.39 min. ¹H NMR (500 MHz, DMSO-d₆) δ ppm 11.01 (s, 1H) 8.54 (d, J=4.58 Hz, 1H) 8.12 (s, 1H) 8.04 (br d, J=7.63 Hz, 1H) 7.87 (d, J=7.93 Hz, 1H) 7.54 (t, J=4.88 Hz, 1H) 5.15 (dd, J=13.28, 5.04 Hz, 1H) 4.57 (br d, J=17.09 Hz, 1H) 4.44 (br d, J=17.70 Hz, 1H) 2.84-2.97 (m, 3H) 2.79 (br d, J=11.29 Hz, 2H) 2.63 (br d, J=16.17 Hz, 1H) 2.38-2.48 (m, 1H) 2.14 (br t, J=11.60 Hz, 2H) 2.03-2.10 (m, 1H) 1.92 (s, 3H) 1.81-1.87 (m, 2H) 1.71-1.78 (m, 2H) 1.20 (br d, J=11.60 Hz, 2H).

### Example 203

### methyl 8-((2-(2-(2,6-dioxopiperidin-3-yl)-1-oxoisoindolin-5-yl)-3-fluoropyridin-4-yl) methyl)-1,8-diazaspiro[4.5]decane-1-carboxylate

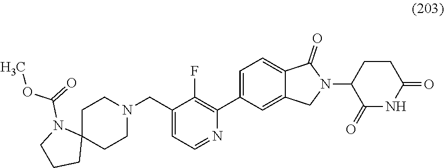 [SMILES: COC(=O)N1CCCC12CCN(Cc1ccnc(-c3ccc4c(c3)CN(C3CCC(=O)NC3=O)C4=O)c1F)CC2]

A 1-dram via was charged with 3-(5-(4-((1,8-diazaspiro[4.5]decan-8-yl)methyl)-3-fluoropyridin-2-yl)-1-oxoisoindolin-2-yl)piperidine-2,6-dione, TFA (25 mg, 0.041 mmol), dimethyl dicarbonate (16.61 mg, 0.124 mmol), DMF (1.5 mL) and N-ethyl-N-isopropylpropan-2-amine (0.072 mL, 0.413 mmol) and then stirred for 1 h at room temperature. The mixture was purified by Preparative HPLC Method 1 to obtain 81% yield of the titled compound. HPLC method A results: Purity: 100%; Observed Mass: 550.10; Retention Time: 1.08 min. ¹H NMR (500 MHz, DMSO-d₆) δ ppm 11.00 (s, 1H) 8.55 (d, J=4.88 Hz, 1H) 8.12 (s, 1H) 8.04 (m, J=7.93 Hz, 1H) 7.95 (s, 1H) 7.87 (m, J=7.93 Hz, 1H) 7.54 (t, J=4.58 Hz, 1H) 5.15 (dd, J=13.28, 5.04 Hz, 1H) 4.57 (d, J=17.70 Hz, 1H) 4.44 (d, J=17.40 Hz, 1H) 3.36 (br t, J=6.87 Hz, 1H) 2.91-2.97 (m, 1H) 2.90 (s, 2H) 2.81 (br d, J=10.99 Hz, 2H) 2.74 (s, 2H) 2.60-2.72 (m, 3H) 2.55 (s, 2H) 2.36-2.48 (m, 2H) 2.12-2.20 (m, 2H) 2.02-2.09 (m, 1H) 1.91 (s, 3H) 1.84 (br s, 2H) 1.71 (quin, J=6.64 Hz, 2H) 1.25 (br d, J=11.29 Hz, 2H).

### Examples 204-275

The compounds in Table 25 were prepared according to the general procedures described for Example 203, replacing 4-fluoro-4-methylpiperidine with the appropriate amine. Compounds with an acetate or a methyl formate were made following the general procedures for Examples 202 and 203, respectively.

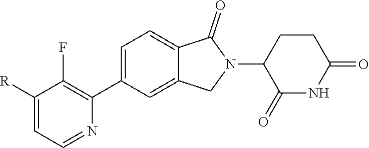

TABLE 25

| | | LC/MS | Retention | HPLC |
|---|---|---|---|---|
| Ex. No. | R | [M + H]⁺ | Time (min) | Method |
| 204 | 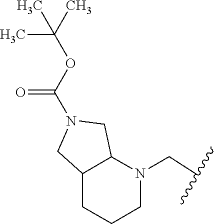 [SMILES: *CN1CCCC2CN(C(=O)OC(C)(C)C)CC21] | 578.40 | 1.15 | A |
| 205 | 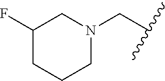 [SMILES: *CN1CCCC(F)C1] | 455.20 | 0.86 | A |
| 206 | 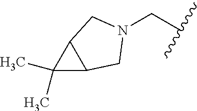 [SMILES: *CN1CC2C(C1)C2(C)C] | 463.20 | 2.06 | A |
| 207 | 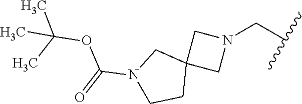 [SMILES: *CN1CC2(CCN(C(=O)OC(C)(C)C)C2)C1] | 564.20 | 1.67 | A |
| 208 | 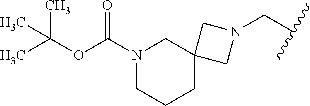 [SMILES: *CN1CC2(CCCN(C(=O)OC(C)(C)C)C2)C1] | 578.30 | 1.82 | A |
| 209 | 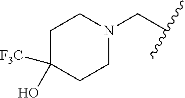 [SMILES: *CN1CCC(O)(C(F)(F)F)CC1] | 521.00 | 1.39 | A |
| 210 | 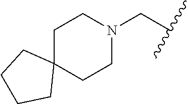 [SMILES: *CN1CCC2(CCCC2)CC1] | 491.10 | 2.08 | A |
| 211* | 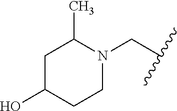 [SMILES: *CN1CCC(O)CC1C] | 467.200 | 1.07 | A |
| 212* | 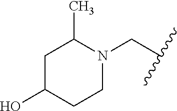 [SMILES: *CN1CCC(O)CC1C] | 467.100 | 1.19 | A |
| 213 | 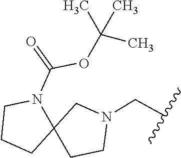 [SMILES: *CN1CCC2(CCCN2C(=O)OC(C)(C)C)C1] | 578.30 | 1.87 | A |
| 214* | 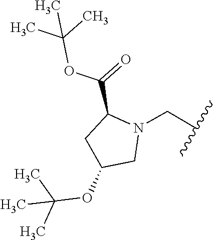 [SMILES: *CN1C[C@H](OC(C)(C)C)C[C@H]1C(=O)OC(C)(C)C] | 595.20 | 2.14 | A |
| 215* | 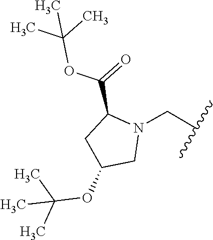 [SMILES: *CN1C[C@H](OC(C)(C)C)C[C@H]1C(=O)OC(C)(C)C] | 595.20 | 2.10 | A |
| 216 | 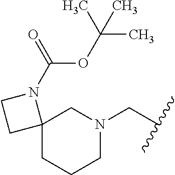 [SMILES: *CN1CCCC2(CCN2C(=O)OC(C)(C)C)C1] | 578.30 | 1.90 | A |
| 217 | 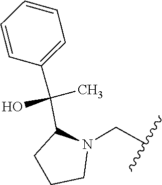 | 543.20 | 1.96 | A |
| 218 | 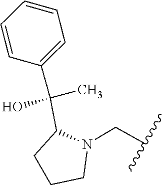 | 543.30 | 1.96 | A |
| 219 | 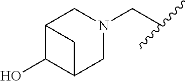 [SMILES: *CN1CC2CC(C1)C2O] | 465.20 | 1.41 | A |
| 220 | 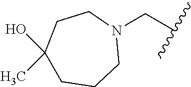 [SMILES: *CN1CCCC(C)(O)CC1] | 481.20 | 1.27 | A |
| 221 | 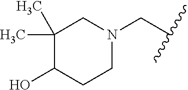 [SMILES: *CN1CCC(O)C(C)(C)C1] | 481.20 | 1.38 | A |
| 222 | 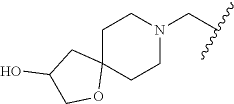 [SMILES: *CN1CCC2(CC1)CC(O)CO2] | 509.10 | 1.09 | A |
| 223 | 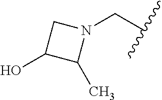 [SMILES: *CN1CC(O)C1C] | 439.10 | 1.08 | A |
| 224 | 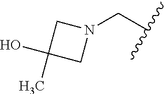 [SMILES: *CN1CC(C)(O)C1] | 439.20 | 1.07 | A |
| 225 | 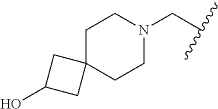 [SMILES: *CN1CCC2(CC1)CC(O)C2] | 493.20 | 1.20 | A |
| 226 | 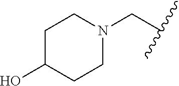 [SMILES: *CN1CCC(O)CC1] | 453.10 | 1.10 | A |
| 227 | 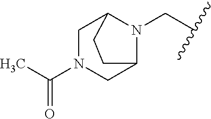 [SMILES: *CN1C2CCC1CN(C(C)=O)C2] | 506.10 | 1.22 | A |
| 228 | 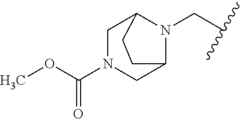 [SMILES: *CN1C2CCC1CN(C(=O)OC)C2] | 521.00 | 1.47 | A |
| 229 | 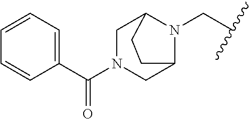 [SMILES: *CN1C2CCC1CN(C(=O)c1ccccc1)C2] | 568.20 | 1.45 | A |
| 230 | 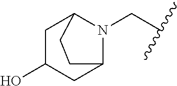 [SMILES: *CN1C2CCC1CC(O)C2] | 479.10 | 1.00 | A |
| 231 | 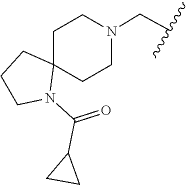 [SMILES: *CN1CCC2(CCCN2C(=O)C2CC2)CC1] | 560.10 | 1.58 | A |
| 232 | 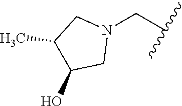 [SMILES: *CN1C[C@@H](C)[C@H](O)C1] | 453.10 | 1.10 | A |
| 233 | 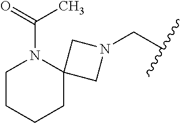 [SMILES: *CN1CC2(CCCCN2C(C)=O)C1] | 520.20 | 0.93 | A |
| 234 | 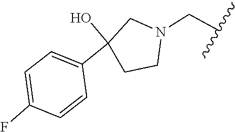 [SMILES: *CN1CCC(O)(c2ccc(F)cc2)C1] | 533.20 | 1.48 | A |
| 235 | 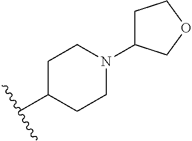 [SMILES: *C1CCN(C2CCOC2)CC1] | 543.10 | 1.55 | A |
| 236 | 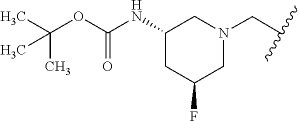 [SMILES: *CN1C[C@@H](F)C[C@H](NC(=O)OC(C)(C)C)C1] | 570.10 | 1.63 | A |
| 237 | 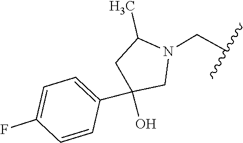 [SMILES: *CN1CC(O)(c2ccc(F)cc2)CC1C] | 547.10 | 1.72 | A |
| 238 | 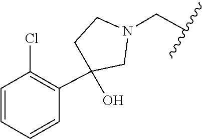 [SMILES: *CN1CCC(O)(c2ccccc2Cl)C1] | 549.10 | 1.64 | A |
| 239 | 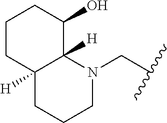 [SMILES: *CN1CCC[C@H]2CCC[C@@H](O)[C@@H]21] | 507.00 | 1.38 | A |
| 240 | 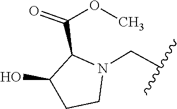 [SMILES: *CN1CC[C@@H](O)[C@H]1C(=O)OC] | 497.00 | 1.06 | A |
| 241 | 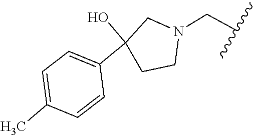 [SMILES: *CN1CCC(O)(c2ccc(C)cc2)C1] | 529.10 | 1.63 | A |
| 242 | 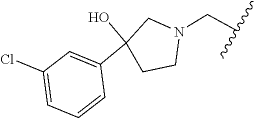 [SMILES: *CN1CCC(O)(c2cccc(Cl)c2)C1] | 549.20 | 1.16 | A |
| 243 | 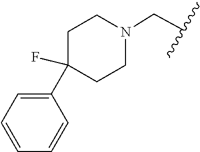 [SMILES: *CN1CCC(F)(c2ccccc2)CC1] | 530.30 | 2.02 | A |
| 244 | 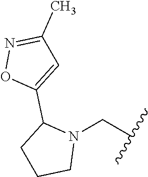 [SMILES: *CN1CCCC1c1cc(C)no1] | 504.00 | 1.48 | A |
| 245 | 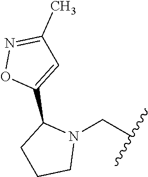 [SMILES: *CN1CCC[C@H]1c1cc(C)no1] | 504.00 | 1.48 | A |
| 246 | 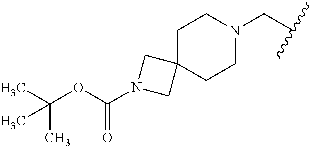 [SMILES: *CN1CCC2(CC1)CN(C(=O)OC(C)(C)C)C2] | 578.10 | 1.78 | A |
| 247 | 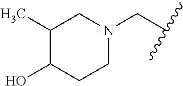 [SMILES: *CN1CCC(O)C(C)C1] | 467.00 | 1.21 | A |
| 248 | 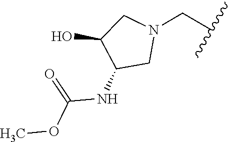 [SMILES: *CN1C[C@H](NC(=O)OC)[C@@H](O)C1] | 512.20 | 0.93 | A |
| 249 | 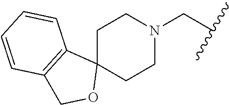 [SMILES: *CN1CCC2(CC1)OCc1ccccc12] | 541.40 | 1.77 | A |
| 250 | 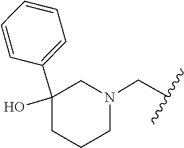 [SMILES: *CN1CCCC(O)(c2ccccc2)C1] | 529.30 | 1.15 | A |
| 251 | 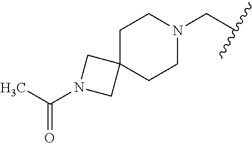 [SMILES: *CN1CCC2(CC1)CN(C(C)=O)C2] | 520.30 | 1.16 | A |
| 252 | 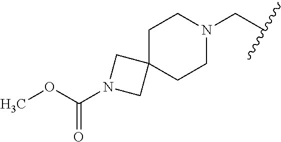 [SMILES: *CN1CCC2(CC1)CN(C(=O)OC)C2] | 536.30 | 1.36 | A |
| 253 | 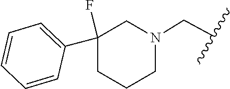 [SMILES: *CN1CCCC(F)(c2ccccc2)C1] | 531.10 | 1.90 | A |
| 254 | 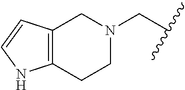 [SMILES: *CN1CCc2[nH]ccc2C1] | 475.10 | 1.17 | A |
| 255 | 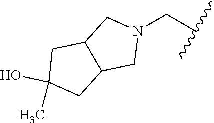 [SMILES: *CN1CC2CC(C)(O)CC2C1] | 493.20 | 1.44 | A |
| 256 | 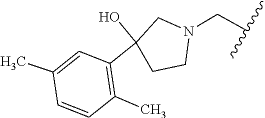 [SMILES: *CN1CCC(O)(c2cc(C)ccc2C)C1] | 543.10 | 1.77 | A |
| 257 | 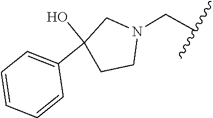 [SMILES: *CN1CCC(O)(c2ccccc2)C1] | 515.20 | 1.49 | A |
| 258 | 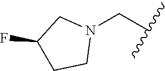 [SMILES: *CN1CC[C@@H](F)C1] | 441.10 | 1.36 | A |
| 259 | 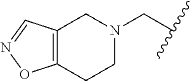 [SMILES: *CN1CCc2oncc2C1] | 476.10 | 1.39 | A |
| 260 | 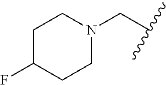 [SMILES: *CN1CCC(F)CC1] | 455.10 | 1.52 | A |
| 261 | 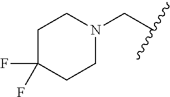 [SMILES: *CN1CCC(F)(F)CC1] | 473.10 | 1.57 | A |
| 262 | 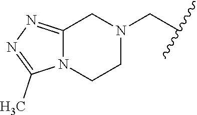 [SMILES: *CN1CCn2c(C)nnc2C1] | 490.10 | 0.97 | A |
| 263 | 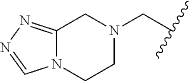 [SMILES: *CN1CCn2cnnc2C1] | 476.10 | 0.95 | A |
| 264 | 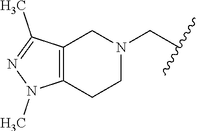 [SMILES: *CN1CCc2c(c(C)nn2C)C1] | 503.10 | 1.24 | A |
| 265 | 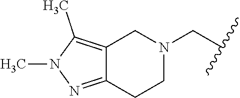 [SMILES: *CN1CCc2nn(C)c(C)c2C1] | 503.10 | 1.32 | A |
| 266 | 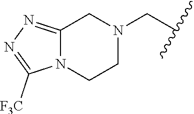 [SMILES: *CN1CCn2c(nnc2C(F)(F)F)C1] | 544.10 | 1.30 | A |
| 267 | 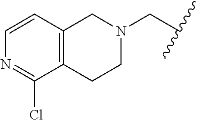 [SMILES: *CN1CCc2c(ccnc2Cl)C1] | 520.00 | 1.57 | A |
| 268 | 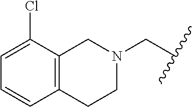 [SMILES: *CN1CCc2cccc(Cl)c2C1] | 519.00 | 2.01 | A |
| 269 | 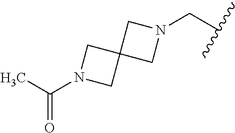 [SMILES: *CN1CC2(C1)CN(C(C)=O)C2] | 492.30 | 1.05 | A |
| 270 | 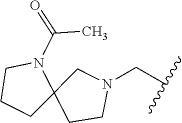 [SMILES: *CN1CCC2(CCCN2C(C)=O)C1] | 520.30 | 1.16 | A |
| 271 | 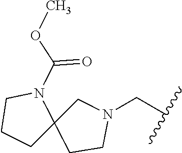 [SMILES: *CN1CCC2(CCCN2C(=O)OC)C1] | 536.40 | 1.45 | A |
| 272 | 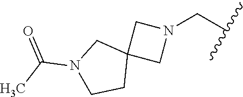 [SMILES: *CN1CC2(CCN(C(C)=O)C2)C1] | 506.30 | 1.07 | A |
| 273 | 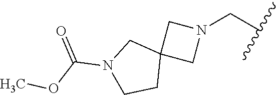 [SMILES: *CN1CC2(CCN(C(=O)OC)C2)C1] | 522.30 | 1.24 | A |
| 274 | 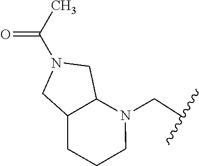 [SMILES: *CN1CCCC2CN(C(C)=O)CC21] | 520.20 | 1.37 | A |
| 275 | 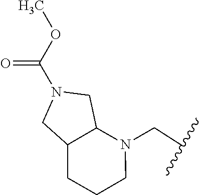 [SMILES: *CN1CCCC2CN(C(=O)OC)CC21] | 536.20 | 1.53 | A |

Compound marked with * is a diastereomer of another compound in the table.

### Example 276

### 3-(4-fluoro-5-(3-fluoro-4-((4-(2-hydroxypropan-2-yl)piperidin-1-yl)methyl)pyridin-2-yl)-1-oxoisoindolin-2-yl)piperidine-2,6-dione

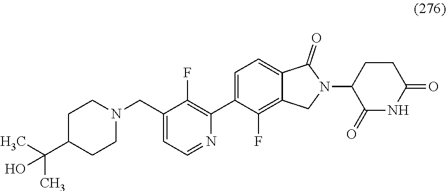 [SMILES: CC(C)(O)C1CCN(Cc2ccnc(-c3ccc4c(c3F)CN(C3CCC(=O)NC3=O)C4=O)c2F)CC1]

### Intermediate 276A: 5-Bromo-4-fluoro-3-hydroxyisobenzofuran-1(3H)-one

To a stirred solution of 2,2,6,6-tetramethylpiperidine (7.07 mL, 41.6 mmol) in THE (150 mL) was added 2.5 M solution of n-BuLi in hexanes (16 mL, 40.0 mmol) at 0° C. The reaction mixture was stirred for 30 min at 0° C. To this reaction mixture, a solution of 4-bromo-3-fluorobenzoic acid (3.5 g, 15.98 mmol) in anhydrous THE (100 mL) was added dropwise at −50° C. The reaction mixture was stirred for 3 h at the same temperature. Anhydrous DMF (2.48 mL, 32.0 mmol) was added at −50° C. and the reaction mixture was allowed to attain room temperature and stirred for 16 h. The reaction was quenched with 1.5 N HCl (100 mL). The reaction mixture was extracted with ethyl acetate (3×30 mL). The combined organic layer was washed with brine, dried over anhydrous Na₂SO₄, filtered and evaporated under reduced pressure. The residue was purified by flash chromatography (SiO₂, 120 g column, 0-50% EtOAc/Pet-ether) to give 5-bromo-4-fluoro-3-hydroxyisobenzofuran-1(3H)-one (1.0 g, 23% yield) as a yellow solid. LCMS (Method A): retention time 0.48 min, [M+H]⁺ 245.1, 247.1; ¹H NMR (400 MHz, ACETONITRILE-d₃) δ 7.93 (dd, J=8.0, 5.5 Hz, 1H), 7.59 (d, J 8.0 Hz, 1H), 6.74 (br s, 1H), 5.94 (br s, 1H).

### Intermediate 276B: tert-butyl (s)-5-amino-4-(5-bromo-4-fluoro-1-oxoisoindolin-2-yl)-5-oxopentanoate

To a stirred solution of 5-bromo-4-fluoro-3-hydroxyisobenzofuran-1(3H)-one (15.0 g, 60.7 mmol) in DMF (200 mL) was added tert-butyl (S)-4,5-diamino-5-oxopentanoate hydrochloride (14.50 g, 60.7 mmol) and sodium triacetoxyborohydride (32.2 g, 152 mmol) at 0° C. The reaction mixture was allowed to stir at room temperature for 48 h. The reaction mixture was diluted with ice water (250 mL). The solid precipitates were filtered and washed with water then dried under reduced pressure to afford the titled compound in 79% yield. ESI MS (M+H)⁺=415.1.

### Intermediate 276C: tert-butyl (s)-5-amino-4-(4-fluoro-1-oxo-5-(4,4,5,5-tetramethyl-1,3,2-dioxaborolan-2-yl)isoindolin-2-yl)-5-oxopentanoate

A dry 500 mL round bottom flask was charged with tert-butyl (S)-5-amino-4-(5-bromo-4-fluoro-1-oxoisoindolin-2-yl)-5-oxopentanoate (5.0 g, 12.04 mmol), 4,4,4′,4′,5,5,5′,5′-octamethyl-2,2′-bi(1,3,2-dioxaborolane) (3.67 g, 14.45 mmol), and potassium acetate (3.55 g, 36.1 mmol) and flushed with nitrogen. The solids were suspended in dioxane (80 mL) and degassed with a stream of nitrogen for 5 min with stirring. The reaction mixture was treated with Pd(dppf)Cl₂ (0.352 g, 0.482 mmol), degassed for 5 min, sealed, and heated to 60° C. for 18 h under nitrogen. The reaction mixture was cooled to room temperature, diluted with EtOAc, washed with brine, dried over MgSO₄ and the filtrate was concentrated. The yield was assumed to be theoretical. ESI MS (M+H)⁺=463.2.

### Intermediate 276D: tert-butyl (S)-5-amino-4-(4-fluoro-5-(3-fluoro-4-(hydroxymethyl) pyridin-2-yl)-1-oxoisoindolin-2-yl)-5-oxopentanoate

A 100 mL flask was charged with tert-butyl (S)-5-amino-4-(4-fluoro-1-oxo-5-(4,4,5,5-tetramethyl-1,3,2-dioxaborolan-2-yl)isoindolin-2-yl)-5-oxopentanoate (1.85 g, 4.00 mmol) dissolved in dioxane (40 mL), (2-chloro-3-fluoropyridin-4-yl)methanol (0.679 g, 4.20 mmol), Pd(dtbpf)Cl₂ (0.078 g, 0.120 mmol), and aqueous K₃PO₄ (6.67 mL, 20.01 mmol). The reaction mixture was sealed and the air was replaced with nitrogen, and then heated overnight at 75° C. The reaction mixture was cooled to room temperature, and diluted with EtOAc, washed with brine, and the organic layer separated, dried over MgSO₄ and concentrated. The mixture was purified using a 120 g silica gel column by ISCO eluting with 0-10% DCM/MeOH to obtain 68% yield of the titled compound.

### Intermediate 276E: 3-(5-(4-(chloromethyl)-3-fluoropyridin-2-yl)-4-fluoro-1-oxoisoindolin-2-yl)piperidine-2,6-dione, HCl

To tert-butyl 5-amino-4-(4-fluoro-5-(3-fluoro-4-(hydroxymethyl)pyridin-2-yl)-1-oxoisoindolin-2-yl)-5-oxopentanoate (1.240 g, 2.69 mmol) dissolved in DCM (35 mL) and cooled in an ice-water bath was added thionyl chloride (0.585 mL, 8.06 mmol) (dropwise). After 10 minutes, the ice-bath was removed and the reaction mixture was allowed to warm to room temperature. After 0.5 h, the mixture was concentrated to dryness. The residue obtained was dissolved in acetic acid (18 mL) and then PhSO₃H (0.935 g, 5.91 mmol) was added and refluxed for 3 h. The mixture was concentrated to dryness, and 30 mL of 3 M HCl in MeOH was added and stirred at 0° C. until there was complete dissolution. Next, 70 mL of EtOAc was added, and the reaction mixture was stirred for about 5 min. The mixture was allowed to stay still in the ice-water bath for about 0.5 h. The precipitates were filtered, washed with EtOAc, and then air-dried to obtain the titled product in 58% yield.

### Example 276

A 2-dram vial was charged with 3-(5-(4-(chloromethyl)-3-fluoropyridin-2-yl)-4-fluoro-1-oxoisoindolin-2-yl)piperidine-2,6-dione, HCl (20 mg, 0.045 mmol), 2-(piperidin-4-yl)propan-2-ol, HCl (10.16 mg, 0.057 mmol), DMF (1 mL) and N-ethyl-N-isopropylpropan-2-amine (0.079 mL, 0.452 mmol). The reaction mixture was heated for 1.5 hour at 80° C. The mixture was cooled to room temperature, and acidified with a few drops of AcOH and then purified by Preparative HPLC Method 1 to obtain 39% yield of the titled compound. HPLC method A results: Purity: 95.7%; Observed Mass: 513.20; Retention Time: 0.92 min. ¹H NMR (500 MHz, DMSO-d₆) δ ppm 11.03 (s, 1H) 8.56 (br d, J=4.27 Hz, 1H) 7.73-7.82 (m, 2H) 7.61 (br t, J=4.27 Hz, 1H) 5.11-5.20 (m, 1H) 4.63-4.71 (m, 1H) 4.46-4.55 (m, 1H) 2.91 (br d, J=10.68 Hz, 2H) 2.59-2.68 (m, 1H) 2.43-2.49 (m, 1H) 2.03-2.10 (m, 1H) 1.93-2.01 (m, 2H) 1.67 (br d, J=10.68 Hz, 2H) 1.23-1.33 (m, 2H) 1.14-1.21 (m, 1H) 1.04 (s, 6H).

### Examples 277-284

The compounds in Table 26 were prepared according to the general procedures described for Example 276, replacing 2-(piperidin-4-yl)propan-2-ol with the appropriate amine.

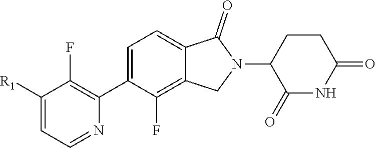

TABLE 26

| Ex. | | LC/MS | Retention | |
|---|---|---|---|---|
| No. | R₁ | [M + H]⁺ | Time (min) | HPLC Method |
| 277 | 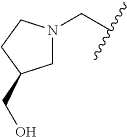 [SMILES: *CN1CC[C@H](CO)C1] | 471.20 | 1.03 | A |
| 278 | 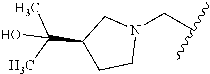 [SMILES: *CN1CC[C@@H](C(C)(C)O)C1] | 499.10 | 1.30 | A |
| 279 | 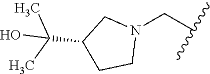 [SMILES: *CN1CC[C@H](C(C)(C)O)C1] | 499.10 | 1.30 | a |
| 280 | 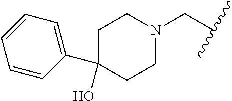 [SMILES: *CN1CCC(O)(c2ccccc2)CC1] | 547.10 | 1.51 | a |
| 281 | 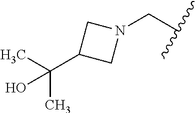 [SMILES: *CN1CC(C(C)(C)O)C1] | 485.20 | 1.15 | a |
| 282 | 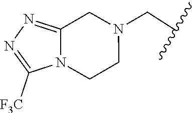 [SMILES: *CN1CCn2c(nnc2C(F)(F)F)C1] | 562.00 | 1.32 | a |
| 283 | 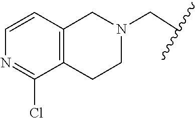 [SMILES: *CN1CCc2c(ccnc2Cl)C1] | 538.00 | 1.59 | a |
| 284 | 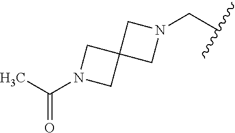 [SMILES: *CN1CC2(C1)CN(C(C)=O)C2] | 510.20 | 1.06 | a |

### Example 285

### 3-(5-(6-amino-5-((8-benzoyl-3,8-diazabicyclo[3.2.1]octan-3-yl)methyl)pyridin-2-yl)-1-oxoisoindolin-2-yl)piperidine-2,6-dione

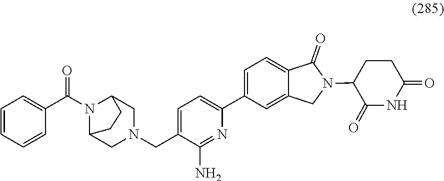 [SMILES: Nc1nc(-c2ccc3c(c2)CN(C2CCC(=O)NC2=O)C3=O)ccc1CN1CC2CCC(C1)N2C(=O)c1ccccc1]

### Intermediate 285A: tert-butyl 3-((2-amino-6-chloropyridin-3-yl)methyl)-3,8-diazabicyclo[3.2.1]octane-8-carboxylate

To a stirred solution of 2-amino-6-chloronicotinaldehyde (500 mg, 3.19 mmol) and tert-butyl (1R,5S)-3,8-diazabicyclo[3.2.1]octane-8-carboxylate (678 mg, 3.19 mmol) in THE (20 mL), was added acetic acid (0.548 mL, 9.58 mmol) at 0° C. The reaction mixture was allowed to stir for 30 min at ambient temperature and then sodium triacetoxyborohydride (1354 mg, 6.39 mmol) at 0° C. was added. The reaction mixture was stirred at ambient temperature for 16 h. After completion of the reaction, the reaction mixture was cooled to 0° C. The reaction was quenched with saturated NH₄C₁ solution. Then reaction mixture was poured in water (50 mL) and extracted with EtOAc (2×100 mL). The combined organic phases were dried anhydrous Na₂SO₄, filtered and the filtrate was concentrated under reduced pressure to afford the crude product. The crude product was purified by flash chromatography (SiO₂, 25 g column, 0-100% EtOAc/pet ether) to afford tert-butyl (1R,5S)-3-((2-amino-6-chloropyridin-3-yl)methyl)-3,8-diazabicyclo[3.2.1]octane-8-carboxylate (620 mg, 1.207 mmol, 38% yield) as a pale brown solid. LCMS (Method B): Retention time 2.74 min, [M+H]⁺353.2.

### Intermediate 285B: 3-((3,8-diazabicyclo[3.2.1]octan-3-yl)methyl)-6-chloropyridin-2-amine

To a well-stirred solution of tert-butyl (1R,5S)-3-((2-amino-6-chloropyridin-3-yl) methyl)-3,8-diazabicyclo[3.2.1]octane-8-carboxylate (600 mg, 1.700 mmol) in anhydrous DCM (8 mL) was added TFA (2 mL) at ambient temperature under nitrogen atmosphere. Then the reaction mixture was stirred for 2 h at room temperature. The reaction mass was concentrated to provide crude product 3-(((1R,5S)-3,8-diazabicyclo[3.2.1]octan-3-yl) methyl)-6-chloropyridin-2-amine (550 mg, 1.696 mmol, 100% yield) as a thick, brownish syrup. LCMS (Method B): Retention time 0.34 min, [M+H]⁺253.2.

### Intermediate 285C: (3-((2-amino-6-chloropyridin-3-yl)methyl)-3,8-diazabicyclo[3.2.1]octan-8-yl)(phenyl) methanone

To a well-stirred solution of 3-(((1R,5S)-3,8-diazabicyclo[3.2.1]octan-3-yl) methyl)-6-chloropyridin-2-amine (400 mg, 1.583 mmol) in DCM (10 mL) at 0° C. were added TEA (1.324 mL, 9.50 mmol) and benzoic anhydride (537 mg, 2.374 mmol) at 0° C. under nitrogen atmosphere. The reaction mixture was stirred for 16 h at room temperature. The solvent was concentrated under reduced pressure to afford a crude product. The residue was purified by flash chromatography (SiO₂, 25 g column, 0-100% EtOAc/pet ether) to afford ((1R,5S)-3-((2-amino-6-chloropyridin-3-yl)methyl)-3,8-diazabicyclo[3.2.1]octan-8-yl)(phen-yl)methanone (560 mg, 1.048 mmol, 66% yield) as an off-white solid. LCMS (Method B): Retention time 2.07 min, [M+H]⁺357.2.

### Intermediate 285D: tert-butyl 5-amino-4-(5-(6-amino-5-((8-benzoyl-3,8-diazabicyclo [3.2.1]octan-3-yl)methyl)pyridin-2-yl)-1-oxoisoindolin-2-yl)-5-oxopentanoate

Into a 30 mL microwave vail containing a well stirred solution of ((1R,5S)-3-((2-amino-6-chloropyridin-3-yl)methyl)-3,8-diazabicyclo[3.2.1]octan-8-yl)(phenyl) methanone (150 mg, 0.420 mmol) and tert-butyl (S)-5-amino-5-oxo-4-(1-oxo-5-(4,4,5,5-tetramethyl-1,3,2-dioxaborolan-2-yl)isoindolin-2-yl)pentanoate (205 mg, 0.462 mmol) in 1,4-dioxane (7 mL) and water (1 mL) was added sodium bicarbonate (88 mg, 1.051 mmol) at ambient temperature under nitrogen atmosphere. The reaction mixture was degassed by bubbling with nitrogen gas into reaction mixture for 20 minutes. Next, Pd(PPh₃)₄ (24.29 mg, 0.021 mmol) was added to the reaction mixture. The reaction mixture was heated to 120° C. under microwave irradiation for 2 h. The reaction vessel was allowed to cool to ambient temperature, diluted with ethyl acetate (50 mL), filtered through a bed of Celite, and concentrated in vacuo to afford the crude product. The crude product was purified by flash chromatography (SiO₂, 25 g column, 0-10% MeOH/DCM) to afford tert-butyl 5-amino-4-(5-(6-amino-5-(((1R,5S)-8-benzoyl-3,8-diazabicyclo[3.2.1]octan-3-yl)methyl)pyridin-2-yl)-1-oxoisoindolin-2-yl)-5-oxopentanoate (120 mg, 0.163 mmol, 39% yield) as an off-white solid. LCMS (Method B): Retention time 1.88 min, [M+H]⁺639.2.

### Example 285

To a stirred solution of tert-butyl 5-amino-4-(5-(6-amino-5-((8-benzoyl-3,8-diazabicyclo[3.2.1]octan-3-yl)methyl)pyridin-2-yl)-1-oxoisoindolin-2-yl)-5-oxopentanoate (120 mg, 0.188 mmol) in acetonitrile (3.0 mL) in microwave vial under N₂ atmosphere was added benzenesulfonic acid (44.6 mg, 0.282 mmol). The reaction mixture was sealed and irradiated in the microwave for 2 h at 130° C. After completion of the reaction, the reaction mass was concentrated to provide crude product. Purification was done by using preparative HPLC (Diluent: THF: Acetonitrile (50:50); Column: XBridge C18 (250×19) mm, 5 micron; Mobile phase A: 10 mM Ammonium acetate in water; Mobile phase B: Acetonitrile; Gradient: 30% A+70% B to 70% A+30% B over 15 min, Flow: 15 mL/min). The fractions containing the desired product were lyophilized to dryness to give 3-(5-(6-amino-5-((8-benzoyl-3,8-diazabicyclo[3.2.1]octan-3-yl)methyl) pyridin-2-yl)-1-oxoisoindolin-2-yl)piperidine-2,6-dione (32 mg, 0.055 mmol, 29% yield) as a white solid. LCMS (method D): Retention time 1.32, 565.2 [M+H]⁺; HPLC (method A): Retention time 5.80 min, purity: 96.38%; ¹H NMR: 400 MHz (DMSO) δ 10.99 (s, 1H), 8.20 (s, 1H), 8.15 (d, J=7.20 Hz, 1H), 7.78 (d, J=7.20 Hz, 1H), 7.55-7.40 (m, 6H), 7.20 (d, J=6.40 Hz, 1H), 6.22 (s, 2H), 5.14 (br d, J=13.20 Hz, 1H), 4.62 (br s, 1H), 4.46 (dd, J=17.20, 51.40 Hz, 2H), 3.98 (br s, 1H), 3.46 (m, 2H), 2.93 (br t, J=13.60 Hz, 1H), 2.75 (m, 1H), 2.68-2.55 (m, 2H), 2.43 (m, 1H), 2.38-2.12 (m, 2H), 2.10-1.98 (m, 1H), 1.93-1.75 (m, 4H).

### Example 286

### 3-(5-(5-((2-azaspiro[3.3]heptan-2-yl)methyl)-6-aminopyridin-2-yl)-1-oxoisoindo-lin-2-yl) piperidine-2,6-dione

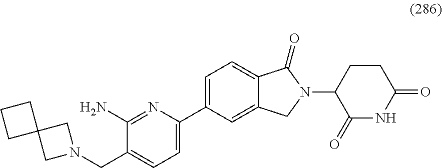 [SMILES: Nc1nc(-c2ccc3c(c2)CN(C2CCC(=O)NC2=O)C3=O)ccc1CN1CC2(CCC2)C1]

### Intermediate 286A: 3-((2-azaspiro[3.3]heptan-2-yl)methyl)-6-chloropyridin-2-amine

To a stirred solution of 2-azaspiro[3.3]heptane hydrochloride (256 mg, 1.916 mmol) and 2-amino-6-chloronicotinaldehyde (300 mg, 1.916 mmol) in THE (15 mL) was added acetic acid (0.329 mL, 5.75 mmol) at 0° C. The reaction mixture was allowed to stir for 30 min at ambient temperature and then sodium triacetoxyborohydride (812 mg, 3.83 mmol) at 0° C. was added. The reaction mixture was stirred at ambient temperature for 16 h. After completion of the reaction, the reaction mass was concentrated to provide crude product. The residue was purified by flash chromatography (SiO₂, 25 g column, 0-100% EtOAc/pet ether) to afford 3-((2-azaspiro[3.3]heptan-2-yl)methyl)-6-chloropyridin-2-amine (220 mg, 0.772 mmol, 40% yield) as a light yellowish solid. LCMS (Method B): Retention time-0.50 min, [M+H]⁺238.0.

### Intermediate 286B: tert-butyl 4-(5-(5-((2-azaspiro[3.3]heptan-2-yl)methyl)-6-aminopyridin-2-yl)-1-oxoisoindolin-2-yl)-5-amino-5-oxopentanoate

Into a 30 mL microwave vail containing a well stirred solution of 3-((2-azaspiro[3.3]heptan-2-yl)methyl)-6-chloropyridin-2-amine (100 mg, 0.421 mmol) and tert-butyl (S)-5-amino-5-oxo-4-(1-oxo-5-(4,4,5,5-tetramethyl-1,3,2-dioxaborolan-2-yl) isoindolin-2-yl)pentanoate (206 mg, 0.463 mmol) in 1,4-dioxane (7.5 mL) and water (1.5 mL) was added sodium bicarbonate (88 mg, 1.052 mmol) at ambient temperature under nitrogen atmosphere. The resulting reaction mixture was degassed by bubbling with nitrogen gas into reaction mixture for 15 minutes. Then Pd(PPh₃)₄ (24.30 mg, 0.021 mmol) was added to the reaction mixture and resulting reaction mixture was heated to 120° C. under microwave irradiation for 90 min. The reaction vessel was allowed to cool to ambient temperature, diluted with ethyl acetate (˜100 mL), filtered through a bed of Celite, and concentrated in vacuo to afford the crude product. The residue was purified by flash chromatography (SiO₂, 25 g column, 0-5% MeOH/DCM) to afford tert-butyl 4-(5-(5-((2-azaspiro[3.3]heptan-2-yl)methyl)-6-aminopyridin-2-yl)-1-oxoisoind-olin-2-yl)-5-amino-5-oxopentanoate (130 mg, 0.221 mmol, 53% yield) as an off-white solid. LCMS (Method B): Retention time 1.10 min, [M+H]⁺ 520.2.

### Example 286

To a stirred solution of tert-butyl 4-(5-(5-((2-azaspiro[3.3]heptan-2-yl)methyl)-6-aminopyridin-2-yl)-1-oxoisoindolin-2-yl)-5-amino-5-oxopentanoate (120 mg, 0.231 mmol) in acetonitrile (3 mL) was added TFA (1 mL) at room temperature. The reaction mixture was stirred at 130° C. for 1 h under microwave. After completion of the reaction, the reaction mass was concentrated to provide crude product which was purified by preparative HPLC (Diluent: THF: acetonitrile (30:70); Column: XSelect C18 (150×19) mm, 5 micron; Mobile phase A: 5 mM ammonium formate in water; Mobile phase B: acetonitrile; Gradient: 95% A+5% B to 65% A+35% B over 18 min, Flow: 15 mL/min). The fractions containing the desired product were lyophilized to dryness to give 3-(5-(5-((2-azaspiro[3.3]heptan-2-yl)methyl)-6-aminopyridin-2-yl)-1-oxoisoindo-lin-2-yl) piperidine-2,6-dione (21 mg, 0.047 mmol, 20% yield) as a white solid. LCMS (method C): Retention time 1.14, 445.6 [M+H]⁺; HPLC (method C): Retention time 5.58 min., purity: 99.05%; ¹HNMR: 400 MHZ (DMSO): δ 11.02 (s, 1H), 8.20-8.12 (m, 3H), 7.78 (d, J=8 Hz, 1H), 7.44 (d, J=7.6 Hz, 1H), 7.19 (d, J=7.6 Hz, 1H), 6.16 (s, 2H), 5.14 (dd, J=4.8, 13.20 Hz, 1H), 4.45 (dd, J=17.6, 53.8 Hz, 2H), 3.15-3.60 (m, 5H-merged in water peak of DMSO), 2.89-2.98 (m, 1H), 2.68-2.54 (m, 2H), 2.42-2.50 (m, 1H), 1.98-2.11 (m, 4H), 1.76 (m, 2H).

### Example 287

### 3-(5-(3,5-difluoro-4-((4-(2-hydroxypropan-2-yl)piperidin-1-yl)methyl)pyridin-2-yl)-1-oxoisoindolin-2-yl)piperidine-2,6-dione

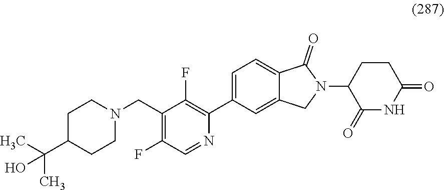 [SMILES: CC(C)(O)C1CCN(Cc2c(F)cnc(-c3ccc4c(c3)CN(C3CCC(=O)NC3=O)C4=O)c2F)CC1]

### Intermediate 287A: Ethyl 2-bromo-3,5-difluoroisonicotinate

Ethyl 2-bromo-3,5-difluoroisonicotinate was prepared according to the procedures described in reference WO2019/014352.

### Intermediate 287B: (2-bromo-3,5-difluoropyridin-4-yl)methanol

To a solution of ethyl 2-bromo-3,5-difluoroisonicotinate (280 mg, 1.052 mmol) in THE (3 mL) was added sodium borohydride (0.119 g, 3.16 mmol) followed by MeOH (1.5 mL). The reaction mixture was stirred at room temperature for 2 h. Water was added to quench the reaction. The solvent was evaporated and the residue was dissolved in EtOAc and washed by brine. The organic phase was dried by sodium sulfate and the solvent was evaporated to afford (2-bromo-3,5-difluoropyridin-4-yl)methanol (0.145 g, 61.5% yield). MS (ES): m/z=223.9 [M+H]⁺.

### Intermediate 287C: tert-butyl 5-amino-4-(5-(3,5-difluoro-4-(hydroxymethyl)pyridin-2-yl)-1-oxoisoindolin-2-yl)-5-oxopentanoate

To a pressure-relief vial was added (2-bromo-3,5-difluoropyridin-4-yl)methanol (0.145 g, 0.647 mmol), tert-butyl (S)-5-amino-5-oxo-4-(1-oxo-5-(4,4,5,5-tetramethyl-1,3,2-dioxaborolan-2-yl)isoindolin-2-yl)pentanoate (0.316 g, 0.712 mmol), Pd(dtbpf)Cl₂ (0.013 g, 0.019 mmol), dioxane (2 mL) and 2 M K₃PO₄ (0.971 mL, 1.942 mmol). The reaction mixture was bubbled with nitrogen for 5 min. The vial was sealed and the reaction mixture was heated to 70° C. for 1 h. The reaction mixture was cooled down to room temperature, diluted by ethyl acetate and washed with brine. The organic phase was dried over Na₂SO₄, and the solvent was evaporated to give a residue, which was purified by flash column chromatography (0-20% MeOH/DCM) to give tert-butyl 5-amino-4-(5-(3,5-difluoro-4-(hydroxymethyl)pyridin-2-yl)-1-oxoisoindolin-2-yl)-5-oxopentanoate (0.156 g, 52.2% yield). The enantiomeric excess of tert-butyl 5-amino-4-(5-(3,5-difluoro-4-(hydroxymethyl)pyridin-2-yl)-1-oxoisoindolin-2-yl)-5-oxopentanoate and subsequent intermediates were not determined. MS (ES): m/z=462.2 [M+H]⁺.

### Intermediate 276D: 3-(5-(4-(chloromethyl)-3,5-difluoropyridin-2-yl)-1-oxoisoindolin-2-yl)piperidine-2,6-dione

To tert-butyl 5-amino-4-(5-(3,5-difluoro-4-(hydroxymethyl)pyridin-2-yl)-1-oxoisoindolin-2-yl)-5-oxopentanoate (156 mg, 0.338 mmol) in DCM (4 mL) at 0° C. was added thionyl chloride (0.074 mL, 1.014 mmol) in DCM (1 mL) dropwise. The reaction mixture was warmed up to room temperature and stirred at room temperature for 30 min. The solvent was evaporated to give tert-butyl 5-amino-4-(5-(4-(chloromethyl)-3,5-difluoropyridin-2-yl)-1-oxoisoindolin-2-yl)-5-oxopentanoate. MS (ES): m/z=480.1 [M+H]⁺. To tert-butyl 5-amino-4-(5-(4-(chloromethyl)-3,5-difluoropyridin-2-yl)-1-oxoisoindolin-2-yl)-5-oxopentanoate was added benzenesulfonic acid (0.118 g, 0.744 mmol) in acetic acid (2 mL) and the reaction mixture was heated at reflux for 2 h. The reaction mixture was cooled down and the solvent was evaporated to afford 3-(5-(4-(chloromethyl)-3,5-difluoropyridin-2-yl)-1-oxoisoindolin-2-yl)piperidine-2,6-dione (0.12 g, 87% yield). MS (ES): m/z=406.1 [M+H]⁺.

### Example 287

To 3-(5-(4-(chloromethyl)-3,5-difluoropyridin-2-yl)-1-oxoisoindolin-2-yl) piperidine-2,6-dione (20 mg, 0.045 mmol), 2-(4-piperidyl)-2-propanol (9.72 mg, 0.068 mmol) was added DMF (2 mL) followed by N-ethyl-N-isopropylpropan-2-amine (0.063 mL, 0.362 mmol). The reaction mixture was heated at 80° C. for 2 h. The reaction mixture was cooled down to room temperature, filtered and purified by preparative HPLC to afford 3-(5-(3,5-difluoro-4-((4-(2-hydroxypropan-2-yl)piperidin-1-yl)methyl)pyridin-2-yl)-1-oxoisoindolin-2-yl)piperidine-2,6-dione (11.8 mg, 49.6% yield). Preparative HPLC condition: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: 11-51% B over 20 minutes; Flow rate: 20 mL/min; Column temperature: 25° C. The enantiomeric excess of 3-(5-(3,5-difluoro-4-((4-(2-hydroxypropan-2-yl)piperidin-1-yl)methyl)pyridin-2-yl)-1-oxoisoindolin-2-yl)piperidine-2,6-dione was not determined. LCMS (Method B): retention time 0.94 min. MS (ES): m/z=513.2 [M+H]⁺. ¹H NMR (500 MHz, DMSO-d₆) δ 11.01 (s, 1H), 8.66 (s, 1H), 8.08 (s, 1H), 7.99 (br d, J=7.9 Hz, 1H), 7.88 (d, J=7.9 Hz, 1H), 5.14 (dd, J=13.2, 5.3 Hz, 1H), 4.56 (br d, J=17.4 Hz, 1H), 4.43 (d, J=17.4 Hz, 1H), 3.70 (s, 2H), 3.46 (s, 1H), 3.00-2.84 (m, 3H), 2.63 (br d, J=17.5 Hz, 1H), 2.47-2.38 (m, 1H), 2.10-1.90 (m, 3H), 1.64 (br d, J=11.8 Hz, 2H), 1.29-1.15 (m, 2H), 1.14-1.05 (m, 1H), 1.00 (s, 6H).

### Examples 288-289

The compounds in Table 27 were prepared according to the general procedures described for Example 287.

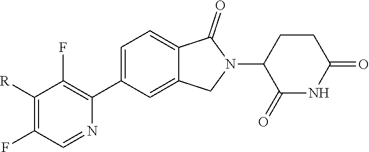

TABLE 27

| | | LC/MS | Retention | |
|---|---|---|---|---|
| Ex. No. | R | [M + H]⁺ | Time (min) | HPLC Method |
| 288 | 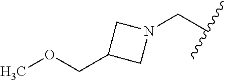 [SMILES: *CN1CC(COC)C1] | 471.2 | 0.96 | B |
| 289 | 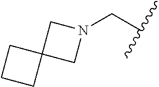 [SMILES: *CN1CC2(CCC2)C1] | 467.0 | 1.07 | B |

### Biological Assays

The pharmacological properties of the compounds of this invention may be confirmed by a number of biological assays. The exemplified biological assays, which follow, have been carried out with compounds of the invention.

### Helios Cellular Degradation Assay

Jurkat cells were plated at 80,000 cells/well in 40 μL RPMI+10% FBS in a 384 well cell culture plate prior to using acoustic dispensing technology for adding compound of interest. Cell cultures were incubated for 72 h at 37° C. and 5% CO₂. In order to facilitate analysis, cell cultures were spun down at 200 rpm for 5 min and the supernatant was discarded. After shaking the plate to dislodge the cell pellet, cells were resuspended in 50 μL of Fixation Buffer (eBioScience FoxP3 buffer set 00-5523-00) for 60 min at room temperature. After centrifuging and discarding the supernatant, cells were permeabilized with 50 μL of Permeabilization buffer (eBioScience FoxP3 buffer set 00-5523-00) for 10 min at room temperature. Following permeabilization, cells were spun down and the supernatant was replaced with 20 μL fluorescently labelled antibodies against Helios, Ikaros and Aiolos or corresponding Isotype controls in 1× Permeabilization buffer (Ikaros-Alexa488 [Biolegend, Cat #368408, 1:50], Helios-PE [CST, Cat #29360, 1:50], Aiolos-Alexa647 [Biolegend, Cat #371106Biolegend, 1:25]) and staining reactions were incubated for 1 h at room temperature; protected from light. Subsequently, 30 μL of 1× Permeabilization buffer was added prior to centrifuging the cells and discarding the supernatant. Stained cells were resuspended in 25 μL of flow cytometry staining buffer (PBS+0.2% BSA) and analyzed using an Intellicyt Ique Plus flow cytometer.

TABLE A

| | Helios Jurkat |
|---|---|
| Ex. No. | IC₅₀ (nM) |
| 1 | >10,000 |
| 2 | 15.3 |
| 3 | 192 |
| 4 | 23.0 |
| 5 | 4,930 |
| 6 | >10,000 |
| 7 | 48.0 |
| 8 | >10,000 |
| 9 | >10,000 |
| 11 | 26.2 |
| 12 | 20.2 |
| 13 | 19.2 |
| 14 | 26.3 |
| 15 | 37.3 |
| 16 | 57.2 |
| 17 | 119 |
| 19 | 142 |
| 20 | 34.8 |
| 21 | 34.7 |
| 22 | 14.8 |
| 24 | 49.6 |
| 25 | >10,000 |
| 26 | >10,000 |
| 27 | 26.9 |
| 28 | 1,320 |
| 28 | 64.8 |
| 29 | 3,610 |
| 30 | >10,000 |
| 32 | 182 |
| 33 | 82.6 |
| 34 | 80.3 |
| 36 | 340 |
| 37 | >10,000 |
| 38 | 21 |
| 39 | 22.9 |
| 40 | 473 |
| 41 | 277 |
| 42 | 19.7 |
| 43 | 459 |
| 44 | >10,000 |
| 45 | 60.3 |
| 46 | 100 |
| 47 | 103 |
| 49 | 471 |
| 50 | >10,000 |
| 51 | >10,000 |
| 52 | >10,000 |
| 53 | 20.7 |
| 54 | 6.18 |
| 55 | 6.09 |
| 56 | 18.4 |
| 57 | 18.0 |
| 58 | >10,000 |
| 59 | >10,000 |
| 60 | >10,000 |
| 62 | 9,300 |
| 65 | >10,000 |
| 66 | 226 |
| 68 | 9,680 |
| 69 | >10,000 |
| 71 | 13.9 |
| 75 | 900 |
| 76 | >10,000 |
| 77 | 47.3 |
| 78 | 90.3 |
| 79 | 736 |
| 81 | 6.49 |
| 82 | 28.7 |
| 84 | >10,000 |
| 85 | >10,000 |
| 86 | 44.7 |
| 87 | 8.21 |

### Helios Human T Regulatory Cells Degradation Assay

Cryopreserved human T regulatory cells were thawed in RPMI+10% FBS+20 ng/mL IL-2. After being spun at 1200 rpm for 5 mins, the cells were resuspended in RPMI+10% FBS+20 ng/mL and rested at 37° C. with 5% CO₂ for 3 hours. The cells were then plated at 40,000 cells/well in 40 μL RPMI+10% FBS+20 ng/mL human IL-2 in a 384 well cell culture plate prior to using acoustic dispensing technology (ECHO 555) for adding compounds of interest. Cell cultures were incubated for 20 hours at 37° C. and 5% CO₂. In order to facilitate analysis, cell cultures were spun down at 1200 rpm for 5 minutes and the supernatant was discarded using an EL406 plate washer. After washing three times with 70 μL PBS, cell pellets were resuspended in 50 μL of near IR viability staining solution (Life Technologies, Cat #L34975) and incubated for 30 minutes on ice protected from light. Cells were washed three times with 70 μL PBS+0.5% BSA using an EL406 plate washer. After shaking the plate to dislodge the cell pellet, cells were resuspended in 50 μL of Fixation Buffer (eBioScience FoxP3 buffer set 00-5523-00) for 60 minutes at room temperature. After centrifuging and discarding the supernatant, cells were permeabilized with 50 μL of permeabilization buffer (eBioScience FoxP3 buffer set 00-5523-00) for 10 minutes at room temperature. Following permeabilization, cells were spun down and the supernatant was replaced with 30 μL fluorescently labelled antibodies against Helios in 1× Permeabilization buffer (Helios-APC [BioLegend, Cat #137222, 1:50]) and staining reactions were incubated for 1 hour at room temperature; protected from light. Subsequently, 30 μL of 1× Permeabilization buffer was added prior to centrifuging the cells and discarding the supernatant. Stained cells were resuspended in 30 μL of flow cytometry staining buffer (PBS+0.5% BSA) and analyzed using an Intellicyt Ique Plus flow cytometer.

TABLE B

| | hTreg Helios |
|---|---|
| Ex. No. | DC₅₀ (μM) |
| 88 | 0.0034 |
| 89 | 0.0184 |
| 90 | 0.454 |
| 91 | 10 |
| 92 | 0.0012 |
| 93 | 0.0017 |
| 95 | 0.0116 |
| 96 | 0.0066 |
| 97 | 0.0171 |
| 99 | 0.002 |
| 100 | 0.0032 |
| 101 | 0.0042 |
| 102 | 0.0081 |
| 103 | 4.11 |
| 104 | 0.0042 |
| 105 | 0.0004 |
| 106 | 0.006 |
| 107 | 0.0058 |
| 108 | 0.0014 |
| 109 | 0.0213 |
| 110 | 0.0098 |
| 111 | 0.0104 |
| 112 | 0.0095 |
| 113 | 0.0139 |
| 114 | 0.0235 |
| 119 | 1.51 |
| 120 | 0.761 |
| 122 | 5.09 |
| 125 | 3.22 |
| 126 | 2.2003 |
| 133 | 0.0231 |
| 139 | 0.367 |
| 140 | 0.0791 |
| 141 | 10 |
| 142 | 0.706 |
| 145 | 0.0007 |
| 146 | 0.003 |
| 147 | 0.0115 |
| 148 | 0.0037 |
| 149 | 0.0067 |
| 150 | 10 |
| 151 | 0.167 |
| 152 | 0.0188 |
| 153 | 0.0153 |
| 154 | 0.284 |
| 155 | 0.0109 |
| 156 | 10 |
| 157 | 0.0025 |
| 158 | 0.0032 |
| 159 | 0.0146 |
| 160 | 0.0619 |
| 161 | 0.223 |
| 162 | 0.0287 |
| 164 | 0.0159 |
| 165 | 0.178 |
| 166 | 10 |
| 167 | 0.001 |
| 168 | 0.0037 |
| 169 | 0.004 |
| 172 | 0.0052 |
| 173 | 0.0004 |
| 174 | 0.0015 |
| 175 | 0.0002 |
| 180 | 10 |
| 181 | 10 |
| 183 | 10 |
| 184 | 3.16 |
| 185 | 10 |
| 186 | 10 |
| 187 | 10 |
| 188 | 0.0287 |
| 189 | 0.0064 |
| 190 | 0.0497 |
| 191 | 0.0536 |
| 192 | 0.0077 |
| 193 | 0.0111 |
| 194 | 0.0141 |
| 195 | 0.0235 |
| 196 | 0.0026 |
| 197 | 10 |
| 198 | 0.0519 |
| 199 | 10 |
| 204 | 10 |
| 205 | 10 |
| 206 | 0.0111 |
| 207 | 10 |
| 208 | 10 |
| 209 | 10 |
| 210 | — |
| 212 | 0.653 |
| 213 | 10 |
| 214 | 0.0035 |
| 216 | 10 |
| 217 | 0.390 |
| 218 | 4.13 |
| 219 | 0.0098 |
| 220 | 0.0342 |
| 221 | 13.9 |
| 222 | 2.76 |
| 224 | 0.0729 |
| 225 | 0.0293 |
| 226 | 0.0515 |
| 227 | 10 |
| 230 | 0.0126 |
| 232 | 0.0386 |
| 234 | 8.41 |
| 235 | 0.330 |
| 236 | 10 |
| 237 | 10 |
| 238 | 0.172 |
| 244 | 0.242 |
| 248 | 10 |
| 249 | 10 |
| 250 | 10 |
| 251 | 10 |
| 252 | 10 |
| 253 | 10 |
| 254 | 10 |
| 255 | 0.492 |
| 256 | 1.14 |
| 257 | 10 |
| 258 | 10 |
| 259 | 10 |
| 260 | 0.140 |
| 261 | 10 |
| 262 | 10 |
| 263 | 1.23 |
| 265 | 10 |
| 266 | 10 |
| 267 | 10 |
| 268 | 10 |
| 270 | 10 |
| 271 | 10 |
| 272 | 0.280 |
| 273 | 0.280 |
| 274 | 10 |
| 275 | 10 |
| 276 | 0.028 |
| 277 | 0.0319 |
| 278 | 0.0318 |
| 279 | 0.0343 |
| 280 | 10 |
| 281 | 0.0221 |
| 282 | 10 |
| 283 | 10 |

What is claimed is:

1. A compound of Formula I 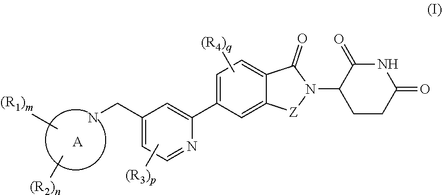
or a salt thereof, wherein:
Z is CR₆R₆ or C═O;
Ring A is azetidinyl, pyrrolidinyl, piperidinyl, azepanyl, tetrahydropyridazinyl, 1,4-azaphosphinane 4-oxide, pyrazolyl, isoindolinyl, dihydropyrrolo[3,4-c]pyridinyl, decahydroquinolinyl, tetrahydropyridinyl, tetrahydroisoquinolinyl, tetrahydronaphthyridinyl, hexahydrocyclopenta[c]pyrrolyl, hexahydrofuro[3,4-c]pyrrolyl, tetrahydropyrazolo[4,3-c]pyridinyl, tetrahydroisoxazolo[4,5-c]pyridinyl, tetrahydro[1,2,4]triazolo[4,3-a]pyrazinyl, octahydroisoindolyl, octahydropyrrolo[3,4-b]pyridinyl, octahydrocyclopenta[c]pyrrolyl, octahydropyrrolo[3,4-c]pyrrolyl, azaspiro[3.3]heptanyl, diazaspiro[3.3]heptanyl, oxaazaspiro[3.3]heptanyl, oxaazabicyclo[3.1.1]heptanyl, diazaspiro[3.4]octanyl, oxaazaspiro[3.4]octanyl, azaspiro[3.5]nonanyl, oxaazaspiro[3.5]nonanyl, diazaspiro[3.5]nonanyl, diazaspiro[4.4]nonanyl, azaspiro[4.5]decanyl, diazaspiro[4.5]decanyl, oxaazaspiro[4.5]decanyl, diazaspiro[4.5]decanonyl, oxadiazaspiro[4.5]decanyl, spiro[indoline-3,4′-piperidinyl], 2H-spiro[benzofuran-3,4′-piperidinyl], 3H-spiro[isobenzofuran-1,4′-piperidinyl], 2,3-dihydrospiro[indene-1,4′-piperidinyl], azabicyclo[3.1.0]hexanyl, azabicyclo[3.1.1]heptanyl, oxaazabicyclo[3.1.1]heptanyl, azabicyclo[3.2.1]octanyl, diazabicyclo[3.2.1]octanyl, or oxaazabicyclo[3.2.1]octanyl;
R₁ is F, —CN, —OH, C₁₋₄ alkyl, C₁₋₃ fluoroalkyl, C₁₋₃ hydroxyalkyl, —CH₂OCH₃, —C(O)(C₁₋₄ alkyl), —C(O)OH, —C(O)O(C₁₋₄ alkyl), —C(O)NR_{y}R_{y}, —CH₂NRₓC(O)OC(CH₃)₃, —NRₓC(O)OCH₃, —NRₓC(O)OC(CH₃)₃, —C(O)NRₓ(cyclohexyl), —S(O)₂CH₃, —P(O)(OH)O(phenyl), —CH₂(phenyl), —C(O)(cyclopropyl), —C(O)(phenyl), —NRₓ(phenyl), —CH₂NRₓC(O)(phenyl), —NRₓC(O)(phenyl), —CH₂O(fluorophenyl), —CH₂O(chloropyridinyl), —NRₓS(O)₂CH₃, —NRₓS(O)₂(phenyl), cyclopropyl, methyl oxadiazolyl, methylisoxazolyl, thiophenyl, —O(phenyl), —O(tert-butoxycarbonyl)phenyl), —O((tert-butoxycarbonyl)amino)phenyl), or phenyl substituted with zero, 1, or 2 R₁ₐ;
each R₁ₐ is independently F, Cl, —OH, —CH₃, —CHF₂, —CF₃, or —S(O)₂CH₃;
each R₂ is independently F, —CN, —OH, —CH₃, —CF₃, —C(CH₃)₂OH, or —C(O)C(CH₃)₃;
each R₃ is independently F, Cl, —CH₃, —CF₃, —OCH₃, —OCF₃, or —NH₂;
each R₄ is independently F, Cl, or —CH₃;
each R₆ is independently hydrogen or C₁₋₃ alkyl;
Rₓ is hydrogen or —CH₃;
each R_{y} is independently hydrogen or C₁₋₄ alkyl;
m is zero or 1;
n is zero, 1, or 2;
p is zero, 1, or 2; and
q is zero, 1, 2, or 3;
with the provisos that:
(1) if Ring A is azetidinyl, pyrrolidinyl, or piperidinyl, then m is 1;
(2) if Ring A is azetidinyl, pyrrolidinyl, or piperidinyl; m is 1; and R₁ is C₁₋₄ alkyl, C₁₋₂ fluoroalkyl, —C(O)(C₁₋₃ alkyl), —C(O)NR_{y}R_{y}, —CH₂(phenyl), phenyl, or methylphenyl; then R₂ is F, —CN, —OH, —CF₃, —C(CH₃)₂OH, or —C(O)C(CH₃)₃.

2. The compound according to claim 1 or a salt thereof, wherein Z is CR₆R₆.

3. The compound according to claim 1 or a salt thereof, wherein Z is C═O.

4. The compound according to claim 1 or a salt thereof, wherein R₁ is F, —CN, —OH, —CH₃, —C(CH₃)₃, —CF₂CH₂CH₃, —CH₂OH, —CH(CH₃)OH, —C(CH₃)₂OH, —CH₂OCH₃, —C(O)CH₃, —C(O)OH, —C(O)OCH₃, —C(O)OCH₂CH₃, —C(O)OCH(CH₃)₂, —C(O)OC(CH₃)₃, —C(O)NH₂, —CH₂NHC(O)OC(CH₃)₃, —NHC(O)OCH₃, —NHC(O)OC(CH₃)₃, —N(CH₃)C(O)OCH₃, —N(CH₃)C(O)OC(CH₃)₃, —C(O)NHC(CH₃)₃, —C(O)NH(cyclohexyl), —S(O)₂CH₃, —P(O)(OH)O(phenyl), —CH₂(phenyl), —C(O)(cyclopropyl), —C(O)(phenyl), —NH(phenyl), —N(CH₃)(phenyl), —CH₂NHC(O)(phenyl), —CH₂N(CH₃)C(O)(phenyl), —N(CH₃)C(O)(phenyl), —CH₂O(fluorophenyl), —CH₂O(chloropyridinyl), —N(CH₃)S(O)₂CH₃, —N(CH₃)S(O)₂(phenyl), cyclopropyl, methyl oxadiazolyl, methylisoxazolyl, thiophenyl, —O(phenyl), —O(tert-butoxycarbonyl)phenyl), —O((tert-butoxycarbonyl)amino)phenyl), or phenyl substituted with zero, 1, or 2 R₁ₐ.

5. The compound according to claim 1 or a salt thereof, wherein:
Ring A is azetidinyl, pyrrolidinyl, or piperidinyl;
m is 1;
R₁ is F, —CN, —OH, —CH₃, —C(CH₃)₃, —CF₂CH₂CH₃, —CH₂OH, —CH(CH₃)OH, —C(CH₃)₂OH, —CH₂OCH₃, —C(O)CH₃, —C(O)OH, —C(O)OCH₃, —C(O)OCH₂CH₃, —C(O)OCH(CH₃)₂, —C(O)OC(CH₃)₃, —C(O)NH₂, —CH₂NHC(O)OC(CH₃)₃, —NHC(O)OCH₃, —NHC(O)OC(CH₃)₃, —N(CH₃)C(O)OCH₃, —N(CH₃)C(O)OC(CH₃)₃, —C(O)NHC(CH₃)₃, —C(O)NH(cyclohexyl), —S(O)₂CH₃, —P(O)(OH)O(phenyl), —CH₂(phenyl), —C(O)(cyclopropyl), —C(O)(phenyl), —NH(phenyl), —N(CH₃)(phenyl), —CH₂NHC(O)(phenyl), —CH₂N(CH₃)C(O)(phenyl), —N(CH₃)C(O)(phenyl), —CH₂O(fluorophenyl), —CH₂O(chloropyridinyl), —N(CH₃)S(O)₂CH₃, —N(CH₃)S(O)₂(phenyl), cyclopropyl, methyl oxadiazolyl, methylisoxazolyl, thiophenyl, —O(phenyl), —O(tert-butoxycarbonyl)phenyl), —O((tert-butoxycarbonyl)amino)phenyl), or phenyl substituted with zero, 1, or 2 R₁ₐ; and
R₂ is F, —CN, —OH, —CF₃, —C(CH₃)₂OH, or —C(O)C(CH₃)₃.

6. The compound according to claim 1 or a salt thereof, wherein Ring A is azetidinyl.

7. The compound according to claim 1 or a salt thereof, wherein Ring A is pyrrolyl.

8. The compound according to claim 1 or a salt thereof, wherein Ring A is piperidinyl.

9. The compound according to claim 1 or a salt thereof, wherein:
R₁ is —C(CH₃)₃, —C(CH₃)₂OH, —CH₂OCH₃, —C(O)OH, —C(O)OCH₃, —C(O)OC(CH₃)₃, —C(O)NH₂, —CH₂NHC(O)OC(CH₃)₃, —NHC(O)OC(CH₃)₃, —N(CH₃)C(O)OC(CH₃)₃, —S(O)₂CH₃, or —P(O)(OH)O(phenyl); and
m is 1.

10. The compound according to claim 1 or a salt thereof, wherein:
R₁ is phenyl substituted with zero, 1, or 2 R₁ₐ; and
m is 1.

11. The compound according to claim 1 or a salt thereof, wherein:
R₁ is:

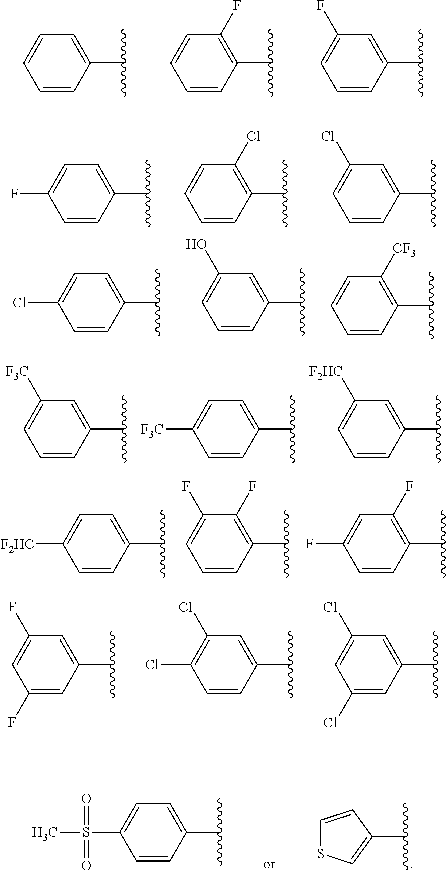

12. The compound according to claim 1 or a salt thereof, wherein said compound is:
3-(5-(5-chloro-4-((3-(3,4-difluorophenyl)azetidin-1-yl)methyl)pyridin-2-yl)-1-oxoisoindolin-2-yl)piperidine-2,6-dione (1);
3-(5-(5-fluoro-4-((3-(methoxymethyl)azetidin-1-yl)methyl)pyridin-2-yl)-1-oxoisoindolin-2-yl)piperidine-2,6-dione (2);
3-(5-(4-((3-(3,4-difluorophenyl)azetidin-1-yl)methyl)-5-fluoropyridin-2-yl)-1-oxoisoindolin-2-yl)piperidine-2,6-dione (3);
3-(5-(5-fluoro-4-((3-(4-fluorophenyl)azetidin-1-yl)methyl)pyridin-2-yl)-1-oxoisoindolin-2-yl)piperidine-2,6-dione (4);
3-(5-(5-fluoro-4-((3-hydroxy-3-phenylazetidin-1-yl)methyl)pyridin-2-yl)-1-oxoisoindolin-2-yl)piperidine-2,6-dione (5);
3-(5-(4-((3-(3,4-difluorophenyl)azetidin-1-yl)methyl)-3-fluoropyridin-2-yl)-1-oxoisoindolin-2-yl)piperidine-2,6-dione (6);
3-(5-(3-fluoro-4-((3-(methoxymethyl)azetidin-1-yl)methyl)pyridin-2-yl)-1-oxoisoindolin-2-yl)piperidine-2,6-dione (7);
3-(5-(3-fluoro-4-((2-(4-fluorophenyl)azetidin-1-yl)methyl)pyridin-2-yl)-1-oxoisoindolin-2-yl)piperidine-2,6-dione (8);
3-(5-(3-fluoro-4-((3-hydroxy-3-phenylazetidin-1-yl)methyl)pyridin-2-yl)-1-oxoisoindolin-2-yl)piperidine-2,6-dione (9);
3-(5-(4-((2-(3-fluorophenyl)azetidin-1-yl)methyl)pyridin-2-yl)-1-oxoisoindolin-2-yl) piperidine-2,6-dione (10);
3-(5-(4-((3-(3-fluorophenyl)azetidin-1-yl)methyl)pyridin-2-yl)-1-oxoisoindolin-2-yl) piperidine-2,6-dione (11);
3-(5-(4-((3-(2-fluorophenyl)azetidin-1-yl)methyl)pyridin-2-yl)-1-oxoisoindolin-2-yl) piperidine-2,6-dione (12);
3-(5-(4-((3-(4-fluorophenyl)azetidin-1-yl)methyl)pyridin-2-yl)-1-oxoisoindolin-2-yl) piperidine-2,6-dione (13);
3-(5-(4-((3-(2-chlorophenyl)azetidin-1-yl)methyl)pyridin-2-yl)-1-oxoisoindolin-2-yl) piperidine-2,6-dione (14);
3-(5-(4-((3-(3-chlorophenyl)azetidin-1-yl)methyl)pyridin-2-yl)-1-oxoisoindolin-2-yl) piperidine-2,6-dione (15);
3-(5-(4-((3-(4-chlorophenyl)azetidin-1-yl)methyl)pyridin-2-yl)-1-oxoisoindolin-2-yl) piperidine-2,6-dione (16);
3-(1-oxo-5-(4-((3-(2-(trifluoromethyl)phenyl)azetidin-1-yl)methyl)pyridin-2-yl) isoindolin-2-yl)piperidine-2,6-dione (17);
3-(1-oxo-5-(4-((3-(3-(trifluoromethyl)phenyl)azetidin-1-yl)methyl)pyridin-2-yl) isoindolin-2-yl)piperidine-2,6-dione (18);
3-(1-oxo-5-(4-((3-(4-(trifluoromethyl)phenyl)azetidin-1-yl)methyl)pyridin-2-yl) isoindolin-2-yl)piperidine-2,6-dione (19);
3-(5-(4-((3-(3-(difluoromethyl)phenyl)azetidin-1-yl)methyl)pyridin-2-yl)-1-oxoisoindolin-2-yl)piperidine-2,6-dione (20);
3-(5-(4-((3-(4-(difluoromethyl)phenyl)azetidin-1-yl)methyl)pyridin-2-yl)-1-oxoisoindolin-2-yl)piperidine-2,6-dione (21);
3-(5-(4-((3-(2,4-difluorophenyl)azetidin-1-yl)methyl)pyridin-2-yl)-1-oxoisoindolin-2-yl)piperidine-2,6-dione (22);
3-(5-(4-((3-(2,3-difluorophenyl)azetidin-1-yl)methyl)pyridin-2-yl)-1-oxoisoindolin-2-yl)piperidine-2,6-dione (23);
3-(5-(4-((3-(3,5-difluorophenyl)azetidin-1-yl)methyl)pyridin-2-yl)-1-oxoisoindolin-2-yl)piperidine-2,6-dione (24);
3-(5-(4-((3-(3,4-dichlorophenyl)azetidin-1-yl)methyl)pyridin-2-yl)-1-oxoisoindolin-2-yl)piperidine-2,6-dione (25);
3-(5-(4-((3-(3,5-dichlorophenyl)azetidin-1-yl)methyl)pyridin-2-yl)-1-oxoisoindolin-2-yl)piperidine-2,6-dione (26);
3-(1-oxo-5-(4-((3-(thiophen-3-yl)azetidin-1-yl)methyl)pyridin-2-yl)isoindolin-2-yl) piperidine-2,6-dione (27);
tert-butyl (1-((2-(2-(2,6-dioxopiperidin-3-yl)-1-oxoisoindolin-5-yl)pyridin-4-yl) methyl)azetidin-3-yl)carbamate (28);
3-(1-oxo-5-(4-((3-(phenylamino)azetidin-1-yl)methyl)pyridin-2-yl)isoindolin-2-yl) piperidine-2,6-dione (29);
3-(5-(4-((3-(methyl(phenyl)amino)azetidin-1-yl)methyl)pyridin-2-yl)-1-oxoisoindolin-2-yl)piperidine-2,6-dione (30);
3-(5-(4-((3-fluoro-3-phenylazetidin-1-yl)methyl)pyridin-2-yl)-1-oxoisoindolin-2-yl) piperidine-2,6-dione (31);
tert-butyl (4-((7-((2-(2-(2,6-dioxopiperidin-3-yl)-1-oxoisoindolin-5-yl)pyridin-4-yl) methyl)-7-azaspiro[3.5]nonan-2-yl)oxy)phenyl)carbamate (32);
3-(5-(4-((3-hydroxyazetidin-1-yl)methyl)pyridin-2-yl)-1-oxoisoindolin-2-yl) piperidine-2,6-dione (33);
3-(5-(4-((3-hydroxy-3-methylazetidin-1-yl)methyl)pyridin-2-yl)-1-oxoisoindolin-2-yl)piperidine-2,6-dione (34);
3-(5-(4-(((2S,3R)-3-hydroxy-2-methylazetidin-1-yl)methyl)pyridin-2-yl)-1-oxoisoindolin-2-yl)piperidine-2,6-dione (35);
3-(5-(4-((3,3-difluoroazetidin-1-yl)methyl)pyridin-2-yl)-1-oxoisoindolin-2-yl) piperidine-2,6-dione (36);
3-(5-(4-((3-hydroxy-3-phenylazetidin-1-yl)methyl)pyridin-2-yl)-1-oxoisoindolin-2-yl)piperidine-2,6-dione (37);
3-(1-oxo-5-(4-((3-phenoxyazetidin-1-yl)methyl)pyridin-2-yl)isoindolin-2-yl) piperidine-2,6-dione (38);
3-(5-(4-(((S)-3-hydroxypyrrolidin-1-yl)methyl)pyridin-2-yl)-1-oxoisoindolin-2-yl) piperidine-2,6-dione (39);
3-(5-(4-(((R)-3-hydroxypyrrolidin-1-yl)methyl)pyridin-2-yl)-1-oxoisoindolin-2-yl) piperidine-2,6-dione (40);
3-(5-(4-(((S)-3-fluoropyrrolidin-1-yl)methyl)pyridin-2-yl)-1-oxoisoindolin-2-yl) piperidine-2,6-dione (41);
3-(5-(4-(((R)-3-fluoropyrrolidin-1-yl)methyl)pyridin-2-yl)-1-oxoisoindolin-2-yl) piperidine-2,6-dione (42);
3-(5-(4-((3-hydroxy-3-phenylpyrrolidin-1-yl)methyl)pyridin-2-yl)-1-oxoisoindolin-2-yl)piperidine-2,6-dione (43);
3-(1-oxo-5-(4-((1-phenyl-3-azabicyclo[3.1.0]hexan-3-yl)methyl)pyridin-2-yl) isoindolin-2-yl)piperidine-2,6-dione (44);
tert-butyl 2-((2-(2-(2,6-dioxopiperidin-3-yl)-1-oxoisoindolin-5-yl)pyridin-4-yl) methyl)-6-oxa-2,9-diazaspiro[4.5]decane-9-carboxylate (45);
3-(1-oxo-5-(4-((3-phenoxypyrrolidin-1-yl)methyl)pyridin-2-yl)isoindolin-2-yl) piperidine-2,6-dione (46);
3-(5-(4-((4-(3-methyl-1,2,4-oxadiazol-5-yl)piperidin-1-yl)methyl)pyridin-2-yl)-1-oxoisoindolin-2-yl)piperidine-2,6-dione (47);
3-(5-(4-((4-hydroxy-4-phenylpiperidin-1-yl)methyl)pyridin-2-yl)-1-oxoisoindolin-2-yl)piperidine-2,6-dione (48);
1-((2-(2-(2,6-dioxopiperidin-3-yl)-1-oxoisoindolin-5-yl)pyridin-4-yl)methyl)-4-phenylpiperidine-4-carbonitrile (49);
3-(1-oxo-5-(4-((4-phenoxypiperidin-1-yl)methyl)pyridin-2-yl)isoindolin-2-yl) piperidine-2,6-dione (50);
3-(1-oxo-5-(4-(spiro[indoline-3,4′-piperidin]-1′-ylmethyl)pyridin-2-yl)isoindolin-2-yl) piperidine-2,6-dione (51);
3-(5-(4-((2H-spiro[benzofuran-3,4′-piperidin]-1′-yl)methyl)pyridin-2-yl)-1-oxoisoindolin-2-yl)piperidine-2,6-dione (52);
3-(5-(4-((2,3-dihydrospiro[indene-1,4′-piperidin]-1′-yl)methyl)pyridin-2-yl)-1-oxoisoindolin-2-yl)piperidine-2,6-dione (53);
3-(5-(4-((4-hydroxypiperidin-1-yl)methyl)pyridin-2-yl)-1-oxoisoindolin-2-yl) piperidine-2,6-dione (54);
3-(5-(4-((4-(2-hydroxypropan-2-yl)piperidin-1-yl)methyl)pyridin-2-yl)-1-oxoisoindolin-2-yl)piperidine-2,6-dione (55);
3-(5-(4-((2-hydroxy-7-azaspiro[3.5]nonan-7-yl)methyl)pyridin-2-yl)-1-oxoisoindolin-2-yl)piperidine-2,6-dione (56);
3-(5-(4-((4-(4-(methylsulfonyl)phenyl)piperidin-1-yl)methyl)pyridin-2-yl)-1-oxoisoindolin-2-yl)piperidine-2,6-dione (57);
3-(5-(4-((4-(3-hydroxyphenyl)piperidin-1-yl)methyl)pyridin-2-yl)-1-oxoisoindolin-2-yl)piperidine-2,6-dione (58);
3-(1-oxo-5-(4-((4-(2-(trifluoromethyl)phenyl)piperidin-1-yl)methyl)pyridin-2-yl) isoindolin-2-yl)piperidine-2,6-dione (59);
3-(5-(4-((4-(3-chlorophenyl)piperidin-1-yl)methyl)pyridin-2-yl)-1-oxoisoindolin-2-yl)piperidine-2,6-dione (60);
3-(5-(4-((4-(4-chlorophenyl)piperidin-1-yl)methyl)pyridin-2-yl)-1-oxoisoindolin-2-yl)piperidine-2,6-dione (61);
3-(5-(4-((4-(2-fluorophenyl)piperidin-1-yl)methyl)pyridin-2-yl)-1-oxoisoindolin-2-yl) piperidine-2,6-dione (62);
3-(5-(4-((4-(3-fluorophenyl)piperidin-1-yl)methyl)pyridin-2-yl)-1-oxoisoindolin-2-yl) piperidine-2,6-dione (63);
3-(5-(4-((3-oxa-6-azabicyclo[3.1.1]heptan-6-yl)methyl)pyridin-2-yl)-1-oxoisoindolin-2-yl)piperidine-2,6-dione (64);
3-(5-(4-((6-oxa-3-azabicyclo[3.1.1]heptan-3-yl)methyl)pyridin-2-yl)-1-oxoisoindolin-2-yl)piperidine-2,6-dione (65);
3-(5-(4-((8-oxa-3-azabicyclo[3.2.1]octan-3-yl)methyl)pyridin-2-yl)-1-oxoisoindolin-2-yl)piperidine-2,6-dione (66);
methyl 4-((7-((2-(2-(2,6-dioxopiperidin-3-yl)-1-oxoisoindolin-5-yl)pyridin-4-yl) methyl)-7-azaspiro[3.5]nonan-2-yl)oxy)benzoate (67);
3-(5-(4-((6-oxa-2,9-diazaspiro[4.5]decan-2-yl)methyl)pyridin-2-yl)-1-oxoisoindolin-2-yl)piperidine-2,6-dione (68);
3-(5-(4-((9-benzoyl-6-oxa-2,9-diazaspiro[4.5]decan-2-yl)methyl)pyridin-2-yl)-1-oxoisoindolin-2-yl)piperidine-2,6-dione (69);
3-(5-(4-((2-(2-hydroxypropan-2-yl)azetidin-1-yl)methyl)pyridin-2-yl)-1-oxoisoindolin-2-yl)piperidine-2,6-dione (70);
3-(5-(4-((3-(methoxymethyl)azetidin-1-yl)methyl)pyridin-2-yl)-1-oxoisoindolin-2-yl) piperidine-2,6-dione (71);
3-(5-(4-((3,6-dihydropyridin-1(2H)-yl)methyl)pyridin-2-yl)-1-oxoisoindolin-2-yl) piperidine-2,6-dione (72);
phenyl hydrogen (1-((2-(2-(2,6-dioxopiperidin-3-yl)-1-oxoisoindolin-5-yl)pyridin-4-yl)methyl)pyrrolidin-2-yl)phosphonate (73);
3-(5-(4-((4-methyl-4-oxido-1,4-azaphosphinan-1-yl)methyl)pyridin-2-yl)-1-oxoisoindolin-2-yl)piperidine-2,6-dione (74);
1-((2-(2-(2,6-dioxopiperidin-3-yl)-1-oxoisoindolin-5-yl)pyridin-4-yl) methyl)azetidine-2-carboxylic acid (75);
3-(5-(4-((3,3-difluoro-2-phenylazetidin-1-yl)methyl)pyridin-2-yl)-1-oxoisoindolin-2-yl)piperidine-2,6-dione (76);
3-(5-(4-((2-(4-fluorophenyl)azetidin-1-yl)methyl)pyridin-2-yl)-1-oxoisoindolin-2-yl) piperidine-2,6-dione (77);
3-(5-(4-((3-(3,4-difluorophenyl)azetidin-1-yl)methyl)pyridin-2-yl)-1-oxoisoindolin-2-yl)piperidine-2,6-dione (78);
1-((2-(2-(2,6-dioxopiperidin-3-yl)-1-oxoisoindolin-5-yl)pyridin-4-yl) methyl)azetidine-2-carboxamide (79);
methyl 1-((2-(2-(2,6-dioxopiperidin-3-yl)-1-oxoisoindolin-5-yl)pyridin-4-yl) methyl)azetidine-2-carboxylate (80);
3-(5-(4-((2-azaspiro[3.3]heptan-2-yl)methyl)pyridin-2-yl)-1-oxoisoindolin-2-yl)piperidine-2,6-dione (81);
3-(5-(4-((2-oxa-6-azaspiro[3.3]heptan-6-yl)methyl)pyridin-2-yl)-1-oxoisoindolin-2-yl)piperidine-2,6-dione (82);
3-(5-(4-((6-oxa-2-azaspiro[3.4]octan-2-yl)methyl)pyridin-2-yl)-1-oxoisoindolin-2-yl)piperidine-2,6-dione (83);
3-(5-(4-((3-(methylsulfonyl)azetidin-1-yl)methyl)pyridin-2-yl)-1-oxoisoindolin-2-yl)piperidine-2,6-dione (84);
tert-butyl ((1-((2-(2-(2,6-dioxopiperidin-3-yl)-1-oxoisoindolin-5-yl)pyridin-4-yl)methyl)azetidin-3-yl)methyl)carbamate (85);
tert-butyl (1-((2-(2-(2,6-dioxopiperidin-3-yl)-1-oxoisoindolin-5-yl)pyridin-4-yl)methyl)azetidin-3-yl)(methyl)carbamate (86);
3-(5-(3-fluoro-4-((4-(2-hydroxypropan-2-yl)piperidin-1-yl)methyl)pyridin-2-yl)-1-oxoisoindolin-2-yl)piperidine-2,6-dione (87);
3-(5-(4-((2-azaspiro[3.3]heptan-2-yl)methyl)-3-fluoropyridin-2-yl)-1-oxoisoindolin-2-yl)piperidine-2,6-dione (88);
3-(5-(3-fluoro-4-((3-(2-hydroxypropan-2-yl)azetidin-1-yl)methyl)pyridin-2-yl)-1-oxoisoindolin-2-yl)piperidine-2,6-dione (89);
3-(5-(3-fluoro-4-(((2S,3R)-3-hydroxy-2-methylazetidin-1-yl)methyl)pyridin-2-yl)-1-oxoisoindolin-2-yl)piperidine-2,6-dione (90);
3-(5-(3-fluoro-4-((4-hydroxy-4-phenylpiperidin-1-yl)methyl)pyridin-2-yl)-1-oxoisoindolin-2-yl)piperidine-2,6-dione (91);
3-(5-(5-fluoro-4-((4-(2-hydroxypropan-2-yl)piperidin-1-yl)methyl)pyridin-2-yl)-1-oxoisoindolin-2-yl)piperidine-2,6-dione (92);
3-(5-(4-((2-azaspiro[3.3]heptan-2-yl)methyl)-5-fluoropyridin-2-yl)-1-oxoisoindolin-2-yl) piperidine-2,6-dione (93);
3-(5-(3-chloro-4-((4-(2-hydroxypropan-2-yl)piperidin-1-yl)methyl)pyridin-2-yl)-1-oxoisoindolin-2-yl)piperidine-2,6-dione (94);
3-(5-(3-chloropyridin-2-yl)-1-oxoisoindolin-2-yl)piperidine-2,6-dione (95);
3-(5-(3-chloro-4-(((R)-2-(2-hydroxypropan-2-yl)pyrrolidin-1-yl)methyl)pyridin-2-yl)-1-oxoisoindolin-2-yl)piperidine-2,6-dione (96);
3-(5-(3-chloro-4-((3-(2-hydroxypropan-2-yl)azetidin-1-yl)methyl)pyridin-2-yl)-1-oxoisoindolin-2-yl)piperidine-2,6-dione (97);
tert-butyl (4-((7-((2-(2-(2,6-dioxopiperidin-3-yl)-1-oxoisoindolin-5-yl)pyridin-4-yl) methyl)-7-azaspiro[3.5]nonan-2-yl)oxy)phenyl)carbamate (98);
3-(5-(4-((3-(methoxymethyl)piperidin-1-yl)methyl)pyridin-2-yl)-1-oxoisoindolin-2-yl)piperidine-2,6-dione (99);
3-(5-(4-((2-oxa-7-azaspiro[3.5]nonan-7-yl)methyl)pyridin-2-yl)-1-oxoisoindolin-2-yl)piperidine-2,6-dione (100);
3-(5-(4-(((1R,5S,6r)-6-(hydroxymethyl)-3-azabicyclo[3.1.0]hexan-3-yl)methyl)pyridin-2-yl)-1-oxoisoindolin-2-yl)piperidine-2,6-dione (101);
3-(5-(4-(((R)-3-(hydroxymethyl)pyrrolidin-1-yl)methyl)pyridin-2-yl)-1-oxoisoindolin-2-yl)piperidine-2,6-dione (102);
3-(5-(4-((6-chloro-1,3-dihydro-2H-pyrrolo[3,4-c]pyridin-2-yl)methyl)pyridin-2-yl)-1-oxoisoindolin-2-yl)piperidine-2,6-dione (103);
3-(5-(4-(((S)-3-(2-hydroxypropan-2-yl)pyrrolidin-1-yl)methyl)pyridin-2-yl)-1-oxoisoindolin-2-yl)piperidine-2,6-dione (104);
3-(5-(4-(((S)-3-(hydroxymethyl)pyrrolidin-1-yl)methyl)pyridin-2-yl)-1-oxoisoindolin-2-yl)piperidine-2,6-dione (105);
3-(5-(4-(((R)-3-(2-hydroxypropan-2-yl)pyrrolidin-1-yl)methyl)pyridin-2-yl)-1-oxoisoindolin-2-yl)piperidine-2,6-dione (106);
3-(5-(4-((3-(1-hydroxyethyl)pyrrolidin-1-yl)methyl)pyridin-2-yl)-1-oxoisoindolin-2-yl)piperidine-2,6-dione (107);
3-(5-(4-((4-(1-hydroxyethyl)piperidin-1-yl)methyl)pyridin-2-yl)-1-oxoisoindolin-2-yl)piperidine-2,6-dione (108);
3-(5-(3-fluoro-4-(((S)-3-(hydroxymethyl)pyrrolidin-1-yl)methyl)pyridin-2-yl)-1-oxoisoindolin-2-yl)piperidine-2,6-dione (109);
3-(5-(3-fluoro-4-(((S)-3-(2-hydroxypropan-2-yl)pyrrolidin-1-yl)methyl)pyridin-2-yl)-1-oxoisoindolin-2-yl)piperidine-2,6-dione (110);
3-(5-(3-fluoro-4-(((R)-3-(2-hydroxypropan-2-yl)pyrrolidin-1-yl)methyl)pyridin-2-yl)-1-oxoisoindolin-2-yl)piperidine-2,6-dione (111);
3-(5-(3-fluoro-4-((3-(1-hydroxyethyl)pyrrolidin-1-yl)methyl)pyridin-2-yl)-1-oxoisoindolin-2-yl)piperidine-2,6-dione (112);
3-(5-(3-fluoro-4-((4-(1-hydroxyethyl)piperidin-1-yl)methyl)pyridin-2-yl)-1-oxoisoindolin-2-yl)piperidine-2,6-dione (113);
3-(5-(3-fluoro-4-(((S)-3-hydroxypyrrolidin-1-yl)methyl)pyridin-2-yl)-1-oxoisoindolin-2-yl)piperidine-2,6-dione (114);
3-(5-(3-fluoro-4-(((R)-3-hydroxypyrrolidin-1-yl)methyl)pyridin-2-yl)-1-oxoisoindolin-2-yl)piperidine-2,6-dione (115);
2-(2,6-dioxopiperidin-3-yl)-5-(4-(((S)-3-(hydroxymethyl)pyrrolidin-1-yl)methyl) pyridin-2-yl)isoindoline-1,3-dione (116);
3-(5-(4-(((R)-3-(2-hydroxypropan-2-yl)pyrrolidin-1-yl)methyl)pyridin-2-yl)-1-oxoisoindolin-2-yl)piperidine-2,6-dione (117);
3-(5-(4-(((S)-3-(2-hydroxypropan-2-yl)pyrrolidin-1-yl)methyl)pyridin-2-yl)-1-oxoisoindolin-2-yl)piperidine-2,6-dione (118);
3-(5-(4-((3-(1-hydroxyethyl)pyrrolidin-1-yl)methyl)pyridin-2-yl)-1-oxoisoindolin-2-yl)piperidine-2,6-dione (119);
3-(5-(4-((4-(2-hydroxypropan-2-yl)piperidin-1-yl)methyl)pyridin-2-yl)-1-oxoisoindolin-2-yl)piperidine-2,6-dione (120);
3-(5-(4-((4-(1-hydroxyethyl)piperidin-1-yl)methyl)pyridin-2-yl)-1-oxoisoindolin-2-yl)piperidine-2,6-dione (121);
3-(5-(4-((2-oxa-7-azaspiro[3.5]nonan-7-yl)methyl)pyridin-2-yl)-1-oxoisoindolin-2-yl) piperidine-2,6-dione (122);
3-(5-(4-(((S)-3-hydroxypyrrolidin-1-yl)methyl)pyridin-2-yl)-1-oxoisoindolin-2-yl) piperidine-2,6-dione (123);
3-(5-(4-(((R)-3-hydroxypyrrolidin-1-yl)methyl)pyridin-2-yl)-1-oxoisoindolin-2-yl) piperidine-2,6-dione (124);
3-(5-(4-((3-hydroxy-3-phenylpyrrolidin-1-yl)methyl)pyridin-2-yl)-1-oxoisoindolin-2-yl)piperidine-2,6-dione (125);
3-(5-(4-((3-hydroxy-3-methylpyrrolidin-1-yl)methyl)pyridin-2-yl)-1-oxoisoindolin-2-yl)piperidine-2,6-dione (126);
(R)-3-((R)-4-fluoro-5-(4-(((S)-3-hydroxypyrrolidin-1-yl)methyl)pyridin-2-yl)-3-methyl-1-oxoisoindolin-2-yl)piperidine-2,6-dione (127);
(S)-3-((R)-4-fluoro-5-(4-(((R)-3-hydroxypyrrolidin-1-yl)methyl)pyridin-2-yl)-3-methyl-1-oxoisoindolin-2-yl)piperidine-2,6-dione (128);
(S)-3-((R)-4-fluoro-5-(4-(((S)-3-(2-hydroxypropan-2-yl)pyrrolidin-1-yl)methyl) pyridin-2-yl)-3-methyl-1-oxoisoindolin-2-yl)piperidine-2,6-dione (129);
(S)-3-((R)-4-fluoro-5-(4-(((R)-3-(2-hydroxypropan-2-yl)pyrrolidin-1-yl)methyl) pyridin-2-yl)-3-methyl-1-oxoisoindolin-2-yl)piperidine-2,6-dione (130);
(S)-3-((R)-4-fluoro-5-(4-(((S)-3-(hydroxymethyl)pyrrolidin-1-yl)methyl)pyridin-2-yl)-3-methyl-1-oxoisoindolin-2-yl)piperidine-2,6-dione (131);
(S)-3-((R)-4-fluoro-5-(4-((4-(2-hydroxypropan-2-yl)piperidin-1-yl)methyl)pyridin-2-yl)-3-methyl-1-oxoisoindolin-2-yl)piperidine-2,6-dione (132);
(S)-3-((R)-4-fluoro-5-(4-(((R)-3-(hydroxymethyl)pyrrolidin-1-yl)methyl)pyridin-2-yl)-3-methyl-1-oxoisoindolin-2-yl)piperidine-2,6-dione (133);
(3S)-3-((3R)-4-fluoro-5-(4-((3-(1-hydroxyethyl)pyrrolidin-1-yl)methyl)pyridin-2-yl)-3-methyl-1-oxoisoindolin-2-yl)piperidine-2,6-dione (134);
(3S)-3-((3R)-4-fluoro-5-(4-((4-(1-hydroxyethyl)piperidin-1-yl)methyl)pyridin-2-yl)-3-methyl-1-oxoisoindolin-2-yl)piperidine-2,6-dione (135);
(S)-3-((R)-5-(4-((2-oxa-7-azaspiro[3.5]nonan-7-yl)methyl)pyridin-2-yl)-4-fluoro-3-methyl-1-oxoisoindolin-2-yl)piperidine-2,6-dione (136);
(S)-3-((R)-4-fluoro-5-(4-((3-hydroxyazetidin-1-yl)methyl)pyridin-2-yl)-3-methyl-1-oxoisoindolin-2-yl)piperidine-2,6-dione (137);
(S)-3-((R)-4-fluoro-5-(4-((3-hydroxy-3-methylazetidin-1-yl)methyl)pyridin-2-yl)-3-methyl-1-oxoisoindolin-2-yl)piperidine-2,6-dione (138);
(3S)-3-((3R)-4-fluoro-5-(4-((3-hydroxy-3-phenylpyrrolidin-1-yl)methyl)pyridin-2-yl)-3-methyl-1-oxoisoindolin-2-yl)piperidine-2,6-dione (139);
(3S)-3-((3R)-4-fluoro-5-(4-((3-hydroxy-3-methylpyrrolidin-1-yl)methyl)pyridin-2-yl)-3-methyl-1-oxoisoindolin-2-yl)piperidine-2,6-dione (140);
(S)-3-((R)-4-fluoro-3-methyl-1-oxo-5-(4-(((R)-3-phenoxypyrrolidin-1-yl)methyl) pyridin-2-yl)isoindolin-2-yl)piperidine-2,6-dione (141);
(S)-3-((R)-4-fluoro-5-(4-(((S)-3-fluoropyrrolidin-1-yl)methyl)pyridin-2-yl)-3-methyl-1-oxoisoindolin-2-yl)piperidine-2,6-dione (142);
3-((R)-4-fluoro-5-(3-fluoro-4-((4-(2-hydroxypropan-2-yl)piperidin-1-yl)methyl) pyridin-2-yl)-3-methyl-1-oxoisoindolin-2-yl)piperidine-2,6-dione (143);
(S)-3-((R)-4-fluoro-5-(3-fluoro-4-(((S)-3-hydroxypyrrolidin-1-yl)methyl)pyridin-2-yl)-3-methyl-1-oxoisoindolin-2-yl)piperidine-2,6-dione (144);
(S)-3-(5-(4-((6-hydroxy-2-azaspiro[3.3]heptan-2-yl)methyl)pyridin-2-yl)-1-oxoisoindolin-2-yl)piperidine-2,6-dione (145);
(3S)-3-(5-(4-((5-hydroxyhexahydrocyclopenta[c]pyrrol-2(1H)-yl)methyl)pyridin-2-yl)-1-oxoisoindolin-2-yl)piperidine-2,6-dione (146);
(3S)-3-(5-(4-((5-hydroxy-5-methylhexahydrocyclopenta[c]pyrrol-2(1H)-yl)methyl) pyridin-2-yl)-1-oxoisoindolin-2-yl)piperidine-2,6-dione (147);
(3S)-3-(5-(4-((5-methyl-3,3a,4,6a-tetrahydrocyclopenta[c]pyrrol-2(1H)-yl)methyl) pyridin-2-yl)-1-oxoisoindolin-2-yl)piperidine-2,6-dione (148);
(3S)-3-(5-(3-fluoro-4-(((3aR,7aS)-5-hydroxyoctahydro-2H-isoindol-2-yl)methyl) pyridin-2-yl)-1-oxoisoindolin-2-yl)piperidine-2,6-dione (149);
(S)-3-(5-(3-fluoro-4-((1-oxo-2,8-diazaspiro[4.5]decan-8-yl)methyl)pyridin-2-yl)-1-oxoisoindolin-2-yl)piperidine-2,6-dione (150);
(S)-3-(5-(3-fluoro-4-(((3aR,6aS)-tetrahydro-1H-furo[3,4-c]pyrrol-5(3H)-yl)methyl) pyridin-2-yl)-1-oxoisoindolin-2-yl)piperidine-2,6-dione (151);
(S)-3-(1-oxo-5-(4-(((3aR,6aS)-tetrahydro-1H-furo[3,4-c]pyrrol-5(3H)-yl)methyl) pyridin-2-yl)isoindolin-2-yl)piperidine-2,6-dione (152);
(S)-3-(5-(3-fluoro-4-((6-hydroxy-2-azaspiro[3.3]heptan-2-yl)methyl)pyridin-2-yl)-1-oxoisoindolin-2-yl)piperidine-2,6-dione (153);
tert-butyl (3aS,6aS)-5-((2-(2-((S)-2,6-dioxopiperidin-3-yl)-1-oxoisoindolin-5-yl)-3-fluoropyridin-4-yl)methyl)hexahydropyrrolo[3,4-c]pyrrole-2(1H)-carboxylate (154);
(3S)-3-(5-(3-fluoro-4-((hexahydrocyclopenta[c]pyrrol-2(1H)-yl)methyl)pyridin-2-yl)-1-oxoisoindolin-2-yl)piperidine-2,6-dione (155);
tert-butyl (3aR,6aS)-5-((2-(2-((S)-2,6-dioxopiperidin-3-yl)-1-oxoisoindolin-5-yl)-3-fluoropyridin-4-yl)methyl)hexahydropyrrolo[3,4-c]pyrrole-2(1H)-carboxylate (156);
3-(5-(4-((2-azaspiro[3.5]nonan-2-yl)methyl)pyridin-2-yl)-1-oxoisoindolin-2-yl) piperidine-2,6-dione (157);
(S)-3-(5-(4-((7-oxa-2-azaspiro[3.5]nonan-2-yl)methyl)pyridin-2-yl)-1-oxoisoindolin-2-yl)piperidine-2,6-dione (158);
N-((1-((2-(2-(2,6-dioxopiperidin-3-yl)-1-oxoisoindolin-5-yl)pyridin-4-yl)methyl) azetidin-3-yl)methyl)benzamide (159);
(S)—N-(1-((2-(2-(2,6-dioxopiperidin-3-yl)-1-oxoisoindolin-5-yl)pyridin-4-yl)methyl) azetidin-3-yl)-N-methylbenzamide (160);
(S)—N-(1-((2-(2-(2,6-dioxopiperidin-3-yl)-1-oxoisoindolin-5-yl)pyridin-4-yl)methyl) azetidin-3-yl)-N-methylmethanesulfonamide (161);
(S)—N-(1-((2-(2-(2,6-dioxopiperidin-3-yl)-1-oxoisoindolin-5-yl)pyridin-4-yl)methyl) azetidin-3-yl)-N-methylbenzenesulfonamide (162);
methyl (S)-(1-((2-(2-(2,6-dioxopiperidin-3-yl)-1-oxoisoindolin-5-yl)pyridin-4-yl) methyl)azetidin-3-yl)(methyl)carbamate (163);
(S)—N-((1-((2-(2-(2,6-dioxopiperidin-3-yl)-1-oxoisoindolin-5-yl)pyridin-4-yl)methyl) azetidin-3-yl)methyl)-N-methylbenzamide (164);
3-(5-(4-((1H-pyrazol-1-yl)methyl)pyridin-2-yl)-1-oxoisoindolin-2-yl)piperidine-2,6-dione (165);
(S)-3-(5-(4-((4-methyl-1H-pyrazol-1-yl)methyl)pyridin-2-yl)-1-oxoisoindolin-2-yl) piperidine-2,6-dione (166);
3-(5-(4-((4-(2-hydroxypropan-2-yl)piperidin-1-yl)methyl)-5-methylpyridin-2-yl)-1-oxoisoindolin-2-yl)piperidine-2,6-dione (167);
3-(5-(4-((2-azaspiro[3.3]heptan-2-yl)methyl)-5-methylpyridin-2-yl)-1-oxoisoindolin-2-yl)piperidine-2,6-dione (168);
3-(5-(4-((3-cyclopropylpyrrolidin-1-yl)methyl)-5-methylpyridin-2-yl)-1-oxoisoindolin-2-yl)piperidine-2,6-dione (169);
3-(5-(4-((2-azaspiro[3.3]heptan-2-yl)methyl)-3-methylpyridin-2-yl)-1-oxoisoindolin-2-yl)piperidine-2,6-dione (170);
3-(5-(4-((4-(2-hydroxypropan-2-yl)piperidin-1-yl)methyl)-3-methylpyridin-2-yl)-1-oxoisoindolin-2-yl)piperidine-2,6-dione (171);
3-(5-(4-((4-(2-hydroxypropan-2-yl)piperidin-1-yl)methyl)-3-methoxypyridin-2-yl)-1-oxoisoindolin-2-yl)piperidine-2,6-dione (172);
3-(5-(4-((3-cyclopropylpyrrolidin-1-yl)methyl)-3-methoxypyridin-2-yl)-1-oxoisoindolin-2-yl)piperidine-2,6-dione (173);
3-(5-(4-((2-azaspiro[3.3]heptan-2-yl)methyl)-6-methoxypyridin-2-yl)-1-oxoisoindolin-2-yl)piperidine-2,6-dione (174);
3-(5-(4-((3-cyclopropylpyrrolidin-1-yl)methyl)-6-methoxypyridin-2-yl)-1-oxoisoindolin-2-yl)piperidine-2,6-dione (175);
3-(5-(4-((4-(2-hydroxypropan-2-yl)piperidin-1-yl)methyl)-3-(trifluoromethyl)pyridin-2-yl)-1-oxoisoindolin-2-yl)piperidine-2,6-dione (176);
3-(5-(4-((3-(methoxymethyl)azetidin-1-yl)methyl)-3-(trifluoromethyl)pyridin-2-yl)-1-oxoisoindolin-2-yl)piperidine-2,6-dione (177);
3-(5-(4-((2-azaspiro[3.3]heptan-2-yl)methyl)-3-(trifluoromethyl)pyridin-2-yl)-1-oxoisoindolin-2-yl)piperidine-2,6-dione (178);
3-(5-(4-((4-(2-hydroxypropan-2-yl)piperidin-1-yl)methyl)-5-(trifluoromethyl)pyridin-2-yl)-1-oxoisoindolin-2-yl)piperidine-2,6-dione (179);
3-(5-(4-((2-azaspiro[3.3]heptan-2-yl)methyl)-5-(trifluoromethyl)pyridin-2-yl)-1-oxoisoindolin-2-yl)piperidine-2,6-dione (180);
3-(5-(4-((3-(methoxymethyl)azetidin-1-yl)methyl)-5-(trifluoromethyl)pyridin-2-yl)-1-oxoisoindolin-2-yl)piperidine-2,6-dione (181);
3-(5-(3-fluoro-4-((4-(trifluoromethoxy)piperidin-1-yl)methyl)pyridin-2-yl)-1-oxoisoindolin-2-yl)piperidine-2,6-dione (182);
3-(5-(3-fluoro-4-((3-(trifluoromethoxy)pyrrolidin-1-yl)methyl)pyridin-2-yl)-1-oxoisoindolin-2-yl)piperidine-2,6-dione (183);
3-(5-(3-fluoro-4-(((S)-3-fluoropyrrolidin-1-yl)methyl)pyridin-2-yl)-1-oxoisoindolin-2-yl)piperidine-2,6-dione (184);
3-(5-(3-fluoro-4-((3-hydroxy-3-(trifluoromethyl)pyrrolidin-1-yl)methyl)pyridin-2-yl)-1-oxoisoindolin-2-yl)piperidine-2,6-dione (185);
3-(5-(3-fluoro-4-((4-fluoroisoindolin-2-yl)methyl)pyridin-2-yl)-1-oxoisoindolin-2-yl) piperidine-2,6-dione (186);
3-(5-(4-((5-chloroisoindolin-2-yl)methyl)-3-fluoropyridin-2-yl)-1-oxoisoindolin-2-yl) piperidine-2,6-dione (187);
N-(tert-butyl)-1-((2-(2-(2,6-dioxopiperidin-3-yl)-1-oxoisoindolin-5-yl)pyridin-4-yl) methyl)pyrrolidine-2-carboxamide (188);
3-(5-(4-((1-(methoxymethyl)-2-azaspiro[3.3]heptan-2-yl)methyl)pyridin-2-yl)-1-oxoisoindolin-2-yl)piperidine-2,6-dione (189);
(2S)—N-cyclohexyl-1-((2-(2-(2,6-dioxopiperidin-3-yl)-1-oxoisoindolin-5-yl)pyridin-4-yl)methyl)pyrrolidine-2-carboxamide (190);
3-(5-(4-(((R)-2-(((6-chloropyridin-3-yl)oxy)methyl)azetidin-1-yl)methyl)pyridin-2-yl)-1-oxoisoindolin-2-yl)piperidine-2,6-dione (191);
3-(5-(4-((2-((4-fluorophenoxy)methyl)azetidin-1-yl)methyl)pyridin-2-yl)-1-oxoisoindolin-2-yl)piperidine-2,6-dione (192);
ethyl 3-((2-(2-(2,6-dioxopiperidin-3-yl)-1-oxoisoindolin-5-yl)pyridin-4-yl)methyl)-3-azabicyclo[3.1.0]hexane-2-carboxylate (193);
3-(5-(4-((2-azabicyclo[3.1.0]hexan-2-yl)methyl)pyridin-2-yl)-1-oxoisoindolin-2-yl) piperidine-2,6-dione (194);
tert-butyl (((2S)-1-((2-(2-(2,6-dioxopiperidin-3-yl)-1-oxoisoindolin-5-yl)pyridin-4-yl) methyl)pyrrolidin-2-yl)methyl)carbamate (195);
3-(5-(4-((2-(isopropoxymethyl)azetidin-1-yl)methyl)pyridin-2-yl)-1-oxoisoindolin-2-yl)piperidine-2,6-dione (196);
3-(5-(4-((3-fluoro-3-phenylpiperidin-1-yl)methyl)pyridin-2-yl)-1-oxoisoindolin-2-yl) piperidine-2,6-dione (197);
3-(5-(3-fluoro-4-((4-fluoro-4-methylpiperidin-1-yl)methyl)pyridin-2-yl)-1-oxoisoindolin-2-yl)piperidine-2,6-dione (198);
3-(5-(4-((2-acetyltetrahydropyridazin-1(2H)-yl)methyl)-3-fluoropyridin-2-yl)-1-oxoisoindolin-2-yl)piperidine-2,6-dione (199);
3-(5-(4-((4-(1,1-difluoropropyl)piperidin-1-yl)methyl)-3-fluoropyridin-2-yl)-1-oxoisoindolin-2-yl)piperidine-2,6-dione (200);
tert-butyl 2-((2-(2-(2,6-dioxopiperidin-3-yl)-1-oxoisoindolin-5-yl)-3-fluoropyridin-4-yl)methyl)-2,5-diazaspiro[3.5]nonane-5-carboxylate (201);
3-(5-(4-((1-acetyl-1,8-diazaspiro[4.5]decan-8-yl)methyl)-3-fluoropyridin-2-yl)-1-oxoisoindolin-2-yl)piperidine-2,6-dione (202);
methyl 8-((2-(2-(2,6-dioxopiperidin-3-yl)-1-oxoisoindolin-5-yl)-3-fluoropyridin-4-yl) methyl)-1,8-diazaspiro[4.5]decane-1-carboxylate (203);
tert-butyl 1-((2-(2-(2,6-dioxopiperidin-3-yl)-1-oxoisoindolin-5-yl)-3-fluoropyridin-4-yl)methyl)octahydro-6H-pyrrolo[3,4-b]pyridine-6-carboxylate (204);
3-(5-(3-fluoro-4-((3-fluoropiperidin-1-yl)methyl)pyridin-2-yl)-1-oxoisoindolin-2-yl) piperidine-2,6-dione (205);
3-(5-(4-((6,6-dimethyl-3-azabicyclo[3.1.0]hexan-3-yl)methyl)-3-fluoropyridin-2-yl)-1-oxoisoindolin-2-yl)piperidine-2,6-dione (206);
tert-butyl 2-((2-(2-(2,6-dioxopiperidin-3-yl)-1-oxoisoindolin-5-yl)-3-fluoropyridin-4-yl)methyl)-2,6-diazaspiro[3.4]octane-6-carboxylate (207);
tert-butyl 2-((2-(2-(2,6-dioxopiperidin-3-yl)-1-oxoisoindolin-5-yl)-3-fluoropyridin-4-yl)methyl)-2,6-diazaspiro[3.5]nonane-6-carboxylate (208);
3-(5-(3-fluoro-4-((4-hydroxy-4-(trifluoromethyl)piperidin-1-yl)methyl)pyridin-2-yl)-1-oxoisoindolin-2-yl)piperidine-2,6-dione (209);
3-(5-(4-((8-azaspiro[4.5]decan-8-yl)methyl)-3-fluoropyridin-2-yl)-1-oxoisoindolin-2-yl)piperidine-2,6-dione (210);
3-(5-(3-fluoro-4-((4-hydroxy-2-methylpiperidin-1-yl)methyl)pyridin-2-yl)-1-oxoisoindolin-2-yl)piperidine-2,6-dione (211-212);
tert-butyl 7-((2-(2-(2,6-dioxopiperidin-3-yl)-1-oxoisoindolin-5-yl)-3-fluoropyridin-4-yl)methyl)-1,7-diazaspiro[4.4]nonane-1-carboxylate (213);
tert-butyl (2S,4R)-4-(tert-butoxy)-1-((2-(2-(2,6-dioxopiperidin-3-yl)-1-oxoisoindolin-5-yl)-3-fluoropyridin-4-yl)methyl)pyrrolidine-2-carboxylate (214-215);
tert-butyl 6-((2-(2-(2,6-dioxopiperidin-3-yl)-1-oxoisoindolin-5-yl)-3-fluoropyridin-4-yl)methyl)-1,6-diazaspiro[3.5]nonane-1-carboxylate (216);
3-(5-(3-fluoro-4-(((R)-2-((R)-1-hydroxy-1-phenylethyl)pyrrolidin-1-yl)methyl) pyridin-2-yl)-1-oxoisoindolin-2-yl)piperidine-2,6-dione (217);
3-(5-(3-fluoro-4-(((R)-2-((S)-1-hydroxy-1-phenylethyl)pyrrolidin-1-yl)methyl) pyridin-2-yl)-1-oxoisoindolin-2-yl)piperidine-2,6-dione (218);
3-(5-(3-fluoro-4-((6-hydroxy-3-azabicyclo[3.1.1]heptan-3-yl)methyl)pyridin-2-yl)-1-oxoisoindolin-2-yl)piperidine-2,6-dione (219);
3-(5-(3-fluoro-4-((4-hydroxy-4-methylazepan-1-yl)methyl)pyridin-2-yl)-1-oxoisoindolin-2-yl)piperidine-2,6-dione (220);
3-(5-(3-fluoro-4-((4-hydroxy-3,3-dimethylpiperidin-1-yl)methyl)pyridin-2-yl)-1-oxoisoindolin-2-yl)piperidine-2,6-dione (221);
3-(5-(3-fluoro-4-((3-hydroxy-1-oxa-8-azaspiro[4.5]decan-8-yl)methyl)pyridin-2-yl)-1-oxoisoindolin-2-yl)piperidine-2,6-dione (222);
3-(5-(3-fluoro-4-((3-hydroxy-2-methylazetidin-1-yl)methyl)pyridin-2-yl)-1-oxoisoindolin-2-yl)piperidine-2,6-dione (223);
3-(5-(3-fluoro-4-((3-hydroxy-3-methylazetidin-1-yl)methyl)pyridin-2-yl)-1-oxoisoindolin-2-yl)piperidine-2,6-dione (224);
3-(5-(3-fluoro-4-((2-hydroxy-7-azaspiro[3.5]nonan-7-yl)methyl)pyridin-2-yl)-1-oxoisoindolin-2-yl)piperidine-2,6-dione (225);
3-(5-(3-fluoro-4-((4-hydroxypiperidin-1-yl)methyl)pyridin-2-yl)-1-oxoisoindolin-2-yl)piperidine-2,6-dione (226);
3-(5-(4-((3-acetyl-3,8-diazabicyclo[3.2.1]octan-8-yl)methyl)-3-fluoropyridin-2-yl)-1-oxoisoindolin-2-yl)piperidine-2,6-dione (227);
methyl 8-((2-(2-(2,6-dioxopiperidin-3-yl)-1-oxoisoindolin-5-yl)-3-fluoropyridin-4-yl) methyl)-3,8-diazabicyclo[3.2.1]octane-3-carboxylate (228);
3-(5-(4-((3-benzoyl-3,8-diazabicyclo[3.2.1]octan-8-yl)methyl)-3-fluoropyridin-2-yl)-1-oxoisoindolin-2-yl)piperidine-2,6-dione (229);
3-(5-(3-fluoro-4-((3-hydroxy-3,8-diazabicyclo[3.2.1]octan-8-yl)methyl)pyridin-2-yl)-1-oxoisoindolin-2-yl)piperidine-2,6-dione (230);
3-(5-(4-((1-(cyclopropanecarbonyl)-1,8-diazaspiro[4.5]decan-8-yl)methyl)-3-fluoropyridin-2-yl)-1-oxoisoindolin-2-yl)piperidine-2,6-dione (231);
3-(5-(3-fluoro-4-(((3S,4R)-3-hydroxy-4-methylpyrrolidin-1-yl)methyl)pyridin-2-yl)-1-oxoisoindolin-2-yl)piperidine-2,6-dione (232);
3-(5-(4-((5-acetyl-2,5-diazaspiro[3.5]nonan-2-yl)methyl)-3-fluoropyridin-2-yl)-1-oxoisoindolin-2-yl)piperidine-2,6-dione (233);
3-(5-(3-fluoro-4-((3-(4-fluorophenyl)-3-hydroxypyrrolidin-1-yl)methyl)pyridin-2-yl)-1-oxoisoindolin-2-yl)piperidine-2,6-dione (234);
3-(5-(4-((4-benzyl-4-hydroxypiperidin-1-yl)methyl)-3-fluoropyridin-2-yl)-1-oxoisoindolin-2-yl)piperidine-2,6-dione (235);
tert-butyl ((3S,5S)-1-((2-(2-(2,6-dioxopiperidin-3-yl)-1-oxoisoindolin-5-yl)-3-fluoropyridin-4-yl)methyl)-5-fluoropiperidin-3-yl)carbamate (236);
3-(5-(3-fluoro-4-((4-(4-fluorophenyl)-4-hydroxy-2-methylpyrrolidin-1-yl)methyl) pyridin-2-yl)-1-oxoisoindolin-2-yl)piperidine-2,6-dione (237);
3-(5-(4-((3-(2-chlorophenyl)-3-hydroxypyrrolidin-1-yl)methyl)-3-fluoropyridin-2-yl)-1-oxoisoindolin-2-yl)piperidine-2,6-dione (238);
3-(5-(3-fluoro-4-(((4aR,8R,8aR)-8-hydroxyoctahydroquinolin-1(2H)-yl)methyl) pyridin-2-yl)-1-oxoisoindolin-2-yl)piperidine-2,6-dione (239);
methyl (2S,3R)-1-((2-(2-(2,6-dioxopiperidin-3-yl)-1-oxoisoindolin-5-yl)-3-fluoropyridin-4-yl)methyl)-3-hydroxypyrrolidine-2-carboxylate (240);
3-(5-(3-fluoro-4-((3-hydroxy-3-(p-tolyl)pyrrolidin-1-yl)methyl)pyridin-2-yl)-1-oxoisoindolin-2-yl)piperidine-2,6-dione (241);
3-(5-(4-((3-(3-chlorophenyl)-3-hydroxypyrrolidin-1-yl)methyl)-3-fluoropyridin-2-yl)-1-oxoisoindolin-2-yl)piperidine-2,6-dione (242);
3-(5-(3-fluoro-4-((4-fluoro-4-phenylpiperidin-1-yl)methyl)pyridin-2-yl)-1-oxoisoindolin-2-yl)piperidine-2,6-dione (243);
3-(5-(3-fluoro-4-((2-(3-methylisoxazol-5-yl)pyrrolidin-1-yl)methyl)pyridin-2-yl)-1-oxoisoindolin-2-yl)piperidine-2,6-dione (244);
3-(5-(3-fluoro-4-(((S)-2-(3-methylisoxazol-5-yl)pyrrolidin-1-yl)methyl)pyridin-2-yl)-1-oxoisoindolin-2-yl)piperidine-2,6-dione (245);
tert-butyl 7-((2-(2-(2,6-dioxopiperidin-3-yl)-1-oxoisoindolin-5-yl)-3-fluoropyridin-4-yl)methyl)-2,7-diazaspiro[3.5]nonane-2-carboxylate (246);
3-(5-(3-fluoro-4-((4-hydroxy-3-methylpiperidin-1-yl)methyl)pyridin-2-yl)-1-oxoisoindolin-2-yl)piperidine-2,6-dione (247);
methyl ((3S,4S)-1-((2-(2-(2,6-dioxopiperidin-3-yl)-1-oxoisoindolin-5-yl)-3-fluoropyridin-4-yl)methyl)-4-hydroxypyrrolidin-3-yl)carbamate (248);
3-(5-(4-((3H-spiro[isobenzofuran-1,4′-piperidin]-1′-yl)methyl)-3-fluoropyridin-2-yl)-1-oxoisoindolin-2-yl)piperidine-2,6-dione (249);
3-(5-(3-fluoro-4-((3-hydroxy-3-phenylpiperidin-1-yl)methyl)pyridin-2-yl)-1-oxoisoindolin-2-yl)piperidine-2,6-dione (250);
3-(5-(4-((2-acetyl-2,7-diazaspiro[3.5]nonan-7-yl)methyl)-3-fluoropyridin-2-yl)-1-oxoisoindolin-2-yl)piperidine-2,6-dione (251);
methyl 7-((2-(2-(2,6-dioxopiperidin-3-yl)-1-oxoisoindolin-5-yl)-3-fluoropyridin-4-yl) methyl)-2,7-diazaspiro[3.5]nonane-2-carboxylate (252);
3-(5-(3-fluoro-4-((3-fluoro-3-phenylpiperidin-1-yl)methyl)pyridin-2-yl)-1-oxoisoindolin-2-yl)piperidine-2,6-dione (253);
3-(5-(3-fluoro-4-((1,4,6,7-tetrahydro-5H-pyrazolo[4,3-c]pyridin-5-yl)methyl)pyridin-2-yl)-1-oxoisoindolin-2-yl)piperidine-2,6-dione (254);
3-(5-(3-fluoro-4-((5-hydroxy-5-methylhexahydrocyclopenta[c]pyrrol-2(1H)-yl) methyl)pyridin-2-yl)-1-oxoisoindolin-2-yl)piperidine-2,6-dione (255);
3-(5-(4-((3-(2,5-dimethylphenyl)-3-hydroxypyrrolidin-1-yl)methyl)-3-fluoropyridin-2-yl)-1-oxoisoindolin-2-yl)piperidine-2,6-dione (256);
3-(5-(3-fluoro-4-((3-hydroxy-3-phenylpyrrolidin-1-yl)methyl)pyridin-2-yl)-1-oxoisoindolin-2-yl)piperidine-2,6-dione (257);
3-(5-(3-fluoro-4-(((R)-3-fluoropyrrolidin-1-yl)methyl)pyridin-2-yl)-1-oxoisoindolin-2-yl)piperidine-2,6-dione (258);
3-(5-(4-((6,7-dihydroisoxazolo[4,5-c]pyridin-5(4H)-yl)methyl)-3-fluoropyridin-2-yl)-1-oxoisoindolin-2-yl)piperidine-2,6-dione (259);
3-(5-(3-fluoro-4-((4-fluoropiperidin-1-yl)methyl)pyridin-2-yl)-1-oxoisoindolin-2-yl) piperidine-2,6-dione (260);
3-(5-(4-((4,4-difluoropiperidin-1-yl)methyl)-3-fluoropyridin-2-yl)-1-oxoisoindolin-2-yl)piperidine-2,6-dione (261);
3-(5-(3-fluoro-4-((3-methyl-5,6-dihydro-[1,2,4]triazolo[4,3-a]pyrazin-7(8H)-yl) methyl)pyridin-2-yl)-1-oxoisoindolin-2-yl)piperidine-2,6-dione (262);
3-(5-(4-((5,6-dihydro-[1,2,4]triazolo[4,3-a]pyrazin-7(8H)-yl)methyl)-3-fluoropyridin-2-yl)-1-oxoisoindolin-2-yl)piperidine-2,6-dione (263);
3-(5-(4-((1,3-dimethyl-1,4,6,7-tetrahydro-5H-pyrazolo[4,3-c]pyridin-5-yl)methyl)-3-fluoropyridin-2-yl)-1-oxoisoindolin-2-yl)piperidine-2,6-dione (264);
3-(5-(4-((2,3-dimethyl-2,4,6,7-tetrahydro-5H-pyrazolo[4,3-c]pyridin-5-yl)methyl)-3-fluoropyridin-2-yl)-1-oxoisoindolin-2-yl)piperidine-2,6-dione (265);
3-(5-(3-fluoro-4-((3-(trifluoromethyl)-5,6-dihydro-[1,2,4]triazolo[4,3-a]pyrazin-7(8H)-yl)methyl)pyridin-2-yl)-1-oxoisoindolin-2-yl)piperidine-2,6-dione (266);
3-(5-(4-((5-chloro-3,4-dihydro-2,6-naphthyridin-2(1H)-yl)methyl)-3-fluoropyridin-2-yl)-1-oxoisoindolin-2-yl)piperidine-2,6-dione (267);
3-(5-(4-((8-chloro-3,4-dihydroisoquinolin-2(1H)-yl)methyl)-3-fluoropyridin-2-yl)-1-oxoisoindolin-2-yl)piperidine-2,6-dione (268);
3-(5-(4-((6-acetyl-2,6-diazaspiro[3.3]heptan-2-yl)methyl)-3-fluoropyridin-2-yl)-1-oxoisoindolin-2-yl)piperidine-2,6-dione (269);
3-(5-(4-((1-acetyl-1,7-diazaspiro[4.4]nonan-7-yl)methyl)-3-fluoropyridin-2-yl)-1-oxoisoindolin-2-yl)piperidine-2,6-dione (270);
methyl 7-((2-(2-(2,6-dioxopiperidin-3-yl)-1-oxoisoindolin-5-yl)-3-fluoropyridin-4-yl) methyl)-1,7-diazaspiro[4.4]nonane-1-carboxylate (271);
3-(5-(4-((6-acetyl-2,6-diazaspiro[3.4]octan-2-yl)methyl)-3-fluoropyridin-2-yl)-1-oxoisoindolin-2-yl)piperidine-2,6-dione (272);
methyl 2-((2-(2-(2,6-dioxopiperidin-3-yl)-1-oxoisoindolin-5-yl)-3-fluoropyridin-4-yl) methyl)-2,6-diazaspiro[3.4]octane-6-carboxylate (273);
3-(5-(4-((6-acetyloctahydro-1H-pyrrolo[3,4-b]pyridin-1-yl)methyl)-3-fluoropyridin-2-yl)-1-oxoisoindolin-2-yl)piperidine-2,6-dione (274);
methyl 1-((2-(2-(2,6-dioxopiperidin-3-yl)-1-oxoisoindolin-5-yl)-3-fluoropyridin-4-yl) methyl)octahydro-6H-pyrrolo[3,4-b]pyridine-6-carboxylate (275);
3-(4-fluoro-5-(3-fluoro-4-((4-(2-hydroxypropan-2-yl)piperidin-1-yl)methyl)pyridin-2-yl)-1-oxoisoindolin-2-yl)piperidine-2,6-dione (276);
3-(4-fluoro-5-(3-fluoro-4-(((S)-3-(hydroxymethyl)pyrrolidin-1-yl)methyl)pyridin-2-yl)-1-oxoisoindolin-2-yl)piperidine-2,6-dione (277);
3-(4-fluoro-5-(3-fluoro-4-(((R)-3-(2-hydroxypropan-2-yl)pyrrolidin-1-yl)methyl) pyridin-2-yl)-1-oxoisoindolin-2-yl)piperidine-2,6-dione (278);
3-(4-fluoro-5-(3-fluoro-4-(((S)-3-(2-hydroxypropan-2-yl)pyrrolidin-1-yl)methyl) pyridin-2-yl)-1-oxoisoindolin-2-yl)piperidine-2,6-dione (279);
3-(4-fluoro-5-(3-fluoro-4-((4-hydroxy-4-phenylpiperidin-1-yl)methyl)pyridin-2-yl)-1-oxoisoindolin-2-yl)piperidine-2,6-dione (280);
3-(4-fluoro-5-(3-fluoro-4-((3-(2-hydroxypropan-2-yl)azetidin-1-yl)methyl)pyridin-2-yl)-1-oxoisoindolin-2-yl)piperidine-2,6-dione (281);
3-(4-fluoro-5-(3-fluoro-4-((3-(trifluoromethyl)-5,6-dihydro-[1,2,4]triazolo[4,3-a]pyrazin-7(8H)-yl)methyl)pyridin-2-yl)-1-oxoisoindolin-2-yl)piperidine-2,6-dione (282);
3-(5-(4-((5-chloro-3,4-dihydro-2,6-naphthyridin-2(1H)-yl)methyl)-3-fluoropyridin-2-yl)-4-fluoro-1-oxoisoindolin-2-yl)piperidine-2,6-dione (283);
3-(5-(4-((6-acetyl-2,6-diazaspiro[3.3]heptan-2-yl)methyl)-3-fluoropyridin-2-yl)-4-fluoro-1-oxoisoindolin-2-yl)piperidine-2,6-dione (284);
3-(5-(6-amino-5-((8-benzoyl-3,8-diazabicyclo[3.2.1]octan-3-yl)methyl)pyridin-2-yl)-1-oxoisoindolin-2-yl)piperidine-2,6-dione (285);
3-(5-(5-((2-azaspiro[3.3]heptan-2-yl)methyl)-6-aminopyridin-2-yl)-1-oxoisoindo-lin-2-yl)piperidine-2,6-dione (286);
3-(5-(3,5-difluoro-4-((4-(2-hydroxypropan-2-yl)piperidin-1-yl)methyl)pyridin-2-yl)-1-oxoisoindolin-2-yl)piperidine-2,6-dione (287);
3-(5-(3,5-difluoro-4-((3-(methoxymethyl)azetidin-1-yl)methyl)pyridin-2-yl)-4-fluoro-1-oxoisoindolin-2-yl)piperidine-2,6-dione (288); or
3-(5-(4-((2-azaspiro[3.3]heptan-2-yl)methyl)-3,5-difluoropyridin-2-yl)-4-fluoro-1-oxoisoindolin-2-yl)piperidine-2,6-dione (289).

13. A pharmaceutical composition comprising a compound according to claim 1 or a pharmaceutically-acceptable salt thereof, and a pharmaceutically acceptable carrier.

14. (canceled)

15. (canceled)

16. A method of decreasing Helios protein levels, Helios activity level, or Helios expression level in the cells comprising contacting said Helios protein with a compound according to claim 1, or a pharmaceutically acceptable salt thereof.

17. The method according to claim 16, wherein Helios protein is the amino acid sequence encoded by SEQ ID NOs: 1, 2, 3, 4, or 5.

18. A method of decreasing Eos protein levels, Eos activity level, or Eos expression level in the cells comprising contacting said Eos protein with a compound according to claim 1, or a pharmaceutically acceptable salt thereof.

19. The method according to claim 18, wherein Eos protein is the amino acid sequence encoded by SEQ ID NOs: 7 or 8.
